SEQUENCE LISTING (56 sequences)

SEQ ID NO: 1 - protein, 179 aa, Homo sapiens
RFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 2 - protein, 201 aa, Homo sapiens
ARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 3 - protein, 112 aa, synthetic construct
  REGION 1..112: note = Synthetic Construct
GPELVKPGASVKISCKASGYAFSYSWMNWVKLRPGQGLEWIGRIFPGDGDTDYNGKFKGKATLTADKSSNTAYMQLTSLTSVDSAVYLCARNVFDGYWLVYWGQGTLVTVSA

SEQ ID NO: 4 - protein, 103 aa, synthetic construct
  REGION 1..103: note = Synthetic Construct
NPVTLGTSASISCRSSKSLLHSNGITYLYWYLQKPGQSPQLLIYQMSNLVSGVPDRFSSSGSGTDFTLRISRVEAEDVGVYYCAQNLELPYTFGGGTKLEIKR

SEQ ID NO: 5 - protein, 6 aa, synthetic construct
  REGION 1..6: note = Synthetic Construct
GYAFSY

SEQ ID NO: 6 - protein, 8 aa, synthetic construct
  REGION 1..8: note = Synthetic Construct
FPGDGDTD

SEQ ID NO: 7 - protein, 10 aa, synthetic construct
  REGION 1..10: note = Synthetic Construct
NVFDGYWLVY

SEQ ID NO: 8 - protein, 16 aa, synthetic construct
  REGION 1..16: note = Synthetic Construct
RSSKSLLHSNGITYLY

SEQ ID NO: 9 - protein, 7 aa, synthetic construct
  REGION 1..7: note = Synthetic Construct
QMSNLVS

SEQ ID NO: 10 - protein, 9 aa, synthetic construct
  REGION 1..9: note = Synthetic Construct
AQNLELPYT

SEQ ID NO: 11 - protein, 119 aa, synthetic construct
  REGION 1..119: note = Synthetic Construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 12 - protein, 112 aa, synthetic construct
  REGION 1..112: note = Synthetic Construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 13 - protein, 448 aa, synthetic construct
  REGION 1..448: note = Synthetic Construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 219 aa, synthetic construct
  REGION 1..219: note = Synthetic Construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 119 aa, synthetic construct
  REGION 1..119: note = Synthetic Construct
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 16 - protein, 119 aa, synthetic construct
  REGION 1..119: note = Synthetic Construct
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYLCARNVFDGYWLVYWGQGTLVTVSS

SEQ ID NO: 17 - protein, 448 aa, synthetic construct; identical to SEQ ID NO: 13
  REGION 1..448: note = Synthetic Construct

SEQ ID NO: 18 - protein, 219 aa, synthetic construct; identical to SEQ ID NO: 14
  REGION 1..219: note = Synthetic Construct

SEQ ID NO: 19 - protein, 117 aa, Homo sapiens [sequence omitted]

SEQ ID NO: 20 - protein, 112 aa, Homo sapiens
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKR

SEQ ID NO: 21 - protein, 10 aa, synthetic construct
  REGION 1..10: note = Synthetic Construct
GYTFSSYWIE

SEQ ID NO: 22 - protein, 18 aa, synthetic construct
  REGION 1..18: note = Synthetic Construct
GEILPGGGDTNYNEIFKG

SEQ ID NO: 23 - protein, 10 aa, synthetic construct
  REGION 1..10: note = Synthetic Construct
TRRVPIRLDY

SEQ ID NO: 24 - protein, 15 aa, synthetic construct
  REGION 1..15: note = Synthetic Construct
KASQSVDYEGDSFLN

SEQ ID NO: 25 - protein, 7 aa, synthetic construct
  REGION 1..7: note = Synthetic Construct
AASNLES

SEQ ID NO: 26 - protein, 9 aa, synthetic construct
  REGION 1..9: note = Synthetic Construct
QQSNEDPLT

SEQ ID NO: 27 - protein, 25 aa, synthetic construct [sequence omitted]

SEQ ID NO: 28 - protein, 13 aa, synthetic construct
  REGION 1..13: note = Synthetic Construct
WVRQAPGKGLEWI

SEQ ID NO: 29 - protein, 30 aa, synthetic construct
  REGION 1..30: note = Synthetic Construct
RATFSADTSKNTAYLQMNSLRAEDTAVYYC

SEQ ID NO: 30 - protein, 11 aa, synthetic construct
  REGION 1..11: note = Synthetic Construct
WGQGTLVTVSS

SEQ ID NO: 31 - protein, 23 aa, synthetic construct
  REGION 1..23: note = Synthetic Construct
DIQLTQSPSSLSASVGDRVTITC

SEQ ID NO: 32 - protein, 15 aa, synthetic construct
  REGION 1..15: note = Synthetic Construct
WYQQKPGKAPKLLIY

SEQ ID NO: 33 - protein, 32 aa, synthetic construct
  REGION 1..32: note = Synthetic Construct
GVPSRFSGSGSGTDFTLTISSLQPEDFATYYC

SEQ ID NO: 34 - protein, 11 aa, synthetic construct
  REGION 1..11: note = Synthetic Construct
FGQGTKVEIKR

SEQ ID NO: 35 - protein, 218 aa, synthetic construct
  REGION 1..218: note = Synthetic Construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 446 aa, synthetic construct
  REGION 1..446: note = Synthetic Construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 446 aa, synthetic construct
  REGION 1..446: note = Synthetic Construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 218 aa, synthetic construct
  REGION 1..218: note = Synthetic Construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 39 - protein, 447 aa, synthetic construct
  REGION 1..447: note = Synthetic Construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 40 - protein, 119 aa, synthetic construct
  REGION 1..119: note = Synthetic Construct
QVQLVQSGAEVKKPGASVKVSCKVSGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 41 - protein, 119 aa, synthetic construct
  REGION 1..119: note = Synthetic Construct
QVQLVQSGAEVKKPGSSVKVSCKASGYAFSYSWMSWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 42 - protein, 119 aa, synthetic construct; identical to SEQ ID NO: 11
  REGION 1..119: note = Synthetic Construct

SEQ ID NO: 43 - protein, 119 aa, synthetic construct
  REGION 1..119: note = Synthetic Construct
QVQLVQSGAEVKKPGSSVKVSCKASGYAFSYSWISWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 44 - protein, 119 aa, synthetic construct
  REGION 1..119: note = Synthetic Construct
QVQLVQSGAEVKKPGASVKVSCKASGYTFTYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 45 - protein, 119 aa, synthetic construct
  REGION 1..119: note = Synthetic Construct
QVQLVQSGAEVKKPGASVKVSCKASGYTFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 46 - protein, 119 aa, synthetic construct
  REGION 1..119: note = Synthetic Construct
EVQLVESGGGLVKPGGSLRLSCAASGFTFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 47 - protein, 119 aa, synthetic construct
  REGION 1..119: note = Synthetic Construct
EVQLVESGGGLVKPGGSLRLSCAASGFAFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 48 - protein, 119 aa, synthetic construct
  REGION 1..119: note = Synthetic Construct
QVQLVESGGGLVKPGGSLRLSCAASGFTFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 49 - protein, 119 aa, synthetic construct
  REGION 1..119: note = Synthetic Construct
EVQLVESGAGLVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 50 - protein, 119 aa, synthetic construct
  REGION 1..119: note = Synthetic Construct
EVQLVESGGGVVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 51 - protein, 119 aa, synthetic construct
  REGION 1..119: note = Synthetic Construct
EVQLVESGGGLKKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 52 - protein, 119 aa, synthetic construct
  REGION 1..119: note = Synthetic Construct
EVQLVESGGGLVKPGSSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 53 - protein, 119 aa, synthetic construct
  REGION 1..119: note = Synthetic Construct
EVQLVESGGGLVKPGGSLRVSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 54 - protein, 119 aa, synthetic construct
  REGION 1..119: note = Synthetic Construct
EVQLVESGGGLVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 55 - protein, 115 aa, synthetic construct
  REGION 1..115: note = Synthetic Construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

SEQ ID NO: 56 - protein, 447 aa, synthetic construct
  REGION 1..447: note = Synthetic Construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0336697 A1
(43) Pub. Date: Oct. 10, 2024

(54) METHODS OF USING ANTI-CD79B IMMUNOCONJUGATES TO TREAT DIFFUSE LARGE B-CELL LYMPHOMA

(71) Applicants: Genentech, Inc., South San Francisco, CA (US); Hoffmann-La Roche Inc., Little Falls, NJ (US)

(72) Inventors: Jamie Harue HIRATA, San Carlos, CA (US); Calvin Ka Yin LEE, San Francisco, CA (US); Huan JIN, San Carlos, CA (US); Lijia WANG, San Francisco, CA (US); Jiaheng QIU, Pacifica, CA (US); Qingxiang YAN, Ontario (CA)

(73) Assignees: Genentech, Inc., South San Francisco, CA (US); Hoffmann-La Roche Inc., Little Falls, NJ (US)

(21) Appl. No.: 18/681,787

(22) PCT Filed: Aug. 2, 2022
(86) PCT No.: PCT/US2022/074634

§ 371 Date: Feb. 6, 2024

Related U.S. Application Data
(60) Provisional application No. 63/282,002, filed on Nov. 22, 2021.
(60) Provisional application No. 63/230,735, filed on Aug. 7, 2021.
(60) Provisional application No. 63/230,725, filed on Aug. 7, 2021.

(51) Int. Cl. C07K 16/28; A61K 9/00; A61K 31/573; A61K 31/675; A61K 31/704; A61K 39/00; A61P 35/00

(52) U.S. Cl. CPC C07K 16/2887; A61K 9/0019; A61K 31/573; A61K 31/675; A61K 31/704; A61P 35/00; A61K 2039/507; A61K 2039/545; A61K 2039/55

(57) ABSTRACT

Provided herein are methods of treating B-cell proliferative disorders (such as diffuse large B-cell lymphoma “DLBCL”) using immunoconjugates comprising anti-CD79b antibodies in combination with an anti-CD20 antibody, chemotherapy and a corticosteroid.

### CROSS-REFERENCE TO RELATED APPLICATION

This application claims the benefit of U.S. Provisional Application No. 63/282,002, filed Nov. 22, 2021, U.S. Provisional Application No. 63/230,735, filed Aug. 7, 2021, and U.S. Provisional Application No. 63/230,725, filed Aug. 7, 2021, each of which is hereby incorporated by reference in its entirety.

### REFERENCE TO AN ELECTRONIC SEQUENCE LISTING

The contents of the electronic sequence listing (146392054640SEQLIST.xml; Size: 75,734 bytes; and Date of Creation: Aug. 4, 2022) are herein incorporated by reference in their entirety.

### FIELD OF THE INVENTION

The present disclosure relates to methods of treating B-cell proliferative disorders, e.g., diffuse large B-cell lymphoma (DLBCL), by administering an immunoconjugate comprising an anti-CD79b antibody in combination with an anti-CD20 antibody, one or more chemotherapeutic agents, and a corticosteroid.

### BACKGROUND OF THE INVENTION

Non-Hodgkin lymphoma (NHL) is the most common hematologic malignancy in the world, and the thirteenth most common cancer overall (Bray et al., (2018) CA Cancer J Clin, 68:394-424). Diffuse large B-cell lymphoma (DLBCL) is an aggressive subtype of NHL, accounting for approximately 32.5% of all NHL cases. Patients with DLBCL present with rapidly enlarging masses, often with local and systemic symptoms of fever, recurrent night sweats, and/or weight loss. Approximately 45% to 60% of patients present with advanced-stage disease (Ann Arbor Stage III or IV). The incidence of DLBCL increases with age, with a median age of 64 years at presentation (Armitage and Weisenburger, J Clin Oncol (1998) 6:2780-95). If left untreated, patients with DLBCL have a median survival of approximately 6 months.

Rituximab plus cyclophosphamide, doxorubicin, vincristine, and prednisone (R-CHOP) was established as the standard of care (SoC) therapy for DLBCL over 20 years ago. Approaches to improve on the current SoC therapy for DLBCL have largely been unsuccessful. This includes attempts at maximizing dose density of R-CHOP (Cunningham et al., Lancet (2013) 381:1817-26; Delarue et al., Lancet Oncol (2013) 14:525-33), and experimental treatments such as those tested in large studies in DLBCL, including BO21005/GOYA (Vitolo et al., Blood (2016) 128:470), DA-EPOCH-R (Wilson et al., Blood (2016) 128:469), and REMARC (Thieblemont et al., Blood (2016) 128:471). In total, since the establishment of R-CHOP as the SoC therapy for DLBLC, 11 randomized Phase III studies have failed to show any benefit in first-line DLBCL compared to R-CHOP.

For patients who are not cured by therapy for previously untreated DLBCL, high-dose chemotherapy followed by autologous stem cell transplantation offers a second chance for cure. However, approximately half of these patients will not respond to subsequent therapy because of refractory disease (Gisselbrecht et al., J Clin Oncol (2010) 28:4184-90), and a significant number of patients are ineligible for this aggressive therapy because of age or comorbidities. Patients who either relapse after, or are ineligible for stem cell transplantation because of refractory disease or frailty have poor outcomes. Responses to subsequent therapies range from 10% to 35% in most cases (Seyfarth et al., Br J Haematol (2006) 133 (1): 3-18), with only occasional durable responses. The fact that most patients who are not cured by standard front-line R-CHOP or comparable chemoimmunotherapy treatments will die of lymphoma underscores the need for novel approaches in front-line therapy for this aggressive disease.

Accordingly, there is a need in the art for new therapeutic approaches in patients with DLBCL, such as previously untreated DLBCL.

All references cited herein, including patent applications and publications, are hereby incorporated by reference in their entirety.

### SUMMARY

In some aspects, provided herein is a method for treating diffuse large B-cell lymphoma (DLBCL) in a human patient in need thereof, comprising administering to the human patient an effective amount of: (a) an immunoconjugate comprising the formula:

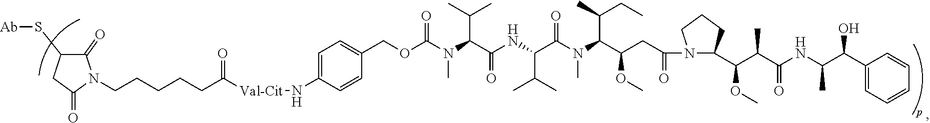

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) rituximab, (c) cyclophosphamide, (d) doxorubicin, and (e) prednisone, prednisolone, or methylprednisolone; wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising: (a) rituximab, (b) cyclophosphamide, (c) doxorubicin, (d) vincristine, and (c) prednisone, prednisolone, or methylprednisolone, in the absence of immunoconjugate. In some embodiments, the PFS or the reference PFS is measured: (a) starting from the start of the corresponding treatment to the time of a first occurrence of disease progression, relapse, or death; or (b) starting from up to 7 days prior to the start of the corresponding treatment to the time of a first occurrence of disease progression, relapse, or death; or (c) starting from the time from randomization to the time of a first occurrence of disease progression, relapse, or death. In some embodiments, the PFS or the reference PFS is the median PFS of the plurality of human patients receiving the corresponding treatment. In some embodiments, the improvement in PFS is statistically significant. In some embodiments, the improvement in PFS is statistically significant with a hazard ratio of no more than 0.75 (95% confidence interval: 0.57, 0.97). In some embodiments, the improvement in PFS is statistically significant with a hazard ratio of no more than 0.78 (95% confidence interval: 0.60, 1.00). In some embodiments, the improvement in PFS is statistically significant with a hazard ratio of no more than 0.79 (95% confidence interval: 0.61, 1.02).

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein administering such treatment to a plurality of human patients results in an improvement in progression-free survival (PFS) of the plurality of human patients as compared to a reference PFS, wherein the reference PFS is the PFS of a plurality of human patients who have received a control treatment comprising: (a) rituximab, (b) cyclophosphamide, (c) doxorubicin, (d) vincristine, and (c) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (c) prednisone, prednisolone, or methylprednisolone; wherein the human patient has an age of greater than 60 years, wherein administering such treatment to a plurality of human patients having an age of greater than 60 years results in an improvement in progression-free survival (PFS) of the plurality of human patients as compared to a reference PFS, and wherein the reference PFS is the PFS of a plurality of human patients having an age of greater than 60 years who have received a control treatment comprising: (a) rituximab, (b) cyclophosphamide, (c) doxorubicin, (d) vincristine, and (e) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein the human patient has an age of greater than 65 years, wherein administering such treatment to a plurality of human patients having an age of greater than 65 years results in an improvement in progression-free survival (PFS) of the plurality of human patients as compared to a reference PFS, and wherein the reference PFS is the PFS of a plurality of human patients having an age of greater than 65 years who have received a control treatment comprising: (a) rituximab, (b) cyclophosphamide, (c) doxorubicin, (d) vincristine, and (e) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein the human patient has an International Prognostic Index (IPI) score between 3 and 5, wherein administering such treatment to a plurality of human patients having an IPI score between 3 and 5 results in an improvement in progression-free survival (PFS) of the plurality of human patients as compared to a reference PFS, and wherein the reference PFS is the PFS of a plurality of human patients having an IPI score between 3 and 5 who have received a control treatment comprising: (a) rituximab, (b) cyclophosphamide, (c) doxorubicin, (d) vincristine, and (e) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein the human patient has an age of greater than 60 years and an International Prognostic Index (IPI) score between 3 and 5, wherein administering such treatment to a plurality of human patients having an age of greater than 60 years and an IPI score between 3 and 5 results in an improvement in progression-free survival (PFS) of the plurality of human patients as compared to a reference PFS, and wherein the reference PFS is the PFS of a plurality of human patients having an age of greater than 60 years and an IPI score between 3 and 5 who have received a control treatment comprising: (a) rituximab, (b) cyclophosphamide, (c) doxorubicin, (d) vincristine, and (c) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (c) prednisone, prednisolone, or methylprednisolone; wherein the human patient has an age of greater than 65 years and an International Prognostic Index (IPI) score between 3 and 5, wherein administering such treatment to a plurality of human patients having an age of greater than 65 years and an IPI score between 3 and 5 results in an improvement in progression-free survival (PFS) of the plurality of human patients as compared to a reference PFS, and wherein the reference PFS is the PFS of a plurality of human patients having an age of greater than 65 years and an IPI score between 3 and 5 who have received a control treatment comprising: (a) rituximab, (b) cyclophosphamide, (c) doxorubicin, (d) vincristine, and (c) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein the human patient has an activated B-cell (ABC) type DLBCL, wherein administering such treatment to a plurality of human patients having an ABC type DLBCL results in an improvement in progression-free survival (PFS) of the plurality of human patients as compared to a reference PFS, and wherein the reference PFS is the PFS of a plurality of human patients having an ABC type DLBCL who have received a control treatment comprising: (a) rituximab, (b) cyclophosphamide, (c) doxorubicin, (d) vincristine, and (e) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) in prednisone, prednisolone, or methylprednisolone; wherein the human patient has a double expressing lymphoma (DEL) type DLBCL, wherein administering such treatment to a plurality of human patients having a DEL type DLBCL results in an improvement in progression-free survival (PFS) of the plurality of human patients as compared to a reference PFS, and wherein the reference PFS is the PFS of a plurality of human patients having a DEL type DLBCL who have received a control treatment comprising: (a) rituximab, (b) cyclophosphamide, (c) doxorubicin, (d) vincristine, and (e) prednisone, prednisolone, or methylprednisolone, in the absence of polatuzumab vedotin.

In some embodiments, which may be combined with any of the preceding aspects or embodiments, the PFS or the reference PFS is measured: (a) starting from the start of the corresponding treatment to the time of a first occurrence of disease progression, relapse, or death; or (b) starting from up to 7 days prior to the start of the corresponding treatment to the time of a first occurrence of disease progression, relapse, or death; or (c) starting from the time from randomization to the time of a first occurrence of disease progression, relapse, or death. In some embodiments, which may be combined with any of the preceding aspects or embodiments, the PFS or the reference PFS is the median PFS of the plurality of human patients receiving the corresponding treatment. In some embodiments, which may be combined with any of the preceding aspects or embodiments, the improvement in PFS is statistically significant.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein administering such treatment to a plurality of human patients results in at least a 25% reduction in the risk of disease progression, relapse, or death in the plurality of human patients as compared to a control treatment comprising: (a) rituximab, (b) cyclophosphamide, (c) doxorubicin, (d) vincristine, and (e) prednisone, prednisolone, or methylprednisolone, in the absence of polatuzumab vedotin.

In other aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein administering such treatment to a plurality of human patients results in at least a 20%, 21%, 22%, 23%, or 24% reduction in the risk of disease progression, relapse, or death in the plurality of human patients as compared to a control treatment comprising: (a) rituximab, (b) cyclophosphamide, (c) doxorubicin, (d) vincristine, and (e) prednisone, prednisolone, or methylprednisolone, in the absence of polatuzumab vedotin.

In some embodiments, said disease progression, relapse, or death are measured: (a) starting from the start of the corresponding treatment to the time of a first occurrence of disease progression, relapse, or death; or (b) starting from up to 7 days prior to the start of the corresponding treatment to the time of a first occurrence of disease progression, relapse, or death; or (c) starting from the time from randomization to the time of a first occurrence of disease progression, relapse, or death. In some embodiments, the reduction in the risk of disease progression, relapse, or death is calculated at 12 months, 24 months, or more, measured starting from: (a) the start of the corresponding treatment; or (b) up to 7 days prior to the start of the corresponding treatment; or (c) the time from randomization to the time of a first occurrence of disease progression, relapse, or death.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein administering such treatment to a plurality of human patients results in a stratified hazard ratio of no more than 0.75 in progression-free survival (PFS) of the plurality of human patients as compared to a control treatment comprising: (a) rituximab, (b) cyclophosphamide, (c) doxorubicin, (d) vincristine, and (e) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein administering such treatment to a plurality of human patients results in a stratified hazard ratio of no more than 0.78 in progression-free survival (PFS) of the plurality of human patients as compared to a control treatment comprising: (a) rituximab, (b) cyclophosphamide, (c) doxorubicin, (d) vincristine, and (e) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein administering such treatment to a plurality of human patients results in an unstratified hazard ratio of no more than 0.79 in progression-free survival (PFS) of the plurality of human patients as compared to a control treatment comprising: (a) rituximab, (b) cyclophosphamide, (c) doxorubicin, (d) vincristine, and (c) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein: (i) the human patient has an age greater than 60 years, and wherein administering such treatment to a plurality of human patients having an age greater than 60 years results in a stratified hazard ratio of no more than 0.72 in progression-free survival (PFS) of the plurality of human patients as compared to a control treatment, or (ii) the human patient has an age greater than 65 years, and wherein administering such treatment to a plurality of human patients having an age greater than 65 years results in a stratified hazard ratio of no more than 0.79 in PFS of the plurality of human patients as compared to a control treatment; wherein the control treatment comprises: (a) rituximab, (b) cyclophosphamide, (c) doxorubicin, (d) vincristine, and (c) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (c) prednisone, prednisolone, or methylprednisolone; wherein the human patient has an International Prognostic Index (IPI) score of between 3 and 5, and wherein administering such treatment to a plurality of human patients having an IPI score of between 3 and 5 results in a stratified hazard ratio of no more than 0.68 in progression-free survival (PFS) of the plurality of human patients, as compared to a control treatment comprising: (a) rituximab, (b) cyclophosphamide, (c) doxorubicin, (d) vincristine, and (c) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (c) prednisone, prednisolone, or methylprednisolone; wherein: (i) the human patient has an activated B-cell (ABC) type DLBCL, and wherein administering such treatment to a plurality of human patients having an ABC type DLBCL results in a stratified hazard ratio of no more than 0.31 in progression-free survival (PFS) of the plurality of human patients as compared to a control treatment, or (ii) the human patient has a double expressing lymphoma (DEL) type DLBCL, and wherein administering such treatment to a plurality of human patients having a DEL type DLBCL results in a stratified hazard ratio of no more than 0.62 in PFS of the plurality of human patients as compared to a control treatment; wherein the control treatment comprises: (a) rituximab, (b) cyclophosphamide, (c) doxorubicin, (d) vincristine, and (e) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein: (i) the human patient has an age greater than 60 years, and wherein administering such treatment to a plurality of human patients having an age greater than 60 years results in an unstratified hazard ratio of no more than 0.72 in progression-free survival (PFS) of the plurality of human patients as compared to a control treatment, or (ii) the human patient has an age greater than 65 years, and wherein administering such treatment to a plurality of human patients having an age greater than 65 years results in an unstratified hazard ratio of no more than 0.77 in PFS of the plurality of human patients as compared to a control treatment; wherein the control treatment comprises: (a) rituximab, (b) cyclophosphamide, (c) doxorubicin, (d) vincristine, and (e) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein: (i) the human patient has an age greater than 60 years, and wherein administering such treatment to a plurality of human patients having an age greater than 60 years results in an unstratified hazard ratio of no more than 0.76 in progression-free survival (PFS) of the plurality of human patients as compared to a control treatment, or (ii) the human patient has an age greater than 65 years, and wherein administering such treatment to a plurality of human patients having an age greater than 65 years results in an unstratified hazard ratio of no more than 0.78 in PFS of the plurality of human patients as compared to a control treatment; wherein the control treatment comprises: (a) rituximab, (b) cyclophosphamide, (c) doxorubicin, (d) vincristine, and (e) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein the human patient has an International Prognostic Index (IPI) score between 3 and 5, and wherein administering such treatment to a plurality of human patients having an IPI score between 3 and 5 results in an unstratified hazard ratio of no more than 0.71 in progression-free survival (PFS) of the plurality of human patients, as compared to a control treatment comprising: (a) rituximab, (b) cyclophosphamide, (c) doxorubicin, (d) vincristine, and (e) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein the human patient has an International Prognostic Index (IPI) score between 3 and 5, and wherein administering such treatment to a plurality of human patients having an IPI score between 3 and 5 results in an unstratified hazard ratio of no more than 0.75 in progression-free survival (PFS) of the plurality of human patients, as compared to a control treatment comprising: (a) rituximab, (b) cyclophosphamide, (c) doxorubicin, (d) vincristine, and (e) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein: (i) the human patient has an activated B-cell (ABC) type DLBCL, and wherein administering such treatment to a plurality of human patients having an ABC type DLBCL results in an unstratified hazard ratio of no more than 0.36 in progression-free survival (PFS) of the plurality of human patients as compared to a control treatment, or (ii) the human patient has a double expressing lymphoma (DEL) type DLBCL, and wherein administering such treatment to a plurality of human patients having a DEL type DLBCL results in an unstratified hazard ratio of no more than 0.67 in PFS of the plurality of human patients as compared to a control treatment; wherein the control treatment comprises: (a) rituximab, (b) cyclophosphamide, (c) doxorubicin, (d) vincristine, and (e) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (c) prednisone, prednisolone, or methylprednisolone; wherein: (i) the human patient has an activated B-cell (ABC) type DLBCL, and wherein administering such treatment to a plurality of human patients having an ABC type DLBCL results in an unstratified hazard ratio of no more than 0.39 in progression-free survival (PFS) of the plurality of human patients as compared to a control treatment, or (ii) the human patient has a double expressing lymphoma (DEL) type DLBCL, and wherein administering such treatment to a plurality of human patients having a DEL type DLBCL results in an unstratified hazard ratio of no more than 0.65 in PFS of the plurality of human patients as compared to a control treatment; wherein the control treatment comprises: (a) rituximab, (b) cyclophosphamide, (c) doxorubicin, (d) vincristine, and (c) prednisone, prednisolone, or methylprednisolone, in the absence of polatuzumab vedotin.

In some embodiments, which may be combined with any of the preceding aspects or embodiments, the PFS is measured: (a) starting from the start of the corresponding treatment to the time of a first occurrence of disease progression, relapse, or death; or (b) starting from up to 7 days prior to the start of the corresponding treatment to the time of a first occurrence of disease progression, relapse, or death; or (c) starting from the time from randomization to the time of a first occurrence of disease progression, relapse, or death. In some embodiments, which may be combined with any of the preceding aspects or embodiments, the stratified hazard ratio is stratified by: (a) geographical region selected from the group consisting of (i) Asia, (ii) Western Europe, United States of America, Canada, or Australia, and (iii) the rest of the world excluding (i)-(ii); (b) International Prognostic Index (IPI) score of 2 versus between 3 and 5; and/or (c) the presence or absence of bulky disease. In some embodiments, which may be combined with any of the preceding aspects or embodiments, administering such treatment results in a statistically significant improvement in the PFS as compared to the control treatment with a stratified hazard ratio of no more than 0.75 (95% confidence interval: 0.57, 0.97). In some embodiments, which may be combined with any of the preceding aspects or embodiments, administering such treatment results in a statistically significant improvement in the PFS as compared to the control treatment with a stratified hazard ratio of no more than 0.78 (95% confidence interval: 0.60, 1.00). In some embodiments, which may be combined with any of the preceding aspects or embodiments, administering such treatment results in a statistically significant improvement in the PFS as compared to the control treatment with an unstratified hazard ratio of no more than 0.79 (95% confidence interval: 0.61, 1.02). In some embodiments, which may be combined with any of the preceding aspects or embodiments, (a) administering such treatment to a plurality of human patients having an age greater than 60 years results in an improvement in the PFS as compared to the control treatment with a stratified hazard ratio of no more than 0.72 (95% confidence interval: 0.52, 0.99); or (b) administering such treatment to a plurality of human patients having an age greater than 65 years results in an improvement in the PFS as compared to the control treatment with a stratified hazard ratio of no more than 0.79 (95% confidence interval: 0.54, 1.14). In some embodiments, which may be combined with any of the preceding aspects or embodiments, administering such treatment to a plurality of human patients having an IPI score between 3 and 5 results in an improvement in the PFS as compared to the control treatment with a stratified hazard ratio of no more than 0.68 (95% confidence interval: 0.50, 0.94). In some embodiments, which may be combined with any of the preceding aspects or embodiments. (a) administering such treatment to a plurality of human patients having an ABC type DLBCL results in an improvement in the PFS as compared to the control treatment with a stratified hazard ratio of no more than 0.31 (95% confidence interval: 0.17, 0.56); or (b) administering such treatment to a plurality of human patients having a DEL type DLBCL results in an improvement in the PFS as compared to the control treatment with a stratified hazard ratio of no more than 0.62 (95% confidence interval: 0.40, 0.97). In some embodiments, which may be combined with any of the preceding aspects or embodiments, (a) administering such treatment to a plurality of human patients having an age greater than 60 years results in an improvement in the PFS as compared to the control treatment with an unstratified hazard ratio of no more than 0.72 (95% confidence interval: 0.53, 0.99); or (b) administering such treatment to a plurality of human patients having an age greater than 65 years results in an improvement in the PFS as compared to the control treatment with an unstratified hazard ratio of no more than 0.77 (95% confidence interval: 0.54, 1.10). In some embodiments, which may be combined with any of the preceding aspects or embodiments, (a) administering such treatment to a plurality of human patients having an age greater than 60 years results in an improvement in the PFS as compared to the control treatment with an unstratified hazard ratio of no more than 0.76 (95% confidence interval: 0.56, 1.02); or (b) administering such treatment to a plurality of human patients having an age greater than 65 years results in an improvement in the PFS as compared to the control treatment with an unstratified hazard ratio of no more than 0.78 (95% confidence interval: 0.56, 1.10). In some embodiments, which may be combined with any of the preceding aspects or embodiments, administering such treatment to a plurality of human patients having an IPI score between 3 and 5 results in an improvement in the PFS as compared to the control treatment with an unstratified hazard ratio of no more than 0.71 (95% confidence interval: 0.51, 0.97). In some embodiments, which may be combined with any of the preceding aspects or embodiments, administering such treatment to a plurality of human patients having an IPI score between 3 and 5 results in an improvement in the PFS as compared to the control treatment with an unstratified hazard ratio of no more than 0.75 (95% confidence interval: 0.55, 1.01). In some embodiments, which may be combined with any of the preceding aspects or embodiments, (a) administering such treatment to a plurality of human patients having an ABC type DLBCL results in an improvement in the PFS as compared to the control treatment with an unstratified hazard ratio of no more than 0.36 (95% confidence interval: 0.21, 0.62); or (b) administering such treatment to a plurality of human patients having a DEL type DLBCL results in an improvement in the PFS as compared to the control treatment with an unstratified hazard ratio of no more than 0.67 (95% confidence interval: 0.44, 1.02). In some embodiments, which may be combined with any of the preceding aspects or embodiments, (a) administering such treatment to a plurality of human patients having an ABC type DLBCL results in an improvement in the PFS as compared to the control treatment with an unstratified hazard ratio of no more than 0.39 (95% confidence interval: 0.23, 0.65); or (b) administering such treatment to a plurality of human patients having a DEL type DLBCL results in an improvement in the PFS as compared to the control treatment with an unstratified hazard ratio of no more than 0.65 (95% confidence interval: 0.43, 0.98). In some embodiments, which may be combined with any of the preceding aspects or embodiments, the stratified or unstratified hazard ratio is calculated at 12 months, 24 months, or more, measured starting from: (a) the start of the corresponding treatment; or (b) up to 7 days prior to the start of the corresponding treatment; or (c) the time from randomization to the time of a first occurrence of disease progression, relapse, or death.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein administering such treatment to a plurality of human patients results in a 24-month progression-free survival rate (PFS24) of at least 75%. In some embodiments, the PFS24 is calculated at 24 months, measured starting from: (a) the start of treatment; or (b) up to 7 days prior to the start of treatment; or (c) the time from randomization to the time of a first occurrence of disease progression, relapse, or death.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (c) prednisone, prednisolone, or methylprednisolone; wherein administering such treatment to a plurality of human patients results in an improvement in a 24-month progression-free survival rate (PFS24) of the plurality of human patients as compared to a reference PFS24, wherein the reference PFS24 is the 24-month progression-free survival rate of a plurality of human patients who have received a control treatment comprising: (a) rituximab, (b) cyclophosphamide, (c) doxorubicin, (d) vincristine, and (e) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein administering such treatment to a plurality of human patients results in an improvement in a 24-month progression-free survival rate (PFS24) of the plurality of human patients of at least about 6%, as compared to a reference PFS24, wherein the reference PFS24 is the 24-month progression-free survival rate of a plurality of human patients who have received a control treatment comprising: (a) rituximab, (b) cyclophosphamide, (c) doxorubicin, (d) vincristine, and (e) prednisone, prednisolone, or methylprednisolone, in the absence of polatuzumab vedotin.

In some embodiments, which may be combined with any of the preceding aspects or embodiments, the PFS24 or the reference PFS24 is calculated at 24 months, measured starting from: (a) the start of the corresponding treatment; or (b) up to 7 days prior to the start of the corresponding treatment; or (c) starting from the time from randomization to the time of a first occurrence of disease progression, relapse, or death. In some embodiments, which may be combined with any of the preceding aspects or embodiments, the PFS24 or the reference PFS24 is a progression-free survival (PFS) rate calculated using a Kaplan-Meier method.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein administering such treatment to a plurality of human patients results in a 12-month progression-free survival (PFS) rate of at least 83%. In some embodiments, the 12-month PFS is calculated at 12 months, measured starting from: (a) the start of treatment; or (b) up to 7 days prior to the start of treatment; or (c) the time from randomization to the time of a first occurrence of disease progression, relapse, or death.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein administering such treatment to a plurality of human patients results in an improvement in a 12-month progression-free survival (PFS) rate of the plurality of human patients as compared to a reference 12-month PFS rate, wherein the reference 12-month PFS rate is the 12-month PFS rate of a plurality of human patients who have received a control treatment comprising: (a) rituximab, (b) cyclophosphamide, (c) doxorubicin, (d) vincristine, and (e) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein administering such treatment to a plurality of human patients results in an improvement in a 12-month progression-free survival (PFS) rate of the plurality of human patients of at least about 3%, as compared to a reference 12-month PFS rate, wherein the reference 12-month PFS rate is the 12-month PFS rate of a plurality of human patients who have received a control treatment comprising: (a) rituximab, (b) cyclophosphamide, (c) doxorubicin, (d) vincristine, and (e) prednisone, prednisolone, or methylprednisolone, in the absence of polatuzumab vedotin.

In some embodiments, which may be combined with any of the preceding aspects or embodiments, the 12-month PFS rate or the reference 12-month PFS rate is calculated at 12 months, measured starting from: (a) the start of the corresponding treatment; or (b) up to 7 days prior to the start of the corresponding treatment; or (c) starting from the time from randomization to the time of a first occurrence of disease progression, relapse, or death. In some embodiments, which may be combined with any of the preceding aspects or embodiments, the 12-month PFS rate or the reference 12-month PFS rate is a progression-free survival (PFS) rate calculated using a Kaplan-Meier method.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein administering such treatment to a plurality of human patients results in an improvement in event-free survival-efficacy (EFS_{eff}) of the plurality of human patients as compared to a reference EFS_{eff}, wherein the reference EFS_{eff} is the EFS_{eff} of a plurality of human patients who have received a control treatment comprising: (a) rituximab, (b) cyclophosphamide, (c) doxorubicin, (d) vincristine, and (e) prednisone, prednisolone, or methylprednisolone, in the absence of polatuzumab vedotin. In some embodiments, the EFS_{eff} or the reference EFS_{eff} is measured; (a) starting from the start of the corresponding treatment to the time of a first occurrence of an EFS_{eff} event; or (b) starting from up to 7 days prior to the start of the corresponding treatment to the time of a first occurrence of an EFS_{eff} event; or (c) starting from the time from randomization to the time of a first occurrence of an EFS_{eff} event. In some embodiments, the improvement in EFS_{eff} is statistically significant. In some embodiments, the improvement in EFS_{eff} is calculated at 12 months, 24 months, or more, measured starting from: (a) the start of the corresponding treatment; or (b) up to 7 days prior to the start of the corresponding treatment; or (c) the time from randomization to the time of a first occurrence of an EFS_{eff} event.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein administering such treatment to a plurality of human patients results in a stratified hazard ratio of no more than 0.77 in event-free survival-efficacy (EFS_{eff}) in the plurality of human patients as compared to a control treatment comprising: (a) rituximab, (b) cyclophosphamide, (c) doxorubicin, (d) vincristine, and (e) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein administering such treatment to a plurality of human patients results in a stratified hazard ratio of no more than 0.81 in event-free survival-efficacy (EFS_{eff}) in the plurality of human patients as compared to a control treatment comprising: (a) rituximab, (b) cyclophosphamide, (c) doxorubicin, (d) vincristine, and (e) prednisone, prednisolone, or methylprednisolone, in the absence of polatuzumab vedotin.

In some embodiments, the EFS_{eff} is measured: (a) starting from the start of the corresponding treatment to the time of a first occurrence of an EFS_{eff} event; or (b) starting from up to 7 days prior to the start of the corresponding treatment to the time of a first occurrence of an EFS_{eff} event; or (c) starting from the time from randomization to the time of a first occurrence of an EFS_{eff} event. In some embodiments, administering such treatment results in a statistically significant improvement in the EFS_{eff} as compared to the control treatment with a stratified hazard ratio of no more than 0.77 (95% confidence interval: 0.59, 1.00). In some embodiments, administering such treatment results in a statistically significant improvement in the EFS_{eff} as compared to the control treatment with a stratified hazard ratio of no more than 0.81 (95% confidence interval: 0.63, 1.04). In some embodiments, the hazard ratio is calculated at 12 months, 24 months, or more, measured starting from: (a) the start of the corresponding treatment; or (b) up to 7 days prior to the start of the corresponding treatment; or (c) the time from randomization to the time of a first occurrence of an EFS_{eff} event.

In some embodiments, which may be combined with any of the preceding aspects or embodiments, the EFS_{eff} event is: (a) disease progression; (b) disease relapse; (c) death; (d) a primary efficacy reason that leads to initiation of a non-protocol specified anti-lymphoma treatment (NALT), and that is not disease progression or relapse; or (e) a biopsy positive for residual disease. In some embodiments, which may be combined with any of the preceding aspects or embodiments, the stratified hazard ratio is stratified by: (a) geographical region selected from the group consisting of (i) Asia, (ii) Western Europe, United States of America, Canada, or Australia, and (iii) the rest of the world excluding (i)-(ii); (b) International Prognostic Index (IPI) score of 2 versus between 3 and 5; and/or (c) the presence or absence of bulky disease.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein administering such treatment to a plurality of human patients results in a rate of complete response (CR) at end of treatment (EOT) in the plurality of human patients of at least about 77%, wherein the rate of CR is assessed by positron emission tomography-computed tomography (PET-CT). In some embodiments, the CR is assessed by an investigator or by blinded independent central review (BICR). In some embodiments, administering such treatment to a plurality of human patients results in an improvement in the rate of CR of at least about 3% in the plurality of human patients, as compared to a plurality of human patients who have received a control treatment comprising: (a) rituximab, (b) cyclophosphamide, (c) doxorubicin, (d) vincristine, and (e) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein administering such treatment to a plurality of human patients results in an objective response rate (ORR) at end of treatment (EOT) in the plurality of human patients of at least about 85%, wherein the ORR is assessed by positron emission tomography-computed tomography (PET-CT).

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein administering such treatment to a plurality of human patients results in an objective response rate (ORR) at end of treatment (EOT) in the plurality of human patients of at least about 84%, wherein the ORR is assessed by positron emission tomography-computed tomography (PET-CT).

In some embodiments, the ORR is assessed by an investigator or by blinded independent central review (BICR). In some embodiments, administering such treatment to a plurality of human patients results in an improvement in ORR of at least about 2% in the plurality of human patients, as compared to a plurality of human patients who have received a control treatment comprising: (a) rituximab, (b) cyclophosphamide, (c) doxorubicin, (d) vincristine, and (e) prednisone, prednisolone, or methylprednisolone, in the absence of polatuzumab vedotin.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein the human patient has an age of greater than 60 years.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein the human patient has an age of greater than 65 years.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein the human patient has an International Prognostic Index (IPI) score between 3 and 5.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein the human patient has an age of greater than 60 years and an International Prognostic Index (IPI) score between 3 and 5.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein the human patient has an age of greater than 65 years and an International Prognostic Index (IPI) score between 3 and 5.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein the human patient has an activated B-cell (ABC) type DLBCL.

In certain aspects, provided herein is a method for treating diffuse large B-cell lymphoma (DLBCL) in a human patient in need thereof, comprising administering to the human patient an effective amount of: (a) polatuzumab vedotin, (b) rituximab, (c) cyclophosphamide, (d) doxorubicin, and (e) prednisone, prednisolone, or methylprednisolone; wherein the human patient has a double expressing lymphoma (DEL) type DLBCL.

In some embodiments, which may be combined with any of the preceding aspects or embodiments, polatuzumab vedotin is administered at a dose of about 1.8 mg/kg. In some embodiments, which may be combined with any of the preceding aspects or embodiments, rituximab is administered at a dose of about 375 mg/m². In some embodiments, which may be combined with any of the preceding aspects or embodiments, cyclophosphamide is administered at a dose of about 750 mg/m². In some embodiments, which may be combined with any of the preceding aspects or embodiments, doxorubicin is administered at a dose of about 50 mg/m². In some embodiments, which may be combined with any of the preceding aspects or embodiments, vincristine is administered at a dose of about 1.4 mg/m² and up to 2 mg each dose. In some embodiments, which may be combined with any of the preceding aspects or embodiments, (a) prednisone is administered at a dose of about 100 mg; (b) prednisolone is administered at a dose of about 100 mg; or (c) methylprednisolone is administered at a dose of about 80 mg.

In some embodiments, which may be combined with any of the preceding aspects or embodiments, (a) the polatuzumab vedotin is administered to the human patient at a dose of about 1.8 mg/kg; (b) the rituximab is administered to the human patient at a dose of about 375 mg/m²; (c) the cyclophosphamide is administered to the human patient at a dose of about 750 mg/m²; (d) the doxorubicin is administered to the human patient at a dose of about 50 mg/m²; and (e) the prednisone is administered to the human patient at a dose of about 100 mg: the prednisolone is administered to the human patient at a dose of about 100 mg; or the methylprednisolone is administered to the human patient at a dose of about 80 mg.

In some embodiments, which may be combined with any of the preceding aspects or embodiments, (a) the polatuzumab vedotin is administered to the human patient intravenously at a dose of about 1.8 mg/kg; (b) the rituximab is administered to the human patient intravenously at a dose of about 375 mg/m²; (c) the cyclophosphamide is administered to the human patient intravenously at a dose of about 750 mg/m²; (d) the doxorubicin is administered to the human patient intravenously at a dose of about 50 mg/m²; and (e) the prednisone is administered to the human patient orally at a dose of about 100 mg: the prednisolone is administered to the human patient orally at a dose of about 100 mg; or the methylprednisolone is administered to the human patient intravenously at a dose of about 80 mg.

In some embodiments, which may be combined with any of the preceding aspects or embodiments, (a) the polatuzumab vedotin is administered to the human patient at a dose of about 1.0 mg/kg to about 1.8 mg/kg; (b) the rituximab is administered to the human patient at a dose of about 375 mg/m²; (c) the cyclophosphamide is administered to the human patient at a dose of about 375 mg/m² to about 750 mg/m²; (d) the doxorubicin is administered to the human patient at a dose of about 25 mg/m² to about 50 mg/m²; and (e) the prednisone is administered to the human patient at a dose of about 100 mg; the prednisolone is administered to the human patient at a dose of about 100 mg; or the methylprednisolone is administered to the human patient at a dose of about 80 mg.

In some embodiments, which may be combined with any of the preceding aspects or embodiments, (a) the polatuzumab vedotin is administered to the human patient intravenously at a dose of about 1.0 mg/kg to about 1.8 mg/kg; (b) the rituximab is administered to the human patient intravenously at a dose of about 375 mg/m²; (c) the cyclophosphamide is administered to the human patient intravenously at a dose of about 375 mg/m² to about 750 mg/m²; (d) the doxorubicin is administered to the human patient intravenously at a dose of about 25 mg/m² to about 50 mg/m²; and (e) the prednisone is administered to the human patient orally at a dose of about 100 mg: the prednisolone is administered to the human patient orally at a dose of about 100 mg; or the methylprednisolone is administered to the human patient intravenously at a dose of about 80 mg.

In some embodiments, which may be combined with any of the preceding aspects or embodiments, the polatuzumab vedotin, rituximab, cyclophosphamide, doxorubicin, and prednisone, prednisolone, or methylprednisolone are administered to the human patient in 21-day cycles. In some embodiments, the polatuzumab vedotin, the rituximab, the cyclophosphamide, and the doxorubicin are administered on day 1 of each 21-day cycle; and the prednisone, prednisolone, or methylprednisolone is administered on days 1-5 of each 21-day cycle. In some embodiments, (a) the polatuzumab vedotin is administered to the human patient intravenously at a dose of about 1.8 mg/kg on day 1 of each 21-day cycle; (b) the rituximab is administered to the human patient intravenously at a dose of about 375 mg/m² on day 1 of each 21-day cycle; (c) the cyclophosphamide is administered to the human patient intravenously at a dose of about 750 mg/m² on day 1 of each 21-day cycle; (d) the doxorubicin is administered to the human patient intravenously at a dose of about 50 mg/m² on day 1 of each 21-day cycle; and (e) the prednisone is administered to the human patient orally at a dose of about 100 mg per day on each of days 1-5 of each 21-day cycle; the prednisolone is administered orally at a dose of about 100 mg per day on each of days 1-5 of each 21-day cycle; or the methylprednisolone is administered intravenously at a dose of about 80 mg per day on each of days 1-5 of each 21-day cycle.

In some embodiments, which may be combined with any of the preceding aspects or embodiments, the polatuzumab vedotin, the rituximab, the cyclophosphamide, the doxorubicin, and the prednisone, prednisolone, or methylprednisolone are administered for one, two, three, four, five, or six 21-day cycles. In some embodiments, which may be combined with any of the preceding aspects or embodiments, the polatuzumab vedotin, the rituximab, the cyclophosphamide, the doxorubicin, and the prednisone, prednisolone, or methylprednisolone are administered for at least six 21-day cycles. In some embodiments, which may be combined with any of the preceding aspects or embodiments, the polatuzumab vedotin, the rituximab, the cyclophosphamide, the doxorubicin, and the prednisone, prednisolone, or methylprednisolone are administered for six 21-day cycles.

In some embodiments, which may be combined with any of the preceding aspects or embodiments, the rituximab, cyclophosphamide, doxorubicin, vincristine, and prednisone, prednisolone, or methylprednisolone of the control treatment are administered in 21-day cycles. In some embodiments, which may be combined with any of the preceding aspects or embodiments, the rituximab, cyclophosphamide, doxorubicin, and vincristine are administered on day 1 of each 21-day cycle; and the prednisone, prednisolone, or methylprednisolone is administered on days 1-5 of each 21-day cycle. In some embodiments, (a) the rituximab is administered intravenously at a dose of about 375 mg/m² on day 1 of each 21-day cycle; (b) the cyclophosphamide is administered intravenously at a dose of about 750 mg/m² on day 1 of each 21-day cycle; (c) the doxorubicin is administered intravenously at a dose of about 50 mg/m² on day 1 of each 21-day cycle; (d) the vincristine is administered intravenously at a dose of about 1.4 mg/m² and up to 2 mg each dose on day 1 of each 21-day cycle; and (e) the prednisone is administered orally at a dose of about 100 mg per day on each of days 1-5 of each 21-day cycle; the prednisolone is administered orally at a dose of about 100 mg per day on each of days 1-5 of each 21-day cycle; or the methylprednisolone is administered intravenously at a dose of about 80 mg per day on each of days 1-5 of each 21-day cycle. In some embodiments, which may be combined with any of the preceding aspects or embodiments, the rituximab, the cyclophosphamide, the doxorubicin, the vincristine, and the prednisone, prednisolone, or methylprednisolone are administered for one, two, three, four, five, or six 21-day cycles. In some embodiments, which may be combined with any of the preceding aspects or embodiments, the rituximab, the cyclophosphamide, the doxorubicin, the vincristine, and the prednisone, prednisolone, or methylprednisolone are administered for at least six 21-day cycles. In some embodiments, which may be combined with any of the preceding aspects or embodiments, the rituximab, the cyclophosphamide, the doxorubicin, the vincristine, and the prednisone, prednisolone, or methylprednisolone are administered for six 21-day cycles.

In some embodiments, which may be combined with any of the preceding aspects or embodiments, the polatuzumab vedotin, the rituximab, the cyclophosphamide, the doxorubicin, and the prednisone are administered to the human patient. In some embodiments, which may be combined with any of the preceding aspects or embodiments, the polatuzumab vedotin, the rituximab, the cyclophosphamide, the doxorubicin, and the prednisolone are administered to the human patient. In some embodiments, which may be combined with any of the preceding aspects or embodiments, the polatuzumab vedotin, the rituximab, the cyclophosphamide, the doxorubicin, and the methylprednisolone are administered to the human patient.

In some embodiments, which may be combined with any of the preceding aspects or embodiments, the polatuzumab vedotin, the rituximab, the cyclophosphamide, the doxorubicin, and the prednisone, prednisolone, or methylprednisolone are administered to the human patient sequentially on day 1 of each 21-day cycle. In some embodiments, (a) the prednisone, prednisolone, or methylprednisolone is administered prior to the rituximab: the rituximab is administered prior to the polatuzumab vedotin; and the polatuzumab vedotin is administered prior to the cyclophosphamide and doxorubicin; or (b) the rituximab, polatuzumab vedotin, cyclophosphamide and doxorubicin are administered in any order after administration of the prednisone, prednisolone, or methylprednisolone.

In some embodiments, which may be combined with any of the preceding aspects or embodiments, the method further comprises (a) administering rituximab monotherapy to the human patient during a seventh and eighth 21-day cycle after the sixth 21-day cycle; or (b) administering rituximab, cyclophosphamide, doxorubicin, and prednisone, prednisolone, or methylprednisolone to the human patient during a seventh and eighth 21-day cycle after the sixth 21-day cycle. In some embodiments, the method comprises administering rituximab monotherapy to the human patient intravenously at a dose of about 375 mg/m² on day 1 of each of the seventh and eighth 21-day cycles. In some embodiments, the method comprises administering rituximab, cyclophosphamide, doxorubicin, and prednisone, prednisolone, or methylprednisolone to the human patient, wherein: (a) the rituximab is administered intravenously at a dose of about 375 mg/m² on day 1 of each of the seventh and eighth 21-day cycles; (b) the cyclophosphamide is administered intravenously at a dose of about 750 mg/m² on day 1 of each of the seventh and eighth 21-day cycles; (c) the doxorubicin is administered intravenously at a dose of about 50 mg/m² on day 1 of each of the seventh and eighth 21-day cycles; and (d) the prednisone is administered orally at a dose of about 100 mg per day on each of days 1-5 of each of the seventh and eighth 21-day cycles; the prednisolone is administered orally at a dose of about 100 mg per day on each of days 1-5 of each of the seventh and eighth 21-day cycles; or the methylprednisolone is administered intravenously at a dose of about 80 mg per day on each of days 1-5 of each of the seventh and eighth 21-day cycles.

In some embodiments, which may be combined with any of the preceding aspects or embodiments, the rituximab, the cyclophosphamide, the doxorubicin, the vincristine, and the prednisone, prednisolone, or methylprednisolone of the control treatment are administered sequentially on day 1 of each 21-day cycle. In some embodiments, (a) the prednisone, prednisolone, or methylprednisolone is administered prior to the rituximab; and the rituximab is administered prior to the cyclophosphamide, doxorubicin and vincristine; or (b) the rituximab, cyclophosphamide, doxorubicin and vincristine are administered in any order after administration of the prednisone, prednisolone, or methylprednisolone. In some embodiments, which may be combined with any of the preceding aspects or embodiments, the control treatment further comprises: (a) rituximab monotherapy during a seventh and eighth 21-day cycle after the sixth 21-day cycle; or (b) rituximab, cyclophosphamide, doxorubicin, vincristine, and prednisone, prednisolone, or methylprednisolone during a seventh and eighth 21-day cycle after the sixth 21-day cycle. In some embodiments, the control treatment further comprises rituximab monotherapy administered intravenously at a dose of about 375 mg/m² on day 1 of each of the seventh and eighth 21-day cycles. In some embodiments, the control treatment further comprises rituximab, cyclophosphamide, doxorubicin, vincristine, and prednisone, prednisolone, or methylprednisolone during a seventh and eighth 21-day cycle after the sixth 21-day cycle, wherein: (a) the rituximab is administered intravenously at a dose of about 375 mg/m² on day 1 of each of the seventh and eighth 21-day cycles; (b) the cyclophosphamide is administered intravenously at a dose of about 750 mg/m² on day 1 of each of the seventh and eighth 21-day cycles; (c) the doxorubicin is administered intravenously at a dose of about 50 mg/m² on day 1 of each of the seventh and eighth 21-day cycles; (d) the vincristine is administered intravenously at a dose of about 1.4 mg/m² and up to 2 mg each dose on day 1 of each of the seventh and eighth 21-day cycles; and (e) the prednisone is administered orally at a dose of about 100 mg per day on each of days 1-5 of each of the seventh and eighth 21-day cycles; the prednisolone is administered orally at a dose of about 100 mg per day on each of days 1-5 of each of the seventh and eighth 21-day cycles; or the methylprednisolone is administered intravenously at a dose of about 80 mg per day on each of days 1-5 of each of the seventh and eighth 21-day cycles.

In some embodiments, which may be combined with any of the preceding aspects or embodiments, the method further comprises administering to the human patient an antihistamine drug, an analgesic, and/or an anti-pyretic drug. In some embodiments, which may be combined with any of the preceding aspects or embodiments, the method further comprises administering to the human patient a prophylactic therapy for neutropenia. In some embodiments, the method comprises administering to the human patient granulocyte colony-stimulating factor (G-CSF). In some embodiments, the G-CSF is filgrastim, or lenograstim, or peg-filgrastim.

In some embodiments, which may be combined with any of the preceding aspects or embodiments, the human patient has a high tumor burden. In some embodiments, which may be combined with any of the preceding aspects or embodiments, the human patient has a lymphocyte count of at least about 25×10⁹/L. In some embodiments, which may be combined with any of the preceding aspects or embodiments, the human patient has bulky lymphadenopathy. In some embodiments, which may be combined with any of the preceding aspects or embodiments, the human patient is at risk for developing tumor lysis syndrome. In some embodiments, which may be combined with any of the preceding aspects or embodiments, the method further comprises administering to the human patient a prophylactic therapy for tumor lysis syndrome. In some embodiments, the prophylactic therapy for tumor lysis syndrome comprises administering allopurinol or rasburicase to the human patient. In some embodiments, the prophylactic therapy for tumor lysis syndrome comprises a hydration regimen. In some embodiments, the hydration regimen comprises administering to the human patient about 3 liters per day of fluids starting at between 1 and 2 days prior to the start of treatment for DLBCL.

In some embodiments, which may be combined with any of the preceding aspects or embodiments, the human patient has previously untreated DLBCL.

In some embodiments, which may be combined with any of the preceding aspects or embodiments, the DLBCL is CD20 positive. In some embodiments, which may be combined with any of the preceding aspects or embodiments, the DLBCL is a DLBCL, not otherwise specified (NOS). In some embodiments, which may be combined with any of the preceding aspects or embodiments, the DLBCL is a germinal center B-cell type DLBCL. In some embodiments, which may be combined with any of the preceding aspects or embodiments, the DLBCL is an activated B-cell (ABC) type DLBCL. In some embodiments, which may be combined with any of the preceding aspects or embodiments, the DLBCL is a double expressing (DEL) type DLBCL. In some embodiments, which may be combined with any of the preceding aspects or embodiments, the DLBCL is: (a) a T-cell/histiocyte-rich large B-cell lymphoma; (b) an Epstein-Barr virus-positive DLBCL, NOS; (c) an ALK-positive large B-cell lymphoma; (d) an HHV8-positive DLBCL, NOS; (e) a high-grade B-cell lymphoma comprising a MYC, a BCL2, and/or a BCL6 rearrangement (double-hit lymphoma or a triple-hit lymphoma); or (h) a high-grade B-cell lymphoma, NOS.

In some embodiments, which may be combined with any of the preceding aspects or embodiments, the human patient has an International Prognostic Index (IPI) score of between 2 and 5. In some embodiments, which may be combined with any of the preceding aspects or embodiments, the human patient has an IPI score of 2. In some embodiments, which may be combined with any of the preceding aspects or embodiments, the human patient has an IPI score of between 3 and 5.

In some embodiments, which may be combined with any of the preceding aspects or embodiments, the human patient is an adult. In some embodiments, which may be combined with any of the preceding aspects or embodiments, the human patient has an Eastern Cooperative Oncology Group (ECOG) Performance Status of 0, 1, or 2. In some embodiments, which may be combined with any of the preceding aspects or embodiments, the human patient has at least one bi-dimensionally measurable lesion. In some embodiments, the at least one bi-dimensionally measurable lesion has a size greater than 1.5 cm in its longest dimension, as measured by computed tomography (CT) or magnetic resonance imaging (MRI).

In some embodiments, which may be combined with any of the preceding aspects or embodiments, the human patient does not have peripheral neuropathy of grade greater than 1 prior to the start of treatment for DLBCL. In some embodiments, which may be combined with any of the preceding aspects or embodiments, the human patient does not have a demyelinating form of Charcot-Marie Tooth disease prior to the start of treatment for DLBCL. In some embodiments, which may be combined with any of the preceding aspects or embodiments, the human patient does not have history of indolent lymphoma prior to the start of treatment for DLBCL. In some embodiments, which may be combined with any of the preceding aspects or embodiments, the human patient does not have: (a) follicular lymphoma grade 3B, (b) B-cell lymphoma, unclassifiable, with features intermediate between DLBCL and classical Hodgkin lymphoma, (c) grey-zone lymphoma, (d) primary mediastinal (thymic) large B-cell lymphoma, (e) Burkitt lymphoma, (f) central nervous system (CNS) lymphoma, primary or secondary involvement, (g) primary effusion DLBCL, or (h) primary cutaneous DLBCL, prior to the start of treatment for DLBCL.

In some embodiments, which may be combined with any of the preceding aspects or embodiments, the human patient has not been previously treated for DLBCL.

In some embodiments, which may be combined with any of the preceding aspects or embodiments, the disease progression or relapse is assessed using the 2014 Lugano Classification for Malignant Lymphoma, and the death is from any cause.

In certain aspects, provide herein is a kit comprising polatuzumab vedotin for use in combination with rituximab, cyclophosphamide, doxorubicin, and prednisone, prednisolone or methylprednisolone for treating a human patient in need thereof having diffuse large B-cell lymphoma (DLBCL) according to any of the methods provided herein. In some embodiments, the DLBCL is previously untreated DLBCL.

In certain aspects, provided herein is polatuzumab vedotin for use in combination with rituximab, cyclophosphamide, doxorubicin, and prednisone, prednisolone or methylprednisolone for treating a human patient in need thereof having diffuse large B-cell lymphoma (DLBCL) according any of the methods provided herein. In some embodiments, the DLBCL is previously untreated DLBCL.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 is a diagram of the design of the study described in Example 1. DLBCL=diffuse large B-cell lymphoma: ECOG PS=Eastern Cooperative Oncology Group Performance Status: IPI=International Prognostic Index: Q21D=every 21 days: R=randomization: R-CHOP=rituximab plus cyclophosphamide, doxorubicin, vincristine, and prednisone: R-CHP=rituximab plus cyclophosphamide, doxorubicin, and prednisone.

FIG. 2 is a diagram of the treatment regimens used in the study described in the Example(s).

FIG. 3 is a Kaplan-Meier Plot of progression-free survival (PFS), assessed by the investigator, in the intent-to-treat (ITT) population of patients as described in the Example(s).

FIGS. 4A and 4B and FIGS. 5A and 5B provide a time to event summary for PFS, assessed by the investigator in the ITT population of patients as described in the Example(s).

FIG. 6 provides a forest plot of stratified hazard ratio for PFS by biomarker subgroups, assessed by the investigator in the ITT population of patients as described in the Example(s).

FIG. 7 and FIG. 8 provide a forest plot of stratified hazard ratio for PFS by baseline characteristics subgroup, assessed by the investigator in the ITT population of patients as described in the Example(s).

FIGS. 9A and 9B provide an overview of the overall AE profile in the safety-evaluable population of patients as described in the Example(s).

FIGS. 10A and 10B provide forest plots of unstratified hazard ratio for PFS by baseline characteristics subgroup, assessed by the investigator in the ITT population of patients as described in the Example(s).

FIG. 11 provides a forest plot of unstratified hazard ratio for PFS by biomarker subgroup, assessed by the investigator in the ITT population of patients as described in the Example(s).

FIGS. 12A-12E provide a summary of demographics and baseline characteristics of patients in the ITT population as described in the Example(s). FIG. 12A provides the age, sex, and race of patients in the ITT population. FIG. 12B provides the ethnicity, baseline weight, baseline height, baseline Eastern Cooperative Oncology Group (ECOG) status, and Ann Arbor Sage of patients in the ITT population.

FIG. 12C provides the stratification International Prognostic Index (IPI) score, the screening IPI score, stratification bulky disease status, baseline bulky disease status, and stratification geographic region of patients in the ITT population. FIG. 12D provides baseline lactate dehydrogenase (LDH) status, bone marrow involvement status at diagnosis, number of extranodal sites, time from diagnosis to the time of administration of study treatment, and Non-Hodgkin lymphoma (NHL) histology diagnosis of patients in the ITT population. FIG. 12E provides the cell of origin (COO), double-expressor lymphoma status (by immunohistochemistry [IHC]), and double/triple-hit (DH/TH) lymphoma status of patients in the ITT population.

FIG. 13 provides a summary of best overall responses (BOR) assessed by the investigator (INV) in the ITT population of patients as described in the Example(s). Stratification factors included International Prognostic Index (IPI) score, bulky disease status and geographic region. 95% confidence intervals (CI) for rates were constructed using the Clopper-Pearson method. 95% CIs for differences in response rates were constructed using the Wilson method.

FIGS. 14A-14C provide a time to event summary for disease free survival (DFS; assessed by INV) for best overall response (BOR) complete responders in the ITT population of patients as described in the Example(s). FIG. 14A provides the percent of patients with and without DFS events, the time to event (in months), and the stratified and unstratified hazard ratios. FIGS. 14B-14C provide the number of patients remaining at risk, the event free rate and 95% confidence interval (CI), and the difference in the event free rate and 95% CI, at the 6-month, 12-month, 18-month and 24-month duration timepoints (FIG. 14B), and at the 30-month and 36-month duration timepoints (FIG. 14C). In FIGS. 14A-14C, summaries of DFS by INV (median, percentiles) are Kaplan-Meier estimates. 95% CIs for the medians were computed using the method of Brookmeyer and Crowley. Hazard ratios were estimated by Cox regression. Stratifications factors were geographical region, International Prognostic Index (IPI) score, and bulky disease (defined as one lesion ≥7.5 cm). Asterisks (*) indicate censored observations.

FIGS. 15A-15C provide a time to event summary for duration of response (DOR; assessed by INV) for best overall response (BOR) complete responders in the ITT population of patients as described in the Example(s). FIG. 15A provides the percent of patients with and without DOR events, the time to event (in months), and the stratified and unstratified hazard ratios. FIGS. 15B-15C provide the number of patients remaining at risk, the event free rate and 95% confidence interval (CI), and the difference in the event free rate and 95% CI, at the 6-month, 12-month, 18-month and 24-month duration timepoints (FIG. 15B), and at the 30-month and 36-month duration timepoints (FIG. 15C). In FIGS. 15A-15C, summaries of DOR by INV (median, percentiles) are Kaplan-Meier estimates. 95% CIs for the medians were computed using the method of Brookmeyer and Crowley. Hazard ratios were estimated by Cox regression. Stratifications factors were geographical region, International Prognostic Index (IPI) score, and bulky disease (defined as one lesion ≥7.5 cm). Asterisks (*) indicate censored observations.

FIGS. 16A-16C provide a time to event summary for overall survival (OS) in the ITT population of patients as described in the Example(s). FIG. 16A provides the percent of patients with and without OS events, the time to event (in months), and the stratified and unstratified p-values and hazard ratios. FIGS. 16B-16C provide the number of patients remaining at risk, the event free rate and 95% CI, and the difference in the event free rate and 95% CI, at the 6-month, 12-month, 18-month and 24-month duration timepoints (FIG. 16B), and at the 30-month and 36-month duration timepoints (FIG. 16C). In FIGS. 16A-16C, summaries of OS (median, percentiles) are Kaplan-Meier estimates. 95% CIs for the medians were computed using the method of Brookmeyer and Crowley. Hazard ratios were estimated by Cox regression. Stratifications factors were geographical region, International Prognostic Index (IPI) score, and bulky disease (defined as one lesion ≥7.5 cm). Asterisks (*) indicate censored observations.

FIG. 17 provides a forest plot of unstratified hazard ratios for PFS by biomarker subgroups, assessed by the investigator in the intent-to-treat (ITT) population of patients as described in the Example(s).

FIG. 18A and FIG. 18B provide forest plots of unstratified hazard ratios for PFS by baseline characteristics subgroups, assessed by the investigator in the intent-to-treat (ITT) population of patients as described in the Example(s).

FIG. 19 is a Kaplan-Meier Plot of event-free survival-efficacy (EFS_{eff}), assessed by the investigator in the intent-to-treat (ITT) population of patients as described in the Example(s).

FIG. 20 is a Kaplan-Meier Plot of overall survival (OS) in the intent-to-treat (ITT) population of patients as described in the Example(s).

FIG. 21 is a Kaplan-Meier Plot of progression-free survival (PFS), assessed by the investigator in the intent-to-treat (ITT) population of patients as described in the Example(s).

FIG. 22A and FIG. 22B provide a time to event summary for event-free survival-efficacy (EFS_{eff}), assessed by the investigator in the intent-to-treat (ITT) population of patients as described in the Example(s).

FIG. 23A and FIG. 23B provide a time to event summary for overall survival (OS) in the intent-to-treat (ITT) population of patients as described in the Example(s).

FIG. 24A and FIG. 24B provide a time to event summary for progression-free survival (PFS), assessed by the investigator in the intent-to-treat (ITT) population of patients as described in the Example(s).

FIG. 25 provides a summary of best overall responses (BOR) assessed by the investigator (INV) in the intent-to-treat (ITT) population of patients as described in the Example(s).

FIG. 26A and FIG. 26B provide a time to event summary for disease-free survival (DFS), assessed by the investigator for BOR complete responders in the intent-to-treat (ITT) population of patients as described in the Example(s).

FIG. 27A and FIG. 27B provide a time to event summary for duration of response (DOR), assessed by the investigator for BOR responders in the intent-to-treat (ITT) population of patients as described in the Example(s).

FIG. 28 provides a summary of new anti-lymphoma therapies (NALT) administered to the intent-to-treat (ITT) population of patients as described in the Example(s).

FIG. 29 provides an overview of the overall adverse event (AE) profile in the safety-evaluable population of patients as described in the Example(s).

FIG. 30 provides an overview of peripheral neuropathy, an AE of particular interest (AEPI), in the safety-evaluable population of patients as described in the Example(s).

### DETAILED DESCRIPTION

As used herein, the term “polatuzumab vedotin” refers to an anti-CD79b immunoconjugate having the IUPHAR/BPS Number 8404, the KEGG Number D10761, or the CAS Registry Number 1313206-42-6. Polatuzumab vedotin is also interchangeably referred to as “polatuzumab vedotin-piiq”, “huMA 79bv28-MC-vc-PAB-MMAE”, “DCDS4501A”, or “RG7596.” The term “polatuzumab vedotin” also refers to all corresponding anti-CD79b immunoconjugates that fulfill the requirements necessary for obtaining a marketing authorization as an identical or biosimilar product in a country or territory selected from the group of countries consisting of the USA, Europe and Japan.

Provided herein are methods for treating or delaying progression of lymphoma (such as diffuse large B-cell lymphoma (DLBCL)) in an individual (e.g., a human patient), comprising administering to the individual an effective amount of an anti-CD79b immunoconjugate (e.g., huMA79bv28-MC-vc-PAB-MMAE, which is also known as polatuzumab vedotin), an anti-CD20 agent (e.g., an anti-CD20 antibody such as obinutuzumab or rituximab), one or more chemotherapeutic agents (e.g., cyclophosphamide and/or doxorubicin), and a corticosteroid (e.g., prednisone, prednisolone, or methylprednisolone). In some embodiments, the methods comprise treating an individual having diffuse large B-cell lymphoma (DLBCL), by administering to the individual: (a) an immunoconjugate comprising the formula:

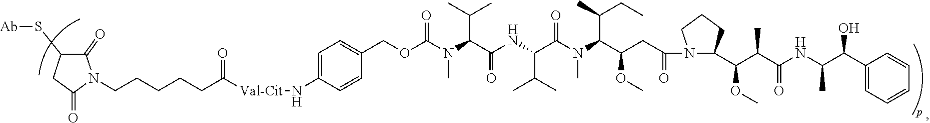

wherein Ab is an anti-CD79b antibody comprising (i) an HVR-H1 that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8 (e.g., between 2 and 5, or between 3 and 4), (b) an anti-CD20 antibody (e.g., obinutuzumab or rituximab), (c) one or more chemotherapeutic agents (e.g., cyclophosphamide and/or doxorubicin), and (d) a corticosteroid (e.g., prednisone, prednisolone, or methylprednisolone). In some embodiments, the immunoconjugate is administered at a dose between about 1.0 mg/kg and about 1.8 mg/kg (e.g., 1.0 mg/kg, 1.4 mg/kg, or 1.8 mg/kg). In some embodiments, the immunoconjugate is administered at a dose of about 1.8 mg/kg. In some embodiments, the anti-CD20 antibody (e.g., rituximab) is administered at a dose of about 375 mg/m². In some embodiments, the anti-CD20 antibody (e.g., obinutuzumab) is administered at a dose of about 1000 mg. In some embodiments, the one or more chemotherapeutic agents comprise cyclophosphamide and doxorubicin. In some embodiments, the cyclophosphamide is administered at a dose of between about 375 mg/m² and about 750 mg/m² (e.g., 375 mg/m², 563 mg/m², or 750 mg/m²). In some embodiments, the cyclophosphamide is administered at a dose of about 750 mg/m². In some embodiments, the doxorubicin is administered at a dose of between about 25 mg/m² and about 50 mg/m² (e.g., 25 mg/m², 37.5 mg/m², or 50 mg/m²). In some embodiments, the doxorubicin is administered at a dose of about 50 mg/m². In some embodiments, the corticosteroid is prednisone, prednisolone, or methylprednisolone. In some embodiments, the corticosteroid is prednisone, administered at a dose of about 100 mg. In some embodiments, the corticosteroid is prednisolone, administered at a dose of about 100 mg. In some embodiments, the corticosteroid is methylprednisolone, administered intravenously at a dose of about 80 mg.

### I. General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989): “Oligonucleotide Synthesis” (M. J. Gait, ed., 1984): “Animal Cell Culture” (R. I. Freshney, ed., 1987): “Methods in Enzymology” (Academic Press, Inc.); “Current Protocols in Molecular Biology” (F. M. Ausubel et al., eds., 1987, and periodic updates): “PCR: The Polymerase Chain Reaction”, (Mullis et al., ed., 1994): “A Practical Guide to Molecular Cloning” (Perbal Bernard V., 1988): “Phage Display: A Laboratory Manual” (Barbas et al., 2001).

### II. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the context clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

The term “CD79b,” as used herein, refers to any native CD79b from any vertebrate source, including mammals such as primates (e.g., humans, cynomolgus monkey (“cyno”)) and rodents (e.g., mice and rats), unless otherwise indicated. Human CD79b is also referred to herein as “Igβ,” “B29,” “DNA225786” or “PRO36249.” An exemplary CD79b sequence including the signal sequence is shown in SEQ ID NO: 1. An exemplary CD79b sequence without the signal sequence is shown in SEQ ID NO: 2. The term “CD79b” encompasses “full-length,” unprocessed CD79b as well as any form of CD79b that results from processing in the cell. The term also encompasses naturally occurring variants of CD79b, e.g., splice variants, allelic variants and isoforms. The CD79b polypeptides described herein may be isolated from a variety of sources, such as from human tissue types or from another source, or prepared by recombinant or synthetic methods. A “native sequence CD79b polypeptide” comprises a polypeptide having the same amino acid sequence as the corresponding CD79b polypeptide derived from nature. Such native sequence CD79b polypeptides can be isolated from nature or can be produced by recombinant or synthetic means. The term “native sequence CD79b polypeptide” specifically encompasses naturally occurring truncated or secreted forms of the specific CD79b polypeptide (e.g., an extracellular domain sequence), naturally occurring variant forms (e.g., alternatively spliced forms) and naturally occurring allelic variants of the polypeptide.

“CD20” as used herein refers to the human B-lymphocyte antigen CD20 (also known as CD20, B-lymphocyte surface antigen B1, Leu-16, Bp35, BM5, and LF5: the sequence is characterized by the SwissProt database entry P11836) is a hydrophobic transmembrane protein with a molecular weight of approximately 35 kD located on pre-B and mature B lymphocytes. (Valentine, M. A., et al., J. Biol. Chem. 264 (19) (1989 11282-11287: Tedder, T. F., et al, Proc. Natl. Acad. Sci. U.S.A. 85 (1988) 208-12; Stamenkovic, I., et al., J. Exp. Med. 167 (1988) 1975-80: Einfeld, D. A. et al., EMBO J. 7 (1988) 711-7; Tedder, T. F., et al., J. Immunol. 142 (1989) 2560-8). The corresponding human gene is Membrane-spanning 4-domains, subfamily A, member 1, also known as MS4A1. This gene encodes a member of the membrane-spanning 4A gene family. Members of this nascent protein family are characterized by common structural features and similar intron/exon splice boundaries and display unique expression patterns among hematopoietic cells and nonlymphoid tissues. This gene encodes the B-lymphocyte surface molecule which plays a role in the development and differentiation of B-cells into plasma cells. This family member is localized to 11q12, among a cluster of family members. Alternative splicing of this gene results in two transcript variants which encode the same protein.

The terms “CD20” and “CD20 antigen” are used interchangeably herein, and include any variants, isoforms and species homologs of human CD20 which are naturally expressed by cells or are expressed on cells transfected with the CD20 gene. Binding of an antibody of the invention to the CD20 antigen mediates the killing of cells expressing CD20 (e.g., a tumor cell) by inactivating CD20. The killing of the cells expressing CD20 may occur by one or more of the following mechanisms: Cell death/apoptosis induction, ADCC and CDC. Synonyms of CD20, as recognized in the art, include B-lymphocyte antigen CD20, B-lymphocyte surface antigen B1, Leu-16, Bp35, BM5, and LF5.

The term “expression of the CD20” antigen is intended to indicate a significant level of expression of the CD20 antigen in a cell, e.g., a T- or B-Cell. In one embodiment, patients to be treated according to the methods of this invention express significant levels of CD20 on a B-cell tumor or cancer. Patients having a “CD20 expressing cancer” can be determined by standard assays known in the art. E.g., CD20 antigen expression is measured using immunohistochemical (IHC) detection, FACS or via PCR-based detection of the corresponding mRNA.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “antibody” herein is used in the broadest sense and encompasses various antibody structures, including but not limited to, monoclonal antibodies, polyclonal antibodies, multispecific antibodies (e.g., bispecific antibodies), and antibody fragments, so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab-SH, F (ab′)₂; diabodies; linear antibodies; single-chain antibody molecules (e.g., scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “epitope” refers to the particular site on an antigen molecule to which an antibody binds.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “anti-CD79b antibody” or “an antibody that binds to CD79b” refers to an antibody that is capable of binding CD79b with sufficient affinity such that the antibody is useful as a diagnostic and/or therapeutic agent in targeting CD79b. Preferably, the extent of binding of an anti-CD79b antibody to an unrelated, non-CD79b protein is less than about 10% of the binding of the antibody to CD79b as measured, e.g., by a radioimmunoassay (RIA). In certain embodiments, an antibody that binds to CD79b has a dissociation constant (Kd) of ≤1 μM, ≤100 nM, ≤10 nM, ≤1 nM, or ≤0.1 nM. In certain embodiments, an anti-CD79b antibody binds to an epitope of CD79b that is conserved among CD79b from different species.

The term “anti-CD20 antibody” according to the invention refers to an antibody that is capable of binding CD20 with sufficient affinity such that the antibody is useful as a diagnostic and/or therapeutic agent in targeting CD20. Preferably, the extent of binding of an anti-CD20 antibody to an unrelated, non-CD20 protein is less than about 10% of the binding of the antibody to CD20 as measured, e.g., by a radioimmunoassay (RIA). In certain embodiments, an antibody that binds to CD20 has a dissociation constant (Kd) of ≤1 μM, ≤100 nM, ≤10 nM, ≤1 nM, or ≤0.1 nM. In certain embodiments, anti-CD20 antibody binds to an epitope of CD20 that is conserved among CD20 from different species.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For review of methods for assessment of antibody purity, see. e.g., Flatman et al., J. Chromatogr. B 848:79-87 (2007). The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

“Isolated nucleic acid encoding an anti-CD79b antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL); FR1-H1 (L1)-FR2-H2 (L2)-FR3-H3 (L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell, or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al . . . supra. In one embodiment, for the VH, the subgroup is subgroup III as in Kabat et al . . . supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs: three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987). Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3 (Kabat et al., Sequences of Proteins of Immunological Interest, 5th Ed. Public Health Service, National Institutes of Health, Bethesda, MD (1991). With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008). Unless otherwise indicated, HVR residues and other residues in the variable domain (e.g., FR residues) are numbered herein according to Kabat et al., supra.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs) (See. e.g., Kindt et al. Kuby Immunology, 6^{th} ed., W.H. Freeman and Co., page 91 (2007). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See. e.g., Portolano et al., J. Immunol. 150:880-887 (1993); Clarkson et al., Nature 352:624-628 (1991).

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B-cell receptor); and B-cell activation.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

An “immunoconjugate” is an antibody conjugated to one or more heterologous molecule(s), including but not limited to a cytotoxic agent.

In the context of the formulas provided herein, “p” refers to the average number of drug moieties per antibody, which can range, e.g., from about 1 to about 20 drug moieties per antibody, and in certain embodiments, from 1 to about 8 drug moieties per antibody. The invention includes a composition comprising a mixture of antibody-drug compounds of Formula I where the average drug loading per antibody is about 2 to about 5, or about 3 to about 4, (e.g., about 3.4 or about 3.5).

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

The terms “cancer” and “cancerous” refer to or describe the physiological condition in mammals that is typically characterized by unregulated cell growth. Examples of cancer include but are not limited to, B-cell lymphoma (including low grade/follicular non-Hodgkin's lymphoma (NHL): small lymphocytic (SL) NHL: intermediate grade/follicular NHL: intermediate grade diffuse NHL: high grade immunoblastic NHL: high grade lymphoblastic NHL: high grade small non-cleaved cell NHL: bulky disease NHL: mantle cell lymphoma: AIDS-related lymphoma; and Waldenstrom's Macroglobulinemia); chronic lymphocytic leukemia (CLL): acute lymphoblastic leukemia (ALL): Hairy cell leukemia: chronic myeloblastic leukemia; and post-transplant lymphoproliferative disorder (PTLD), as well as abnormal vascular proliferation associated with phakomatoses, edema (such as that associated with brain tumors), and Meigs' syndrome. More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B-cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B-cell lymphoma, splenic marginal zone lymphoma, extranodal marginal zone-MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), follicular lymphoma (e.g., relapsed/refractory follicular lymphoma), intermediate grade diffuse NHL, diffuse large B-cell lymphoma (DLBCL; e.g., relapsed/refractory DLBCL), aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and/or Sezary syndrome, skin (cutaneous) lymphomas, anaplastic large cell lymphoma, angiocentric lymphoma.

An “individual” or “subject” is a mammal. Mammals include, but are not limited to, domesticated animals (e.g., cows, sheep, cats, dogs, and horses), primates (e.g., humans, such as a human patient, e.g., having DLBCL, and non-human primates such as monkeys), rabbits, and rodents (e.g., mice and rats). In certain embodiments, the individual or subject is a human.

An “effective amount” of an agent, e.g., a pharmaceutical formulation, refers to an amount effective, at dosages and for periods of time necessary, to achieve the desired therapeutic or prophylactic result.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, reduction of free light chain, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, the methods described herein are used to delay development of a disease or to slow the progression of a disease.

The term “CD79b-positive cancer” refers to a cancer comprising cells that express CD79b on their surface. In some embodiments, expression of CD79b on the cell surface is determined, for example, using antibodies to CD79b in a method such as immunohistochemistry, FACS, etc. Alternatively, CD79b mRNA expression is considered to correlate to CD79b expression on the cell surface and can be determined by a method selected from in situ hybridization and RT-PCR (including quantitative RT-PCR).

As used herein, “in conjunction with” refers to administration of one treatment modality in addition to another treatment modality. As such, “in conjunction with” refers to administration of one treatment modality before, during, or after administration of the other treatment modality to the individual.

A “chemotherapeutic agent” is a chemical compound useful in the treatment of cancer.

The term “package insert” is used to refer to instructions customarily included in commercial packages of therapeutic products, that contain information about the indications, usage, dosage, administration, combination therapy, contraindications and/or warnings concerning the use of such therapeutic products.

“Alkyl” is C₁-C₁₈ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃) CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃) CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃) CH(CH₃)₂), 3-methyl-1-butyl (—CH₂CH₂CH(CH₃)₂), 2-methyl-1-butyl (—CH₂CH(CH₃) CH₂CH₃), 1-hexyl (—CH₂CH₂CH₂CH₂CH₂CH₃), 2-hexyl (—CH(CH₃) CH₂CH₂CH₂CH₃), 3-hexyl (—CH(CH₂CH₃) (CH₂CH₂CH₃)), 2-methyl-2-pentyl (—C(CH₃)₂CH₂CH₂CH₃), 3-methyl-2-pentyl (—CH(CH₃) CH(CH₃) CH₂CH₃), 4-methyl-2-pentyl (—CH(CH₃) CH₂CH(CH₃)₂), 3-methyl-3-pentyl (—C(CH₃) (CH₂CH₃)₂), 2-methyl-3-pentyl (—CH(CH₂CH₃) CH(CH₃)₂), 2,3-dimethyl-2-butyl (—C(CH₃)₂— CH(CH₃)₂), 3,3-dimethyl-2-butyl (—CH(CH₃) C(CH₃) 3.

The term “C₁-C₈ alkyl,” as used herein refers to a straight chain or branched, saturated or unsaturated hydrocarbon having from 1 to 8 carbon atoms. Representative “C₁-C₈ alkyl” groups include, but are not limited to, -methyl, -ethyl, -n-propyl, -n-butyl, -n-pentyl, -n-hexyl, -n-heptyl, -n-octyl, -n-nonyl and -n-decyl; while branched C₁-C₈ alkyls include, but are not limited to, -isopropyl, -sec-butyl, -isobutyl, -tert-butyl, -isopentyl, 2-methylbutyl, unsaturated C₁-C₈ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, -3-methyl-1 butynyl. A C₁-C₈ alkyl group can be unsubstituted or substituted with one or more groups including, but not limited to, —C₁-C₈ alkyl, —O—(C₁-C₈ alkyl), -aryl, —C(O)R′, —OC (O) R″, —C(O) OR″, —C(O) NH₂, —C(O) NHR″, —C(O) N(R′)₂—NHC (O)R′, —SO₃R″, —S(O)₂R′, —S(O)R′, —OH, -halogen, —N₃, —NH₂, —NH (R′), —N(R)₂ and —CN; where each R′ is independently selected from H, —C₁-C₈ alkyl and aryl.

The term “C₁-C₁₂ alkyl,” as used herein refers to a straight chain or branched, saturated or unsaturated hydrocarbon having from 1 to 12 carbon atoms. A C₁-C₁₂ alkyl group can be unsubstituted or substituted with one or more groups including, but not limited to, —C₁-C₈ alkyl, —O—(C₁-C₈ alkyl), -aryl, —C(O) R″, —OC (O)R′, —C(O) OR″, —C(O) NH₂, —C(O) NHR″, —C(O) N(R′)₂—NHC (O)R′, —SO₃R′, —S(O)₂R′, —S(O)R′, —OH, -halogen, —N₃, —NH₂, —NH (R′), —N(R)₂ and —CN; where each R′ is independently selected from H, —C₁-C₈ alkyl and aryl.

The term “C₁-C₆ alkyl,” as used herein refers to a straight chain or branched, saturated or unsaturated hydrocarbon having from 1 to 6 carbon atoms. Representative “C₁-C₆ alkyl” groups include, but are not limited to, -methyl, -ethyl, -n-propyl, -n-butyl, -n-pentyl, —and n-hexyl; while branched C₁-C₆ alkyls include, but are not limited to, -isopropyl, -sec-butyl, -isobutyl, -tert-butyl, -isopentyl, and 2-methylbutyl; unsaturated C₁-C₆ alkyls include, but are not limited to, -vinyl, -allyl, -1-butenyl, -2-butenyl, and -isobutylenyl, -1-pentenyl, -2-pentenyl, -3-methyl-1-butenyl, -2-methyl-2-butenyl, -2,3-dimethyl-2-butenyl, 1-hexyl, 2-hexyl, and 3-hexyl. A C₁-C₆ alkyl group can be unsubstituted or substituted with one or more groups, as described above for C₁-C₈ alkyl group.

The term “C₁-C₄ alkyl,” as used herein refers to a straight chain or branched, saturated or unsaturated hydrocarbon having from 1 to 4 carbon atoms. Representative “C₁-C₄ alkyl” groups include, but are not limited to, -methyl, -ethyl, -n-propyl, -n-butyl; while branched C₁-C₄ alkyls include, but are not limited to, -isopropyl, -sec-butyl, -isobutyl, -tert-butyl; unsaturated C₁-C₄ alkyls include, but are not limited to, -vinyl, -allyl, -1-butenyl, -2-butenyl, and -isobutylenyl. A C₁-C₄ alkyl group can be unsubstituted or substituted with one or more groups, as described above for C₁-C₈ alkyl group.

“Alkoxy” is an alkyl group singly bonded to an oxygen. Exemplary alkoxy groups include, but are not limited to, methoxy (—OCH₃) and ethoxy (—OCH₂CH₃). A “C₁-C₅ alkoxy” is an alkoxy group with 1 to 5 carbon atoms. Alkoxy groups may can be unsubstituted or substituted with one or more groups, as described above for alkyl groups.

“Alkenyl” is C₂-C₁₈ hydrocarbon containing normal, secondary, tertiary or cyclic carbon atoms with at least one site of unsaturation, i.e. a carbon-carbon, sp² double bond. Examples include, but are not limited to: ethylene or vinyl (—CH—CH₂), allyl (—CH₂CH═CH₂), cyclopentenyl (—C₅H₇), and 5-hexenyl (—CH₂CH₂CH₂CH₂CH═CH₂). A “C₂-C₈ alkenyl” is a hydrocarbon containing 2 to 8 normal, secondary, tertiary or cyclic carbon atoms with at least one site of unsaturation, i.e. a carbon-carbon, sp² double bond.

“Alkynyl” is C₂-C₁₈ hydrocarbon containing normal, secondary, tertiary or cyclic carbon atoms with at least one site of unsaturation, i.e. a carbon-carbon, sp triple bond. Examples include, but are not limited to: acetylenic (—C≡CH) and propargyl (—CH₂C≡CH). A “C₂-C₈ alkynyl” is a hydrocarbon containing 2 to 8 normal, secondary, tertiary or cyclic carbon atoms with at least one site of unsaturation, i.e. a carbon-carbon, sp triple bond.

“Alkylene” refers to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—) 1,2-ethyl (—CH₂CH₂—), 1,3-propyl (—CH₂CH₂CH₂—), 1,4-butyl (—CH₂CH₂CH₂CH₂—), and the like.

A “C₁-C₁₀ alkylene” is a straight chain, saturated hydrocarbon group of the formula-(CH₂)₁₋₁₀₋. Examples of a C₁-C₁₀ alkylene include methylene, ethylene, propylene, butylene, pentylene, hexylene, heptylene, octylene, nonylene and decalene.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to: acetylene (—C≡C—), propargyl (—CH₂C≡C—), and 4-pentynyl (—CH₂CH₂CH₂C≡C—).

“Aryl” refers to a carbocyclic aromatic group. Examples of aryl groups include, but are not limited to, phenyl, naphthyl and anthracenyl. A carbocyclic aromatic group or a heterocyclic aromatic group can be unsubstituted or substituted with one or more groups including, but not limited to, —C₁-C₈ alkyl, —O—(C₁-C₈ alkyl), -aryl, —C(O)R′, —OC (O)R′, —C(O) OR″, —C(O) NH₂, —C(O) NHR′, —C(O) N(R′)₂—NHC (O)R′, —S(O)₂R′, —S(O)R′, —OH, -halogen, —N₃, —NH₂, —NH (R′), —N(R′)₂ and —CN; wherein each R′ is independently selected from H, —C₁-C₈ alkyl and aryl.

A “C₅-C₂₀ aryl” is an aryl group with 5 to 20 carbon atoms in the carbocyclic aromatic rings. Examples of C₅-C₂₀ aryl groups include, but are not limited to, phenyl, naphthyl and anthracenyl. A C₅-C₂₀ aryl group can be substituted or unsubstituted as described above for aryl groups. A “C₅-C₁₄ aryl” is an aryl group with 5 to 14 carbon atoms in the carbocyclic aromatic rings. Examples of C₅-C₁₄ aryl groups include, but are not limited to, phenyl, naphthyl and anthracenyl. A C₅-C₁₄ aryl group can be substituted or unsubstituted as described above for aryl groups.

An “arylene” is an aryl group which has two covalent bonds and can be in the ortho, meta, or para configurations as shown in the following structures:

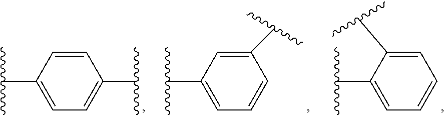

in which the phenyl group can be unsubstituted or substituted with up to four groups including, but not limited to, —C₁-C₈ alkyl, —O—(C₁-C₈ alkyl), -aryl, —C(O)R′, —OC (O) R″, —C(O) OR″, —C(O) NH₂, —C(O) NHR″, —C(O) N(R′)₂—NHC (O)R′, —S(O)₂R′, —S(O) R″, —OH, -halogen, —N₃, —NH₂, —NH (R′), —N(R′)₂ and —CN; wherein each R′ is independently selected from H, —C₁-C₈ alkyl and aryl.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g., the alkyl moiety, including alkanyl, alkenyl or alkynyl groups, of the arylalkyl group is 1 to 6 carbon atoms and the aryl moiety is 5 to 14 carbon atoms.

“Heteroarylalkyl” refers to an acyclic alkyl radical in which one of the hydrogen atoms bonded to a carbon atom, typically a terminal or sp³ carbon atom, is replaced with a heteroaryl radical. Typical heteroarylalkyl groups include, but are not limited to, 2-benzimidazolylmethyl, 2-furylethyl, and the like. The heteroarylalkyl group comprises 6 to 20 carbon atoms, e.g., the alkyl moiety, including alkanyl, alkenyl or alkynyl groups, of the heteroarylalkyl group is 1 to 6 carbon atoms and the heteroaryl moiety is 5 to 14 carbon atoms and 1 to 3 heteroatoms selected from N, O, P, and S. The heteroaryl moiety of the heteroarylalkyl group may be a monocycle having 3 to 7 ring members (2 to 6 carbon atoms or a bicycle having 7 to 10 ring members (4 to 9 carbon atoms and 1 to 3 heteroatoms selected from N, O, P, and S), for example: a bicyclo[4,5], [5,5], [5,6], or [6,6] system.

“Substituted alkyl,” “substituted aryl,” and “substituted arylalkyl” mean alkyl, aryl, and arylalkyl respectively, in which one or more hydrogen atoms are each independently replaced with a substituent. Typical substituents include, but are not limited to, —X, —R, —O—, —OR, —SR, —S⁻, —NR₂, —NR₃, ═NR, —CX₃, —CN, —OCN, —SCN, —N═C═O, —NCS, —NO, —NO₂, ═N₂, —N₃, —NC(═O)R, —C(═O)R, —C(═O) NR₂, —SO₃, —SO₃H, —S(═O)₂R, —OS(═O)₂OR, —S(═O)₂NR, —S(═O)R, —OP(═O) (OR)₂, —P(═O) (OR)₂, —PO₃, —PO₃H₂, —C(═O)R, —C(═O) X, —C(═S)R, —CO₂R, —CO₂, —C(═S) OR, —C(═O) SR, —C(═S) SR, —C(═O) NR₂, —C(═S) NR₂, —C(═NR) NR₂, where each X is independently a halogen: F, Cl, Br, or I; and each R is independently —H, C₂-C₁₈ alkyl, C₆-C₂₀ aryl, C₃-C₁₄ heterocycle, protecting group or prodrug moiety. Alkylene, alkenylene, and alkynylene groups as described above may also be similarly substituted.

“Heteroaryl” and “heterocycle” refer to a ring system in which one or more ring atoms is a heteroatom, e.g., nitrogen, oxygen, and sulfur. The heterocycle radical comprises 3 to 20 carbon atoms and 1 to 3 heteroatoms selected from N, O, P, and S. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo[4,5], [5,5], [5,6], or [6,6] system.

Exemplary heterocycles are described, e.g., in Paquette, Leo A., “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566.

Examples of heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

A “C₃-C₈ heterocycle” refers to an aromatic or non-aromatic C₃-C₈ carbocycle in which one to four of the ring carbon atoms are independently replaced with a heteroatom from the group consisting of O, S and N. Representative examples of a C₃-C₈ heterocycle include, but are not limited to, benzofuranyl, benzothiophene, indolyl, benzopyrazolyl, coumarinyl, isoquinolinyl, pyrrolyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl and tetrazolyl. A C₃-C₈ heterocycle can be unsubstituted or substituted with up to seven groups including, but not limited to, —C₁-C₈ alkyl, —O—(C₁-C₈ alkyl), -aryl, —C(O)R′, —OC (O)R′, —C(O) OR″, —C(O) NH₂, —C(O) NHR″, —C(O) N(R′)₂—NHC (O) R″, —S(O)₂R′, —S(O)R′, —OH, -halogen, —N₃, —NH₂, —NH (R′), —N(R′)₂ and —CN; wherein each R′ is independently selected from H, —C₁-C₈ alkyl and aryl.

“C₃-C₈ heterocyclo” refers to a C₃-C₈ heterocycle group defined above wherein one of the heterocycle group's hydrogen atoms is replaced with a bond. A C₃-C₈ heterocyclo can be unsubstituted or substituted with up to six groups including, but not limited to, —C₁-C₈ alkyl, —O—(C₁-C₈ alkyl), -aryl, —C(O)R′, —OC (O) R″, —C(O) OR″, —C(O) NH₂, —C(O) NHR″, —C(O) N(R′)₂—NHC (O)R′, —S(O)₂R′, —S(O)R′, —OH, -halogen, —N₃, —NH₂, —NH (R′), —N(R′)₂ and —CN; wherein each R′ is independently selected from H, —C₁-C₈ alkyl and aryl.

A “C₃-C₂₀ heterocycle” refers to an aromatic or non-aromatic C₃-C₈ carbocycle in which one to four of the ring carbon atoms are independently replaced with a heteroatom from the group consisting of O, S and N. A C₃-C₂₀ heterocycle can be unsubstituted or substituted with up to seven groups including, but not limited to, —C₁-C₈ alkyl, —O—(C₁-C₈ alkyl), -aryl, —C(O)R′, —OC (O)R′, —C(O) OR″, —C(O)NH₂, —C(O) NHR′, —C(O) N(R′)₂—NHC (O)R′, —S(O)₂R′, —S(O)R′, —OH, -halogen, —N₃, —NH₂, —NH (R′), —N(R′)₂ and —CN; wherein each R′ is independently selected from H, —C₁-C₈ alkyl and aryl.

“C₃-C₂₀ heterocyclo” refers to a C₃-C₂₀ heterocycle group defined above wherein one of the heterocycle group's hydrogen atoms is replaced with a bond.

“Carbocycle” means a saturated or unsaturated ring having 3 to 7 carbon atoms as a monocycle or 7 to 12 carbon atoms as a bicycle. Monocyclic carbocycles have 3 to 6 ring atoms, still more typically 5 or 6 ring atoms. Bicyclic carbocycles have 7 to 12 ring atoms, e.g., arranged as a bicyclo[4,5], [5,5], [5,6] or [6,6] system, or 9 or 10 ring atoms arranged as a bicyclo[5,6] or [6,6] system. Examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cycloheptyl, and cyclooctyl.

A “C₃-C₈ carbocycle” is a 3-, 4-, 5-, 6-, 7- or 8-membered saturated or unsaturated non-aromatic carbocyclic ring. Representative C₃-C₈ carbocycles include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. A C₃-C₈ carbocycle group can be unsubstituted or substituted with one or more groups including, but not limited to, —C₁-C₈ alkyl, —O—(C₁-C₈ alkyl), -aryl, —C(O)R′, —OC (O) R″, —C(O) OR″, —C(O)NH₂, —C(O) NHR″, —C(O) N(R′)₂—NHC (O) R″, —S(O)₂R′, —S(O)R′, —OH, -halogen, —N₃, —NH₂, —NH (R′), —N(R′)₂ and —CN; where each R′ is independently selected from H, —C₁-C₈ alkyl and aryl.

A “C₃-C₈ carbocyclo” refers to a C₃-C₈ carbocycle group defined above wherein one of the carbocycle groups' hydrogen atoms is replaced with a bond.

“Linker” refers to a chemical moiety comprising a covalent bond or a chain of atoms that covalently attaches an antibody to a drug moiety. In various embodiments, linkers include a divalent radical such as an alkyldiyl, an aryldiyl, a heteroaryldiyl, moieties such as: —(CR₂)ₙO(CR₂)ₙ—, repeating units of alkyloxy (e.g., polyethylenoxy, PEG, polymethyleneoxy) and alkylamino (e.g., polyethyleneamino, Jeffamine™); and diacid ester and amides including succinate, succinamide, diglycolate, malonate, and caproamide. In various embodiments, linkers can comprise one or more amino acid residues, such as valine, phenylalanine, lysine, and homolysine.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

“Leaving group” refers to a functional group that can be substituted by another functional group. Certain leaving groups are well known in the art, and examples include, but are not limited to, a halide (e.g., chloride, bromide, iodide), methanesulfonyl (mesyl), p-toluenesulfonyl (tosyl), trifluoromethylsulfonyl (triflate), and trifluoromethylsulfonate.

The term “protecting group” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include, but are not limited to, acetyl, trifluoroacetyl, t-butoxycarbonyl (BOC), benzyloxycarbonyl (CBZ) and 9-fluorenylmethylenoxycarbonyl (Fmoc). For a general description of protecting groups and their use, see T. W. Greene, Protective Groups in Organic Synthesis, John Wiley & Sons, New York, 1991, or a later edition.

### III. Methods

Provided herein are methods of treating a B-cell proliferative disorder (such as diffuse large B-cell lymphoma (DLBCL)) in an individual (a human patient) in need thereof comprising administering to the individual an effective amount of: (a) an immunoconjugate comprising an antibody which binds CD79b linked to a cytotoxic agent, and (b) at least one additional therapeutic agent. In some embodiments, the at least one additional therapeutic agent is a chemotherapeutic agent. In some embodiments, the at least one additional therapeutic agent is a cytotoxic agent. In some embodiments, the at least one additional therapeutic agent is an anti-CD20 agent, such as an anti-CD20 antibody. In some embodiments, the methods comprise administering to the individual an effective amount of: (a) an immunoconjugate comprising an anti-CD79b antibody linked to a cytotoxic agent (i.e., anti-CD79b immunoconjugate), (b) an anti-CD20 antibody, (c) one or more chemotherapeutic agents, and (d) a corticosteroid.

In some embodiments, provided herein are methods of treating diffuse large B-cell lymphoma (DLBCL) in an individual (a human patient) in need thereof comprising administering to the individual an effective amount of: (a) an immunoconjugate comprising the formula:

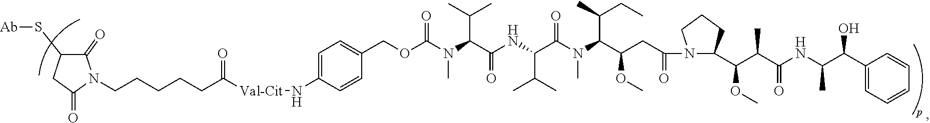

wherein Ab is an anti-CD79b antibody comprising (i) an HVR-H₁ that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8; (b) an anti-CD20 antibody; (c) one or more chemotherapeutic agents; and (d) a corticosteroid.

In some embodiments, the immunoconjugate comprises an anti-CD79b antibody that comprises a heavy chain variable domain (VH) comprising the amino acid sequence of SEQ ID NO: 19 and a light chain variable domain (VL) comprising the amino acid sequence of SEQ ID NO: 20. In some embodiments, the immunoconjugate comprises an anti-CD79b antibody that comprises a heavy chain comprising the amino acid sequence of SEQ ID NO: 37 and a light chain comprising the amino acid sequence of SEQ ID NO: 35. In some embodiments, the immunoconjugate comprises an anti-CD79b antibody that comprises a heavy chain comprising the amino acid sequence of SEQ ID NO: 36 and a light chain comprising the amino acid sequence of SEQ ID NO: 38. In some embodiments, the immunoconjugate comprises an anti-CD79b antibody that comprises a heavy chain comprising the amino acid sequence of SEQ ID NO: 36 and a light chain comprising the amino acid sequence of SEQ ID NO: 35. In some embodiments, p is between 2 and 7, between 2 and 6, between 2 and 5, between 3 and 5, or between 3 and 4. In some embodiments, p is 3.4. In some embodiments, p is 3.5. In some embodiments, the immunoconjugate is polatuzumab vedotin (or huMA 79bv28-MC-vc-PAB-MMAE, or CAS Registry Number 1313206-42-6, e.g., as described in U.S. Pat. No. 8,545,850, which is incorporated herein by reference in its entirety).

In some embodiments, the anti-CD20 antibody is a humanized B-Ly1 antibody. In some embodiments, the humanized B-Ly1 antibody is obinutuzumab. In some embodiments, the anti-CD20 antibody is rituximab. In some embodiments, the anti-CD20 antibody is ofatumumab, ublituximab, and/or ibritumomab tiuxetan.

In some embodiments, the one or more chemotherapeutic agents comprise cyclophosphamide and/or doxorubicin. In some embodiments, the one or more chemotherapeutic agents comprise cyclophosphamide and doxorubicin.

In some embodiments, the corticosteroid is prednisone, prednisolone, or methylprednisolone. In some embodiments, the corticosteroid is not hydrocortisone.

In some embodiments, therapeutic or clinical responses in a human patient or in a plurality of human patients treated according to the methods of the disclosure are compared to a control treatment. In some embodiments, the control treatment comprises rituximab, cyclophosphamide, doxorubicin, vincristine, and prednisone (R-CHOP). In some embodiments, the prednisone may be substituted with prednisolone, or methylprednisolone. Thus, in some embodiments, R-CHOP refers to rituximab, cyclophosphamide, doxorubicin, vincristine, and prednisone; in some embodiments, R-CHOP refers to rituximab, cyclophosphamide, doxorubicin, vincristine, and prednisolone; and in some embodiments, R-CHOP refers to rituximab, cyclophosphamide, doxorubicin, vincristine, and methylprednisolone.

In some embodiments, administration of the immunoconjugate (e.g., huMA79bv28-MC-vc-PAB-MMAE or polatuzumab vedotin), anti-CD20 antibody (e.g., obinutuzumab or rituximab), one or more chemotherapeutic agents (e.g., cyclophosphamide and doxorubicin), and corticosteroid (e.g., prednisone, prednisolone, or methylprednisolone) to a plurality of human patients results in one or more of: (a) an improvement in progression-free survival (PFS) of the plurality of human patients as compared to a reference PFS, wherein the reference PFS is the PFS of a plurality of human patients who have received a control treatment; (b) at least a 25% (e.g., 26%, 27%, 28%, 29%, 30%) reduction in the risk of disease progression, relapse, or death in the plurality of human patients as compared to a control treatment; (c) a hazard ratio of no more than 0.75 (e.g., 0.74, 0.73, 0.72, 0.71, 0.70) in PFS of the plurality of human patients as compared to a control treatment; (d) a hazard ratio of no more than 0.78 (e.g., 0.77, 0.76, 0.75, 0.74, 0.73, 0.72, 0.71, 0.70) in PFS of the plurality of human patients as compared to a control treatment; (e) a hazard ratio of no more than 0.79 (e.g., 0.78, 0.77, 0.76, 0.75, 0.74, 0.73, 0.72, 0.71, 0.70) in PFS of the plurality of human patients as compared to a control treatment; (f) a 24-month progression-free survival rate (PFS24) of at least 75% (e.g., 76%, 77%, 78%, 79%, 80%) in the plurality of human patients; (g) an improvement in a 24-month progression-free survival rate (PFS24) of the plurality of human patients as compared to a reference PFS24, wherein the reference PFS24 is the 24-month progression-free survival rate of a plurality of human patients who have received a control treatment: (h) an improvement in a PFS24 of the plurality of human patients of at least about 6% (e.g., 7%, 8%, 9%, or 10%), as compared to a reference PFS24, wherein the reference PFS24 is the 24-month progression-free survival rate of a plurality of human patients who have received a control treatment; (i) an improvement in a PFS36 of the plurality of human patients of at least about 6% (e.g., 7%, 8%, 9%, or 10%), compared to a reference PFS36, wherein the reference PFS36 is the 36-month progression-free survival rate of a plurality of human patients who have received a control treatment; (j) an improvement in overall survival (OS) of the plurality of human patients as compared to a reference OS, wherein the reference OS is the OS of a plurality of human patients who have received a control treatment: (k) an improvement in event-free survival-efficacy (EFS_{eff}) of the plurality of human patients as compared to a reference EFS_{eff}, wherein the reference EFS_{eff} is the EFS_{eff} of a plurality of human patients who have received a control treatment; (1) an improvement in a 24-month event-free survival-efficacy rate (EFS24) of the plurality of human patients as compared to a reference EFS24, wherein the EFS24 is improved by at least about 5% (e.g., 6%, 7%, 8%, 9%, or 10%); (m) a hazard ratio of no more than 0.77 (e.g., 0.76, 0.75, 0.74, 0.73, 0.72, 0.71, 0.70) in EFS_{eff} in the plurality of human patients as compared to a control treatment; (n) a hazard ratio of no more than 0.81 (e.g., 0.80, 0.79, 0.78, 0.77, 0.76, 0.75, 0.74, 0.73, 0.72, 0.71, 0.70) in EFS_{eff} in the plurality of human patients as compared to a control treatment; (o) an improvement in a 24-month event-free survival rate (EFS24) of the plurality of human patients as compared to a reference EFS24, wherein the reference EFS24 is the 24-month event-free survival rate of a plurality of human patients who have received a control treatment, wherein the improvement is at least about 5% (e.g., 6%, 7%, 8%, 9%, or 10%); (p) a 24-month event-free survival rate of the plurality of human patients of at least about 75% (e.g., 76%, 77%, 78%, 79%, or 80%); (q) a rate of complete response (CR) at end of treatment (EOT) in the plurality of human patients of at least about 77% (e.g., 78%, 79%, or 80%), wherein the rate of CR is assessed by positron emission tomography-computed tomography (PET-CT); (r) an objective response rate (ORR) at EOT in the plurality of human patients of at least about 85% (e.g., 86%, 87%, 88%, 89%, or 90%), wherein the ORR is assessed by PET-CT; (s) an improvement in a 36-month event-free survival rate (EFS36) of the plurality of human patients as compared to a reference EFS36, wherein the reference EFS36 is the 36-month event-free survival rate of a plurality of human patients who have received a control treatment, wherein the improvement is at least about 6% (e.g., 6.5%, 7%, 8%, 9%, or 10%); (t) a 36-month event-free survival rate of the plurality of human patients of at least about 70% (e.g., 71%, 72%, 73%, 74%, 75%, 76%, 77%, 78%, 79%, or 80%); (u) an improvement in a 42-month event-free survival rate (EFS42) of the plurality of human patients as compared to a reference EFS42, wherein the reference EFS42 is the 42-month event-free survival rate of a plurality of human patients who have received a control treatment, wherein the improvement is at least about 5% (e.g., 6%, 7%, 8%, 9%, or 10%); (v) a 42-month event-free survival rate of the plurality of human patients of at least about 68% (e.g., 68.5%, 69%, 70%, 71%, 72%, 73%, 74%, 75%, 76%, 77%, 78%, 79%, or 80%); (w) an improvement in a 42-month progression-free survival rate (PFS42) of the plurality of human patients of at least about 6% (e.g., 7%, 8%, 9%, or 10%), compared to a reference PFS42, wherein the reference PFS42 is the 42-month progression-free survival rate of a plurality of human patients who have received a control treatment; (x) at least a 20% (e.g., 21%, 22%, 23%, or 24%) reduction in the risk of disease progression, relapse, or death in the plurality of human patients as compared to a control treatment; or (y) an objective response rate (ORR) at EOT in the plurality of human patients of at least about 84% (e.g., 85%, 86%, 87%, 88%, 89%, or 90%), wherein the ORR is assessed by PET-CT. In some embodiments, the control treatment comprises rituximab, cyclophosphamide, doxorubicin, vincristine, and prednisone, prednisolone, or methylprednisolone, in the absence of polatuzumab vedotin (R-CHOP).

In some embodiments, administration of the immunoconjugate (e.g., huMA79bv28-MC-vc-PAB-MMAE or polatuzumab vedotin), anti-CD20 antibody (e.g., obinutuzumab or rituximab), one or more chemotherapeutic agents (e.g., cyclophosphamide and doxorubicin), and corticosteroid (e.g., prednisone, prednisolone, or methylprednisolone) to a human patient prolongs the duration of progression-free survival (PFS) or overall survival (OS) in such patient.

Additional details regarding dosing and administration of the immunoconjugate, anti-CD20 antibody, one or more chemotherapeutic agents, and corticosteroid, treatment regimens, and responses to treatment for DLBCL, e.g., progression-free survival, event-free survival, overall survival, complete response, overall response, and other therapeutic responses, are provided herein below.

### A. Dosing and Administration

Anti-CD79b immunoconjugates and additional therapeutic agents (e.g., an anti-CD20 antibody, one or more chemotherapeutic agents, and a corticosteroid) provided herein for use in any of the therapeutic methods described herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The immunoconjugate need not be, but is optionally formulated with one or more agents currently used to prevent or treat the disorder in question. The amount of the anti-CD79b immunoconjugate and the additional therapeutic agents (e.g., an anti-CD20 antibody, one or more chemotherapeutic agents, and a corticosteroid), and the timing of co-administration will depend on the type (species, gender, age, weight, etc.) and condition of the patient being treated and the severity of the disease or condition being treated. The anti-CD79b immunoconjugate and the additional therapeutic agents (e.g., an anti-CD20 antibody, one or more chemotherapeutic agents, and a corticosteroid) are suitably co-administered to the patient at one time or over a series of treatments, e.g., according to any of the treatment regimens described below.

In some embodiments, the dosage of the anti-CD79b immunoconjugate (such as huMA79bv28-MC-vc-PAB-MMAE or polatuzumab vedotin) is between 1.0-1.8 mg/kg. In some embodiments of any of the methods, the dosage of anti-CD79b immunoconjugate is about any of 1.0, 1.4, 1.5, 1.6, 1.7, or 1.8 mg/kg. In some embodiments, the dosage of anti-CD79b immunoconjugate is about 1.0 mg/kg. In some embodiments, the dosage of anti-CD79b immunoconjugate is about 1.4 mg/kg. In some embodiments, the dosage of anti-CD79b immunoconjugate is about 1.8 mg/kg. In some embodiments of any of the methods, the anti-CD79b immunoconjugate is administered q3w (i.e., once every 3 weeks). In some embodiments of any of the methods, the anti-CD79b immunoconjugate is administered once every 21 days. In some embodiments, the anti-CD79b immunoconjugate is administered via intravenous infusion. In some embodiments, the dosage administered via infusion is in the range of about 1 mg to about 1,500 mg per dose. Alternatively, the dosage range is of about 1 mg to about 1,500 mg, about 1 mg to about 1,000 mg, about 400 mg to about 1200 mg, about 600 mg to about 1000 mg, about 10 mg to about 500 mg, about 10 mg to about 300 mg, about 10 mg to about 200 mg, and about 1 mg to about 200 mg. In some embodiments, the dosage administered via infusion is in the range of about 1 μg/m² to about 10,000 μg/m² per dose. Alternatively, the dosage range is of about 1 μg/m² to about 1000 μg/m², about 1 μg/m² to about 800 μg/m², about 1 μg/m² to about 600 μg/m², about 1 μg/m² to about 400 μg/m², about 10 μg/m² to about 500 μg/m², about 10 μg/m² to about 300 μg/m², about 10 μg/m² to about 200 g/m², and about 1 μg/m² to about 200 μg/m². The dose may be administered once per day, once per week, multiple times per week, but less than once per day, multiple times per month but less than once per day, multiple times per month but less than once per week, once per month, once every 21 days, or intermittently to relieve or alleviate symptoms of the disease. In some embodiments, the dosage of the immunoconjugate is 1.8 mg/kg, administered in 21-day cycles. In some embodiments, the dosage of the immunoconjugate is 1.8 mg/kg, administered on day 1 of each 21-day cycle. Administration may continue at any of the disclosed intervals until remission of the tumor or symptoms of the B-cell proliferative disorder being treated. Administration may continue after remission or relief of symptoms is achieved where such remission or relief is prolonged by such continued administration.

In some embodiments, the immunoconjugate is polatuzumab vedotin. In some embodiments, the polatuzumab vedotin is administered at a dose of about 1.0-1.8 mg/kg (e.g., 1.0 mg/kg, 1.4 mg/kg, or 1.8 mg/kg). In some embodiments, the polatuzumab vedotin is administered at a dose of about 1.0 mg/kg. In some embodiments, the polatuzumab vedotin is administered at a dose of about 1.4 mg/kg. In some embodiments, the polatuzumab vedotin is administered at a dose of about 1.8 mg/kg. In some embodiments, the polatuzumab vedotin is administered intravenously. In some embodiments, the polatuzumab vedotin is administered in 21-day cycles. In some embodiments, the polatuzumab vedotin is administered on day 1 of each 21-day cycle. In some embodiments, the polatuzumab vedotin is administered for between one and six 21-day cycles, e.g., any of 1, 2, 3, 4, 5, or 6 21-day cycles. In some embodiments, the polatuzumab vedotin is administered for at least six 21-day cycles. In some embodiments, the polatuzumab vedotin is administered for six 21-day cycles. In some embodiments, the polatuzumab vedotin is administered at a dose of about 1.0-1.8 mg/kg (e.g., 1.0 mg/kg, 1.4 mg/kg, or 1.8 mg/kg) on day 1 of each 21-day cycle for at least six cycles. In some embodiments, the polatuzumab vedotin is administered at a dose of about 1.0-1.8 mg/kg (e.g., 1.0 mg/kg, 1.4 mg/kg, or 1.8 mg/kg) on day 1 of each 21-day cycle for six cycles.

In some embodiments, the dosage of the anti-CD20 agent (e.g., an anti-CD20 antibody, such as rituximab or obinutuzumab) is between about 300-1600 mg/m² and/or 300-2000 mg. In some embodiments, the dosage of the anti-CD20 antibody is about any of 300, 375, 600, 1000, or 1250 mg/m² and/or 300, 1000, or 2000 mg. In some embodiments, the anti-CD20 antibody is rituximab and the dosage administered is 375 mg/m². In some embodiments, the anti-CD20 antibody is obinutuzumab and the dosage administered is 1000 mg. In some embodiments, the anti-CD20 antibody is administered q3w (i.e., every 3 weeks). In some embodiments, the anti-CD20 antibody is administered once every 21 days. In some embodiments, the dosage of an afucosylated anti-CD20 antibody (preferably the afucosylated humanized B-Ly1 antibody) may be 800 to 1600 mg (in one embodiment 800 to 1200 mg, such as 1000 mg). In some embodiments, the dose is a flat 1000 mg dose. In some embodiments, the dosage of rituximab is 375 mg/m², administered on day 1 of each 21-day cycle. In some embodiments, the anti-CD20 antibody is administered via intravenous infusion.

In some embodiments, the anti-CD20 antibody is rituximab. In some embodiments, the rituximab is administered at a dose of about 375 mg/m². In some embodiments, the rituximab is administered intravenously. In some embodiments, the rituximab is administered in 21-day cycles. In some embodiments, the rituximab is administered on day 1 of each 21-day cycle. In some embodiments, the rituximab is administered for between one and eight 21-day cycles, e.g., any of 1, 2, 3, 4, 5, 6, 7, or 8 21-day cycles. In some embodiments, the rituximab is administered for between six and eight 21-day cycles, e.g., any of 6, 7, or 8 21-day cycles. In some embodiments, the rituximab is administered for at least six 21-day cycles. In some embodiments, the rituximab is administered for six 21-day cycles. In some embodiments, the rituximab is administered for seven 21-day cycles. In some embodiments, the rituximab is administered for eight 21-day cycles. In some embodiments, the rituximab is administered for up to eight 21-day cycles. In some embodiments, the rituximab is administered at a dose of about 375 mg/m² on day 1 of each 21-day cycle for at least six cycles. In some embodiments, the rituximab is administered at a dose of about 375 mg/m² on day 1 of each 21-day cycle for six cycles, seven cycles, or eight cycles.

In some embodiments, the one or more chemotherapeutic agents comprise cyclophosphamide. In some embodiments, the dosage of cyclophosphamide is between about 375 mg/m² and about 750 mg/m², between about 375 mg/m² and about 563 mg/m², or between about 563 mg/m² and about 750 mg/m². In some embodiments, the dosage of cyclophosphamide is about 375 mg/m². In some embodiments, the dosage of cyclophosphamide is about 563 mg/m². In some embodiments, the dosage of cyclophosphamide is about 750 mg/m². In some embodiments, the cyclophosphamide is administered q3w (i.e., every 3 weeks). In some embodiments, the cyclophosphamide is administered once every 21 days. In some embodiments, the cyclophosphamide is administered on day 1 of each 21-day cycle. In some embodiments, cyclophosphamide is administered via intravenous infusion. In some embodiments, the cyclophosphamide is administered for between one and eight 21-day cycles, e.g., any of 1, 2, 3, 4, 5, 6, 7, or 8 21-day cycles. In some embodiments, the cyclophosphamide is administered for between six and eight 21-day cycles, e.g., any of 6, 7, or 8 21-day cycles. In some embodiments, the cyclophosphamide is administered for at least six 21-day cycles. In some embodiments, the cyclophosphamide is administered for six 21-day cycles. In some embodiments, the cyclophosphamide is administered for seven 21-day cycles. In some embodiments, the cyclophosphamide is administered for eight 21-day cycles. In some embodiments, the cyclophosphamide is administered for up to eight 21-day cycles. In some embodiments, the cyclophosphamide is administered at a dose of between about 375 mg/m² and about 750 mg/m² (e.g., 375 mg/m², 562.5 mg/m², or 750 mg/m²) on day 1 of each 21-day cycle for at least six cycles. In some embodiments, the cyclophosphamide is administered at a dose of between about 375 mg/m² and about 750 mg/m² (e.g., 375 mg/m², 562.5 mg/m², or 750 mg/m²) on day 1 of each 21-day cycle for six cycles, seven cycles, or eight cycles.

In some embodiments, the one or more chemotherapeutic agents comprise doxorubicin. In some embodiments, the dosage of the doxorubicin is between about 25 mg/m² and about 50 mg/m², between about 25 mg/m² and about 38 mg/m², or between about 38 mg/m² and about 50 mg/m². In some embodiments, the dosage of the doxorubicin is about 25 mg/m². In some embodiments, the dosage of the doxorubicin is about 38 mg/m². In some embodiments, the dosage of the doxorubicin is about 50 mg/m². In some embodiments, the doxorubicin is administered q3w (i.e., every 3 weeks). In some embodiments, doxorubicin is administered once every 21 days. In some embodiments, the doxorubicin is administered on day 1 of each 21-day cycle. In some embodiments, doxorubicin is administered via intravenous infusion. In some embodiments, the doxorubicin is administered for between one and eight 21-day cycles, e.g., any of 1, 2, 3, 4, 5, 6, 7, or 8 21-day cycles. In some embodiments, the doxorubicin is administered for between six and eight 21-day cycles, e.g., any of 6, 7, or 8 21-day cycles. In some embodiments, the doxorubicin is administered for at least six 21-day cycles. In some embodiments, the doxorubicin is administered for six 21-day cycles. In some embodiments, the doxorubicin is administered for seven 21-day cycles. In some embodiments, the doxorubicin is administered for eight 21-day cycles. In some embodiments, the doxorubicin is administered for up to eight 21-day cycles. In some embodiments, the doxorubicin is administered at a dose of between about 25 mg/m² and about 50 mg/m² (e.g., 25 mg/m², 37.5 mg/m², or 50 mg/m²) on day 1 of each 21-day cycle for at least six cycles. In some embodiments, the doxorubicin is administered at a dose of between about 25 mg/m² and about 50 mg/m² (e.g., 25 mg/m², 37.5 mg/m², or 50 mg/m²) on day 1 of each 21-day cycle for six cycles, seven cycles, or eight cycles.

In some embodiments, the dosage of the corticosteroid is between about 50 mg and about 100 mg, between about 50 mg and about 80 mg, or between about 80 mg and about 100 mg. In some embodiments, the dosage of the corticosteroid is about 50 mg. In some embodiments, the dosage of the corticosteroid is about 80 mg. In some embodiments, the dosage of the corticosteroid is about 100 mg. In some embodiments, the corticosteroid is administered in 21-day cycles. In some embodiments, the corticosteroid is administered on days 1, 2, 3, 4, and 5 of each 21-day cycle. In some embodiments, the corticosteroid is administered via intravenous infusion or orally. In some embodiments, the corticosteroid is prednisone. In some embodiments, the dosage of prednisone is about 100 mg. In some embodiments, the prednisone is administered on days 1, 2, 3, 4, and 5 of each 21-day cycle at a dose of about 100 mg per day. In some embodiments, the prednisone is administered orally. In some embodiments, the corticosteroid is prednisolone. In some embodiments, the dosage of prednisolone is about 100 mg. In some embodiments, the prednisolone is administered on days 1, 2, 3, 4, and 5 of each 21-day cycle at a dose of about 100 mg per day. In some embodiments, the prednisolone is administered orally. In some embodiments, the corticosteroid is methylprednisolone. In some embodiments, the dosage of methylprednisolone is about 80 mg. In some embodiments, the methylprednisolone is administered on days 1, 2, 3, 4, and 5 of each 21-day cycle at a dose of about 80 mg per day. In some embodiments, the methylprednisolone is administered intravenously. In some embodiments, the corticosteroid (e.g., prednisone, prednisolone, or methylprednisolone) is administered for between one and eight 21-day cycles, e.g., any of 1, 2, 3, 4, 5, 6, 7, or 8 21-day cycles. In some embodiments, the corticosteroid (e.g., prednisone, prednisolone, or methylprednisolone) is administered for between six and eight 21-day cycles, e.g., any of 6, 7, or 8 21-day cycles. In some embodiments, the corticosteroid (e.g., prednisone, prednisolone, or methylprednisolone) is administered for at least six 21-day cycles. In some embodiments, the corticosteroid (e.g., prednisone, prednisolone, or methylprednisolone) is administered for six 21-day cycles. In some embodiments, the corticosteroid (e.g., prednisone, prednisolone, or methylprednisolone) is administered for seven 21-day cycles. In some embodiments, the corticosteroid (e.g., prednisone, prednisolone, or methylprednisolone) is administered for eight 21-day cycles. In some embodiments, the prednisone is administered on days 1, 2, 3, 4, and 5 of each 21-day cycle at a dose of about 100 mg per day for at least six cycles. In some embodiments, the prednisone is administered on days 1, 2, 3, 4, and 5 of each 21-day cycle at a dose of about 100 mg per day for six cycles, seven cycles, or eight cycles. In some embodiments, the prednisolone is administered on days 1, 2, 3, 4, and 5 of each 21-day cycle at a dose of about 100 mg per day for at least six cycles. In some embodiments, the prednisolone is administered on days 1, 2, 3, 4, and 5 of each 21-day cycle at a dose of about 100 mg per day for six cycles, seven cycles, or eight cycles. In some embodiments, the methylprednisolone is administered on days 1, 2, 3, 4, and 5 of each 21-day cycle at a dose of about 80 mg per day for at least six cycles. In some embodiments, the methylprednisolone is administered on days 1, 2, 3, 4, and 5 of each 21-day cycle at a dose of about 80 mg per day for six cycles, seven cycles, or eight cycles.

An exemplary dosing regimen for the combination therapy of anti-CD79b immunoconjugates (such as huMA79bv28-MC-vc-PAB-MMAE or polatuzumab vedotin) and one or more additional therapeutic agents includes the anti-CD79b immunoconjugate (such as huMA79bv28-MC-vc-PAB-MMAE or polatuzumab vedotin) administered at a dose of about 1.0-1.8 mg/kg (e.g., 1.0 mg/kg, 1.4 mg/kg, or 1.8 mg/kg) once every 21 days, e.g., on day 1 of each 21-day cycle; rituximab at a dose of about 375 mg/m² once every 21 days, e.g., on day 1 of each 21-day cycle; cyclophosphamide at a dose of between about 375 mg/m² and about 750 mg/m² (e.g., 375 mg/m², 562.5 mg/m², or 750 mg/m²) once every 21 days, e.g., on day 1 of each 21-day cycle; doxorubicin at a dose of between about 25 mg/m² and about 50 mg/m² (e.g., 25 mg/m², 37.5 mg/m², or 50 mg/m²) once every 21 days, e.g., on day 1 of each 21-day cycle; and a corticosteroid (e.g., prednisone at a dose of about 100 mg, prednisolone at a dose of about 100 mg, or methylprednisolone at a dose of about 80 mg) on days 1-5 of each 21-day cycle. In some embodiments, the anti-CD79b immunoconjugate is administered at a dose of about any of 1.0 mg/kg, 1.4 mg/kg, or 1.8 mg/kg. In some embodiments, the anti-CD79b immunoconjugate is administered at a dose of about 1.0 mg/kg. In some embodiments, the anti-CD79b immunoconjugate is administered at a dose of about 1.4 mg/kg. In some embodiments, the anti-CD79b immunoconjugate is administered at a dose of about 1.8 mg/kg. In some embodiments, the cyclophosphamide is administered at a dose of about 750 mg/m². In some embodiments, the doxorubicin is administered at a dose of about 50 mg/m². In some embodiments, the corticosteroid is prednisone administered at a dose of about 100 mg. In some embodiments, the corticosteroid is prednisolone administered at a dose of about 100 mg. In some embodiments, the corticosteroid is methylprednisolone administered at a dose of about 80 mg.

Another exemplary dosing regimen for the combination therapy of anti-CD79b immunoconjugates (such as huMA79bv28-MC-vc-PAB-MMAE or polatuzumab vedotin) and one or more additional therapeutic agents includes the anti-CD79b immunoconjugate (such as huMA79bv28-MC-vc-PAB-MMAE or polatuzumab vedotin) administered at a dose of about 1.0-1.8 mg/kg (e.g., 1.0 mg/kg, 1.4 mg/kg, or 1.8 mg/kg) once every 21 days, e.g., on day 1 of each 21-day cycle; rituximab at a dose of about 375 mg/m² once every 21 days, e.g., on day 1 of each 21-day cycle; cyclophosphamide once every 21 days, e.g., on day 1 of each 21-day cycle; doxorubicin once every 21 days, e.g., on day 1 of each 21-day cycle; and a corticosteroid (e.g., prednisone, prednisolone, or methylprednisolone) on days 1-5 of each 21-day cycle. In some embodiments, the anti-CD79b immunoconjugate is administered at a dose of about any of 1.0 mg/kg, 1.4 mg/kg, or 1.8 mg/kg.

Another exemplary dosing regimen for the combination therapy of anti-CD79b immunoconjugates (such as huMA79bv28-MC-vc-PAB-MMAE or polatuzumab vedotin) and one or more additional therapeutic agents includes the anti-CD79b immunoconjugate (such as huMA79bv28-MC-vc-PAB-MMAE or polatuzumab vedotin) administered at a dose of about 1.0-1.8 mg/kg (e.g., 1.0 mg/kg, 1.4 mg/kg, or 1.8 mg/kg) once every 21 days, e.g., on day 1 of each 21-day cycle; obinutuzumab at a dose of about 1000 mg once every 21 days, e.g., on day 1 of each 21-day cycle; cyclophosphamide at a dose of between about 375 mg/m² and about 750 mg/m² (e.g., 375 mg/m², 562.5 mg/m², or 750 mg/m²) once every 21 days, e.g., on day 1 of each 21-day cycle; doxorubicin at a dose of between about 25 mg/m² and about 50 mg/m² (e.g., 25 mg/m², 37.5 mg/m², or 50 mg/m²) once every 21 days, e.g., on day 1 of each 21-day cycle; and a corticosteroid (e.g., prednisone at a dose of about 100 mg, prednisolone at a dose of about 100 mg, or methylprednisolone at a dose of about 80 mg) on days 1-5 of each 21-day cycle. In some embodiments, the anti-CD79b immunoconjugate is administered at a dose of about any of 1.0 mg/kg. 1.4 mg/kg, or 1.8 mg/kg. In some embodiments, the anti-CD79b immunoconjugate is administered at a dose of about 1.0 mg/kg. In some embodiments, the anti-CD79b immunoconjugate is administered at a dose of about 1.4 mg/kg. In some embodiments, the anti-CD79b immunoconjugate is administered at a dose of about 1.8 mg/kg. In some embodiments, the cyclophosphamide is administered at a dose of about 750 mg/m². In some embodiments, the doxorubicin is administered at a dose of about 50 mg/m². In some embodiments, the corticosteroid is prednisone administered at a dose of about 100 mg. In some embodiments, the corticosteroid is prednisolone administered at a dose of about 100 mg. In some embodiments, the corticosteroid is methylprednisolone administered at a dose of about 80 mg.

Another exemplary dosing regimen for the combination therapy of anti-CD79b immunoconjugates (such as huMA79bv28-MC-vc-PAB-MMAE or polatuzumab vedotin) and one or more additional therapeutic agents includes the anti-CD79b immunoconjugate (such as huMA79bv28-MC-vc-PAB-MMAE or polatuzumab vedotin) administered at a dose of about 1.0-1.8 mg/kg (e.g., 1.0 mg/kg. 1.4 mg/kg, or 1.8 mg/kg) once every 21 days, e.g., on day 1 of each 21-day cycle; obinutuzumab at a dose of about 1000 mg once every 21 days, e.g., on day 1 of each 21-day cycle; cyclophosphamide once every 21 days, e.g., on day 1 of each 21-day cycle; doxorubicin once every 21 days, e.g., on day 1 of each 21-day cycle; and a corticosteroid (e.g., prednisone, prednisolone, or methylprednisolone) on days 1-5 of each 21-day cycle. In some embodiments, the anti-CD79b immunoconjugate is administered at a dose of about any of 1.0 mg/kg. 1.4 mg/kg, or 1.8 mg/kg.

The terms “co-administration,” “co-administering,” “combination,” or “in combination,” with respect to administration of two or more therapeutic agents, such as the anti-CD79b immunoconjugate and the at least one additional therapeutic agent (e.g., an anti-CD20 antibody, one or more chemotherapeutic agents, and a corticosteroid), refer to the administration of the two or more therapeutic agents as two (or more) separate formulations, or as one single formulation comprising the two or more therapeutic agents. Where separate formulations are used, the co-administration can be simultaneous (i.e., at the same time) or sequential in any order, wherein preferably there is a time period while all active agents simultaneously exert their biological activities. In some embodiments, the two or more therapeutic agents are co-administered either simultaneously or sequentially. In some embodiments, when all therapeutic agents are co-administered sequentially, the dose of each agent is administered either on the same day in two or more separate administrations, or one of the agents is administered on day 1, the other agent(s) are co-administered on subsequent days, e.g., according to any of the treatment regimens described herein.

An immunoconjugate provided herein (and any additional therapeutic agents, e.g., an anti-CD20 antibody, one or more chemotherapeutic agents, and a corticosteroid) for use in any of the therapeutic methods described herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including, but not limited to, single or multiple administrations over various time-points, bolus administration, and pulse infusion are contemplated herein. The anti-CD79b immunoconjugate (e.g., huMA79bv28-MC-vc-PAB-MMAE or polatuzumab vedotin), the anti-CD20 antibody (such as obinutuzumab or rituximab), the one or more chemotherapeutic agents (e.g., cyclophosphamide and/or doxorubicin), and the corticosteroid (e.g., prednisone, prednisolone, or methylprednisolone) may be administered by the same route of administration or by different routes of administration. In some embodiments, the anti-CD79b immunoconjugate is administered intravenously, intramuscularly, subcutaneously, topically, orally, transdermally, intraperitoneally, intraorbitally, by implantation, by inhalation, intrathecally, intraventricularly, or intranasally. In some embodiments, the anti-CD20 antibody (such as obinutuzumab or rituximab) is administered intravenously, intramuscularly, subcutaneously, topically, orally, transdermally, intraperitoneally, intraorbitally, by implantation, by inhalation, intrathecally, intraventricularly, or intranasally. In some embodiments, the one or more chemotherapeutic agents (e.g., cyclophosphamide and/or doxorubicin) are administered intravenously, intramuscularly, subcutaneously, topically, orally, transdermally, intraperitoneally, intraorbitally, by implantation, by inhalation, intrathecally, intraventricularly, or intranasally. In some embodiments, the corticosteroid (e.g., prednisone, prednisolone, or methylprednisolone) is administered intravenously, intramuscularly, subcutaneously, topically, orally, transdermally, intraperitoneally, intraorbitally, by implantation, by inhalation, intrathecally, intraventricularly, or intranasally. In some embodiments, the anti-CD79b immunoconjugate, the anti-CD20 antibody (such as obinutuzumab or rituximab), and the one or more chemotherapeutic agents (e.g., cyclophosphamide and/or doxorubicin) are each administered via intravenous infusion, and the corticosteroid (e.g., prednisone or prednisolone) is administered orally. In some embodiments, the anti-CD79b immunoconjugate, the anti-CD20 antibody (such as obinutuzumab or rituximab), the one or more chemotherapeutic agents (e.g., cyclophosphamide and/or doxorubicin), and the corticosteroid (e.g., methylprednisolone) are each administered via intravenous infusion. An effective amount of the anti-CD79b immunoconjugate, the anti-CD20 antibody (such as obinutuzumab or rituximab), the one or more chemotherapeutic agents (e.g., cyclophosphamide and/or doxorubicin), and the corticosteroid (e.g., prednisone, prednisolone, or methylprednisolone) may be administered for prevention or treatment of a disease, e.g., DLBCL.

### B. Exemplary Treatment Regimens

In some embodiments, the methods for treating diffuse large B-cell lymphoma (DLBCL) in an individual, e.g., a human patient, in need thereof comprise administering to the individual an anti-CD79b immunoconjugate comprising the formula:

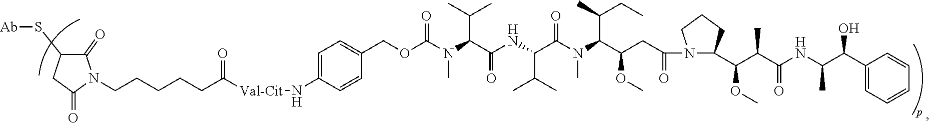

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8; an anti-CD20 antibody (such as obinutuzumab or rituximab); one or more chemotherapeutic agents (e.g., cyclophosphamide and/or doxorubicin); and a corticosteroid (e.g., prednisone, prednisolone, or methylprednisolone). In some embodiments, p is between 2 and 5. In some embodiments, p is between 3 and 4. In some embodiments, p is 3.4. In some embodiments, p is 3.5. In some embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35. In some embodiments, the immunoconjugate is polatuzumab vedotin. In some embodiments, the anti-CD79b immunoconjugate, anti-CD20 antibody (such as obinutuzumab or rituximab), one or more chemotherapeutic agents (e.g., cyclophosphamide and/or doxorubicin), and corticosteroid (e.g., prednisone, prednisolone, or methylprednisolone) are administered to an individual, e.g., a human patient, in 21-day cycles. In some embodiments, the anti-CD20 antibody is rituximab, see. e.g., Section (i) below. In some embodiments, the anti-CD20 antibody is obinutuzumab, see. e.g., Section (ii) below.

In some embodiments of any of the methods provided herein, treatment for DLBCL according to any of the methods of the disclosure is a first-line treatment for DLBCL, i.e., treating human patients with previously untreated DLBCL.

### (i) Treatment Regimens Comprising Rituximab

In some embodiments of any of the methods provided herein, the anti-CD20 anybody is rituximab.

In some embodiments, the corticosteroid is prednisone.

In some embodiments, the anti-CD79b immunoconjugate (e.g., huMA79bv28-MC-vc-PAB-MMAE or polatuzumab vedotin) is administered intravenously at a dose of between about 1.0 mg/kg and about 1.8 mg/kg on day 1 of each 21-day cycle; the anti-CD20 antibody is rituximab administered intravenously at a dose of about 375 mg/m² on day 1 of each 21-day cycle; the one or more chemotherapeutic agents comprise cyclophosphamide administered intravenously at a dose of between about 375 mg/m² and about 750 mg/m² on day 1 of each 21-day cycle, and doxorubicin administered intravenously at a dose of between about 25 mg/m² and about 50 mg/m² on day 1 of each 21-day cycle; and the corticosteroid is prednisone administered orally at a dose of about 100 mg per day on each of days 1-5 of each 21-day cycle. In some embodiments, the anti-CD79b immunoconjugate (e.g., huMA79bv28-MC-vc-PAB-MMAE or polatuzumab vedotin) is administered intravenously at a dose of about 1.0 mg/kg on day 1 of each 21-day cycle. In some embodiments, the anti-CD79b immunoconjugate (e.g., huMA79bv28-MC-vc-PAB-MMAE or polatuzumab vedotin) is administered intravenously at a dose of about 1.4 mg/kg on day 1 of each 21-day cycle. In some embodiments, the anti-CD79b immunoconjugate (e.g., huMA 79bv28-MC-vc-PAB-MMAE or polatuzumab vedotin) is administered intravenously at a dose of about 1.8 mg/kg on day 1 of each 21-day cycle. In some embodiments, the cyclophosphamide is administered intravenously at a dose of about 375 mg/m² on day 1 of each 21-day cycle. In some embodiments, the cyclophosphamide is administered intravenously at a dose of about 563 mg/m² on day 1 of each 21-day cycle. In some embodiments, the cyclophosphamide is administered intravenously at a dose of about 750 mg/m² on day 1 of each 21-day cycle. In some embodiments, the doxorubicin is administered intravenously at a dose of about 25 mg/m² on day 1 of each 21-day cycle. In some embodiments, the doxorubicin is administered intravenously at a dose of about 37.5 mg/m² on day 1 of each 21-day cycle. In some embodiments, the doxorubicin is administered intravenously at a dose of about 50 mg/m² on day 1 of each 21-day cycle.

In some embodiments of any of the methods for treating diffuse large B-cell lymphoma (DLBCL) provided herein, the anti-CD79b immunoconjugate (e.g., huMA 79bv28-MC-vc-PAB-MMAE or polatuzumab vedotin) is administered intravenously at a dose of between about 1.0 mg/kg and about 1.8 mg/kg on day 1 of each of the first, second, third, fourth, fifth, and sixth 21-day cycles; the anti-CD20 antibody is rituximab administered intravenously at a dose of about 375 mg/m² on day 1 of each of the first, second, third, fourth, fifth, and sixth 21-day cycles; the one or more chemotherapeutic agents comprise cyclophosphamide administered intravenously at a dose of between about 375 mg/m² and about 750 mg/m² on day 1 of each of the first, second, third, fourth, fifth, and sixth 21-day cycles, and doxorubicin administered intravenously at a dose of between about 25 mg/m² and about 50 mg/m² on day 1 of each of the first, second, third, fourth, fifth, and sixth 21-day cycles; and the corticosteroid is prednisone administered orally at a dose of about 100 mg per day on each of days 1-5 of each of the first, second, third, fourth, fifth, and sixth 21-day cycles. In some embodiments, the anti-CD79b immunoconjugate (e.g., huMA79bv28-MC-vc-PAB-MMAE or polatuzumab vedotin) is administered intravenously at a dose of about 1.0 mg/kg on day 1 of each of the first, second, third, fourth, fifth, and sixth 21-day cycles. In some embodiments, the anti-CD79b immunoconjugate (e.g., huMA79bv28-MC-vc-PAB-MMAE or polatuzumab vedotin) is administered intravenously at a dose of about 1.4 mg/kg on day 1 of each of the first, second, third, fourth, fifth, and sixth 21-day cycles. In some embodiments, the anti-CD79b immunoconjugate (e.g., huMA79bv28-MC-vc-PAB-MMAE or polatuzumab vedotin) is administered intravenously at a dose of about 1.8 mg/kg on day 1 of each of the first, second, third, fourth, fifth, and sixth 21-day cycles. In some embodiments, the cyclophosphamide is administered intravenously at a dose of about 375 mg/m² on day 1 of each of the first, second, third, fourth, fifth, and sixth 21-day cycles. In some embodiments, the cyclophosphamide is administered intravenously at a dose of about 563 mg/m² on day 1 of each of the first, second, third, fourth, fifth, and sixth 21-day cycles. In some embodiments, the cyclophosphamide is administered intravenously at a dose of about 750 mg/m² on day 1 of each of the first, second, third, fourth, fifth, and sixth 21-day cycles. In some embodiments, the doxorubicin is administered intravenously at a dose of about 25 mg/m² on day 1 of each of the first, second, third, fourth, fifth, and sixth 21-day cycles. In some embodiments, the doxorubicin is administered intravenously at a dose of about 37.5 mg/m² on day 1 of each of the first, second, third, fourth, fifth, and sixth 21-day cycles. In some embodiments, the doxorubicin is administered intravenously at a dose of about 50 mg/m² on day 1 of each of the first, second, third, fourth, fifth, and sixth 21-day cycles.

In some embodiments, the corticosteroid is prednisolone.

In some embodiments, the anti-CD79b immunoconjugate (e.g., huMA79bv28-MC-vc-PAB-MMAE or polatuzumab vedotin) is administered intravenously at a dose of between about 1.0 mg/kg and about 1.8 mg/kg on day 1 of each 21-day cycle; the anti-CD20 antibody is rituximab administered intravenously at a dose of about 375 mg/m² on day 1 of each 21-day cycle; the one or more chemotherapeutic agents comprise cyclophosphamide administered intravenously at a dose of between about 375 mg/m² and about 750 mg/m² on day 1 of each 21-day cycle, and doxorubicin administered intravenously at a dose of between about 25 mg/m² and about 50 mg/m² on day 1 of each 21-day cycle; and the corticosteroid is prednisolone administered orally at a dose of about 100 mg per day on each of days 1-5 of each 21-day cycle. In some embodiments, the anti-CD79b immunoconjugate (e.g., huMA79bv28-MC-vc-PAB-MMAE or polatuzumab vedotin) is administered intravenously at a dose of about 1.0 mg/kg on day 1 of each 21-day cycle. In some embodiments, the anti-CD79b immunoconjugate (e.g., huMA 79bv28-MC-vc-PAB-MMAE or polatuzumab vedotin) is administered intravenously at a dose of about 1.4 mg/kg on day 1 of each 21-day cycle. In some embodiments, the anti-CD79b immunoconjugate (e.g., huMA79bv28-MC-vc-PAB-MMAE or polatuzumab vedotin) is administered intravenously at a dose of about 1.8 mg/kg on day 1 of each 21-day cycle. In some embodiments, the cyclophosphamide is administered intravenously at a dose of about 375 mg/m² on day 1 of each 21-day cycle. In some embodiments, the cyclophosphamide is administered intravenously at a dose of about 563 mg/m² on day 1 of each 21-day cycle. In some embodiments, the cyclophosphamide is administered intravenously at a dose of about 750 mg/m² on day 1 of each 21-day cycle. In some embodiments, the doxorubicin is administered intravenously at a dose of about 25 mg/m² on day 1 of each 21-day cycle. In some embodiments, the doxorubicin is administered intravenously at a dose of about 37.5 mg/m² on day 1 of each 21-day cycle. In some embodiments, the doxorubicin is administered intravenously at a dose of about 50 mg/m² on day 1 of each 21-day cycle.

In some embodiments of any of the methods for treating diffuse large B-cell lymphoma (DLBCL) provided herein, the anti-CD79b immunoconjugate (e.g., huMA 79bv28-MC-vc-PAB-MMAE or polatuzumab vedotin) is administered intravenously at a dose of between about 1.0 mg/kg and about 1.8 mg/kg on day 1 of each of the first, second, third, fourth, fifth, and sixth 21-day cycles; the anti-CD20 antibody is rituximab administered intravenously at a dose of about 375 mg/m² on day 1 of each of the first, second, third, fourth, fifth, and sixth 21-day cycles; the one or more chemotherapeutic agents comprise cyclophosphamide administered intravenously at a dose of between about 375 mg/m² and about 750 mg/m² on day 1 of each of the first, second, third, fourth, fifth, and sixth 21-day cycles, and doxorubicin administered intravenously at a dose of between about 25 mg/m² and about 50 mg/m² on day 1 of each of the first, second, third, fourth, fifth, and sixth 21-day cycles; and the corticosteroid is prednisolone administered orally at a dose of about 100 mg per day on each of days 1-5 of each of the first, second, third, fourth, fifth, and sixth 21-day cycles. In some embodiments, the anti-CD79b immunoconjugate (e.g., huMA 79bv28-MC-vc-PAB-MMAE or polatuzumab vedotin) is administered intravenously at a dose of about 1.0 mg/kg on day 1 of each of the first, second, third, fourth, fifth, and sixth 21-day cycles. In some embodiments, the anti-CD79b immunoconjugate (e.g., huMA79bv28-MC-vc-PAB-MMAE or polatuzumab vedotin) is administered intravenously at a dose of about 1.4 mg/kg on day 1 of each of the first, second, third, fourth, fifth, and sixth 21-day cycles. In some embodiments, the anti-CD79b immunoconjugate (e.g., huMA79bv28-MC-vc-PAB-MMAE or polatuzumab vedotin) is administered intravenously at a dose of about 1.8 mg/kg on day 1 of each of the first, second, third, fourth, fifth, and sixth 21-day cycles. In some embodiments, the cyclophosphamide is administered intravenously at a dose of about 375 mg/m² on day 1 of each of the first, second, third, fourth, fifth, and sixth 21-day cycles. In some embodiments, the cyclophosphamide is administered intravenously at a dose of about 563 mg/m² on day 1 of each of the first, second, third, fourth, fifth, and sixth 21-day cycles. In some embodiments, the cyclophosphamide is administered intravenously at a dose of about 750 mg/m² on day 1 of each of the first, second, third, fourth, fifth, and sixth 21-day cycles. In some embodiments, the doxorubicin is administered intravenously at a dose of about 25 mg/m² on day 1 of each of the first, second, third, fourth, fifth, and sixth 21-day cycles. In some embodiments, the doxorubicin is administered intravenously at a dose of about 37.5 mg/m² on day 1 of each of the first, second, third, fourth, fifth, and sixth 21-day cycles. In some embodiments, the doxorubicin is administered intravenously at a dose of about 50 mg/m² on day 1 of each of the first, second, third, fourth, fifth, and sixth 21-day cycles.

In some embodiments, the corticosteroid is methylprednisolone.

In some embodiments, the anti-CD79b immunoconjugate (e.g., huMA79bv28-MC-vc-PAB-MMAE or polatuzumab vedotin) is administered intravenously at a dose of between about 1.0 mg/kg and about 1.8 mg/kg on day 1 of each 21-day cycle; the anti-CD20 antibody is rituximab administered intravenously at a dose of about 375 mg/m² on day 1 of each 21-day cycle; the one or more chemotherapeutic agents comprise cyclophosphamide administered intravenously at a dose of between about 375 mg/m² and about 750 mg/m² on day 1 of each 21-day cycle, and doxorubicin administered intravenously at a dose of between about 25 mg/m² and about 50 mg/m² on day 1 of each 21-day cycle; and the corticosteroid is methylprednisolone administered intravenously at a dose of about 80 mg per day on each of days 1-5 of each 21-day cycle. In some embodiments, the anti-CD79b immunoconjugate (e.g., huMA79bv28-MC-vc-PAB-MMAE or polatuzumab vedotin) is administered intravenously at a dose of about 1.0 mg/kg on day 1 of each 21-day cycle. In some embodiments, the anti-CD79b immunoconjugate (e.g., huMA 79bv28-MC-vc-PAB-MMAE or polatuzumab vedotin) is administered intravenously at a dose of about 1.4 mg/kg on day 1 of each 21-day cycle. In some embodiments, the anti-CD79b immunoconjugate (e.g., huMA79bv28-MC-vc-PAB-MMAE or polatuzumab vedotin) is administered intravenously at a dose of about 1.8 mg/kg on day 1 of each 21-day cycle. In some embodiments, the cyclophosphamide is administered intravenously at a dose of about 375 mg/m² on day 1 of each 21-day cycle. In some embodiments, the cyclophosphamide is administered intravenously at a dose of about 563 mg/m² on day 1 of each 21-day cycle. In some embodiments, the cyclophosphamide is administered intravenously at a dose of about 750 mg/m² on day 1 of each 21-day cycle. In some embodiments, the doxorubicin is administered intravenously at a dose of about 25 mg/m² on day 1 of each 21-day cycle. In some embodiments, the doxorubicin is administered intravenously at a dose of about 37.5 mg/m² on day 1 of each 21-day cycle. In some embodiments, the doxorubicin is administered intravenously at a dose of about 50 mg/m² on day 1 of each 21-day cycle.

In some embodiments of any of the methods for treating diffuse large B-cell lymphoma (DLBCL) provided herein, the anti-CD79b immunoconjugate (e.g., huMA 79bv28-MC-vc-PAB-MMAE or polatuzumab vedotin) is administered intravenously at a dose of between about 1.0 mg/kg and about 1.8 mg/kg on day 1 of each of the first, second, third, fourth, fifth, and sixth 21-day cycles; the anti-CD20 antibody is rituximab administered intravenously at a dose of about 375 mg/m² on day 1 of each of the first, second, third, fourth, fifth, and sixth 21-day cycles; the one or more chemotherapeutic agents comprise cyclophosphamide administered intravenously at a dose of between about 375 mg/m² and about 750 mg/m² on day 1 of each of the first, second, third, fourth, fifth, and sixth 21-day cycles, and doxorubicin administered intravenously at a dose of between about 25 mg/m² and about 50 mg/m² on day 1 of each of the first, second, third, fourth, fifth, and sixth 21-day cycles; and the corticosteroid is methylprednisolone administered intravenously at a dose of about 80 mg per day on each of days 1-5 of each of the first, second, third, fourth, fifth, and sixth 21-day cycles. In some embodiments, the anti-CD79b immunoconjugate (e.g., huMA 79bv28-MC-vc-PAB-MMAE or polatuzumab vedotin) is administered intravenously at a dose of about 1.0 mg/kg on day 1 of each of the first, second, third, fourth, fifth, and sixth 21-day cycles. In some embodiments, the anti-CD79b immunoconjugate (e.g., huMA79bv28-MC-vc-PAB-MMAE or polatuzumab vedotin) is administered intravenously at a dose of about 1.4 mg/kg on day 1 of each of the first, second, third, fourth, fifth, and sixth 21-day cycles. In some embodiments, the anti-CD79b immunoconjugate (e.g., huMA79bv28-MC-vc-PAB-MMAE or polatuzumab vedotin) is administered intravenously at a dose of about 1.8 mg/kg on day 1 of each of the first, second, third, fourth, fifth, and sixth 21-day cycles. In some embodiments, the cyclophosphamide is administered intravenously at a dose of about 375 mg/m² on day 1 of each of the first, second, third, fourth, fifth, and sixth 21-day cycles. In some embodiments, the cyclophosphamide is administered intravenously at a dose of about 563 mg/m² on day 1 of each of the first, second, third, fourth, fifth, and sixth 21-day cycles. In some embodiments, the cyclophosphamide is administered intravenously at a dose of about 750 mg/m² on day 1 of each of the first, second, third, fourth, fifth, and sixth 21-day cycles. In some embodiments, the doxorubicin is administered intravenously at a dose of about 25 mg/m² on day 1 of each of the first, second, third, fourth, fifth, and sixth 21-day cycles. In some embodiments, the doxorubicin is administered intravenously at a dose of about 37.5 mg/m² on day 1 of each of the first, second, third, fourth, fifth, and sixth 21-day cycles. In some embodiments, the doxorubicin is administered intravenously at a dose of about 50 mg/m² on day 1 of each of the first, second, third, fourth, fifth, and sixth 21-day cycles.

### (ii) Treatment Regimens Comprising Obinutuzumab

In some embodiments of any of the methods provided herein, the anti-CD20 anybody is obinutuzumab.

In some embodiments, the corticosteroid is prednisone.

In some embodiments, the anti-CD79b immunoconjugate (e.g., huMA79bv28-MC-vc-PAB-MMAE or polatuzumab vedotin) is administered intravenously at a dose of between about 1.0 mg/kg and about 1.8 mg/kg on day 1 of each 21-day cycle; the anti-CD20 antibody is obinutuzumab administered intravenously at a dose of about 1000 mg on day 1 of each 21-day cycle; the one or more chemotherapeutic agents comprise cyclophosphamide administered intravenously at a dose of between about 375 mg/m² and about 750 mg/m² on day 1 of each 21-day cycle, and doxorubicin administered intravenously at a dose of between about 25 mg/m² and about 50 mg/m² on day 1 of each 21-day cycle; and the corticosteroid is prednisone administered orally at a dose of about 100 mg per day on each of days 1-5 of each 21-day cycle. In some embodiments, the anti-CD79b immunoconjugate (e.g., huMA79bv28-MC-vc-PAB-MMAE or polatuzumab vedotin) is administered intravenously at a dose of about 1.0 mg/kg on day 1 of each 21-day cycle. In some embodiments, the anti-CD79b immunoconjugate (e.g., huMA79bv28-MC-vc-PAB-MMAE or polatuzumab vedotin) is administered intravenously at a dose of about 1.4 mg/kg on day 1 of each 21-day cycle. In some embodiments, the anti-CD79b immunoconjugate (e.g., huMA79bv28-MC-vc-PAB-MMAE or polatuzumab vedotin) is administered intravenously at a dose of about 1.8 mg/kg on day 1 of each 21-day cycle. In some embodiments, the cyclophosphamide is administered intravenously at a dose of about 375 mg/m² on day 1 of each 21-day cycle. In some embodiments, the cyclophosphamide is administered intravenously at a dose of about 563 mg/m² on day 1 of each 21-day cycle. In some embodiments, the cyclophosphamide is administered intravenously at a dose of about 750 mg/m² on day 1 of each 21-day cycle. In some embodiments, the doxorubicin is administered intravenously at a dose of about 25 mg/m² on day 1 of each 21-day cycle. In some embodiments, the doxorubicin is administered intravenously at a dose of about 37.5 mg/m² on day 1 of each 21-day cycle. In some embodiments, the doxorubicin is administered intravenously at a dose of about 50 mg/m² on day 1 of each 21-day cycle.

In some embodiments of any of the methods for treating diffuse large B-cell lymphoma (DLBCL) provided herein, the anti-CD79b immunoconjugate (e.g., huMA79bv28-MC-vc-PAB-MMAE or polatuzumab vedotin) is administered intravenously at a dose of between about 1.0 mg/kg and about 1.8 mg/kg on day 1 of each of the first, second, third, fourth, fifth, and sixth 21-day cycles; the anti-CD20 antibody is obinutuzumab administered intravenously at a dose of about 1000 mg on day 1 of each of the first, second, third, fourth, fifth, and sixth 21-day cycles; the one or more chemotherapeutic agents comprise cyclophosphamide administered intravenously at a dose of between about 375 mg/m² and about 750 mg/m² on day 1 of each of the first, second, third, fourth, fifth, and sixth 21-day cycles, and doxorubicin administered intravenously at a dose of between about 25 mg/m² and about 50 mg/m² on day 1 of each of the first, second, third, fourth, fifth, and sixth 21-day cycles; and the corticosteroid is prednisone administered orally at a dose of about 100 mg per day on each of days 1-5 of each of the first, second, third, fourth, fifth, and sixth 21-day cycles. In some embodiments, the anti-CD79b immunoconjugate (e.g., huMA79bv28-MC-vc-PAB-MMAE or polatuzumab vedotin) is administered intravenously at a dose of about 1.0 mg/kg on day 1 of each of the first, second, third, fourth, fifth, and sixth 21-day cycles. In some embodiments, the anti-CD79b immunoconjugate (e.g., huMA79bv28-MC-vc-PAB-MMAE or polatuzumab vedotin) is administered intravenously at a dose of about 1.4 mg/kg on day 1 of each of the first, second, third, fourth, fifth, and sixth 21-day cycles. In some embodiments, the anti-CD79b immunoconjugate (e.g., huMA79bv28-MC-vc-PAB-MMAE or polatuzumab vedotin) is administered intravenously at a dose of about 1.8 mg/kg on day 1 of each of the first, second, third, fourth, fifth, and sixth 21-day cycles. In some embodiments, the cyclophosphamide is administered intravenously at a dose of about 375 mg/m² on day 1 of each of the first, second, third, fourth, fifth, and sixth 21-day cycles. In some embodiments, the cyclophosphamide is administered intravenously at a dose of about 563 mg/m² on day 1 of each of the first, second, third, fourth, fifth, and sixth 21-day cycles. In some embodiments, the cyclophosphamide is administered intravenously at a dose of about 750 mg/m² on day 1 of each of the first, second, third, fourth, fifth, and sixth 21-day cycles. In some embodiments, the doxorubicin is administered intravenously at a dose of about 25 mg/m² on day 1 of each of the first, second, third, fourth, fifth, and sixth 21-day cycles. In some embodiments, the doxorubicin is administered intravenously at a dose of about 37.5 mg/m² on day 1 of each of the first, second, third, fourth, fifth, and sixth 21-day cycles. In some embodiments, the doxorubicin is administered intravenously at a dose of about 50 mg/m² on day 1 of each of the first, second, third, fourth, fifth, and sixth 21-day cycles.

In some embodiments, the corticosteroid is prednisolone.

In some embodiments, the anti-CD79b immunoconjugate (e.g., huMA79bv28-MC-vc-PAB-MMAE or polatuzumab vedotin) is administered intravenously at a dose of between about 1.0 mg/kg and about 1.8 mg/kg on day 1 of each 21-day cycle; the anti-CD20 antibody is obinutuzumab administered intravenously at a dose of about 1000 mg on day 1 of each 21-day cycle; the one or more chemotherapeutic agents comprise cyclophosphamide administered intravenously at a dose of between about 375 mg/m² and about 750 mg/m² on day 1 of each 21-day cycle, and doxorubicin administered intravenously at a dose of between about 25 mg/m² and about 50 mg/m² on day 1 of each 21-day cycle; and the corticosteroid is prednisolone administered orally at a dose of about 100 mg per day on each of days 1-5 of each 21-day cycle. In some embodiments, the anti-CD79b immunoconjugate (e.g., huMA79bv28-MC-vc-PAB-MMAE or polatuzumab vedotin) is administered intravenously at a dose of about 1.0 mg/kg on day 1 of each 21-day cycle. In some embodiments, the anti-CD79b immunoconjugate (e.g., huMA 79bv28-MC-vc-PAB-MMAE or polatuzumab vedotin) is administered intravenously at a dose of about 1.4 mg/kg on day 1 of each 21-day cycle. In some embodiments, the anti-CD79b immunoconjugate (e.g., huMA79bv28-MC-vc-PAB-MMAE or polatuzumab vedotin) is administered intravenously at a dose of about 1.8 mg/kg on day 1 of each 21-day cycle. In some embodiments, the cyclophosphamide is administered intravenously at a dose of about 375 mg/m² on day 1 of each 21-day cycle. In some embodiments, the cyclophosphamide is administered intravenously at a dose of about 563 mg/m² on day 1 of each 21-day cycle. In some embodiments, the cyclophosphamide is administered intravenously at a dose of about 750 mg/m² on day 1 of each 21-day cycle. In some embodiments, the doxorubicin is administered intravenously at a dose of about 25 mg/m² on day 1 of each 21-day cycle. In some embodiments, the doxorubicin is administered intravenously at a dose of about 37.5 mg/m² on day 1 of each 21-day cycle. In some embodiments, the doxorubicin is administered intravenously at a dose of about 50 mg/m² on day 1 of each 21-day cycle.

In some embodiments of any of the methods for treating diffuse large B-cell lymphoma (DLBCL) provided herein, the anti-CD79b immunoconjugate (e.g., huMA79bv28-MC-vc-PAB-MMAE or polatuzumab vedotin) is administered intravenously at a dose of between about 1.0 mg/kg and about 1.8 mg/kg on day 1 of each of the first, second, third, fourth, fifth, and sixth 21-day cycles; the anti-CD20 antibody is obinutuzumab administered intravenously at a dose of about 1000 mg on day 1 of each of the first, second, third, fourth, fifth, and sixth 21-day cycles; the one or more chemotherapeutic agents comprise cyclophosphamide administered intravenously at a dose of between about 375 mg/m² and about 750 mg/m² on day 1 of each of the first, second, third, fourth, fifth, and sixth 21-day cycles, and doxorubicin administered intravenously at a dose of between about 25 mg/m² and about 50 mg/m² on day 1 of each of the first, second, third, fourth, fifth, and sixth 21-day cycles; and the corticosteroid is prednisolone administered orally at a dose of about 100 mg per day on each of days 1-5 of each of the first, second, third, fourth, fifth, and sixth 21-day cycles. In some embodiments, the anti-CD79b immunoconjugate (e.g., huMA 79bv28-MC-vc-PAB-MMAE or polatuzumab vedotin) is administered intravenously at a dose of about 1.0 mg/kg on day 1 of each of the first, second, third, fourth, fifth, and sixth 21-day cycles. In some embodiments, the anti-CD79b immunoconjugate (e.g., huMA79bv28-MC-vc-PAB-MMAE or polatuzumab vedotin) is administered intravenously at a dose of about 1.4 mg/kg on day 1 of each of the first, second, third, fourth, fifth, and sixth 21-day cycles. In some embodiments, the anti-CD79b immunoconjugate (e.g., huMA79bv28-MC-vc-PAB-MMAE or polatuzumab vedotin) is administered intravenously at a dose of about 1.8 mg/kg on day 1 of each of the first, second, third, fourth, fifth, and sixth 21-day cycles. In some embodiments, the cyclophosphamide is administered intravenously at a dose of about 375 mg/m² on day 1 of each of the first, second, third, fourth, fifth, and sixth 21-day cycles. In some embodiments, the cyclophosphamide is administered intravenously at a dose of about 563 mg/m² on day 1 of each of the first, second, third, fourth, fifth, and sixth 21-day cycles. In some embodiments, the cyclophosphamide is administered intravenously at a dose of about 750 mg/m² on day 1 of each of the first, second, third, fourth, fifth, and sixth 21-day cycles. In some embodiments, the doxorubicin is administered intravenously at a dose of about 25 mg/m² on day 1 of each of the first, second, third, fourth, fifth, and sixth 21-day cycles. In some embodiments, the doxorubicin is administered intravenously at a dose of about 37.5 mg/m² on day 1 of each of the first, second, third, fourth, fifth, and sixth 21-day cycles. In some embodiments, the doxorubicin is administered intravenously at a dose of about 50 mg/m² on day 1 of each of the first, second, third, fourth, fifth, and sixth 21-day cycles.

In some embodiments, the corticosteroid is methylprednisolone.

In some embodiments, the anti-CD79b immunoconjugate (e.g., huMA79bv28-MC-vc-PAB-MMAE or polatuzumab vedotin) is administered intravenously at a dose of between about 1.0 mg/kg and about 1.8 mg/kg on day 1 of each 21-day cycle; the anti-CD20 antibody is obinutuzumab administered intravenously at a dose of about 1000 mg on day 1 of each 21-day cycle; the one or more chemotherapeutic agents comprise cyclophosphamide administered intravenously at a dose of between about 375 mg/m² and about 750 mg/m² on day 1 of each 21-day cycle, and doxorubicin administered intravenously at a dose of between about 25 mg/m² and about 50 mg/m² on day 1 of each 21-day cycle; and the corticosteroid is methylprednisolone administered intravenously at a dose of about 80 mg per day on each of days 1-5 of each 21-day cycle. In some embodiments, the anti-CD79b immunoconjugate (e.g., huMA79bv28-MC-vc-PAB-MMAE or polatuzumab vedotin) is administered intravenously at a dose of about 1.0 mg/kg on day 1 of each 21-day cycle. In some embodiments, the anti-CD79b immunoconjugate (e.g., huMA 79bv28-MC-vc-PAB-MMAE or polatuzumab vedotin) is administered intravenously at a dose of about 1.4 mg/kg on day 1 of each 21-day cycle. In some embodiments, the anti-CD79b immunoconjugate (e.g., huMA 79bv28-MC-vc-PAB-MMAE or polatuzumab vedotin) is administered intravenously at a dose of about 1.8 mg/kg on day 1 of each 21-day cycle. In some embodiments, the cyclophosphamide is administered intravenously at a dose of about 375 mg/m² on day 1 of each 21-day cycle. In some embodiments, the cyclophosphamide is administered intravenously at a dose of about 563 mg/m² on day 1 of each 21-day cycle. In some embodiments, the cyclophosphamide is administered intravenously at a dose of about 750 mg/m² on day 1 of each 21-day cycle. In some embodiments, the doxorubicin is administered intravenously at a dose of about 25 mg/m² on day 1 of each 21-day cycle. In some embodiments, the doxorubicin is administered intravenously at a dose of about 37.5 mg/m² on day 1 of each 21-day cycle. In some embodiments, the doxorubicin is administered intravenously at a dose of about 50 mg/m² on day 1 of each 21-day cycle.

In some embodiments of any of the methods for treating diffuse large B-cell lymphoma (DLBCL) provided herein, the anti-CD79b immunoconjugate (e.g., huMA 79bv28-MC-vc-PAB-MMAE or polatuzumab vedotin) is administered intravenously at a dose of between about 1.0 mg/kg and about 1.8 mg/kg on day 1 of each of the first, second, third, fourth, fifth, and sixth 21-day cycles; the anti-CD20 antibody is obinutuzumab administered intravenously at a dose of about 1000 mg on day 1 of each of the first, second, third, fourth, fifth, and sixth 21-day cycles; the one or more chemotherapeutic agents comprise cyclophosphamide administered intravenously at a dose of between about 375 mg/m² and about 750 mg/m² on day 1 of each of the first, second, third, fourth, fifth, and sixth 21-day cycles, and doxorubicin administered intravenously at a dose of between about 25 mg/m² and about 50 mg/m² on day 1 of each of the first, second, third, fourth, fifth, and sixth 21-day cycles; and the corticosteroid is methylprednisolone administered intravenously at a dose of about 80 mg per day on each of days 1-5 of each of the first, second, third, fourth, fifth, and sixth 21-day cycles. In some embodiments, the anti-CD79b immunoconjugate (e.g., huMA79bv28-MC-vc-PAB-MMAE or polatuzumab vedotin) is administered intravenously at a dose of about 1.0 mg/kg on day 1 of each of the first, second, third, fourth, fifth, and sixth 21-day cycles. In some embodiments, the anti-CD79b immunoconjugate (e.g., huMA79bv28-MC-vc-PAB-MMAE or polatuzumab vedotin) is administered intravenously at a dose of about 1.4 mg/kg on day 1 of each of the first, second, third, fourth, fifth, and sixth 21-day cycles. In some embodiments, the anti-CD79b immunoconjugate (e.g., huMA79bv28-MC-vc-PAB-MMAE or polatuzumab vedotin) is administered intravenously at a dose of about 1.8 mg/kg on day 1 of each of the first, second, third, fourth, fifth, and sixth 21-day cycles. In some embodiments, the cyclophosphamide is administered intravenously at a dose of about 375 mg/m² on day 1 of each of the first, second, third, fourth, fifth, and sixth 21-day cycles. In some embodiments, the cyclophosphamide is administered intravenously at a dose of about 563 mg/m² on day 1 of each of the first, second, third, fourth, fifth, and sixth 21-day cycles. In some embodiments, the cyclophosphamide is administered intravenously at a dose of about 750 mg/m² on day 1 of each of the first, second, third, fourth, fifth, and sixth 21-day cycles. In some embodiments, the doxorubicin is administered intravenously at a dose of about 25 mg/m² on day 1 of each of the first, second, third, fourth, fifth, and sixth 21-day cycles. In some embodiments, the doxorubicin is administered intravenously at a dose of about 37.5 mg/m² on day 1 of each of the first, second, third, fourth, fifth, and sixth 21-day cycles. In some embodiments, the doxorubicin is administered intravenously at a dose of about 50 mg/m² on day 1 of each of the first, second, third, fourth, fifth, and sixth 21-day cycles.

(iii) Length of Treatment Regimens

In some embodiments of any of the methods for treating diffuse large B-cell lymphoma (DLBCL) provided herein, the anti-CD79b immunoconjugate (e.g., huMA79bv28-MC-vc-PAB-MMAE or polatuzumab vedotin), the anti-CD20 antibody (such as obinutuzumab or rituximab), the one or more chemotherapeutic agents (e.g., cyclophosphamide and/or doxorubicin), and the corticosteroid (e.g., prednisone, prednisolone, or methylprednisolone) are administered to an individual, e.g., a human patient, in 21-day cycles. In some embodiments, the anti-CD79b immunoconjugate (such as huMA 79bv28-MC-vc-PAB-MMAE or polatuzumab vedotin), the anti-CD20 antibody (such as obinutuzumab or rituximab), the one or more chemotherapeutic agents (e.g., cyclophosphamide and/or doxorubicin), and the corticosteroid (e.g., prednisone, prednisolone, or methylprednisolone) are administered for less than one 21-day cycle. In some embodiments, the anti-CD79b immunoconjugate (such as huMA79bv28-MC-vc-PAB-MMAE or polatuzumab vedotin), the anti-CD20 antibody (such as obinutuzumab or rituximab), the one or more chemotherapeutic agents (e.g., cyclophosphamide and/or doxorubicin), and the corticosteroid (e.g., prednisone, prednisolone, or methylprednisolone) are administered to an individual, e.g., a human patient, for at least one, at least two, at least three, at least four, at least five, or at least six 21-day cycles. In some embodiments, the anti-CD79b immunoconjugate (such as huMA79bv28-MC-vc-PAB-MMAE or polatuzumab vedotin), the anti-CD20 antibody (such as obinutuzumab or rituximab), the one or more chemotherapeutic agents (e.g., cyclophosphamide and/or doxorubicin), and the corticosteroid (e.g., prednisone, prednisolone, or methylprednisolone) are administered to an individual, e.g., a human patient, for between one and six 21-day cycles. In some embodiments, the anti-CD79b immunoconjugate (such as huMA79bv28-MC-vc-PAB-MMAE or polatuzumab vedotin), the anti-CD20 antibody (such as obinutuzumab or rituximab), the one or more chemotherapeutic agents (e.g., cyclophosphamide and/or doxorubicin), and the corticosteroid (e.g., prednisone, prednisolone, or methylprednisolone) are administered to an individual, e.g., a human patient, for six 21-day cycles.

In some embodiments of any of the methods for treating diffuse large B-cell lymphoma (DLBCL) provided herein, the anti-CD20 antibody (such as obinutuzumab or rituximab) is administered as a monotherapy after the sixth 21-day cycle of treatment with the anti-CD79b immunoconjugate (such as huMA79bv28-MC-vc-PAB-MMAE or polatuzumab vedotin), the anti-CD20 antibody (such as obinutuzumab or rituximab), the one or more chemotherapeutic agents (e.g., cyclophosphamide and/or doxorubicin), and the corticosteroid (e.g., prednisone, prednisolone, or methylprednisolone). In some embodiments, the methods comprise administering an anti-CD20 antibody (such as obinutuzumab or rituximab) as a monotherapy for one or two additional 21-day cycles after the sixth 21-day cycle of treatment with the anti-CD79b immunoconjugate (such as huMA79bv28-MC-vc-PAB-MMAE or polatuzumab vedotin), the anti-CD20 antibody (such as obinutuzumab or rituximab), the one or more chemotherapeutic agents (e.g., cyclophosphamide and/or doxorubicin), and the corticosteroid (e.g., prednisone, prednisolone, or methylprednisolone). In some embodiments, the methods comprise administering an anti-CD20 antibody (such as obinutuzumab or rituximab) as a monotherapy in a seventh 21-day cycle after the sixth 21-day cycle of treatment with the anti-CD79b immunoconjugate (such as huMA79bv28-MC-vc-PAB-MMAE or polatuzumab vedotin), the anti-CD20 antibody (such as obinutuzumab or rituximab), the one or more chemotherapeutic agents (e.g., cyclophosphamide and/or doxorubicin), and the corticosteroid (e.g., prednisone, prednisolone, or methylprednisolone). In some embodiments, the methods comprise administering an anti-CD20 antibody (such as obinutuzumab or rituximab) as a monotherapy in a seventh and eighth 21-day cycle after the sixth 21-day cycle of treatment with the anti-CD79b immunoconjugate (such as huMA79bv28-MC-vc-PAB-MMAE or polatuzumab vedotin), the anti-CD20 antibody (such as obinutuzumab or rituximab), the one or more chemotherapeutic agents (e.g., cyclophosphamide and/or doxorubicin), and the corticosteroid (e.g., prednisone, prednisolone, or methylprednisolone). In some embodiments, the methods comprise administering an anti-CD20 antibody (such as obinutuzumab or rituximab) on day 1 of each cycle after the sixth 21-day cycle of treatment with the anti-CD79b immunoconjugate (such as huMA79bv28-MC-vc-PAB-MMAE or polatuzumab vedotin), the anti-CD20 antibody (such as obinutuzumab or rituximab), the one or more chemotherapeutic agents (e.g., cyclophosphamide and/or doxorubicin), and the corticosteroid (e.g., prednisone, prednisolone, or methylprednisolone), e.g., during a seventh 21-day cycle, and optionally an eighth 21-day cycle. In some embodiments, the methods comprise administering an anti-CD20 antibody (such as obinutuzumab or rituximab) on day 1 of the seventh and eighth 21-day cycles after the sixth 21-day cycle of treatment with the anti-CD79b immunoconjugate (such as huMA79bv28-MC-vc-PAB-MMAE or polatuzumab vedotin), the anti-CD20 antibody (such as obinutuzumab or rituximab), the one or more chemotherapeutic agents (e.g., cyclophosphamide and/or doxorubicin), and the corticosteroid (e.g., prednisone, prednisolone, or methylprednisolone).

In some embodiments of any of the methods for treating DLBCL provided herein, the method further comprises continuing administration of rituximab monotherapy at a dose of about 375 mg/m² for one or two additional 21-day cycles after the sixth 21-day cycle of treatment with the anti-CD79b immunoconjugate (such as huMA79bv28-MC-vc-PAB-MMAE or polatuzumab vedotin), rituximab, cyclophosphamide, doxorubicin, and corticosteroid (e.g., prednisone, prednisolone, or methylprednisolone). In some embodiments, the methods comprise administering the anti-CD79b immunoconjugate (such as huMA79bv28-MC-vc-PAB-MMAE or polatuzumab vedotin), rituximab, cyclophosphamide, doxorubicin, and corticosteroid (e.g., prednisone, prednisolone, or methylprednisolone) for six 21-day cycles (i.e., Cycles 1-6), followed by administration of rituximab monotherapy at a dose of about 375 mg/m² for one or two additional 21-day cycles after the sixth cycle (i.e., on Cycle 7 and optionally Cycle 8). In some embodiments, rituximab is administered on day 1 of each 21-day cycle.

In some embodiments of any of the methods for treating diffuse large B-cell lymphoma (DLBCL) provided herein, the anti-CD20 antibody (such as obinutuzumab or rituximab), one or more chemotherapeutic agents (e.g., cyclophosphamide and/or doxorubicin), and corticosteroid (e.g., prednisone, prednisolone, or methylprednisolone) are administered for one or two additional 21-day cycles after the sixth 21-day cycle of treatment with the anti-CD79b immunoconjugate (such as huMA79bv28-MC-vc-PAB-MMAE or polatuzumab vedotin), the anti-CD20 antibody (such as obinutuzumab or rituximab), the one or more chemotherapeutic agents (e.g., cyclophosphamide and/or doxorubicin), and the corticosteroid (e.g., prednisone, prednisolone, or methylprednisolone). In some embodiments, the methods comprise administering an anti-CD20 antibody (such as obinutuzumab or rituximab), one or more chemotherapeutic agents (e.g., cyclophosphamide and/or doxorubicin), and corticosteroid (e.g., prednisone, prednisolone, or methylprednisolone) in a seventh 21-day cycle after the sixth 21-day cycle of treatment with the anti-CD79b immunoconjugate (such as huMA79bv28-MC-vc-PAB-MMAE or polatuzumab vedotin), the anti-CD20 antibody (such as obinutuzumab or rituximab), the one or more chemotherapeutic agents (e.g., cyclophosphamide and/or doxorubicin), and the corticosteroid (e.g., prednisone, prednisolone, or methylprednisolone). In some embodiments, the methods comprise administering an anti-CD20 antibody (such as obinutuzumab or rituximab), one or more chemotherapeutic agents (e.g., cyclophosphamide and/or doxorubicin), and corticosteroid (e.g., prednisone, prednisolone, or methylprednisolone) in a seventh and eighth 21-day cycle after the sixth 21-day cycle of treatment with the anti-CD79b immunoconjugate (such as huMA79bv28-MC-vc-PAB-MMAE or polatuzumab vedotin), the anti-CD20 antibody (such as obinutuzumab or rituximab), the one or more chemotherapeutic agents (e.g., cyclophosphamide and/or doxorubicin), and the corticosteroid (e.g., prednisone, prednisolone, or methylprednisolone). In some embodiments, the methods comprise administering an anti-CD20 antibody (such as obinutuzumab or rituximab) and one or more chemotherapeutic agents (e.g., cyclophosphamide and/or doxorubicin) on day 1, and a corticosteroid (e.g., prednisone, prednisolone, or methylprednisolone) on days 1-5, of each cycle after the sixth 21-day cycle of treatment with the anti-CD79b immunoconjugate (such as huMA79bv28-MC-vc-PAB-MMAE or polatuzumab vedotin), the anti-CD20 antibody (such as obinutuzumab or rituximab), the one or more chemotherapeutic agents (e.g., cyclophosphamide and/or doxorubicin), and the corticosteroid (e.g., prednisone, prednisolone, or methylprednisolone). In some embodiments, the methods comprise administering an anti-CD20 antibody (such as obinutuzumab or rituximab) and one or more chemotherapeutic agents (e.g., cyclophosphamide and/or doxorubicin) on day 1, and a corticosteroid (e.g., prednisone, prednisolone, or methylprednisolone) on days 1-5, of the seventh and eighth 21-day cycles after the sixth 21-day cycle of treatment with the anti-CD79b immunoconjugate (such as huMA79bv28-MC-vc-PAB-MMAE or polatuzumab vedotin), the anti-CD20 antibody (such as obinutuzumab or rituximab), the one or more chemotherapeutic agents (e.g., cyclophosphamide and/or doxorubicin), and the corticosteroid (e.g., prednisone, prednisolone, or methylprednisolone).

In some embodiments of any of the methods for treating DLBCL provided herein, the method comprises administering rituximab at a dose of about 375 mg/m², cyclophosphamide at a dose between about 375 mg/m² and about 750 mg/m², doxorubicin at a dose between about 25 mg/m² and about 50 mg/m², and corticosteroid (e.g., prednisone at a dose of about 100 mg, prednisolone at a dose of about 100 mg, or methylprednisolone at a dose of about 80 mg) for one or two additional 21-day cycles after the sixth 21-day cycle of treatment with the anti-CD79b immunoconjugate (such as huMA79bv28-MC-vc-PAB-MMAE or polatuzumab vedotin), rituximab, cyclophosphamide, doxorubicin, and corticosteroid (e.g., prednisone, prednisolone, or methylprednisolone). In some embodiments, the methods comprise administering the anti-CD79b immunoconjugate (such as huMA79bv28-MC-vc-PAB-MMAE or polatuzumab vedotin), rituximab, cyclophosphamide, doxorubicin, and corticosteroid (e.g., prednisone, prednisolone, or methylprednisolone) for six 21-day cycles (i.e., Cycles 1-6), followed by administration of rituximab at a dose of about 375 mg/m², cyclophosphamide at a dose between about 375 mg/m² and about 750 mg/m², doxorubicin at a dose between about 25 mg/m² and about 50 mg/m², and corticosteroid (e.g., prednisone at a dose of about 100 mg, prednisolone at a dose of about 100 mg, or methylprednisolone at a dose of about 80 mg) for one or two additional 21-day cycles after the sixth cycle (i.e., on Cycle 7 and optionally Cycle 8). In some embodiments, rituximab, cyclophosphamide, and doxorubicin are each administered on day 1 of each 21-day cycle, and the corticosteroid (e.g., prednisone, prednisolone, or methylprednisolone) is administered on days 1-5 of each 21-day cycle.

In some embodiments of any of the methods provided herein, rituximab is replaced with obinutuzumab, administered at a dose of 1000 mg.

### (iv) Order and Timing of Administration

In some embodiments, the anti-CD79b immunoconjugate (e.g., huMA79bv28-MC-vc-PAB-MMAE or polatuzumab vedotin), the anti-CD20 antibody (e.g., obinutuzumab or rituximab), the one or more chemotherapeutic agents (e.g., cyclophosphamide and/or doxorubicin), and the corticosteroid (e.g., prednisone, prednisolone, or methylprednisolone) are administered sequentially, e.g., on day 1 of each of the first, second, third, fourth, fifth, and sixth 21-day cycles. In some embodiments, the corticosteroid (e.g., prednisone, prednisolone, or methylprednisolone) is administered prior to the anti-CD20 antibody (e.g., obinutuzumab or rituximab): the anti-CD20 antibody (e.g., obinutuzumab or rituximab) is administered prior to the anti-CD79b immunoconjugate (e.g., huMA 79bv28-MC-vc-PAB-MMAE or polatuzumab vedotin); and the anti-CD79b immunoconjugate (e.g., huMA79bv28-MC-vc-PAB-MMAE or polatuzumab vedotin) is administered prior to the one or more chemotherapeutic agents (e.g., cyclophosphamide and/or doxorubicin), e.g., on day 1 of each of the first, second, third, fourth, fifth, and sixth 21-day cycles. In some embodiments, the anti-CD20 antibody (e.g., obinutuzumab or rituximab), the anti-CD79b immunoconjugate (e.g., huMA79bv28-MC-vc-PAB-MMAE or polatuzumab vedotin), and the one or more chemotherapeutic agents (e.g., cyclophosphamide and/or doxorubicin) are administered in any order after administration of the corticosteroid (e.g., prednisone, prednisolone, or methylprednisolone). In some embodiments, the anti-CD20 antibody (e.g., obinutuzumab or rituximab), the anti-CD79b immunoconjugate (e.g., huMA 79bv28-MC-vc-PAB-MMAE or polatuzumab vedotin), and the one or more chemotherapeutic agents (e.g., cyclophosphamide and/or doxorubicin) are administered in any order after administration of the corticosteroid (e.g., prednisone, prednisolone, or methylprednisolone), e.g., on day 1 of each of the first, second, third, fourth, fifth, and sixth 21-day cycles.

In some embodiments of any of the methods for treating DLBCL provided herein, the corticosteroid (e.g., prednisone, prednisolone, or methylprednisolone) is administered at least about 1 hour prior to each administration of the anti-CD20 antibody (e.g., obinutuzumab or rituximab) and/or prior to each administration of the immunoconjugate (e.g., huMA79bv28-MC-vc-PAB-MMAE or polatuzumab vedotin). In some embodiments, when the anti-CD20 antibody (e.g., obinutuzumab or rituximab) is administered as monotherapy (e.g., on day 1 of the seventh and eighth 21-day cycles), a corticosteroid (e.g., prednisone, prednisolone, or methylprednisolone) is administered to the individual prior to administration of the anti-CD20 antibody (e.g., obinutuzumab or rituximab).

### (v) Pre-Medications and Prophylactic Treatments

In some embodiments of any of the methods for treating DLBCL provided herein, the method further comprises administering premedications to the individual, e.g., a human patient, prior to the start of treatment. In some embodiments, the methods comprise administering an antihistamine drug (for example, 50-100 mg of diphenhydramine), an analgesic and/or an anti-pyretic drug (for example, 650-1000 mg of acetaminophen/paracetamol) to the individual prior to the start of treatment, e.g., at least about 30 minutes prior to each administration of the anti-CD20 antibody (e.g., obinutuzumab or rituximab) and/or prior to each administration of the immunoconjugate (e.g., huMA79bv28-MC-vc-PAB-MMAE or polatuzumab vedotin). In some embodiments, the methods comprise administering an antihistamine drug and an analgesic and/or an anti-pyretic drug (e.g., 500-1000 mg of oral acetaminophen or paracetamol and 50-100 mg diphenhydramine) to the individual prior to each administration of the anti-CD20 antibody (e.g., obinutuzumab or rituximab) and/or prior to each administration of the immunoconjugate (e.g., huMA79bv28-MC-vc-PAB-MMAE or polatuzumab vedotin). In some embodiments, the methods comprise administering an antihistamine drug and an analgesic and/or an anti-pyretic drug (e.g., 650-1000 mg of oral acetaminophen or paracetamol and 50-100 mg diphenhydramine) to the individual prior to each administration of the anti-CD20 antibody (e.g., obinutuzumab or rituximab) and/or prior to each administration of the immunoconjugate (e.g., huMA79bv28-MC-vc-PAB-MMAE or polatuzumab vedotin).

In some embodiments of any of the methods for treating DLBCL provided herein, the method further comprises administering a corticosteroid (e.g., prednisone, prednisolone, or methylprednisolone) to the individual prior to the start of treatment, e.g., up to 7 days of treatment with a corticosteroid (e.g., up to 100 mg oral prednisone per day, or an equivalent) prior to the start of treatment.

In some embodiments of any of the methods for treating DLBCL provided herein, the method further comprises administering a prophylactic therapy for neutropenia to the individual. In some embodiments, the prophylactic therapy for neutropenia is administered as outlined in the American Society of Clinical Oncology (ASCO) recommended guidelines (see, e.g., Smith et al., J Clin Oncol (2015) 33:3199-212). In some embodiments, the prophylactic therapy for neutropenia comprises administration of granulocyte colony-stimulating factor (G-CSF, for example, filgrastim, or lenograstim, or peg-filgrastim) to the individual, e.g., starting at about 1 to about 3 days prior to each administration of the one or more chemotherapeutic agents (e.g., cyclophosphamide and/or doxorubicin) and/or immunoconjugate (e.g., huMA79bv28-MC-vc-PAB-MMAE or polatuzumab vedotin).

In some embodiments of any of the methods for treating DLBCL provided herein, the method further comprises administering central nervous system prophylaxis to the individual, such as intrathecal chemotherapy. In some embodiments, the administered central nervous system prophylaxis is not high-dose IV methotrexate (e.g., 1 g/m² per cycle).

In some embodiments of any of the methods for treating DLBCL provided herein, the method further comprises administering a prophylactic treatment for hemorrhagic cystitis to the individual, e.g., adequate hydration and/or Mesna.

In some embodiments of any of the methods for treating DLBCL provided herein, the method further comprises administering anti-infective prophylaxis (e.g., for viral, fungal, bacterial, or Pneumocystis infections) to the individual. In some embodiments, the anti-infective prophylaxis is administered, e.g., as described in Flowers et al., J Clin Oncol (2013) 31:794-810; National Comprehensive Cancer Network®. NCCN clinical practice guidelines in oncology (NCCN Guidelines®); Prevention and treatment of cancer-related infections, version 2 [resource on the Internet]. 2017 [cited 9 Jun. 2017]. Available from: www[dot]nccn[dot]org/professionals/physician_gls/f_guidelines.asp; and Reddy et al., Gastroenterology (2015) 148:215-19. In some embodiments, anti-viral prophylaxis is administered, e.g., as described in the American Gastroenterology Association guidelines, see, e.g., Reddy et al., Gastroenterology (2015) 148:215-19.

In some embodiments of any of the methods for treating DLBCL provided herein, the method further comprises administering a prophylactic therapy for tumor lysis syndrome to the individual. In some embodiments, prophylactic therapy for tumor lysis syndrome is administered to individuals at risk for developing tumor lysis syndrome. In some embodiments, prophylactic therapy for tumor lysis syndrome is administered to an individual with high tumor burden (e.g., lymphocyte count ≥25×10⁹/L or bulky lymphadenopathy). In some embodiments, the prophylactic therapy for tumor lysis syndrome comprises administering allopurinol (e.g., ≥300 mg/day orally), or a suitable alternative treatment such as rasburicase, prior to administration of treatment according to the methods provided herein, e.g., administered starting about 48-72 hours prior to administration of treatment according to the methods provided herein. In some embodiments, the prophylactic therapy for tumor lysis syndrome comprises adequate hydration, e.g., a fluid intake of approximately 3 L/day starting 1 or 2 days before the start of treatment according the methods provided herein.

### (vi) Treatment Adjustments and Modifications

In some embodiments of any of the methods for treating DLBCL provided herein, the method comprises adjusting the dose of the immunoconjugate (e.g., huMA79bv28-MC-vc-PAB-MMAE or polatuzumab vedotin) after the start of treatment, for example, if the individual experiences an infusion-associated symptom, infusion-related reaction or adverse event, e.g., as described in Example 1 herein. In some embodiments of any of the methods provided herein, the dose of the immunoconjugate (e.g., huMA79bv28-MC-vc-PAB-MMAE or polatuzumab vedotin) administered to the individual is reduced from about 1.8 mg/kg to about 1.4 mg/kg. In some embodiments, the dose of the immunoconjugate administered to the individual is reduced from about 1.4 mg/kg to about 1.0 mg/kg.

In some embodiments of any of the methods for treating DLBCL provided herein, the method comprises adjusting the dose of cyclophosphamide after the start of treatment, for example, if the individual experiences an infusion-associated symptom, infusion related reaction or adverse event, e.g., as described in Example 1 herein. In some embodiments of any of the methods provided herein, the dose of cyclophosphamide administered to the individual is reduced from 100% of the starting dose (e.g., 750 mg/m²) to about 75% of the staring dose, e.g., to about 563 mg/m². In some embodiments of any of the methods provided herein, the dose of cyclophosphamide administered to the individual is reduced from about 75% of the starting dose (e.g., 750 mg/m²) to about 50% of the staring dose, e.g., to about 375 mg/m².

In some embodiments of any of the methods for treating DLBCL provided herein, the method comprises adjusting the dose of doxorubicin after the start of treatment, for example, if the individual experiences an infusion-associated symptom, infusion related reaction or adverse event, e.g., as described in Example 1 herein. In some embodiments of any of the methods provided herein, the dose of doxorubicin administered to the individual is reduced from 100% of the starting dose (e.g., 50 mg/m²) to about 75% of the staring dose, e.g., to about 37.5 mg/m². In some embodiments of any of the methods provided herein, the dose of doxorubicin administered to the individual is reduced from 75% of the starting dose (e.g., 50 mg/m²) to about 50% of the staring dose, e.g., to about 25 mg/m².

In some embodiments, treatment (e.g., a control treatment) with R-CHOP may be adjusted as described above.

In some embodiments, the dose of cyclophosphamide in an R-CHOP treatment may be adjusted, e.g., in the event of infusion-associated symptoms, infusion related reactions or adverse events, e.g., as described in Example 1 herein. In some embodiments, the dose of cyclophosphamide is reduced from 100% of the starting dose (e.g., 750 mg/m²) to about 75% of the staring dose, e.g., to about 563 mg/m². In some embodiments, the dose of cyclophosphamide is reduced from about 75% of the starting dose (e.g., 750 mg/m²) to about 50% of the staring dose, e.g., to about 375 mg/m².

In some embodiments, the dose of doxorubicin in an R-CHOP treatment may be adjusted, e.g., in the event of infusion-associated symptoms, infusion related reactions or adverse events, e.g., as described in Example 1 herein. In some embodiments, the dose of doxorubicin is reduced from 100% of the starting dose (e.g., 50 mg/m²) to about 75% of the staring dose, e.g., to about 37.5 mg/m². In some embodiments, the dose of doxorubicin is reduced from 75% of the starting dose (e.g., 50 mg/m²) to about 50% of the staring dose, e.g., to about 25 mg/m².

In some embodiments, the dose of vincristine in an R-CHOP treatment may be adjusted, e.g., in the event of infusion-associated symptoms, infusion related reactions or adverse events, e.g., as described in Example 1 herein. In some embodiments, the dose of vincristine (which is also known was oncovin) is reduced from 100% of the starting dose (e.g., 1.4 mg/m²) to about 75% of the staring dose, e.g., to about 1.05 mg/m². In some embodiments, the dose of vincristine is reduced from 75% of the starting dose (e.g., 1.4 mg/m²) to about 50% of the staring dose, e.g., to about 0.7 mg/m². In some embodiments of any of the methods provided herein, the dose of vincristine is between about 1.4 mg/m² and about 0.5 mg/m², or between about 1.4 mg/m² and about 0.7 mg/m², but up to 2 mg per dose. In some embodiments of any of the methods provided herein, the dose of vincristine is any of about 0.5 mg/m², 0.55 mg/m², 0.6 mg/m², 0.65 mg/m², 0.7 mg/m², 0.75 mg/m², 0.8 mg/m², 0.85 mg/m², 0.9 mg/m², 0.95 mg/m², 1.0 mg/m², 1.05 mg/m², 1.1 mg/m², 1.15 mg/m², 1.2 mg/m², 1.25 mg/m², 1.3 mg/m², 1.35 mg/m², or 1.4 mg/m². In some embodiments of any of the methods provided herein, 2 mg per dose is the maximum dose of vincristine.

In some embodiments of any of the methods for treating DLBCL provided herein, each dose of anti-CD20 antibody (e.g., obinutuzumab or rituximab) may be administered to the individual over multiple days (e.g., over 2 days), for example, if the individual experiences an infusion-associated symptom, infusion related reaction or adverse event. In some embodiments, if the dose of anti-CD20 antibody (e.g., obinutuzumab or rituximab) is split into two days, the immunoconjugate (e.g., huMA79bv28-MC-vc-PAB-MMAE or polatuzumab vedotin) and the one or more chemotherapeutic agents (e.g., cyclophosphamide and/or doxorubicin) are administered on the second day after completion of administration of the dose of the anti-CD20 antibody (e.g., obinutuzumab or rituximab).

In some embodiments of any of the methods for treating DLBCL provided herein, the anti-CD20 antibody (e.g., obinutuzumab or rituximab) and the one or more chemotherapeutic agents (e.g., cyclophosphamide and/or doxorubicin) are administered on day 1 of each 21-day cycle, and the immunoconjugate (e.g., huMA79bv28-MC-vc-PAB-MMAE or polatuzumab vedotin) is administered on day 2 of each 21-day cycle (e.g., after administration of a corticosteroid, e.g., prednisone, prednisolone, or methylprednisolone).

In some embodiments of any of the methods for treating DLBCL provided herein, the treatment regimen is modified, adjusted, interrupted or delayed as described in Example 1 herein, e.g., in Tables 3, 6, 8 and 9. In some embodiments of any of the methods for treating DLBCL provided herein, the methods comprise managing infusion-related reactions and/or anaphylaxis as described in Example 1 herein, e.g., in Tables 6 and 7.

(vii) Exemplary Biomarkers

In some embodiments of any of the methods for treating DLBCL provided herein, the method further comprises assessing minimal residual disease (MRD) in the individual (e.g., by sequencing such as next-generation sequencing, or by any other suitable method known in the art) before, during and/or after treatment. In some embodiments of any of the methods for treating DLBCL provided herein, the method further comprises assessing circulating tumor DNA (ctDNA), e.g., before, during and/or after treatment, e.g., by sequencing such as next-generation sequencing, or by any other suitable method known in the art. In some embodiments of any of the methods for treating DLBCL provided herein, the method further comprises assessing lymphocyte subsets (e.g., by fluorescence activated cell sorting (FACS) or any other suitable method known in the art) before, during and/or after treatment. In some embodiments of any of the methods for treating DLBCL provided herein, the method further comprises assessing cell of origin of the DLBCL (e.g., by RNA-based gene expression profiling, or by any other suitable method known in the art) before, during and/or after treatment. In some embodiments of any of the methods for treating DLBCL provided herein, the method further comprises assessing the presence or absence of one or more mutations in the DLBCL, such as one or more mutations in MYD88 or CD79B (e.g., by sequencing such as next-generation sequencing, or by any other suitable method known in the art) before, during and/or after treatment. In some embodiments of any of the methods for treating DLBCL provided herein, the method further comprises performing proteomics and/or immunohistochemical analysis of BCL2 and/or MYC in the DLBCL, before, during and/or after treatment. In some embodiments of any of the methods for treating DLBCL provided herein, the method further comprises obtaining translocation profiles of MYC, BCL2 and/or BCL6, before, during and/or after treatment, e.g., using sequencing such as next-generation sequencing, fluorescence in situ hybridization (FISH), or any other suitable method known in the art. In some embodiments of any of the methods for treating DLBCL provided herein, the method further comprises assessing index clone of the DLBCL, e.g., to determine minimal residual disease (MRD), e.g., by sequencing such as next-generation sequencing, or using any other suitable method known in the art.

In some embodiments, an individual, e.g., a human patient, treated according to any of the methods provided herein has an International Prognostic Index (IPI) score of 2. In some embodiments, an individual, e.g., a human patient, treated according to any of the methods provided herein has an International Prognostic Index (IPI) score of between 3 and 5. In some embodiments, an individual, e.g., a human patient, treated according to any of the methods provided herein has bulky disease with one lesion of >7.5 cm. In some embodiments, an individual, e.g., a human patient, treated according to any of the methods provided herein does not have bulky disease. In some embodiments, an individual, e.g., a human patient, treated according to any of the methods provided herein does not have a lesion of ≥7.5 cm.

In some embodiments of any of the methods provided herein, the one or more chemotherapeutic agents (e.g., cyclophosphamide and/or doxorubicin) and/or the corticosteroid (e.g., prednisone, prednisolone, or methylprednisolone) may be replaced with a suitable equivalent. In some embodiments of any of the methods provided herein, cyclophosphamide, doxorubicin, and/or prednisone, prednisolone, or methylprednisolone may be replaced with a suitable equivalent.

### C. Therapeutic Responses and Assessments

In some embodiments, therapeutic responses in an individual, e.g., a human patient, treated according to any of the methods for treating diffuse large B-cell lymphoma (DLBCL) provided herein are assessed according to the Lugano Response Criteria for Malignant Lymphoma (Cheson et al., J Clin Oncol (2014) 32:1-9). In some embodiments, therapeutic responses are assessed as described in Examples 1 or 2 herein, see, e.g., Table 2.

In some embodiments, progression-free survival (PFS) or absence of disease progression is assessed as the time from up to 7 days (e.g., any of 7, 6, 5, 4, 3, 2, 1 or 0 days) prior to initiation of treatment according to the methods provided herein (e.g., on the date of randomization according to the treatment regimen described in Example 1), to the time of a first occurrence of disease progression or relapse, or death from any cause. In some embodiments, PFS or absence of disease progression is assessed from the date of randomization according to treatment regimen described in Example 1, to the time of a first occurrence of disease progression or relapse, or death from any cause. In some embodiments, disease progression or relapse are assessed by an investigator using the 2014 Lugano Classification for Malignant Lymphoma (Cheson et al., 2014). In some embodiments, for individuals who have not progressed, relapsed, or died as of the cutoff date for clinical analysis, PFS is censored on the date of last disease assessment when the individual was known to be progression-free. In some embodiments, if no tumor assessments are performed after baseline or all post-baseline tumor assessment results have overall responses of “not evaluable.” PFS is censored up to 7 days (e.g., any of 7, 6, 5, 4, 3, 2, 1 or 0 days) prior to initiation of treatment according to the methods provided herein (e.g., on the date of randomization according to the treatment regimen described in Example 1). In some embodiments, the PFS is the median PFS of a plurality of human patients treated according to the methods of the disclosure. In some embodiments, PFS of a plurality of human patients treated according to the methods of the disclosure is compared to a reference PFS of a plurality of human patients treated with a control treatment, e.g., R-CHOP. In some embodiments, the PFS and the reference PFS are compared based on a hazard ratio. In some embodiments, the hazard ratio is calculated using any suitable method known in the art, such as a stratified Cox proportional-hazards analysis. In some embodiments, a stratified hazard ratio is calculated using one or more, or all, of the following stratification factors: (a) geographical region (e.g., selected from (i) Asia, (ii) Western Europe, United States of America, Canada, and/or Australia, and (iii) the rest of the world excluding (i) and/or (ii)); (b) International Prognostic Index (IPI) score (e.g., an IPI score of 2 versus between 3 and 5); and/or (c) the presence or absence of bulky disease (e.g., one lesion of ≥7.5 cm). In some embodiments, the PFS and the reference PFS are compared based on a stratified hazard ratio, e.g., as described above. In some embodiments, the PFS and the reference PFS are compared based on an unstratified hazard ratio. In some embodiments, a 95% confidence interval of the hazard ratio is calculated.

In some embodiments, event-free survival-efficacy (EFS_{eff}) is assessed as the time from up to 7 days (e.g., any of 7, 6, 5, 4, 3, 2, 1 or 0) days) prior to initiation of treatment according to the methods provided herein (e.g., on the date of randomization according to the treatment regimen described in Example 1), to the time of the earliest occurrence of an EFS_{eff} event, including any of: disease progression or relapse; death due to any cause: a primary efficacy reason determined by an investigator, other than disease progression or relapse, that leads to initiation of a different anti-lymphoma treatment (NALT); or if a biopsy is obtained after treatment completion, and is positive for residual disease, regardless of whether NALT is initiated or not. In some embodiments, the primary efficacy reason includes instances where a positron emission tomography-computed tomography (PET-CT) scan, bone marrow test, CT/MRI, or physical finding is suggestive of residual disease; or instances where a biopsy confirms residual disease. In some embodiments, event-free survival-efficacy (EFS_{eff}) is assessed from the date of randomization according to the treatment regimen described in Example 1, to the time of the earliest occurrence of an EFS_{eff} event as described above. In some embodiments, EFS_{eff} event timing is at the time of the test or biopsy leading to NALT, rather than the date of NALT initiation. In some embodiments, if an individual does not experience an EFS_{eff} event, then EFS_{eff} is censored on the date of last tumor assessment when the individual is known to be disease progression-free. In some embodiments, for individuals with no EFS_{eff} event, who do not have post-baseline tumor assessments or all post-baseline tumor assessment results have overall responses of ‘not evaluable’, EFS_{eff} is censored up to 7 days (e.g., any of 7, 6, 5, 4, 3, 2, 1 or 0 days) prior to initiation of treatment according to the methods provided herein (e.g., on the date of randomization according to the treatment regimen described in Example 1). In some embodiments, EFS_{eff} of a plurality of human patients treated according to the methods of the disclosure is compared to a reference EFS_{eff} of a plurality of human patients treated with a control treatment, e.g., R-CHOP. In some embodiments, the EFS_{eff} and the reference EFS_{eff} are compared based on a hazard ratio. In some embodiments, the hazard ratio is calculated using any suitable method known in the art, such as using a stratified Cox proportional-hazards analysis. In some embodiments, a stratified hazard ratio is calculated using one or more, or all, of the following stratification factors: (a) geographical region (e.g., selected from (i) Asia, (ii) Western Europe, United States of America, Canada, and/or Australia, and (iii) the rest of the world excluding (i) and/or (ii)); (b) International Prognostic Index (IPI) score (e.g., an IPI score of 2 versus between 3 and 5); and/or (c) the presence or absence of bulky disease (e.g., one lesion of >7.5 cm). In some embodiments, the EFS_{eff} and the reference EFS_{eff} are compared based on a stratified hazard ratio, e.g., as described above. In some embodiments, the EFS_{eff} and the reference EFS_{eff} are compared based on an unstratified hazard ratio. In some embodiments, a 95% confidence interval of the hazard ratio is calculated. In some embodiments, the 12, 24, or 36 month EFS rate (95% CI) is calculated for EFS_{eff}.

In some embodiments, survival is assessed from up to 7 days (e.g., any of 7, 6, 5, 4, 3, 2, 1 or 0 days) prior to initiation of treatment according to the methods provided herein (e.g., on the date of randomization according to the treatment regimen described in Example 1), to death from any cause. In some embodiments, overall survival is assessed from up to 7 days (e.g., any of 7, 6, 5, 4, 3, 2, 1 or 0 days) prior to initiation of treatment according to the methods provided herein (e.g., on the date of randomization according to the treatment regimen described in Example 1), to death from any cause. In some embodiments, survival is assessed from the date of randomization according to treatment regimen described in Example 1, to death from any cause. In some embodiments, overall survival is assessed from the date of randomization according to the treatment regimen described in Example 1, to death from any cause. In some embodiments, for individuals who have not died at the cutoff date for clinical analysis, overall survival is censored on the last date when the individual is known to be alive, e.g., as documented by an investigator. In some embodiments, overall survival (OS) of a plurality of human patients treated according to the methods of the disclosure is compared to a reference OS of a plurality of human patients treated with a control treatment, e.g., R-CHOP. In some embodiments, the OS and the reference OS are compared based on a hazard ratio. In some embodiments, the hazard ratio is calculated using any suitable method known in the art, such as using a stratified Cox proportional-hazards analysis. In some embodiments, a stratified hazard ratio is calculated using one or more, or all, of the following stratification factors: (a) geographical region (e.g., selected from (i) Asia, (ii) Western Europe, United States of America, Canada, and/or Australia, and (iii) the rest of the world excluding (i) and/or (ii)); (b) International Prognostic Index (IPI) score (e.g., an IPI score of 2 versus between 3 and 5); and/or (c) the presence or absence of bulky disease (e.g., one lesion of ≥7.5 cm). In some embodiments, the OS and the reference OS are compared based on a stratified hazard ratio, e.g., as described above. In some embodiments, the OS and the reference OS are compared based on an unstratified hazard ratio. In some embodiments, a 95% confidence interval of the hazard ratio is calculated.

In some embodiments, the complete response rate at the end of treatment is assessed as the proportion of individuals (e.g., among a plurality of individuals treated according to any of the methods provided herein) that exhibits a complete response at the end of treatment according to any of the methods provided herein. In some embodiments, the complete response is assessed by PET-CT by an investigator or by blinded independent central review (BICR), e.g., as described in Example 1 herein.

In some embodiments, the 1-year or 12-month progression-free survival rate is assessed as the proportion of individuals (e.g., among a plurality of individuals treated according to any of the methods provided herein) that exhibits progression-free survival (PFS) at 1 year (i.e., 12 months), assessed starting from up to 7 days (e.g., any of 7, 6, 5, 4, 3, 2, 1 or 0 days) prior to initiation of treatment according to the methods provided herein (e.g., on the date of randomization according to the treatment regimen described in Example 1). In some embodiments, the 1-year (i.e., 12-month) progression-free survival rate is assessed as the proportion of individuals (e.g., among a plurality of individuals treated according to any of the methods provided herein) that exhibits progression-free survival (PFS) at 1 year (i.e., 12 months), assessed starting from the date of randomization according to the treatment regimen described in Example 1.

In some embodiments, the 2-year or 24-month progression-free survival (PFS24) rate is assessed as the proportion of individuals (e.g., among a plurality of individuals treated according to any of the methods provided herein) that exhibits progression-free survival (PFS) at 2 years (i.e., 24 months), assessed starting from up to 7 days (e.g., any of 7, 6, 5, 4, 3, 2, 1 or 0 days) prior to initiation of treatment according to the methods provided herein (e.g., on the date of randomization according to the treatment regimen described in Example 1). In some embodiments, the 2-year progression-free survival (PFS24) rate is assessed as the proportion of individuals (e.g., among a plurality of individuals treated according to any of the methods provided herein) that exhibits progression-free survival (PFS) at 2 years (i.e., 24 months), assessed starting from the date of randomization according to the treatment regimen described in Example 1.

In some embodiments, the 3-year or 36-month progression-free survival (PFS36) rate is assessed as the proportion of individuals (e.g., among a plurality of individuals treated according to any of the methods provided herein) that exhibits progression-free survival (PFS) at 3 years (i.e., 36 months), assessed starting from up to 7 days (e.g., any of 7, 6, 5, 4, 3, 2, 1 or 0 days) prior to initiation of treatment according to the methods provided herein (e.g., on the date of randomization according to the treatment regimen described in Example 1). In some embodiments, the 3-year progression-free survival (PFS36) rate is assessed as the proportion of individuals (e.g., among a plurality of individuals treated according to any of the methods provided herein) that exhibits progression-free survival (PFS) at 3 years (i.e., 36 months), assessed starting from the date of randomization according to the treatment regimen described in Example 1.

In some embodiments, the 42-month progression-free survival (PFS42) rate is assessed as the proportion of individuals (e.g., among a plurality of individuals treated according to any of the methods provided herein) that exhibits progression-free survival (PFS) at 42 months, assessed starting from up to 7 days (e.g., any of 7, 6, 5, 4, 3, 2, 1 or 0 days) prior to initiation of treatment according to the methods provided herein (e.g., on the date of randomization according to the treatment regimen described in Example 1). In some embodiments, the PFS42 rate is assessed as the proportion of individuals (e.g., among a plurality of individuals treated according to any of the methods provided herein) that exhibits PFS at 42 months, assessed starting from the date of randomization according to the treatment regimen described in Example 1.

In some embodiments, disease-free survival (DFS) is assessed as the time from a first occurrence of a complete response in an individual treated according to any of the methods provided herein, to the time of disease relapse or death from any cause for individuals with a best overall response (BOR) of complete response. In some embodiments, for individuals who achieve a complete response but who have not relapsed or died at the time of analysis, DFS is censored on the date of last tumor assessment when the individual is known to be disease-free. In some embodiments, DFS of a plurality of human patients treated according to the methods of the disclosure is compared to a reference DFS of a plurality of human patients treated with a control treatment, e.g., R-CHOP. In some embodiments, the DFS and the reference DFS are compared based on a hazard ratio. In some embodiments, the hazard ratio is calculated using any suitable method known in the art, such as using a stratified Cox proportional-hazards analysis. In some embodiments, a stratified hazard ratio is calculated using one or more, or all, of the following stratification factors: (a) geographical region (e.g., selected from (i) Asia, (ii) Western Europe, United States of America, Canada, and/or Australia, and (iii) the rest of the world excluding (i) and/or (ii)); (b) International Prognostic Index (IPI) score (e.g., an IPI score of 2 versus between 3 and 5); and/or (c) the presence or absence of bulky disease (e.g., one lesion of ≥7.5 cm). In some embodiments, the DFS and the reference DFS are compared based on a stratified hazard ratio, e.g., as described above. In some embodiments, the DFS and the reference DFS are compared based on an unstratified hazard ratio. In some embodiments, a 95% confidence interval of the hazard ratio is calculated.

In some embodiments, duration of response (DOR) is assessed from the time of a first occurrence of a response (e.g., a complete response or a partial response) in an individual treated according to any of the methods provided herein to the time of progression, relapse, or death from any cause for individuals with a best overall response of complete response or partial response. In some embodiments, for individuals achieving a response but who have not progressed, relapsed, or died at the time of analysis, DOR is censored on the date of last tumor assessment when the patient is known to be progression-free. In some embodiments, DOR of a plurality of human patients treated according to the methods of the disclosure is compared to a reference DOR of a plurality of human patients treated with a control treatment, e.g., R-CHOP. In some embodiments, the DOR and the reference DOR are compared based on a hazard ratio. In some embodiments, the hazard ratio is calculated using any suitable method known in the art, such as using a stratified Cox proportional-hazards analysis. In some embodiments, a stratified hazard ratio is calculated using one or more, or all, of the following stratification factors: (a) geographical region (e.g., selected from (i) Asia, (ii) Western Europe, United States of America, Canada, and/or Australia, and (iii) the rest of the world excluding (i) and/or (ii)); (b) International Prognostic Index (IPI) score (e.g., an IPI score of 2 versus between 3 and 5); and/or (c) the presence or absence of bulky disease (e.g., one lesion of ≥7.5 cm). In some embodiments, the DOR and the reference DOR are compared based on a stratified hazard ratio, e.g., as described above. In some embodiments, the DOR and the reference DOR are compared based on an unstratified hazard ratio. In some embodiments, a 95% confidence interval of the hazard ratio is calculated.

In some embodiments, best overall responses (BOR) are assessed as the best response in an individual treated according to any of the methods provided herein. In some embodiments, responses are assessed based on the Lugano Response Criteria for Malignant Lymphoma (Cheson et al., J Clin Oncol (2014) 32:1-9). In some embodiments, responses are assessed by an investigator. In some embodiments, an individual treated according to any of the methods provided herein that does not exhibit a response according to the Lugano Response Criteria for Malignant Lymphoma (Cheson et al., 2014) is considered a non-responder. In some embodiments, best overall responses (BOR) are assessed starting from up to 7 days (e.g., any of 7, 6, 5, 4, 3, 2, 1 or 0 days) prior to initiation of treatment according to the methods provided herein. In some embodiments, best overall responses (BOR) are assessed starting from the date of randomization according to the treatment regimen described in Example 1. In some embodiments, best overall responses (BOR) are assessed according to methods and criteria for assessing the objective response rate (ORR), e.g., as described herein.

In some embodiments, EFS-all causes (EFSₐₗₗ) is assessed from up to 7 days (e.g., any of 7, 6, 5, 4, 3, 2, 1 or 0 days) prior to initiation of treatment according to the methods provided herein to disease progression or relapse, e.g., assessed by an investigator, death from any cause, or initiation of any new anti-lymphoma therapy (NALT). In some embodiments, EFS-all causes (EFSₐₗₗ) is assessed from the date of randomization according to the treatment regimen described in Example 1 to disease progression or relapse, e.g., assessed by an investigator, death from any cause, or initiation of any new anti-lymphoma therapy (NALT). In some embodiments, if disease progression or relapse, death, or initiation of a NALT does not occur, EFSₐₗₗ is censored at the date of last tumor assessment. In some embodiments, for individuals without disease progression or relapse, death, or initiation of a NALT, who have not had post-baseline tumor assessments, EFSₐₗₗ is censored up to 7 days (e.g., any of 7, 6, 5, 4, 3, 2, 1 or 0 days) prior to initiation of treatment according to the methods provided herein (e.g., on the date of randomization according to the treatment regimen described in Example 1).

In some embodiments, the objective response rate (ORR) at the end of treatment is assessed as the proportion of individuals (e.g., among a plurality of individuals treated according to any of the methods provided herein) that exhibits a complete response or a partial response at the end of treatment according to any of the methods provided herein. In some embodiments, the complete response or partial response is assessed by PET-CT by an investigator or by blinded independent central review (BICR), e.g., as described in Example 1 herein.

In some embodiments of any of the methods described herein, PET-CT refers to fluorodeoxyglucose positron emission tomography (FDG-PET), e.g., as described in Example 1 herein.

In some embodiments of any of the methods described herein, therapeutic or clinical responses are assessed by an investigator or by blinded independent central review (BICR). In some embodiments, an investigator refers to a doctor, an oncologist, a radiologist, a nuclear medicine specialist or any other health care professional that is qualified to assess therapeutic or clinical responses in DLBCL. In some embodiments, BICR refers to the assessment of therapeutic or clinical responses in a standardized manner by blinded radiologists, nuclear medicine specialists, and oncologists.

In some embodiments of any of the methods provided herein, therapeutic responses in an individual, e.g., a human patient, are assessed according to the RECIL 2017 Criteria (see, Younes et al., International Working Group consensus response evaluation criteria in lymphoma (RECIL 2017). Ann Oncol (2017) 28 (7): 1436-1447).

Further details regarding clinical staging of and response criteria for lymphomas such as DLBCL are provided in, e.g., Van Heertum et al. (2017) Drug Des. Devel. Ther. 11:1719-1728; Cheson et al. (2016) Blood. 128:2489-2496; Cheson et al. (2014) J. Clin. Oncol. 32 (27): 3059-3067; Barrington et al. (2017) J. Clin. Oncol. 32 (27): 3048-3058; Gallamini et al. (2014) Haematologica. 99 (6): 1107-1113; Barrinton et al. (2010) Eur. J. Nucl. Med. Mol. Imaging. 37 (10): 1824-33; Moskwitz (2012) Hematology Am Soc. Hematol. Educ. Program 2012:397-401; and Follows et al. (2014) Br. J. Haematology 166:34-49. The progress of any one of the methods of treatment provided herein can be monitored by techniques known in the art.

In some embodiments, an individual, e.g., a human patient, treated according to any of the methods described herein achieves an improved response compared to an individual treated with a treatment comprising a single agent, e.g., a treatment with only an immunoconjugate (such as huMA79bv28-MC-vc-PAB-MMAE or polatuzumab vedotin), a treatment with only an anti-CD20 antibody (e.g., obinutuzumab or rituximab), a treatment with only one or more chemotherapeutic drugs (e.g., cyclophosphamide, doxorubicin, and/or vincristine), or a treatment with only a corticosteroid (e.g., prednisone, prednisolone, or methylprednisolone). In some embodiments, an individual, e.g., a human patient, treated according to any of the methods described herein achieves an improved response compared to an individual treated with a treatment comprising an anti-CD20 antibody (e.g., obinutuzumab or rituximab) and one or more chemotherapeutic drugs (e.g., cyclophosphamide, doxorubicin, and/or vincristine). In some embodiments, an individual, e.g., a human patient, treated according to any of the methods described herein achieves an improved response compared to an individual treated with a treatment comprising an anti-CD20 antibody (e.g., obinutuzumab or rituximab), one or more chemotherapeutic drugs (e.g., cyclophosphamide, doxorubicin, and/or vincristine) and a corticosteroid (e.g., prednisone, prednisolone, or methylprednisolone). In some embodiments, an individual, e.g., a human patient, treated according to any of the methods described herein achieves an improved response compared to an individual treated with a treatment comprising rituximab, cyclophosphamide, doxorubicin, vincristine, and a corticosteroid (e.g., prednisone, prednisolone, or methylprednisolone). In some embodiments, an individual, e.g., a human patient, treated according to any of the methods described herein achieves an improved response compared to an individual treated with a control treatment comprising rituximab, cyclophosphamide, doxorubicin, vincristine, and prednisone, prednisolone, or methylprednisolone (R-CHOP). In some embodiments, an individual, e.g., a human patient, treated according to any of the methods described herein achieves an improved response compared to an individual treated with a standard of care treatment for DLBCL, e.g., rituximab plus cyclophosphamide, doxorubicin, vincristine, and prednisone, prednisolone, or methylprednisolone (R-CHOP); or cyclophosphamide, doxorubicin, vincristine, and prednisone, prednisolone, or methylprednisolone (CHOP); or a CHOP-like chemotherapy.

In some embodiments, therapeutic or clinical responses in an individual with DLBCL, e.g., a human patient, or in a plurality of individuals with DLBCL, e.g., a plurality of human patients, treated according to any of the methods described herein are compared to therapeutic or clinical responses in an individual with DLBCL (e.g., a human) or plurality of individuals with DLBCL (e.g., a plurality of humans) treated with a control treatment, wherein the control treatment is R-CHOP. In some embodiments, an individual or a plurality of individuals with DLBCL treated according to any of the methods described herein achieve improved therapeutic or clinical responses as compared to therapeutic or clinical responses in an individual or plurality of individuals with DLBCL treated with R-CHOP. In some embodiments, the individual or plurality of individuals with DLBCL treated with R-CHOP are treated according to the treatment regimen described below and in Example 1 herein.

In some embodiments, the individual or plurality of individuals with DLBCL treated with R-CHOP are treated with rituximab at a dose of about 375 mg/m² intravenously (IV), cyclophosphamide at a dose of about 750 mg/m² IV, doxorubicin at a dose of about 50 mg/m² IV, and vincristine at a dose of about 1.4 mg/m² IV (maximum 2 mg/dose), each given on Day 1 of each 21-day cycle for at least 6 cycles (e.g., Cycles 1-6); and (a) prednisone at a dose of about 100 mg/day orally (PO) given on Days 1-5 of every 21-day cycle for at least 6 cycles (e.g., Cycles 1-6), (b) prednisolone at a dose of about 100 mg/day PO given on Days 1-5 of every 21-day cycle for at least 6 cycles (e.g., Cycles 1-6), or (c) methylprednisolone at a dose of about 80 mg/day IV given on Days 1-5 of every 21-day cycle for at least 6 cycles (e.g., Cycles 1-6). In some embodiments, the individual or plurality of individuals with DLBCL treated with R-CHOP are further administered rituximab at a dose of about 375 mg/m² IV, given as monotherapy in 21-day Cycles 7 and 8 after Cycles 1-6, e.g., on day 1 of each cycle.

In some embodiments, the individual or plurality of individuals with DLBCL treated with R-CHOP are treated with rituximab at a dose of about 375 mg/m² intravenously (IV), cyclophosphamide at a dose of about 750 mg/m² IV, doxorubicin at a dose of about 50 mg/m² IV, and vincristine at a dose of about 1.4 mg/m² IV (maximum 2 mg/dose), each given on Day 1 of each 21-day cycle for 6 cycles (e.g., Cycles 1-6); and (a) prednisone at a dose of about 100 mg/day orally (PO) given on Days 1-5 of every 21-day cycle for 6 cycles (e.g., Cycles 1-6), (b) prednisolone at a dose of about 100 mg/day PO given on Days 1-5 of every 21-day cycle for 6 cycles (e.g., Cycles 1-6), or (c) methylprednisolone at a dose of about 80 mg/day IV given on Days 1-5 of every 21-day cycle for 6 cycles (e.g., Cycles 1-6). In some embodiments, the individual or plurality of individuals with DLBCL treated with R-CHOP are further administered rituximab at a dose of about 375 mg/m² IV, given as monotherapy in 21-day cycles for two additional 21-day cycles (e.g., on Cycles 7 and 8), e.g., on day 1 of each cycle.

In some embodiments, the individual or plurality of individuals with DLBCL treated with R-CHOP are treated with rituximab at a dose of about 375 mg/m² intravenously (IV), cyclophosphamide at a dose of about 750 mg/m² IV, doxorubicin at a dose of about 50 mg/m² IV, and vincristine at a dose of about 1.4 mg/m² IV (maximum 2 mg/dose), each given on Day 1 of each 21-day cycle for 8 cycles (e.g., Cycles 1-8); and (a) prednisone at a dose of about 100 mg/day orally (PO) given on Days 1-5 of every 21-day cycle for 8 cycles (e.g., Cycles 1-8), (b) prednisolone at a dose of about 100 mg/day PO given on Days 1-5 of every 21-day cycle for 8 cycles (e.g., Cycles 1-8), or (c) methylprednisolone at a dose of about 80 mg/day IV given on Days 1-5 of every 21-day cycle for 8 cycles (e.g., Cycles 1-8).

In some embodiments, the individual or plurality of individuals with DLBCL treated with R-CHOP are treated with rituximab at a dose of about 375 mg/m² intravenously (IV), cyclophosphamide at a dose of about 750 mg/m² IV, doxorubicin at a dose of about 50 mg/m² IV, and vincristine at a dose of about 1.4 mg/m² IV (maximum 2 mg/dose), each given on Day 1 of each 21-day cycle for between 6 and 8 cycles (e.g., Cycles 1-6, Cycles 1-7, or Cycles 1-8); and (a) prednisone at a dose of about 100 mg/day orally (PO) given on Days 1-5 of every 21-day cycle for between 6 and 8 cycles (e.g., Cycles 1-6, Cycles 1-7, or Cycles 1-8), (b) prednisolone at a dose of about 100 mg/day PO given on Days 1-5 of every 21-day cycle for between 6 and 8 cycles (e.g., Cycles 1-6, Cycles 1-7, or Cycles 1-8), or (c) methylprednisolone at a dose of about 80 mg/day IV given on Days 1-5 of every 21-day cycle for between 6 and 8 cycles (e.g., Cycles 1-6, Cycles 1-7, or Cycles 1-8).

In some embodiments, treatment of a plurality of human patients according to the methods of the disclosure results in an improvement in progression-free survival (PFS) of the plurality of human patients as compared to a reference PFS, wherein the reference PFS is the PFS of a plurality of human patients who have received treatment with R-CHOP. In some embodiments, treatment of a plurality of human patients having an age greater than 60 years according to the methods of the disclosure results in an improvement in progression-free survival (PFS) of the plurality of human patients as compared to a reference PFS, wherein the reference PFS is the PFS of a plurality of human patients having an age greater than 60 years who have received treatment with R-CHOP. In some embodiments, treatment of a plurality of human patients having an age greater than 65 years according to the methods of the disclosure results in an improvement in progression-free survival (PFS) of the plurality of human patients as compared to a reference PFS, wherein the reference PFS is the PFS of a plurality of human patients having an age greater than 65 years who have received treatment with R-CHOP. In some embodiments, treatment of a plurality of human patients having an International Prognostic Index (IPI) score between 3 and 5 according to the methods of the disclosure results in an improvement in progression-free survival (PFS) of the plurality of human patients as compared to a reference PFS, wherein the reference PFS is the PFS of a plurality of human patients having an IPI score between 3 and 5 who have received treatment with R-CHOP. In some embodiments, treatment of a plurality of human patients having an age greater than 60 years and an International Prognostic Index (IPI) score between 3 and 5 according to the methods of the disclosure results in an improvement in progression-free survival (PFS) of the plurality of human patients as compared to a reference PFS, wherein the reference PFS is the PFS of a plurality of human patients having an age greater than 60 years and an IPI score between 3 and 5 who have received treatment with R-CHOP. In some embodiments, treatment of a plurality of human patients having an age greater than 65 years and an International Prognostic Index (IPI) score between 3 and 5 according to the methods of the disclosure results in an improvement in progression-free survival (PFS) of the plurality of human patients as compared to a reference PFS, wherein the reference PFS is the PFS of a plurality of human patients having an age greater than 65 years and an IPI score between 3 and 5 who have received treatment with R-CHOP. In some embodiments, treatment of a plurality of human patients having an ABC type DLBCL according to the methods of the disclosure results in an improvement in progression-free survival (PFS) of the plurality of human patients as compared to a reference PFS, wherein the reference PFS is the PFS of a plurality of human patients having an ABC type DLBCL who have received treatment with R-CHOP. In some embodiments, treatment of a plurality of human patients having a DEL type DLBCL according to the methods of the disclosure results in an improvement in progression-free survival (PFS) of the plurality of human patients as compared to a reference PFS, wherein the reference PFS is the PFS of a plurality of human patients having a DEL type DLBCL who have received treatment with R-CHOP. In some embodiments, treatment of a plurality of human patients according to the methods of the disclosure results in a hazard ratio of no more than 0.75 (e.g., 0.74, 0.73, 0.72, 0.71, 0.70) in progression-free survival (PFS) of the plurality of human patients as compared to treatment with R-CHOP. In some embodiments, treatment of a plurality of human patients according to the methods of the disclosure results in a hazard ratio of no more than 0.78 (e.g., 0.77, 0.76, 0.75, 0.74, 0.73, 0.72, 0.71, 0.70) in progression-free survival (PFS) of the plurality of human patients as compared to treatment with R-CHOP. In some embodiments, treatment of a plurality of human patients according to the methods of the disclosure results in a hazard ratio of no more than 0.79 (e.g., 0.78, 0.77, 0.76, 0.75, 0.74, 0.73, 0.72, 0.71, 0.70) in progression-free survival (PFS) of the plurality of human patients as compared to treatment with R-CHOP. In some embodiments, PFS or the reference PFS is measured starting from up to 7 days (e.g., any of 7, 6, 5, 4, 3, 2, 1 or 0 days) prior to the start of treatment to the time of a first occurrence of disease progression, relapse, or death. In some embodiments, PFS or the reference PFS is measured starting from the start of treatment to the time of a first occurrence of disease progression, relapse, or death. In some embodiments, PFS or the reference PFS is measured starting from the date of randomization according to the treatment regimen described in Example 1, to the time of a first occurrence of disease progression, relapse, or death. In some embodiments, the PFS is the median PFS of the plurality of human patients receiving treatment according to the methods described herein. In some embodiments, the reference PFS is the median PFS of the plurality of human patients receiving R-CHOP. In some embodiments, the improvement in PFS is statistically significant. In some embodiments, the improvement in PFS is statistically significant with a stratified hazard ratio of no more than 0.75 (95% confidence interval: 0.57, 0.97). In some embodiments, the improvement in PFS is statistically significant with a stratified hazard ratio of no more than 0.78 (95% confidence interval: 0.60, 1.00). In some embodiments, the improvement in PFS is statistically significant with an unstratified hazard ratio of no more than 0.79 (95% confidence interval: 0.61, 1.02). In some embodiments, treatment of a plurality of human patients having an age greater than 60 years according to the methods of the disclosure results in an improvement in progression-free survival (PFS) of the plurality of human patients as compared to a reference PFS with a hazard ratio of no more than 0.8 (e.g., any of 0.8, 0.78, 0.75, 0.76, 0.7, 0.65, 0.6, 0.55, 0.5, 0.45, 0.4 or less), wherein the reference PFS is the PFS of a plurality of human patients having an age greater than 60 years who have received treatment with R-CHOP. In some embodiments, treatment of a plurality of human patients having an age greater than 60 years according to the methods of the disclosure results in an improvement in progression-free survival (PFS) of the plurality of human patients as compared to a reference PFS with a stratified hazard ratio of no more than 0.72 (95% confidence interval: 0.52, 0.99), wherein the reference PFS is the PFS of a plurality of human patients having an age greater than 60 years who have received treatment with R-CHOP. In some embodiments, treatment of a plurality of human patients having an age greater than 60 years according to the methods of the disclosure results in an improvement in progression-free survival (PFS) of the plurality of human patients as compared to a reference PFS with an unstratified hazard ratio of no more than 0.72 (95% confidence interval: 0.53, 0.99), wherein the reference PFS is the PFS of a plurality of human patients having an age greater than 60 years who have received treatment with R-CHOP. In some embodiments, treatment of a plurality of human patients having an age greater than 60 years according to the methods of the disclosure results in an improvement in progression-free survival (PFS) of the plurality of human patients as compared to a reference PFS with an unstratified hazard ratio of no more than 0.76 (95% confidence interval: 0.56, 1.02), wherein the reference PFS is the PFS of a plurality of human patients having an age greater than 60 years who have received treatment with R-CHOP. In some embodiments, treatment of a plurality of human patients having an age greater than 65 years according to the methods of the disclosure results in an improvement in progression-free survival (PFS) of the plurality of human patients with a hazard ratio of no more than 0.9 (e.g., any of 0.9, 0.85, 0.8, 0.78, 0.76, 0.75, 0.7, 0.65, 0.6, 0.55, 0.5, 0.45, 0.4 or less) as compared to a reference PFS, wherein the reference PFS is the PFS of a plurality of human patients having an age greater than 65 years who have received treatment with R-CHOP. In some embodiments, treatment of a plurality of human patients having an age greater than 65 years according to the methods of the disclosure results in an improvement in progression-free survival (PFS) of the plurality of human patients with a stratified hazard ratio of no more than 0.79 (95% confidence interval: 0.54, 1.14) as compared to a reference PFS, wherein the reference PFS is the PFS of a plurality of human patients having an age greater than 65 years who have received treatment with R-CHOP. In some embodiments, treatment of a plurality of human patients having an age greater than 65 years according to the methods of the disclosure results in an improvement in progression-free survival (PFS) of the plurality of human patients with a unstratified hazard ratio of no more than 0.77 (95% confidence interval: 0.54, 1.10) as compared to a reference PFS, wherein the reference PFS is the PFS of a plurality of human patients having an age greater than 65 years who have received treatment with R-CHOP. In some embodiments, treatment of a plurality of human patients having an age greater than 65 years according to the methods of the disclosure results in an improvement in progression-free survival (PFS) of the plurality of human patients with a unstratified hazard ratio of no more than 0.78 (95% confidence interval: 0.56, 1.10) as compared to a reference PFS, wherein the reference PFS is the PFS of a plurality of human patients having an age greater than 65 years who have received treatment with R-CHOP. In some embodiments, treatment of a plurality of human patients having an International Prognostic Index (IPI) score between 3 and 5 according to the methods of the disclosure results in an improvement in progression-free survival (PFS) of the plurality of human patients with a hazard ratio of no more than 0.8 (e.g., any of 0.8, 0.75, 0.7, 0.65, 0.6, 0.55, 0.5, 0.45, 0.4 or less) as compared to a reference PFS, wherein the reference PFS is the PFS of a plurality of human patients having an IPI score between 3 and 5 who have received treatment with R-CHOP. In some embodiments, treatment of a plurality of human patients having an International Prognostic Index (IPI) score between 3 and 5 according to the methods of the disclosure results in an improvement in progression-free survival (PFS) of the plurality of human patients with a stratified hazard ratio of no more than 0.68 (95% confidence interval: 0.50, 0.94) as compared to a reference PFS, wherein the reference PFS is the PFS of a plurality of human patients having an IPI score between 3 and 5 who have received treatment with R-CHOP. In some embodiments, treatment of a plurality of human patients having an International Prognostic Index (IPI) score between 3 and 5 according to the methods of the disclosure results in an improvement in progression-free survival (PFS) of the plurality of human patients with an unstratified hazard ratio of no more than 0.71 (95% confidence interval: 0.51, 0.97) as compared to a reference PFS, wherein the reference PFS is the PFS of a plurality of human patients having an IPI score between 3 and 5 who have received treatment with R-CHOP. In some embodiments, treatment of a plurality of human patients having an International Prognostic Index (IPI) score between 3 and 5 according to the methods of the disclosure results in an improvement in progression-free survival (PFS) of the plurality of human patients with an unstratified hazard ratio of no more than 0.75 (95% confidence interval: 0.55, 1.01) as compared to a reference PFS, wherein the reference PFS is the PFS of a plurality of human patients having an IPI score between 3 and 5 who have received treatment with R-CHOP. In some embodiments, treatment of a plurality of human patients having an ABC type DLBCL according to the methods of the disclosure results in an improvement in progression-free survival (PFS) of the plurality of human patients as compared to a reference PFS with a hazard ratio of no more than 0.4 (e.g., any of 0.4, 0.39, 0.38, 0.37, 0.36, 0.35, 0.3, 0.25, 0.2, 0.15, 0.1, or less), wherein the reference PFS is the PFS of a plurality of human patients having an ABC type DLBCL who have received treatment with R-CHOP. In some embodiments, treatment of a plurality of human patients having an ABC type DLBCL according to the methods of the disclosure results in an improvement in progression-free survival (PFS) of the plurality of human patients as compared to a reference PFS with a stratified hazard ratio of no more than 0.31 (95% confidence interval: 0.17, 0.56), wherein the reference PFS is the PFS of a plurality of human patients having an ABC type DLBCL who have received treatment with R-CHOP. In some embodiments, treatment of a plurality of human patients having an ABC type DLBCL according to the methods of the disclosure results in an improvement in progression-free survival (PFS) of the plurality of human patients as compared to a reference PFS with an unstratified hazard ratio of no more than 0.36 (95% confidence interval: 0.21, 0.62), wherein the reference PFS is the PFS of a plurality of human patients having an ABC type DLBCL who have received treatment with R-CHOP. In some embodiments, treatment of a plurality of human patients having an ABC type DLBCL according to the methods of the disclosure results in an improvement in progression-free survival (PFS) of the plurality of human patients as compared to a reference PFS with an unstratified hazard ratio of no more than 0.39 (95% confidence interval: 0.23, 0.65), wherein the reference PFS is the PFS of a plurality of human patients having an ABC type DLBCL who have received treatment with R-CHOP. In some embodiments, treatment of a plurality of human patients having a DEL type DLBCL according to the methods of the disclosure results in an improvement in progression-free survival (PFS) of the plurality of human patients as compared to a reference PFS with a hazard ratio of no more than 0.7 (e.g., any of 0.7, 0.65, 0.6, 0.55, 0.5, 0.45, 0.4 or less), wherein the reference PFS is the PFS of a plurality of human patients having a DEL type DLBCL who have received treatment with R-CHOP. In some embodiments, treatment of a plurality of human patients having a DEL type DLBCL according to the methods of the disclosure results in an improvement in progression-free survival (PFS) of the plurality of human patients as compared to a reference PFS with a stratified hazard ratio of no more than 0.62 (95% confidence interval: 0.40, 0.97), wherein the reference PFS is the PFS of a plurality of human patients having a DEL type DLBCL who have received treatment with R-CHOP. In some embodiments, treatment of a plurality of human patients having a DEL type DLBCL according to the methods of the disclosure results in an improvement in progression-free survival (PFS) of the plurality of human patients as compared to a reference PFS with an unstratified hazard ratio of no more than 0.65 (95% confidence interval: 0.43, 0.98), wherein the reference PFS is the PFS of a plurality of human patients having a DEL type DLBCL who have received treatment with R-CHOP. In some embodiments, treatment of a plurality of human patients having a DEL type DLBCL according to the methods of the disclosure results in an improvement in progression-free survival (PFS) of the plurality of human patients as compared to a reference PFS with an unstratified hazard ratio of no more than 0.67 (95% confidence interval: 0.44, 1.02), wherein the reference PFS is the PFS of a plurality of human patients having a DEL type DLBCL who have received treatment with R-CHOP. In some embodiments, the hazard ratio has a 95% confidence interval. In some embodiments, the hazard ratio is calculated at 12 months or more, 24 months or more, or 36 months or more, measured starting from: (a) the start of the corresponding treatment (i.e., the treatment according to the methods of the disclosure, or R-CHOP); (b) up to 7 days prior to the start of the corresponding treatment (i.e., the treatment according to the methods of the disclosure, or R-CHOP); or (c) the date of randomization according to the treatment regimen described in Example 1. In some embodiments, such treatment results in a statistically significant improvement in the PFS as compared to the control treatment with a stratified hazard ratio of no more than 0.75 (95% confidence interval: 0.57, 0.97). In some embodiments, such treatment results in a statistically significant improvement in the PFS as compared to the control treatment with a stratified hazard ratio of no more than 0.78 (95% confidence interval: 0.60, 1.00). In some embodiments, such treatment results in a statistically significant improvement in the PFS as compared to the control treatment with an unstratified hazard ratio of no more than 0.79 (95% confidence interval: 0.61, 1.02).

In some embodiments, treatment of a plurality of human patients according to the methods of the disclosure results in at least a 20% (e.g., 21%, 22%, 23%, or 24%) reduction in the risk of disease progression, relapse, or death in the plurality of human patients as compared to treatment with R-CHOP. In other embodiments, treatment of a plurality of human patients according to the methods of the disclosure results in at least a 25% (e.g., 26%, 27%, 28%, 29%, or 30%) reduction in the risk of disease progression, relapse, or death in the plurality of human patients as compared to treatment with R-CHOP. In some embodiments, disease progression, relapse, or death is measured starting from up to 7 days (e.g., any of 7, 6, 5, 4, 3, 2, 1 or 0) days) prior to the start of treatment to the time of a first occurrence of disease progression, relapse, or death. In some embodiments, disease progression, relapse, or death is measured starting from the start of treatment to the time of a first occurrence of disease progression, relapse, or death. In some embodiments, disease progression, relapse, or death is measured starting from the date of randomization according to the treatment regimen described in Example 1, to the time of a first occurrence of disease progression, relapse, or death. In some embodiments, the reduction of risk has a 95% confidence interval. In some embodiments, the reduction in the risk of disease progression, relapse, or death is statistically significant. In some embodiments, the reduction in the risk of disease progression, relapse, or death is calculated at 12 months or more, 24 months or more, or 36 months or more, measured starting from: (a) the start of the corresponding treatment (i.e., the treatment according to the methods of the disclosure, or R-CHOP); (b) up to 7 days prior to the start of the corresponding treatment (i.e., the treatment according to the methods of the disclosure, or R-CHOP); or (c) the date of randomization according to the treatment regimen described in Example 1. In some embodiments, such treatment results in a statistically significant improvement in the PFS as compared to the control treatment with at least a 20% (e.g., 21%, 22%, 23%, or 24%) reduction in the risk of disease progression, relapse, or death. In some embodiments, such treatment results in a statistically significant improvement in the PFS as compared to the control treatment with at least a 25% (e.g., 26%, 27%, 28%, 29%, or 30%) reduction in the risk of disease progression, relapse, or death.

In some embodiments, treatment of a plurality of human patients according to the methods of the disclosure results in a 12-month progression-free survival rate of at least about 83% (e.g., any of 83%, 84%, 85%, 86%, 87%, 88%, 90%, or more). In some embodiments, treatment of a plurality of human patients according to the methods of the disclosure results in an improvement in a 12-month progression-free survival rate of the plurality of human patients as compared to a reference 12-month progression-free survival rate, wherein the reference 12-month progression-free survival rate is the 12-month progression-free survival rate of a plurality of human patients who have received treatment with R-CHOP. In some embodiments, treatment of a plurality of human patients according to the methods of the disclosure results in an improvement in a 12-month progression-free survival rate of the plurality of human patients of at least about 3% (e.g., any of at least about 3%, 4%, 5%, 6%, 7%, 8%, 9%, 10%, or more), as compared to a reference 12-month progression-free survival rate, wherein the reference 12-month progression-free survival rate is the 12-month progression-free survival rate of a plurality of human patients who have received treatment with R-CHOP. In some embodiments, the 12-month progression-free survival rate or the reference 12-month progression-free survival rate is calculated at 12 months, measured starting from up to 7 days (e.g., any of 7, 6, 5, 4, 3, 2, 1 or 0 days) prior to the start of the corresponding treatment (i.e., the treatment according to the methods of the disclosure, or R-CHOP). In some embodiments, the 12-month progression-free survival rate or the reference 12-month progression-free survival rate is calculated at 12 months, measured starting from the start of the corresponding treatment (i.e., the treatment according to the methods of the disclosure, or R-CHOP). In some embodiments, the 12-month progression-free survival rate or the reference 12-month progression-free survival rate is calculated at 12 months, measured starting from the date of randomization according to the treatment regimen described in Example 1. In some embodiments, the 12-month progression-free survival rate or the reference 12-month progression-free survival rate is a progression-free survival (PFS) rate calculated using a Kaplan-Meier method. In some embodiments, the improvement in 12-month progression-free survival rate is statistically significant.

In some embodiments, treatment of a plurality of human patients according to the methods of the disclosure results in a 24-month progression-free survival rate (PFS24) of at least about 75% (e.g., 76%, 77%, 78%, 79%, 80%). In some embodiments, treatment of a plurality of human patients according to the methods of the disclosure results in an improvement in a 24-month progression-free survival rate (PFS24) of the plurality of human patients as compared to a reference PFS24, wherein the reference PFS24 is the 24-month progression-free survival rate of a plurality of human patients who have received treatment with R-CHOP. In some embodiments, treatment of a plurality of human patients according to the methods of the disclosure results in an improvement in a 24-month progression-free survival rate (PFS24) of the plurality of human patients of at least about 5% (e.g., any of about 5%, 5.1%, 5.2%, 5.3%, 5.4%, 5.5%, 5.6%, 5.7%, 5.8%, 5.9%, 6%, or more), as compared to a reference PFS24, wherein the reference PFS24 is the 24-month progression-free survival rate of a plurality of human patients who have received treatment with R-CHOP. In some embodiments, treatment of a plurality of human patients according to the methods of the disclosure results in an improvement in a 24-month progression-free survival rate (PFS24) of the plurality of human patients of at least about 6% (e.g., 6%, 7%, 8%, 9%, 10%), as compared to a reference PFS24, wherein the reference PFS24 is the 24-month progression-free survival rate of a plurality of human patients who have received treatment with R-CHOP. In some embodiments, the PFS24 or the reference PFS24 is calculated at 24 months, measured starting from up to 7 days (e.g., any of 7, 6, 5, 4, 3, 2, 1 or 0 days) prior to the start of the corresponding treatment (i.e., the treatment according to the methods of the disclosure, or R-CHOP). In some embodiments, the PFS24 or the reference PFS24 is calculated at 24 months, measured starting from the start of the corresponding treatment (i.e., the treatment according to the methods of the disclosure, or R-CHOP). In some embodiments, the PFS24 or the reference PFS24 is calculated at 24 months, measured starting from the date of randomization according to the treatment regimen described in Example 1. In some embodiments, the PFS24 or the reference PFS24 is a progression-free survival (PFS) rate calculated using a Kaplan-Meier method. In some embodiments, the improvement in PFS24 is statistically significant.

In some embodiments, treatment of a plurality of human patients according to the methods of the disclosure results in a 36-month progression-free survival rate (PFS36) of at least about 70% (e.g., 71%, 72%, 73%, 74%, 75%, 76%, 77%, 78%, 79%, or 80%). In some embodiments, treatment of a plurality of human patients according to the methods of the disclosure results in an improvement in a 36-month progression-free survival rate (PFS36) of the plurality of human patients as compared to a reference PFS36, wherein the reference PFS36 is the 36-month progression-free survival rate of a plurality of human patients who have received treatment with R-CHOP. In some embodiments, treatment of a plurality of human patients according to the methods of the disclosure results in an improvement in a 36-month progression-free survival rate (PFS36) of the plurality of human patients of at least about 5% (e.g., 6%, 7%, 8%, 9%, 10%, 11%, 12%, 13%, 14%, or 15%) as compared to a reference PFS36, wherein the reference PFS36 is the 36-month progression-free survival rate of a plurality of human patients who have received treatment with R-CHOP. In some embodiments, the PFS36 or the reference PFS36 is calculated at 36 months, measured starting from up to 7 days (e.g., any of 7, 6, 5, 4, 3, 2, 1 or 0 days) prior to the start of the corresponding treatment (i.e., the treatment according to the methods of the disclosure, or R-CHOP). In some embodiments, the PFS36 or the reference PFS36 is calculated at 36 months, measured starting from the start of the corresponding treatment (i.e., the treatment according to the methods of the disclosure, or R-CHOP). In some embodiments, the PFS36 or the reference PFS36 is calculated at 36 months, measured starting from the date of randomization according to the treatment regimen described in Example 1. In some embodiments, the PFS36 or the reference PFS36 is a progression-free survival (PFS) rate calculated using a Kaplan-Meier method. In some embodiments, the improvement in PFS36 is statistically significant.

In some embodiments, treatment of a plurality of human patients according to the methods of the disclosure results in a 42-month progression-free survival rate (PFS42) of at least about 65% (e.g., 66%, 67%, 68, %, 69%, 70% 71%, 72%, 73%, 74%, 75%, 76%, 77%, 78%, 79%, or 80%). In some embodiments, treatment of a plurality of human patients according to the methods of the disclosure results in an improvement in a 42-month progression-free survival rate (PFS42) of the plurality of human patients as compared to a reference PFS42, wherein the reference PFS42 is the 42-month progression-free survival rate of a plurality of human patients who have received treatment with R-CHOP. In some embodiments, treatment of a plurality of human patients according to the methods of the disclosure results in an improvement in PFS42 of the plurality of human patients of at least about 5% (e.g., 6%, 7%, 8%, 9%, 10%, 11%, 12%, 13%, 14%, or 15%) as compared to a reference PFS42, wherein the reference PFS42 is the 42-month progression-free survival rate of a plurality of human patients who have received treatment with R-CHOP. In some embodiments, the PFS42 or the reference PFS42 is calculated at 42 months, measured starting from up to 7 days (e.g., any of 7, 6, 5, 4, 3, 2, 1 or 0 days) prior to the start of the corresponding treatment (i.e., the treatment according to the methods of the disclosure, or R-CHOP). In some embodiments, the PFS42 or the reference PFS42 is calculated at 42 months, measured starting from the start of the corresponding treatment (i.e., the treatment according to the methods of the disclosure, or R-CHOP). In some embodiments, the PFS42 or the reference PFS42 is calculated at 42 months, measured starting from the date of randomization according to the treatment regimen described in Example 1. In some embodiments, the PFS42 or the reference PFS42 is a progression-free survival (PFS) rate calculated using a Kaplan-Meier method. In some embodiments, the improvement in PFS42 is statistically significant.

In some embodiments, treatment of a plurality of human patients according to the methods of the disclosure results in an improvement in overall survival (OS) of the plurality of human patients as compared to a reference OS, wherein the reference OS is the OS of a plurality of human patients who have received treatment with R-CHOP. In some embodiments, treatment of a plurality of human patients according to the methods of the disclosure results in a hazard ratio of no more than 1.0 (e.g., 0.99, 0.98, 0.97, 0.96, 0.95, 0.90, 0.85, 0.80, 0.75, 0.7, 0.65, or 0.6) in overall survival (OS) of the plurality of human patients, as compared to treatment with R-CHOP. In some embodiments, the OS or the reference OS is measured starting from the start of the corresponding treatment (i.e., the treatment according to the methods of the disclosure, or R-CHOP) to the time of death from any cause. In some embodiments, the OS or the reference OS is measured starting from up to 7 days (e.g., any of 7, 6, 5, 4, 3, 2, 1 or 0 days) prior to the start of the corresponding treatment (i.e., the treatment according to the methods of the disclosure, or R-CHOP) to the time of death from any cause. In some embodiments, the OS or the reference OS is measured starting from the date of randomization according to the treatment regimen described in Example 1 to the time of death from any cause. In some embodiments, the improvement in OS is statistically significant. In some embodiments, the hazard ratio has a 95% confidence interval. In some embodiments, treatment of a plurality of human patients according to the methods of the disclosure results in a stratified hazard ratio of no more than 1.01 (95% confidence interval: 0.69, 1.49) in overall survival (OS) of the plurality of human patients, as compared to treatment with R-CHOP. In some embodiments, treatment of a plurality of human patients according to the methods of the disclosure results in a stratified hazard ratio of no more than 0.99 (95% confidence interval: 0.69, 1.41) in overall survival (OS) of the plurality of human patients, as compared to treatment with R-CHOP. In some embodiments, treatment of a plurality of human patients according to the methods of the disclosure results in an unstratified hazard ratio of no more than 0.98 (95% confidence interval: 0.69, 1.40) in overall survival (OS) of the plurality of human patients, as compared to treatment with R-CHOP. In some embodiments, treatment of a plurality of human patients according to the methods of the disclosure results in an unstratified hazard ratio of no more than 0.99 (95% confidence interval: 0.67, 1.45) in overall survival (OS) of the plurality of human patients, as compared to treatment with R-CHOP. In some embodiments, the hazard ratio is calculated at 12 months or more, 24 months or more, or 36 months or more, measured starting from the start of the corresponding treatment (i.e., the treatment according to the methods of the disclosure, or R-CHOP). In some embodiments, the hazard ratio is calculated at 12 months or more, 24 months or more, or 36 months or more, measured starting from up to 7 days (e.g., any of 7, 6, 5, 4, 3, 2, 1 or 0 days) prior to the start of the corresponding treatment (i.e., the treatment according to the methods of the disclosure, or R-CHOP). In some embodiments, the hazard ratio is calculated at 12 months or more, 24 months or more, or 36 months or more, measured starting from the date of randomization according to the treatment regimen described in Example 1 to the time of death from any cause.

In some embodiments, treatment of a plurality of human patients according to the methods of the disclosure results in an improvement in disease free survival (DFS) of the plurality of human patients as compared to a reference DFS, wherein the reference DFS is the DFS of a plurality of human patients who have received treatment with R-CHOP. In some embodiments, treatment of a plurality of human patients according to the methods of the disclosure results in a hazard ratio of no more than 0.8 (e.g., any of about 0.8, 0.79, 0.78, 0.77, 0.76, 0.75, 0.74, 0.73, 0.72, 0.71, 0.7, 0.65, 0.6, 0.55, 0.5, 0.45, 0.4, or less) in DFS of the plurality of human patients, as compared to treatment with R-CHOP. In some embodiments, the improvement in DFS is statistically significant. In some embodiments, the hazard ratio has a 95% confidence interval. In some embodiments, treatment of a plurality of human patients according to the methods of the disclosure results in a stratified hazard ratio of no more than 0.72 (95% confidence interval: 0.51, 1.02) in DFS of the plurality of human patients, as compared to treatment with R-CHOP. In some embodiments, treatment of a plurality of human patients according to the methods of the disclosure results in an unstratified hazard ratio of no more than 0.74 (95% confidence interval: 0.52, 1.05) in DFS of the plurality of human patients, as compared to treatment with R-CHOP. In some embodiments, treatment of a plurality of human patients according to the methods of the disclosure results in a stratified hazard ratio of no more than 0.74 (95% confidence interval: 0.53, 1.02) in DFS of the plurality of human patients, as compared to treatment with R-CHOP. In some embodiments, treatment of a plurality of human patients according to the methods of the disclosure results in an unstratified hazard ratio of no more than 0.76 (95% confidence interval: 0.55, 1.05) in DFS of the plurality of human patients, as compared to treatment with R-CHOP. In some embodiments, DFS is measured starting from the time of a first occurrence of a complete response to the time of disease relapse or death from any cause, e.g., for individuals with a best overall response (BOR) of complete response.

In some embodiments, treatment of a plurality of human patients according to the methods of the disclosure results in an improvement in duration of response (DOR) of the plurality of human patients as compared to a reference DOR, wherein the reference DOR is the DOR of a plurality of human patients who have received treatment with R-CHOP. In some embodiments, treatment of a plurality of human patients according to the methods of the disclosure results in a hazard ratio of no more than 0.8 (e.g., any of about 0.8, 0.79, 0.78, 0.77, 0.76, 0.75, 0.7, 0.65, 0.6, 0.55, 0.5, 0.45, 0.4, or less) in DOR of the plurality of human patients, as compared to treatment with R-CHOP. In some embodiments, treatment of a plurality of human patients according to the methods of the disclosure results in a stratified hazard ratio of no more than 0.75 (95% confidence interval: 0.56, 1.00) in DOR of the plurality of human patients, as compared to treatment with R-CHOP. In some embodiments, treatment of a plurality of human patients according to the methods of the disclosure results in an unstratified hazard ratio of no more than 0.77 (95% confidence interval: 0.58, 1.03) in DOR of the plurality of human patients, as compared to treatment with R-CHOP. In some embodiments, treatment of a plurality of human patients according to the methods of the disclosure results in a stratified hazard ratio of no more than 0.78 (95% confidence interval: 0.59, 1.02) in DOR of the plurality of human patients, as compared to treatment with R-CHOP. In some embodiments, treatment of a plurality of human patients according to the methods of the disclosure results in an unstratified hazard ratio of no more than 0.79 (95% confidence interval: 0.60, 1.03) in DOR of the plurality of human patients, as compared to treatment with R-CHOP. In some embodiments, DOR is measured starting from the time of a first occurrence of a response (e.g., a complete response or a partial response) to the time of disease progression, relapse, or death from any cause, e.g., for individual with a response, e.g., a complete response or a partial response.

In some embodiments, treatment of a plurality of human patients according to the methods of the disclosure results in an improvement in event-free survival-efficacy (EFS_{eff}) of the plurality of human patients as compared to a reference EFS_{eff}, wherein the reference EFS_{eff} is the EFS_{eff} of a plurality of human patients who have received treatment with R-CHOP. In some embodiments, treatment of a plurality of human patients according to the methods of the disclosure results in a hazard ratio of no more than 0.77 (e.g., 0.76, 0.75, 0.74, 0.73, 0.72, 0.71, 0.70) in event-free survival-efficacy (EFS_{eff}) in the plurality of human patients as compared to treatment with R-CHOP. In some embodiments, treatment of a plurality of human patients according to the methods of the disclosure results in a hazard ratio of no more than 0.81 (e.g., 0.80, 0.79, 0.78, 0.77, 0.76, 0.75, 0.74, 0.73, 0.72, 0.71, 0.70) in event-free survival-efficacy (EFS_{eff}) in the plurality of human patients as compared to treatment with R-CHOP. In some embodiments, treatment of a plurality of human patients according to the methods of the disclosure results in a stratified hazard ratio of no more than 0.77 (95% confidence interval: 0.59, 1.00) in event-free survival-efficacy (EFS_{eff}) in the plurality of human patients as compared to treatment with R-CHOP. In some embodiments, treatment of a plurality of human patients according to the methods of the disclosure results in a stratified hazard ratio of no more than 0.81 (95% confidence interval: 0.63, 1.04) in event-free survival-efficacy (EFS_{eff}) in the plurality of human patients as compared to treatment with R-CHOP. In some embodiments, the EFS_{eff} or the reference EFS_{eff} is measured starting from the start of the corresponding treatment (i.e., the treatment according to the methods of the disclosure, or R-CHOP) to the time of a first occurrence of an EFS_{eff} event. In some embodiments, the EFS_{eff} or the reference EFS_{eff} is measured starting from up to 7 days (e.g., any of 7, 6, 5, 4, 3, 2, 1 or 0 days) prior to the start of the corresponding treatment (i.e., the treatment according to the methods of the disclosure, or R-CHOP) to the time of a first occurrence of an EFS_{eff} event. In some embodiments, the EFS_{eff} or the reference EFS_{eff} is measured starting from the date of randomization according to the treatment regimen described in Example 1 to the time of a first occurrence of an EFS_{eff} event. In some embodiments, the improvement in EFS_{eff} is statistically significant. In some embodiments, the improvement in EFS_{eff} is calculated at 12 months or more, 24 months or more, or 36 months or more, measured starting from the start of the corresponding treatment (i.e., the treatment according to the methods of the disclosure, or R-CHOP). In some embodiments, the improvement in EFS_{eff} is calculated at 12 months or more, 24 months or more, or 36 months or more, measured starting from up to 7 days (e.g., any of 7, 6, 5, 4, 3, 2, 1 or 0 days) prior to the start of the corresponding treatment (i.e., the treatment according to the methods of the disclosure, or R-CHOP). In some embodiments, the improvement in EFS_{eff} is calculated at 12 months or more, 24 months or more, or 36 months or more, measured starting from the date of randomization according to the treatment regimen described in Example 1. In some embodiments, the hazard ratio has a 95% confidence interval. In some embodiments, the hazard ratio is calculated at 12 months or more, 24 months or more, or 36 months or more, measured starting from the start of the corresponding treatment (i.e., the treatment according to the methods of the disclosure, or R-CHOP). In some embodiments, the hazard ratio is calculated at 12 months or more, 24 months or more, or 36 months or more, measured starting from up to 7 days (e.g., any of 7, 6, 5, 4, 3, 2, 1 or 0 days) prior to the start of the corresponding treatment (i.e., the treatment according to the methods of the disclosure, or R-CHOP). In some embodiments, the hazard ratio is calculated at 12 months or more, 24 months or more, or 36 months or more, measured starting from the date of randomization according to the treatment regimen described in Example 1. In some embodiments, the EFS_{eff} event is any of: (a) disease progression; (b) relapse; (c) death; (d) a primary efficacy reason that leads to initiation of a non-protocol specified anti-lymphoma treatment (NALT; e.g., an anti-lymphoma treatment other than a treatment comprising an anti-CD79b immunoconjugate[e.g., huMA79bv28-MC-vc-PAB-MMAE or polatuzumab vedotin], an anti-CD20 antibody [e.g., obinutuzumab or rituximab], one or more chemotherapeutic agents [e.g., cyclophosphamide and/or doxorubicin], and a corticosteroid [e.g., prednisone, prednisolone, or methylprednisolone] as described herein), and that is not disease progression or relapse; or (e) a biopsy positive for residual disease.

In some embodiments, treatment of a plurality of human patients according to the methods of the disclosure results in a rate of complete response (CR) at end of treatment (EOT) in the plurality of human patients of at least about 77% (e.g., 78%, 79%, or 80%), wherein the rate of CR is assessed by positron emission tomography-computed tomography (PET-CT). In some embodiments, PET-CT refers to fluorodeoxyglucose positron emission tomography (FDG-PET), e.g., as described in Example 1 herein. In some embodiments, CR is assessed by an investigator or by blinded independent central review (BICR). In some embodiments, treatment of a plurality of human patients according to the methods of the disclosure results in an improvement in the rate of CR of at least about 3% (e.g., 4%, 5%, 6%, 7%, 8%, 9%, or 10%) in the plurality of human patients, as compared to a plurality of human patients who have received treatment with R-CHOP. In some embodiments, the improvement in the rate of CR is statistically significant.

In some embodiments, treatment of a plurality of human patients according to the methods of the disclosure results in a best overall response (BOR) rate in the plurality of human patients of at least about 95% (e.g., any of about 95%, 95.5%, 96%, 96.5%, 97%, 97.5%, 98%, or more). In some embodiments, treatment of a plurality of human patients according to the methods of the disclosure results in an improvement in the rate of BOR of at least about 1% (e.g., any of about 1%, 1.2%, 1.3%, 1.4%, 1.5%, 2%, 2.5%, 3%, 3.5%, 4%, 4.5%, 5%, or more) in the plurality of human patients, as compared to a plurality of human patients who have received treatment with R-CHOP. In some embodiments, the improvement in the rate of BOR is statistically significant. In some embodiments, treatment of a plurality of human patients according to the methods of the disclosure results in a rate of BOR of a complete response (CR) in the plurality of human patients of at least about 85% (e.g., any of about 85%, 86%, 87%, 88%, 89%, 90%, or more).

In some embodiments, treatment of a plurality of human patients according to the methods of the disclosure results in an objective response rate (ORR) at end of treatment (EOT) in the plurality of human patients of at least about 85% (e.g., 86%, 87%, 88%, 89%, or 90%), wherein the ORR is assessed by positron emission tomography-computed tomography (PET-CT). In some embodiments, treatment of a plurality of human patients according to the methods of the disclosure results in an objective response rate (ORR) at end of treatment (EOT) in the plurality of human patients of at least about 84% (e.g., 85%, 86%, 87%, 88%, 89%, or 90%), wherein the ORR is assessed by positron emission tomography-computed tomography (PET-CT). In some embodiments, PET-CT refers to fluorodeoxyglucose positron emission tomography (FDG-PET), e.g., as described in Example 1 herein. In some embodiments, ORR is assessed by an investigator or by blinded independent central review (BICR). In some embodiments, treatment of a plurality of human patients according to the methods of the disclosure results in an improvement in ORR of at least about 1.5% (e.g., any of at least about 1.5%, 1.6%, 1.7%, 1.8%, 1.9%, 2%, or more) in the plurality of human patients, as compared to a plurality of human patients who have received treatment with R-CHOP. In some embodiments, treatment of a plurality of human patients according to the methods of the disclosure results in an improvement in ORR of at least about 2% (e.g., 3%, 4%, 5%) in the plurality of human patients, as compared to a plurality of human patients who have received treatment with R-CHOP. In some embodiments, the improvement in the rate of ORR is statistically significant.

In some embodiments of any of the methods provided herein, statistical significance may be assessed using any suitable method known in the art, including, without limitation, the Cox proportional hazards method, a log-rank test, Cochran-Mantel-Haenszel (CMH) test, or a z-test.

In some embodiments, treatment of an individual having DLBCL according to any of the methods provided herein is assessed based on one or more patient-reported outcome assessments and/or quality of life assessments, including, but not limited to, the European Organisation for Research and Treatment of Cancer Quality of Life-Core 30 questionnaire (EORTC QLQ-C30), the Functional Assessment of Cancer Therapy-Lymphoma Lymphoma Subscale (FACT-Lym LymS), the Functional Assessment of Cancer Treatment/Gynecologic Oncology Group-Neurotoxicity (FACT/GOG-NTX), or the Health status based on EuroQo1 5-Dimension, 5-Level questionnaire (EQ-5D-5L).

The EORTC QLQ-C30 is a validated, reliable self-report measure consisting of 30 questions that assess five aspects of patient functioning (physical, emotional, role, cognitive, and social), three symptom scales (fatigue, nausea and vomiting, and pain), global health/quality of life (QoL), and six single items (dyspnea, insomnia, appetite loss, constipation, diarrhea, and financial difficulties) with a recall period of the previous week. Scale scores can be obtained for the multi-item scales. The first 28 items are scored on a 4-point scale that ranges from “not at all” to “very much,” and the last two items are scores on a 7-point scale that ranges from “very poor” to “excellent.” Higher scores indicate higher response levels (i.e., higher health-related quality of life [HRQoL], higher symptom severity). See. e.g., Aaronson et al., J Natl Cancer Inst (1993) 85:365-76; Fitzsimmons et al., Eur J Cancer (1999) 35:939-41.

The FACT-Lym is a validated, reliable self-report measure of health-related quality of life aspects relevant to lymphoma patients. The full measure consists of the FACT-G physical, social/family, emotional, and functional well-being scales (27 items), as well as a lymphoma-specific symptoms scale (15 items). In some embodiments, treatment of DLBCL according to any of the methods provided herein is assessed based on the items that comprise the lymphoma-specific symptoms (LymS) scale. Each item is rated on a 5-point response scale that ranges from “not at all” to “very much,” with higher scores indicative of better health-related quality of life. See. e.g., Hlubocky et al., Lymphoma (2013) 2013:1-9.

The FACT/GOG-NTX is a validated self-report measure for assessing platinum/paclitaxel-induced peripheral neuropathy. The FACT/GOG-NTX assesses polatuzumab vedotin-induced neuropathy, as symptoms of chemotherapy-induced neuropathy caused by microtubule inhibitors overlap with those seen in platinum/paclitaxel-containing regimens. The full measure consists of the FACT-G physical, social/family, emotional, and functional well-being scales (27 items), as well as a peripheral neuropathy symptoms scale (11 items). In some embodiments, treatment of DLBCL according to any of the methods provided herein is assessed based on the items that comprise the peripheral neuropathy scale. The scale contains 4 subscales that assess sensory neuropathy (4 items), hearing neuropathy (2 items), motor neuropathy (3 items), and dysfunction associated with neuropathy (2 items), which can be summed to create a total score. Each item is rated on a 5-point response scale that ranges from “not at all” to “very much,” with higher scores indicative of more extreme neuropathy. See. e.g., Huang et al., Int J Gynecol Cancer (2007) 17:387-93.

The EQ-5D-5L is a validated self-report health status questionnaire that is used to calculate a health status utility score for use in health economic analyses. There are two components to the EQ-5D-5L: a five-item health state profile that assesses mobility, self-care, usual activities, pain or discomfort, and anxiety or depression; and a visual analogue scale that measures overall health state. Published weighting systems allow for creation of a single composite score of the patient's health status. See, e.g., EuroQol Group. EuroQol: a new facility for the measurement of health-related quality of life. Health Policy (1990) 16:199-208; Brooks R., Health Policy (1996) 37:53-72: Herdman et al., Qual Life Res (2011) 20:1727-36: Janssen et al., Qual Life Res (2013) 22:1717-27.

In some embodiments, an individual, e.g., a human patient, treated according to any of the methods provided herein has an International Prognostic Index (IPI) score of 2 and achieves improved therapeutic or clinical responses (e.g., any of the responses described above), as compared to corresponding therapeutic or clinical responses in a corresponding individual treated with R-CHOP. In some embodiments, an individual, e.g., a human patient, treated according to any of the methods provided herein has an International Prognostic Index (IPI) score of between 3 and 5 and achieves improved therapeutic or clinical responses (e.g., any of the responses described above), as compared to corresponding therapeutic or clinical responses in a corresponding individual treated with R-CHOP. In some embodiments, an individual, e.g., a human patient, treated according to any of the methods provided herein has bulky disease with one lesion of ≥7.5 cm and achieves improved therapeutic or clinical responses (e.g., any of the responses described herein), as compared to corresponding therapeutic or clinical responses in a corresponding individual treated with R-CHOP. In some embodiments, an individual, e.g., a human patient, treated according to any of the methods provided herein does not have bulky disease and achieves improved therapeutic or clinical responses (e.g., any of the responses described herein), as compared to corresponding therapeutic or clinical responses in a corresponding individual treated with R-CHOP. In some embodiments, an individual, e.g., a human patient, treated according to any of the methods provided herein does not have a lesion of ≥7.5 cm and achieves improved therapeutic or clinical responses (e.g., any of the responses described herein), as compared to corresponding therapeutic or clinical responses in a corresponding individual treated with R-CHOP.

In some embodiments, treatment of an individual having DLBCL according to any of the methods provided herein results in improved health-related quality of life of the individual, e.g., as compared to a corresponding individual not treated according to the methods of the disclosure (e.g., an individual treated with R-CHOP), wherein health-related quality of life is assessed using the European Organisation for Research and Treatment of Cancer Quality of Life-Core 30 (EORTC QLQ-C30) questionnaire. In some embodiments, treatment of an individual having DLBCL according to any of the methods provided herein results in improved physical functioning and fatigue, e.g., as compared to a corresponding individual not treated according to the methods of the disclosure (e.g., an individual treated with R-CHOP), wherein physical functioning and fatigue are assessed using the EORTC QLQ-C30 questionnaire. In some embodiments, treatment of an individual having DLBCL according to any of the methods provided herein results in an improved score of the EORTC QLQ-C30 questionnaire, e.g., as compared to a corresponding individual not treated according to the methods of the disclosure (e.g., an individual treated with R-CHOP). In some embodiments, treatment of an individual having DLBCL according to any of the methods provided herein results in an improved score of the EORTC QLQ-C30 questionnaire, e.g., as compared to prior to administration of treatment according to the methods provided herein.

In some embodiments, treatment of an individual having DLBCL according to any of the methods provided herein results in an improved score of the Functional Assessment of Cancer Therapy-Lymphoma Lymphoma Subscale (FACT-Lym LymS) assessment, e.g., as compared to a corresponding individual not treated according to the methods of the disclosure (e.g., an individual treated with R-CHOP). In some embodiments, treatment of an individual having DLBCL according to any of the methods provided herein results in an improved score of the FACT-Lym LymS assessment, e.g., as compared to prior to administration of treatment according to the methods provided herein.

In some embodiments, treatment of an individual having DLBCL according to any of the methods provided herein results in an improved score of the EuroQol 5-Dimension, 5-Level questionnaire (EQ-5D-5L) assessment, e.g., as compared to a corresponding individual not treated according to the methods of the disclosure (e.g., an individual treated with R-CHOP). In some embodiments, treatment of an individual having DLBCL according to any of the methods provided herein results in an improved score of the EQ-5D-5L assessment, e.g., as compared to prior to administration of treatment according to the methods provided herein.

In some embodiments, an individual treated according to any of the methods provided herein is a human patient. In some embodiments, the human patient is an adult. In some embodiments, the human patient has an age of greater than 60 years or greater than 65 years. In some embodiments, the individual has CD20-positive DLBCL. In some embodiments, the methods provided herein comprise determining whether DLBCL in an individual is CD20-positive. In some embodiments, the methods provided herein comprise detecting a CD20-positive DLBCL in an individual. In some embodiments, the methods provided herein comprise acquiring knowledge of a CD20-positive DLBCL in an individual, e.g., from a third party, or by detecting the CD20-positive DLBCL in the individual. In some embodiments, the DLBCL has not been previously treated (i.e., the DLBCL is previously untreated DLBCL). In some embodiments, the DLBCL is a DLBCL, not otherwise specified (NOS), including germinal center B-cell type, activated B-cell type: a T-cell/histiocyte-rich large B-cell lymphoma: an Epstein-Barr virus-positive DLBCL, NOS: an ALK-positive large B-cell lymphoma: an HHV8-positive DLBCL, NOS: a high-grade B-cell lymphoma with MYC and BCL2 and/or BCL6 rearrangements (e.g., double-hit lymphoma, i.e., having MYC and BCL2 or BCL6 rearrangements; or triple-hit lymphoma, i.e., having MYC and BCL2 and BCL6 rearrangements); or a high-grade B-cell lymphoma, NOS. See, e.g., 2016 World Health Organization (WHO) classification of lymphoid neoplasms. In some embodiments, the DLBCL is an activated B-cell like (ABC) DLBCL. In some embodiments, the DLBCL is a double expressing lymphoma (DEL: overexpression of BCL2 and MYC) DLBCL. In some embodiments, the individual has an International Prognostic Index (IPI) score of 2-5. In some embodiments, the individual has an International Prognostic Index (IPI) score of 3-5. In some embodiments, the individual has an Eastern Cooperative Oncology Group (ECOG) Performance Status of 0, 1, or 2. In some embodiments, the individual has at least one bi-dimensionally measurable lesion, e.g., a lesion that is >1.5 cm in its longest dimension as measured by computed tomography or magnetic resonance imaging. In some embodiments, the individual has a left ventricular ejection fraction (LVEF)≥50% on cardiac multiple-gated acquisition (MUGA) scan or cardiac echocardiogram (ECHO). In some embodiments, the individual has adequate hematologic function (unless due to underlying DLBCL, as established for example, by extensive bone marrow involvement or due to hypersplenism secondary to the involvement of the spleen by DLBCL). In some embodiments, the individual has a hemoglobin ≥9.0 g/dL without packed red blood cell (RBC) transfusion during 14 days before first treatment. In some embodiments, the individual has an absolute neutrophil count (ANC)≥1,000/μL. In some embodiments, the individual has a platelet count ≥75,000/μL. In some embodiments, the individual does not have a contraindication to any of the individual components of the treatment methods of the disclosure (i.e., the immunoconjugate, anti-CD20 antibody, one or more chemotherapeutic agents, and/or corticosteroid). In some embodiments, the individual does not have a prior organ transplantation. In some embodiments, the individual does not have a Grade >1 peripheral neuropathy by clinical examination or demyelinating form of Charcot-Marie-Tooth disease prior to the start of treatment according to the methods of the disclosure. In some embodiments, the individual does not have history of indolent lymphoma. In some embodiments, the individual does not have a diagnosis of any the following: follicular lymphoma grade 3B: B-cell lymphoma, unclassifiable, with features intermediate between DLBCL and classical Hodgkin lymphoma (grey-zone lymphoma): primary mediastinal (thymic) large B-cell lymphoma: Burkitt lymphoma: central nervous system (CNS) lymphoma (primary or secondary involvement): primary effusion DLBCL; and/or primary cutaneous DLBCL, prior to the start of treatment according to the methods provided herein. In some embodiments, the individual has not been treated with cytotoxic drugs within 5 years prior to the start of treatment according to any of the methods provided herein. In some embodiments, the individual has not been previously treated with an anti-CD20 antibody prior to the start of treatment according to any of the methods provided herein. In some embodiments, the individual has not been previously treated with a monoclonal antibody within 3 months prior to the start of treatment according to any of the methods provided herein: any investigational therapy within 28 days prior to the start of treatment according to any of the methods provided herein; or a vaccination with live vaccines within 28 days prior to the start of treatment according to any of the methods provided herein. In some embodiments, the individual has not had radiotherapy to the mediastinal/pericardial region prior to the start of treatment according to any of the methods provided herein. In some embodiments, the individual has not been treated for DLBCL prior to the start of treatment according to any of the methods provided herein. In some embodiments, the individual has not received corticosteroids at a dose of >30 mg/day (e.g., prednisone or equivalent), for purposes other than lymphoma symptom control prior to the start of treatment according to any of the methods provided herein. In some embodiments, the individual docs not have evidence of significant, uncontrolled, concomitant diseases prior to the start of treatment according to any of the methods provided herein, e.g., significant cardiovascular disease (such as New York Heart Association Class III or IV cardiac disease, myocardial infarction within the last 6 months, unstable arrhythmias, or unstable angina) or pulmonary disease (including obstructive pulmonary disease and history of bronchospasm). In some embodiments, the individual does not have history or presence of a clinically significant abnormal electrocardiogram (ECG), including complete left bundle branch block, second- or third-degree heart block, or evidence of prior myocardial infarction, prior to the start of treatment according to any of the methods provided herein. In some embodiments, the individual does not have an active bacterial, viral, fungal, mycobacterial, parasitic, or other infection (excluding fungal infections of nail beds) prior to the start of treatment according to any of the methods provided herein. In some embodiments, the individual does not have a significant infection within 2 weeks prior to the start of treatment according to any of the methods provided herein. In some embodiments, the individual docs not have clinically significant liver disease, including active viral or other hepatitis, current alcohol abuse, or cirrhosis prior to the start of treatment according to any of the methods provided herein. In some embodiments, the individual does not have International normalized ratio (INR) or prothrombin time (PT)>1.5× upper limit of normal (ULN) in the absence of therapeutic anticoagulation prior to the start of treatment according to any of the methods provided herein. In some embodiments, the individual does not have partial thromboplastin time (PTT) or activated PTT (aPTT)>1.5×ULN in the absence of a lupus anticoagulant prior to the start of treatment according to any of the methods provided herein. In some embodiments, the individual does not have serum aspartate aminotransferase (AST) and alanine aminotransferase (ALT)≥2.5×ULN prior to the start of treatment according to any of the methods provided herein. In some embodiments, the individual does not have total bilirubin ≥1.5×ULN prior to the start of treatment according to any of the methods provided herein. In some embodiments, an individual with Gilbert disease is treated according to the methods provided herein if total bilirubin is ≥3.0×ULN prior to the start of treatment according to any of the methods provided herein. In some embodiments, the individual does not have serum creatinine clearance <40 mL/min (using Cockcroft-Gault formula). In some embodiments, the individual does not have suspected active or latent tuberculosis (e.g., as confirmed by a positive interferon gamma release assay) prior to the start of treatment according to any of the methods provided herein. In some embodiments, the individual does not have a positive test result for chronic hepatitis B infection (defined as positive hepatitis B surface antigen [HBsAg] serology) prior to the start of treatment according to any of the methods provided herein. In some embodiments, the individual does not have a positive test result for hepatitis C prior to the start of treatment according to any of the methods provided herein. In some embodiments, the individual does not have known history of HIV seropositive status prior to the start of treatment according to any of the methods provided herein. In some embodiments, the individual does not have positive results for the human T-lymphotrophic 1 virus (HTLV-1) prior to the start of treatment according to any of the methods provided herein. In some embodiments, an individual positive for HCV antibody is treated according to the methods provided herein if polymerase chain reaction (PCR) is negative for HCV RNA prior to the start of treatment according to any of the methods provided herein. In some embodiments, an individual with occult or prior hepatitis B infection, defined as positive total hepatitis B core antibody and negative HBsAg, is treated according to the methods provided herein if hepatitis B virus (HBV) DNA is undetectable prior to the start of treatment according to any of the methods provided herein. In some embodiments, the individual does not have a history of progressive multifocal leukoencephalopathy prior to the start of treatment according to any of the methods provided herein.

In some embodiments, an individual treated according to any of the methods provided herein is a human patient having one or more of the following characteristics: a) patients <=65 years old, >65 years old, at least 60 years old, or >60 years old: b) the identified histopathologically, high grade b-cell lymphoma, NOS or HGBL with MYC and BCL2 and/or BCL6-rearrangements: c) specific subtypes of DLBCL such as activated B-cell like (ABC) subtype, a double expressing lymphoma (DEL; overexpression of BCL2 and MYC), or a DLBCL that does not have double-hit or triple-hit lymphoma defined by MYC and BCL2 and/or BCL6-rearrangements: d) low Ann-Arbor Stage (I-II) or higher Ann-Arbor Stages (III, IV): c) normal baseline LDH levels or elevated baseline LDH levels; f) bone marrow involvement at baseline; g) 0-1 or 2+ extranodal sites; h) an International Prognostic Index (IPI) score between 3 and 5; and i) absence of bulky disease at baseline.

In some embodiments, the methods provided herein prolong the survival of a human patient that has previously untreated DLBCL without disease advancement (e.g., the absence of disease progression, disease relapse or death), wherein the human patient has one or more of the following characteristics: a) patients <=65 years old, >65 years old, at least 60 years old, or >60 years old; b) the identified histopathologically, high grade b-cell lymphoma, NOS or HGBL with MYC and BCL2 and/or BCL6-rearrangements; c) specific subtypes of DLBCL such as activated B-cell like (ABC) subtype, a double expressing lymphoma (DEL; overexpression of BCL2 and MYC), or a DLBCL that does not have double-hit or triple-hit lymphoma defined by MYC and BCL2 and/or BCL6-rearrangements; d) low Ann-Arbor Stage (I-II) or higher Ann-Arbor Stages (III, IV); c) normal baseline LDH levels or elevated baseline LDH levels; f) bone marrow involvement at baseline; g) 0-1 or 2+ extranodal sites; h) an International Prognostic Index (IPI) score between 3 and 5; and i) absence of bulky disease at baseline.

### IV. Immunoconjugates Comprising an Anti-CD79b Antibody and a Drug/Cytotoxic Agent (“Anti-CD79b Immunoconjugates”)

In some embodiments, the anti-CD79b immunoconjugate comprises an anti-CD79b antibody (Ab) which targets a cancer cell (such as a diffuse large B-cell lymphoma (DLBCL) cell), a drug moiety (D), and a linker moiety (L) that attaches Ab to D. In some embodiments, the anti-CD79b antibody is attached to the linker moiety (L) through one or more amino acid residues, such as lysine and/or cysteine. In some embodiments, the immunoconjugate comprises formula Ab-(L-D)p, wherein: (a) Ab is the anti-CD79b antibody which binds CD79b on the surface of a cancer cell (e.g., a DLBCL cell); (b) L is a linker; (c) D is a cytotoxic agent; and (d) p ranges from 1-8.

An exemplary anti-CD79b immunoconjugate comprises Formula I:

Ab-(L-D)ₚ (I)

wherein p is 1 to about 20 (e.g., 1 to 15, 1 to 10, 1 to 8, 2 to 5, or 3 to 4). In some embodiments, the number of drug moieties that can be conjugated to the anti-CD79b antibody is limited by the number of free cysteine residues. In some embodiments, free cysteine residues are introduced into the antibody amino acid sequence by the methods described elsewhere herein. Exemplary anti-CD79b immunoconjugates of Formula I comprise, but are not limited to, anti-CD79b antibodies that comprise 1, 2, 3, or 4 engineered cysteine amino acids (Lyon, R. et al (2012) Methods in Enzym. 502:123-138). In some embodiments, one or more free cysteine residues are already present in the anti-CD79b antibody, without the use of engineering, in which case the existing free cysteine residues may be used to conjugate the anti-CD79b antibody to the drug/cytotoxic agent. In some embodiments, the anti-CD79b antibody is exposed to reducing conditions prior to conjugation of the antibody to the drug/cytotoxic agent in order to generate one or more free cysteine residues.

### A. Exemplary Linkers

A “linker” (L) is a bifunctional or multifunctional moiety that can be used to link one or more drug moieties (D) to the anti-CD79b antibody (Ab) to form an anti-CD79b immunoconjugate of Formula I. In some embodiments, anti-CD79b immunoconjugates can be prepared using a linker having reactive functionalities for covalently attaching to the drug and to the anti-CD79b antibody. For example, in some embodiments, a cysteine thiol of the anti-CD79b antibody (Ab) can form a bond with a reactive functional group of a linker or a drug-linker intermediate to make the anti-CD79b immunoconjugate.

In one aspect, a linker has a functionality that is capable of reacting with a free cysteine present on the anti-CD79b antibody to form a covalent bond. Exemplary reactive functionalities include, without limitation, e.g., maleimide, haloacetamides, α-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates. See. e.g., the conjugation method at page 766 of Klussman, et al (2004), Bioconjugate Chemistry 15 (4): 765-773, and the Examples herein.

In some embodiments, a linker has a functionality that is capable of reacting with an electrophilic group present on the anti-CD79b antibody. Exemplary electrophilic groups include, without limitation, e.g., aldehyde and ketone carbonyl groups. In some embodiments, a heteroatom of the reactive functionality of the linker can react with an electrophilic group on an antibody and form a covalent bond to an antibody unit. Exemplary reactive functionalities include, but are not limited to, e.g., hydrazide, oxime, amino, hydrazine, thiosemicarbazone, hydrazine carboxylate, and arylhydrazide.

In some embodiments, the linker comprises one or more linker components. Exemplary linker components include, e.g., 6-maleimidocaproyl (“MC”), maleimidopropanoyl (“MP”), valine-citrulline (“val-cit” or “vc”), alanine-phenylalanine (“ala-phe”), p-aminobenzyloxycarbonyl (a “PAB”), N-Succinimidyl 4-(2-pyridylthio) pentanoate (“SPP”), and 4-(N-maleimidomethyl)cyclohexane-1 carboxylate (“MCC”). Various linker components are known in the art, some of which are described below.

In some embodiments, the linker is a “cleavable linker,” facilitating release of a drug. Non-limiting exemplary cleavable linkers include acid-labile linkers (e.g., comprising hydrazone), protease-sensitive (e.g., peptidase-sensitive) linkers, photolabile linkers, or disulfide-containing linkers (Chari et al., Cancer Research 52:127-131 (1992): U.S. Pat. No. 5,208,020).

In certain embodiments, a linker (L) has the following Formula II:

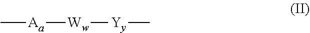

wherein A is a “stretcher unit,” and a is an integer from 0 to 1; W is an “amino acid unit,” and w is an integer from 0 to 12; Y is a “spacer unit,” and y is 0, 1, or 2; and Ab, D, and p are defined as above for Formula I. Exemplary embodiments of such linkers are described in U.S. Pat. No. 7,498,298, which is expressly incorporated herein by reference.

In some embodiments, a linker component comprises a “stretcher unit” that links an antibody to another linker component or to a drug moiety. Nonlimiting exemplary stretcher units are shown below (wherein the wavy line indicates sites of covalent attachment to an antibody, drug, or additional linker components):

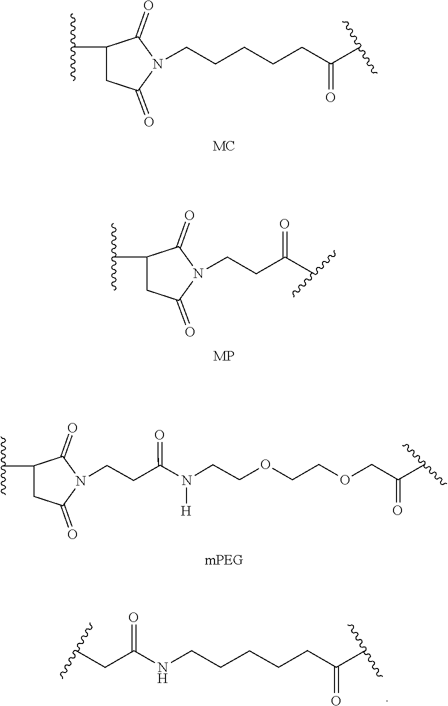 [SMILES: *C(=O)CCCCCN1C(=O)CC(*)C1=O ; *C(=O)CCN1C(=O)CC(*)C1=O ; *C(=O)COCCOCCNC(=O)CCN1C(=O)CC(*)C1=O ; *CC(=O)NCCCCCC(*)=O]

In some embodiments, a linker component comprises an “amino acid unit.” In some such embodiments, the amino acid unit allows for cleavage of the linker by a protease, thereby facilitating release of the drug/cytotoxic agent from the anti-CD79b immunoconjugate upon exposure to intracellular proteases, such as lysosomal enzymes (Doronina et al. (2003) Nat. Biotechnol. 21:778-784). Exemplary amino acid units include, but are not limited to, dipeptides, tripeptides, tetrapeptides, and pentapeptides. Exemplary dipeptides include, but are not limited to, valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Exemplary tripeptides include, but are not limited to, glycine-valine-citrulline (gly-val-cit) and glycine-glycine-glycine (gly-gly-gly). An amino acid unit may comprise amino acid residues that occur naturally and/or minor amino acids and/or non-naturally occurring amino acid analogs, such as citrulline. Amino acid units can be designed and optimized for enzymatic cleavage by a particular enzyme, for example, a tumor-associated protease, cathepsin B, C and D, or a plasmin protease.

In some embodiments, a linker component comprises a “spacer” unit that links the antibody to a drug moiety, either directly or through a stretcher unit and/or an amino acid unit. A spacer unit may be “self-immolative” or a “non-self-immolative.” A “non-self-immolative” spacer unit is one in which part or all of the spacer unit remains bound to the drug moiety upon cleavage of the ADC. Examples of non-self-immolative spacer units include, but are not limited to, a glycine spacer unit and a glycine-glycine spacer unit. In some embodiments, enzymatic cleavage of an ADC containing a glycine-glycine spacer unit by a tumor-cell associated protease results in release of a glycine-glycine-drug moiety from the remainder of the ADC. In some such embodiments, the glycine-glycine-drug moiety is subjected to a hydrolysis step in the tumor cell, thus cleaving the glycine-glycine spacer unit from the drug moiety.

A “self-immolative” spacer unit allows for release of the drug moiety. In certain embodiments, a spacer unit of a linker comprises a p-aminobenzyl unit. In some such embodiments, a p-aminobenzyl alcohol is attached to an amino acid unit via an amide bond, and a carbamate, methylcarbamate, or carbonate is made between the benzyl alcohol and the drug (Hamann et al. (2005) Expert Opin. Ther. Patents (2005) 15:1087-1103). In some embodiments, the spacer unit is p-aminobenzyloxycarbonyl (PAB). In some embodiments, an anti-CD79b immunoconjugate comprises a self-immolative linker that comprises the structure:

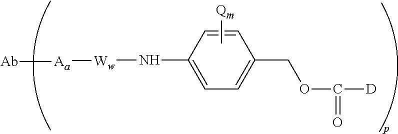

wherein Q is —C₁-C₈ alkyl, —O—(C₁-C₈ alkyl), -halogen, -nitro, or -cyno; m is an integer ranging from 0 to 4; and p ranges from 1 to about 20. In some embodiments, p ranges from 1 to 10, 1 to 7, 1 to 5, or 1 to 4.

Other examples of self-immolative spacers include, but are not limited to, aromatic compounds that are electronically similar to the PAB group, such as 2-aminoimidazol-5-methanol derivatives (U.S. Pat. No. 7,375,078; Hay et al. (1999) Bioorg. Med. Chem. Lett. 9:2237) and ortho- or para-aminobenzylacetals. In some embodiments, spacers can be used that undergo cyclization upon amide bond hydrolysis, such as substituted and unsubstituted 4-aminobutyric acid amides (Rodrigues et al (1995) Chemistry Biology 2:223), appropriately substituted bicyclo[2.2.1] and bicyclo[2.2.2] ring systems (Storm et al (1972) J. Amer. Chem. Soc. 94:5815) and 2-aminophenylpropionic acid amides (Amsberry, et al (1990) J. Org. Chem. 55:5867). Linkage of a drug to the α-carbon of a glycine residue is another example of a self-immolative spacer that may be useful in ADC (Kingsbury et al (1984) J. Med. Chem. 27:1447).

In some embodiments, linker L may be a dendritic type linker for covalent attachment of more than one drug moiety to an antibody through a branching, multifunctional linker moiety (Sun et al (2002) Bioorganic & Medicinal Chemistry Letters 12:2213-2215; Sun et al (2003) Bioorganic & Medicinal Chemistry 11:1761-1768). Dendritic linkers can increase the molar ratio of drug to antibody, i.e. loading, which is related to the potency of the ADC. Thus, where an antibody bears only one reactive cysteine thiol group, a multitude of drug moieties may be attached through a dendritic linker.

Nonlimiting exemplary linkers are shown below in the context of an anti-CD79b immunoconjugates of Formulas III, IV, V:

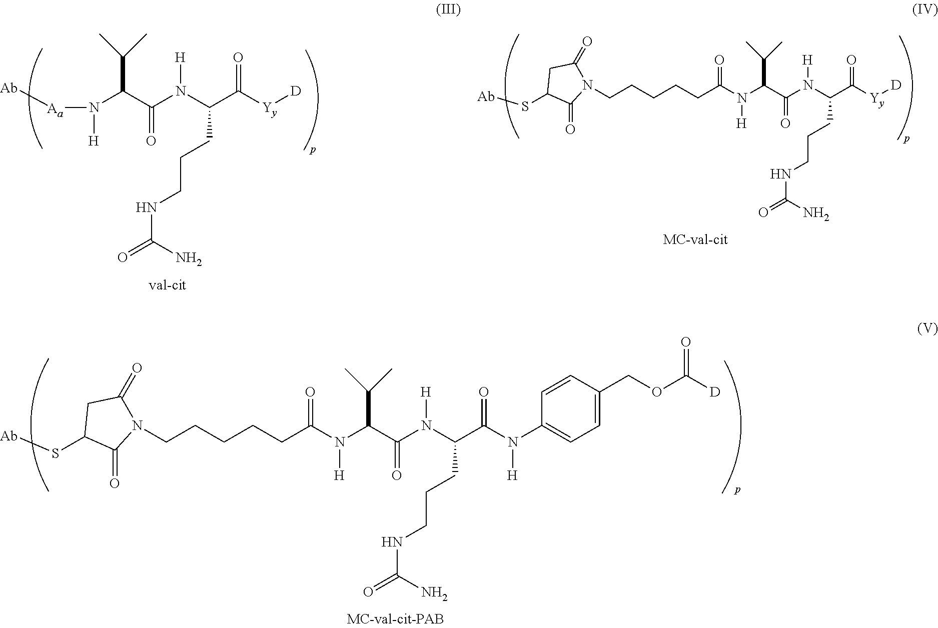

wherein (Ab) is an anti-CD79b antibody, (D) is a drug/cytotoxic agent, “Val-Cit” is a valine-citrulline dipeptide, MC is 6-maleimidocaproyl, PAB is p-aminobenzyloxycarbonyl, and p is 1 to about 20 (e.g., 1 to 15, 1 to 10, 1 to 8, 2 to 5, or 3 to 4).

In some embodiments, the anti-CD79b immunoconjugate comprises a structure of any one of formulas VI-V below:

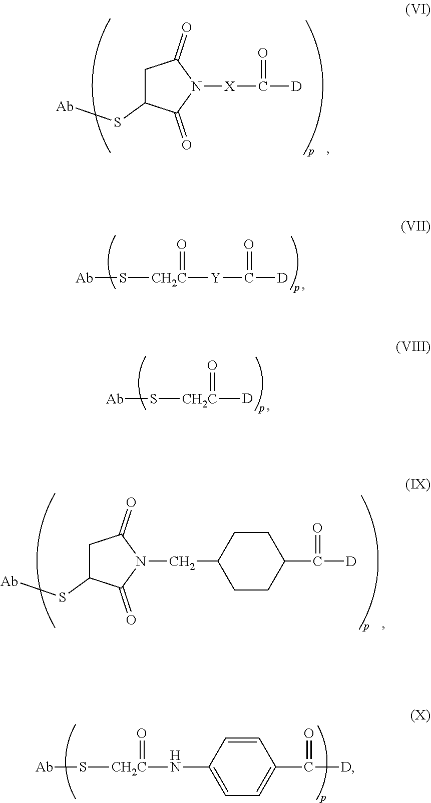

wherein X is:

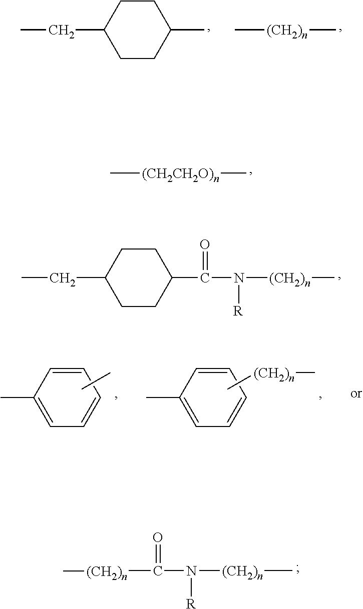

Y is:

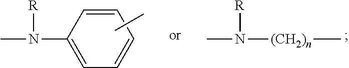

each R is independently H or C₁-C₆ alkyl; and n is 1 to 12.

Typically, peptide-type linkers can be prepared by forming a peptide bond between two or more amino acids and/or peptide fragments. Such peptide bonds can be prepared, for example, according to a liquid phase synthesis method (e.g., E. Schröder and K. Lübke (1965) “The Peptides”, volume 1, pp 76-136, Academic Press).

In some embodiments, a linker is substituted with groups that modulate solubility and/or reactivity. As a non-limiting example, a charged substituent such as sulfonate (—SO₃⁻) or ammonium may increase water solubility of the linker reagent and facilitate the coupling reaction of the linker reagent with the antibody and/or the drug moiety, or facilitate the coupling reaction of Ab-L (anti-CD79b antibody-linker intermediate) with D, or D-L (drug/cytotoxic agent-linker intermediate) with Ab, depending on the synthetic route employed to prepare the anti-CD79b immunoconjugate. In some embodiments, a portion of the linker is coupled to the antibody and a portion of the linker is coupled to the drug, and then the anti-CD79 Ab-(linker portion) ª is coupled to drug/cytotoxic agent-(linker portion) b to form the anti-CD79b immunoconjugate of Formula I. In some such embodiments, the anti-CD79b antibody comprises more than one (linker portion) ª substituents, such that more than one drug/cytotoxic agent is coupled to the anti-CD79b antibody in the anti-CD79b immunoconjugate of Formula I.

The anti-CD79b immunoconjugates provided herein expressly contemplate, but are not limited to, anti-CD79b immunoconjugates prepared with the following linker reagents: bis-maleimido-trioxyethylene glycol (BMPEO), N—(β-maleimidopropyloxy)-N-hydroxy succinimide ester (BMPS), N—(ε-maleimidocaproyloxy)succinimide ester (EMCS), N—[γ-maleimidobutyryloxy]succinimide ester (GMBS), 1,6-hexane-bis-vinylsulfone (HBVS), succinimidyl 4-(N-maleimidomethyl)cyclohexane-1-carboxy-(6-amidocaproate) (LC-SMCC), m-maleimidobenzoyl-N-hydroxysuccinimide ester (MBS), 4-(4-N-Maleimidophenyl) butyric acid hydrazide (MPBH), succinimidyl 3-(bromoacetamido) propionate (SBAP), succinimidyl iodoacetate (SIA), succinimidyl (4-iodoacetyl)aminobenzoate (SIAB), N-succinimidyl-3-(2-pyridyldithio) propionate (SPDP), N-succinimidyl-4-(2-pyridylthio) pentanoate (SPP), succinimidyl 4-(N-maleimidomethyl)cyclohexane-1-carboxylate (SMCC), succinimidyl 4-(p-maleimidophenyl) butyrate (SMPB), succinimidyl 6-[(beta-maleimidopropionamido)hexanoate] (SMPH), iminothiolane (IT), sulfo-EMCS, sulfo-GMBS, sulfo-KMUS, sulfo-MBS, sulfo-SIAB, sulfo-SMCC, and sulfo-SMPB, and succinimidyl-(4-vinylsulfone)benzoate (SVSB), and including bis-maleimide reagents; dithiobismaleimidoethane (DTME), 1,4-Bismaleimidobutane (BMB), 1,4 Bismaleimidyl-2,3-dihydroxy butane (BMDB), bismaleimidohexane (BMH), bismaleimidoethane (BMOE), BM (PEG) 2 (shown below), and BM (PEG) 3 (shown below); bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). In some embodiments, bis-maleimide reagents allow the attachment of the thiol group of a cysteine in the antibody to a thiol-containing drug moiety, linker, or linker-drug intermediate. Other functional groups that are reactive with thiol groups include, but are not limited to, iodoacetamide, bromoacetamide, vinyl pyridine, disulfide, pyridyl disulfide, isocyanate, and isothiocyanate.

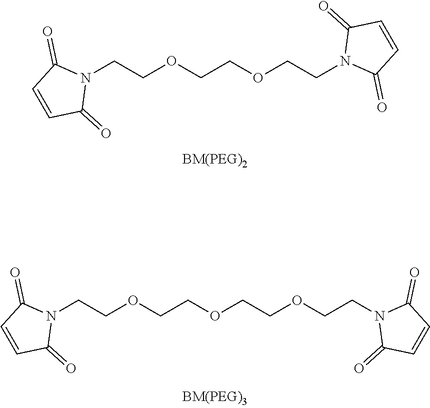 [SMILES: O=C1C=CC(=O)N1CCOCCOCCN1C(=O)C=CC1=O ; O=C1C=CC(=O)N1CCOCCOCCOCCN1C(=O)C=CC1=O]

Certain useful linker reagents can be obtained from various commercial sources, such as Pierce Biotechnology, Inc. (Rockford, IL), Molecular Biosciences Inc. (Boulder, CO), or synthesized in accordance with procedures described in the art; for example, in Toki et al (2002) J. Org. Chem. 67:1866-1872; Dubowchik, et al. (1997) Tetrahedron Letters, 38:5257-60; Walker, M. A. (1995) J. Org. Chem. 60:5352-5355; Frisch et al (1996) Bioconjugate Chem. 7:180-186: U.S. Pat. No. 6,214,345; WO 02/088172; US2003130189; US2003096743; WO 03/026577; WO 03/043583; and WO 04/032828.

Carbon-14-labeled 1-isothiocyanatobenzyl-3-methyldiethylene triaminepentaacetic acid (MX-DTPA) is an exemplary chelating agent for conjugation of radionucleotide to the antibody. See. e.g., WO94/11026.

### B. Anti-CD79b Antibodies

In some embodiments, the immunoconjugate (e.g., anti-CD79b immunoconjugate) comprises an anti-CD79b antibody that comprises at least one, two, three, four, five, or six HVRs selected from (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising an amino acid sequence of SEQ ID NO: 24; (e) HVR-L2 comprising the amino acid sequence of SEQ ID NO: 25; and (f) HVR-L3 comprising the amino acid sequence of SEQ ID NO: 26. In some such embodiments, the immunoconjugate comprises an anti-CD79b antibody comprising at least one of: (i) HVR-H3 comprising the amino acid sequence of SEQ ID NO: 23, and/or (ii) HVR-L1 comprising an amino acid sequence of SEQ ID NO: 24. In some embodiments, the immunoconjugate comprises an anti-CD79b antibody comprising at least one of: (i) HVR-H3 comprising the amino acid sequence of SEQ ID NO: 23, and/or (ii) HVR-L1 comprising the amino acid sequence of SEQ ID NO: 24. In some embodiments, the immunoconjugate comprises an anti-CD79b antibody comprising at least one, at least two, or all three VH HVR sequences selected from (a) HVR-H1 comprising the amino acid sequence of SEQ ID NO: 21; (b) HVR-H2 comprising the amino acid sequence of SEQ ID NO: 22; and (c) HVR-H3 comprising the amino acid sequence of SEQ ID NO: 23. In some embodiments, the immunoconjugate comprises an anti-CD79b antibody that comprises an HVR-H3 comprising the amino acid sequence of SEQ ID NO: 23. In some embodiments, the immunoconjugate comprises an anti-CD79b antibody that comprises an HVR-H3 comprising the amino acid sequence of SEQ ID NO: 23 and an HVR-L3 comprising the amino acid sequence of SEQ ID NO: 26. In some embodiments, the immunoconjugate comprises an anti-CD79b antibody that comprises an HVR-H3 comprising the amino acid sequence of SEQ ID NO: 23, an HVR-L3 comprising the amino acid sequence of SEQ ID NO: 26, and an HVR-H2 comprising the amino acid sequence of SEQ ID NO: 22. In some embodiments, the immunoconjugate comprises an anti-CD79b antibody that comprises (a) HVR-H1 comprising the amino acid sequence of SEQ ID NO: 21; (b) HVR-H2 comprising the amino acid sequence of SEQ ID NO: 22; and (c) HVR-H3 comprising the amino acid sequence of SEQ ID NO: 23.

In some embodiments, the immunoconjugate comprises an anti-CD79b antibody comprising at least one, at least two, or all three VL HVR sequences selected from (a) HVR-L1 comprising an amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the immunoconjugate comprises an anti-CD79b antibody that comprises at least one, at least two, or all three VL HVR sequences selected from (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the immunoconjugate comprises (a) HVR-L1 comprising an amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the immunoconjugate comprises an anti-CD79b antibody that comprises an HVR-L1 comprising the amino acid sequence of SEQ ID NO: 24 In some embodiments, the immunoconjugate comprises an anti-CD79b antibody that comprises (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26.

In some embodiments, the immunoconjugate comprises an anti-CD79b antibody comprising (a) a VH domain comprising at least one, at least two, or all three VH HVR sequences selected from (i) HVR-H1 comprising the amino acid sequence of SEQ ID NO: 21, (ii) HVR-H2 comprising the amino acid sequence of SEQ ID NO: 22, and (iii) HVR-H3 comprising the amino acid sequence of SEQ ID NO: 23; and (b) a VL domain comprising at least one, at least two, or all three VL HVR sequences selected from (i) HVR-L1 comprising an amino acid sequence of SEQ ID NO: 24, (ii) HVR-L2 comprising the amino acid sequence of SEQ ID NO: 25, and (iii) HVR-L3 comprising the amino acid sequence of SEQ ID NO: 26. In some embodiments, the immunoconjugate comprises an anti-CD79b antibody that comprises at least one of: (i) HVR-H3 comprising the amino acid sequence of SEQ ID NO: 23, and/or (ii) HVR-L1 comprising the amino acid sequence of SEQ ID NO: 24.

In some embodiments, the immunoconjugate comprises an anti-CD79b antibody that comprises (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising an amino acid sequence of SEQ ID NO: 24; (c) HVR-L2 comprising the amino acid sequence of SEQ ID NO: 25; and (f) HVR-L3 comprising the amino acid sequence of SEQ ID NO: 26. In some embodiments, the immunoconjugate comprises at least one of: HVR-H3 comprising the amino acid sequence of SEQ ID NO: 23 and/or HVR-L1 comprising an amino acid sequence of SEQ ID NO: 24. In some embodiments, the immunoconjugate comprises an anti-CD79b antibody that comprises (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising the amino acid sequence of SEQ ID NO: 24; (e) HVR-L2 comprising the amino acid sequence of SEQ ID NO: 25; and (f) HVR-L3 comprising the amino acid sequence of SEQ ID NO: 26.

In some embodiments, the anti-CD79b immunoconjugates comprise a humanized anti-CD79b antibody. In some embodiments, an anti-CD79b antibody comprises HVRs as in any of the embodiments provided herein, and further comprises a human acceptor framework, e.g., a human immunoglobulin framework or a human consensus framework. In some embodiments, the human acceptor framework is the human VL kappa 1 (VL_{KI}) framework and/or the VH framework VH_{III}. In some embodiments, a humanized anti-CD79b antibody comprises (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising an amino acid sequence of SEQ ID NO: 24; (e) HVR-L2 comprising the amino acid sequence of SEQ ID NO: 25; and (f) HVR-L3 comprising the amino acid sequence of SEQ ID NO: 26. In some embodiments, a humanized anti-CD79b antibody comprises (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising the amino acid sequence of SEQ ID NO: 24; (e) HVR-L2 comprising the amino acid sequence of SEQ ID NO: 25; and (f) HVR-L3 comprising the amino acid sequence of SEQ ID NO: 26.

In some embodiments, the immunoconjugate (e.g., the anti-CD79b immunoconjugate) comprises an anti-CD79b antibody comprising a heavy chain variable domain (VH) sequence having at least 90%, 91%, 92%, 93%, 94%, 95%, 96%, 97%, 98%, 99%, or 100% sequence identity to the amino acid sequence of SEQ ID NO: 19. In some embodiments, a VH sequence having at least 90%, 91%, 92%, 93%, 94%, 95%, 96%, 97%, 98%, or 99% identity to the amino acid sequence of SEQ ID NO: 19 contains substitutions (e.g., conservative substitutions), insertions, or deletions relative to the reference sequence, but an anti-CD79b immunoconjugate comprising that sequence retains the ability to bind to CD79b. In some embodiments, a total of 1 to 10 amino acids have been substituted, inserted and/or deleted in SEQ ID NO: 19. In some embodiments, a total of 1 to 5 amino acids have been substituted, inserted and/or deleted in SEQ ID NO: 19. In some embodiments, substitutions, insertions, or deletions occur in regions outside the HVRs (i.e., in the FRs). In some embodiments, the immunoconjugate (e.g., the anti-CD79b immunoconjugate) comprises the VH sequence of SEQ ID NO: 19, including post-translational modifications of that sequence. In some embodiments, the VH comprises one, two or three HVRs selected from: (a) HVR-H1 comprising the amino acid sequence of SEQ ID NO: 21, (b) HVR-H2 comprising the amino acid sequence of SEQ ID NO: 22, and (c) HVR-H3 comprising the amino acid sequence of SEQ ID NO: 23.

In some embodiments, the immunoconjugate (e.g., the anti-CD79b immunoconjugate) comprises an anti-CD79b antibody that comprises a light chain variable domain (VL) having at least 90%, 91%, 92%, 93%, 94%, 95%, 96%, 97%, 98%, 99%, or 100% sequence identity to the amino acid sequence of SEQ ID NO: 20. In certain embodiments, a VL sequence having at least 90%, 91%, 92%, 93%, 94%, 95%, 96%, 97%, 98%, or 99% identity to the amino acid sequence of SEQ ID NO: 20 contains substitutions (e.g., conservative substitutions), insertions, or deletions relative to the reference sequence, but an anti-CD79b immunoconjugate comprising that sequence retains the ability to bind to CD79b. In certain embodiments, a total of 1 to 10 amino acids have been substituted, inserted and/or deleted in SEQ ID NO: 20. In certain embodiments, a total of 1 to 5 amino acids have been substituted, inserted and/or deleted in SEQ ID NO: 20. In certain embodiments, the substitutions, insertions, or deletions occur in regions outside the HVRs (i.e., in the FRs). In some embodiments, the anti-CD79b immunoconjugate comprises an anti-CD79b antibody that comprises the VL sequence of SEQ ID NO: 20, including post-translational modifications of that sequence. In some embodiments, the VL comprises one, two or three HVRs selected from (a) HVR-L1 comprising an amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the VL comprises one, two or three HVRs selected from (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26.

In some embodiments, the immunoconjugate (e.g., the anti-CD79b immunoconjugate) comprises an anti-CD79b antibody that comprises VH as in any of the embodiments provided herein, and a VL as in any of the embodiments provided herein. In some embodiments, the immunoconjugate comprises an anti-CD79b antibody that comprises the VH and VL sequences in SEQ ID NO: 19 and SEQ ID NO: 20, respectively, including post-translational modifications of those sequences.

In some embodiments, the immunoconjugate (e.g., anti-CD79b immunoconjugate) comprises an anti-CD79b antibody that binds to the same epitope as an anti-CD79b antibody described herein. For example, in some embodiments, the immunoconjugate (e.g., anti-CD79b immunoconjugate) comprises an anti-CD79b antibody that binds to the same epitope as an anti-CD79b antibody comprising a VH sequence of SEQ ID NO: 19 and a VL sequence of SEQ ID NO: 20.

In some embodiments, the immunoconjugate comprises an anti-CD79b antibody that is a monoclonal antibody, a chimeric antibody, humanized antibody, or human antibody. In some embodiments, immunoconjugate comprises an antigen-binding fragment of an anti-CD79b antibody described herein, e.g., a Fv, Fab, Fab′, scFv, diabody, or F (ab)₂ fragment. In some embodiments, the immunoconjugate comprises a substantially full length anti-CD79b antibody, e.g., an IgG1 antibody or other antibody class or isotype as described elsewhere herein.

In some embodiments, the immunoconjugate comprises an anti-CD79b antibody comprising a heavy chain comprising the amino acid sequence of SEQ ID NO: 36, and wherein the light chain comprises the amino acid sequence of SEQ ID NO: 35. In some embodiments, the immunoconjugate comprises an anti-CD79b antibody that comprises a heavy chain comprising the amino acid sequence of SEQ ID NO: 37 and a light chain comprising the amino acid sequence of SEQ ID NO: 35. In some embodiments, the immunoconjugate comprises an anti-CD79b antibody that comprises a heavy chain comprising the amino acid sequence of SEQ ID NO: 36 and a light chain comprising the amino acid sequence of SEQ ID NO: 38.

In some embodiments, the immunoconjugate is polatuzumab vedotin, as described in WHO Drug Information, Vol. 26, No. 4, 2012 (Proposed INN: List 108), which is expressly incorporated by reference herein in its entirety. As shown in WHO Drug Information, Vol. 26, No. 4, 2012, polatuzumab vedotin has the following structure: immunoglobulin G1-kappa auristatin E conjugate, anti-[Homo sapiens CD79B (immunoglobulin-associated CD79 beta)], humanized monoclonal antibody conjugated to auristatin E; gamma1 heavy chain (1-447) [humanized VH (Homo sapiens IGHV3-66*01 (79.60%)-(IGHD)-IGHJ4*01) [8.8.13] (1-120)-Homo sapiens IGHG1*03 (CH1 R120>K (214) (121-218), hinge (219-233), CH2 (234-343), CH3 (344-448), CHS (449-450)) (121-450)], (220-218′)-disulfide (if not conjugated) with kappa light chain (1′-218′) [humanized V-KAPPA (Homo sapiens IGKV1-39*01 (80.00%)-IGKJ1*01) [11.3.9] (1′-112′)-Homo sapiens IGKC*01 (113′-218′)]; dimer (226-226″: 229-229″)-bisdisulfide; conjugated, on an average of 3 to 4 cysteinyl, to monomethylauristatin E (MMAE), via a cleavable maleimidocaproyl-valyl-citrullinyl-p-aminobenzylcarbamate (mc-val-cit-PABC) linker; the heavy chain of polatuzumab vedotin has the following sequence:

| (SEQ ID NO: 56) | |
|---|---|
| EVQLVESGGG LVQPGGSLRL SCAASGYTFS SYWIEWVRQA PGKGLEWIGE | 50 |
| ILPGGGDTNY NEIFKGRATF SADTSKNTAY LQMNSLRAED TAVYYCTRRV | 100 |
| PIRLDYWGQG TLVTVSSAST KGPSVFPLAP SSKSTSGGTA ALGCLVKDYF | 150 |
| PEPVTVSWNS GALTSGVHTF PAVLQSSGLY SLSSVVTVPS SSLGTQTYIC | 200 |
| NVNHKPSNTK VDKKVEPKSC DKTHTCPPCP APELLGGPSV FLFPPKPKDT | 250 |
| LMISRTPEVT CVVVDVSHED PEVKFNWYVD GVEVHNAKTK PREEQYNSTY | 300 |
| RVVSVLTVLH QDWLNGKEYK CKVSNKALPA PIEKTISKAK GQPREPQVYT | 350 |
| LPPSREEMTK NQVSLTCLVK GFYPSDIAVE WESNGQPENN YKTTPPVLDS | 400 |
| DGSFFLYSKL TVDKSRWQQG NVFSCSVMHE ALHNHYTQKS LSLSPGK; | 447 |

the light chain of polatuzumab vedotin has the following sequence:

| (SEQ ID NO: 35) | |
|---|---|
| DIQLTQSPSS LSASVGDRVT ITCKASQSVD YEGDSFLNWY QQKPGKAPKL | 50 |
| LIYAASNLES GVPSRFSGSG SGTDFTLTIS SLQPEDFATY YCQQSNEDPL | 100 |
| TFGQGTKVEI KRTVAAPSVF IFPPSDEQLK SGTASVVCLL NNFYPREAKV | 150 |
| QWKVDNALQS GNSQESVTEQ DSKDSTYSLS STLTLSKADY EKHKVYACEV | 200 |
| THQGLSSPVT KSFNRGEC; | 218 |

the disulfide bridge locations are:
Intra-H 22-96 144-200 261-321 367-425 22″-96″ 147″-203″ 261″-321″ 367″-425″
Intra-L 23′-92′ 138′-198′ 23″-92″ 138″-198″
Inter-H-L*220-218′ 220″-218″
Inter-H—H*226-226″ 229-229″
Two or three of the inter-chain disulfide bridges are not present, the antibody being conjugated to an average of 3 to 4 drug linkers each via a thioether bond;
the N-glycosylation sites are H CH2 N84.4:297, 297″ but lacking carbohydrate;
and other post-translational modifications are: lacking H chain C-terminal lysine. Thus, in some embodiments, the heavy chain of polatuzumab vedotin has the sequence of SEQ ID NO: 36.

### C. Drugs/Cytotoxic Agents

Anti-CD79b immunoconjugates comprise an anti-CD79b antibody (e.g., an anti-CD79b antibody described herein) conjugated to one or more drugs/cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes (i.e., a radioconjugate). Such immunoconjugates are targeted chemotherapeutic molecules which combine properties of both antibodies and cytotoxic drugs by targeting potent cytotoxic drugs to antigen-expressing cancer cells (such as tumor cells) (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14 (3): 154-169; Chari, R. V. (2008) Acc. Chem. Res. 41:98-107. That is, the anti-CD79b immunoconjugates selectively deliver an effective dose of a drug to cancerous cells/tissues whereby greater selectivity, i.e. a lower efficacious dose, may be achieved while increasing the therapeutic index (“therapeutic window”) (Polakis P. (2005) Current Opinion in Pharmacology 5:382-387).

Anti-CD79b immunoconjugates used in the methods provided herein include those with anticancer activity. In some embodiments, the anti-CD79b immunoconjugate comprises an anti-CD79b antibody conjugated, i.e. covalently attached, to the drug moiety. In some embodiments, the anti-CD79b antibody is covalently attached to the drug moiety through a linker. The drug moiety (D) of the anti-CD79b immunoconjugate may include any compound, moiety or group that has a cytotoxic or cytostatic effect. Drug moieties may impart their cytotoxic and cytostatic effects by mechanisms including but not limited to tubulin binding, DNA binding or intercalation, and inhibition of RNA polymerase, protein synthesis, and/or topoisomerase. Exemplary drug moieties include, but are not limited to, a maytansinoid, dolastatin, auristatin, calicheamicin, anthracycline, duocarmycin, vinca alkaloid, taxane, trichothecene, CC1065, camptothecin, elinafide, and stereoisomers, isosteres, analogs, and derivatives thereof that have cytotoxic activity.

### (1) Maytansine and Maytansinoids

In some embodiments, an anti-CD79b immunoconjugate comprises an anti-CD79b antibody conjugated to one or more maytansinoid molecules. Maytansinoids are derivatives of maytansine, and are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. U.S. Pat. No. 4,151,042). Synthetic maytansinoids are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

Maytansinoid drug moieties are attractive drug moieties in antibody-drug conjugates because they are: (i) relatively accessible to prepare by fermentation or chemical modification or derivatization of fermentation products, (ii) amenable to derivatization with functional groups suitable for conjugation through non-disulfide linkers to antibodies, (iii) stable in plasma, and (iv) effective against a variety of tumor cell lines.

Certain maytansinoids suitable for use as maytansinoid drug moieties are known in the art and can be isolated from natural sources according to known methods or produced using genetic engineering techniques (see. e.g., Yu et al (2002) PNAS 99:7968-7973). Maytansinoids may also be prepared synthetically according to known methods.

Exemplary maytansinoid drug moieties include, but are not limited to, those having a modified aromatic ring, such as: C-19-dechloro (U.S. Pat. No. 4,256,746) (prepared, for example, by lithium aluminum hydride reduction of ansamytocin P2); C-20-hydroxy (or C-20-demethyl)+/−C-19-dechloro (U.S. Pat. Nos. 4,361,650 and 4,307,016) (prepared, for example, by demethylation using Streptomyces or Actinomyces or dechlorination using LAH); and C-20-demethoxy, C-20-acyloxy (—OCOR), +/− dechloro (U.S. Pat. No. 4,294,757) (prepared, for example, by acylation using acyl chlorides), and those having modifications at other positions of the aromatic ring.

Exemplary maytansinoid drug moieties also include those having modifications such as: C-9—SH (U.S. Pat. No. 4,424,219) (prepared, for example, by the reaction of maytansinol with H₂S or P₂S₅); C-14-alkoxymethyl (demethoxy/CH₂OR) (U.S. Pat. No. 4,331,598); C-14-hydroxymethyl or acyloxymethyl (CH₂OH or CH₂OAc) (U.S. Pat. No. 4,450,254) (prepared, for example, from Nocardia); C-15-hydroxy/acyloxy (U.S. Pat. No. 4,364,866) (prepared, for example, by the conversion of maytansinol by Streptomyces); C-15-methoxy (U.S. Pat. Nos. 4,313,946 and 4,315,929) (for example, isolated from Trewia nudlflora); C-18-N-demethyl (U.S. Pat. Nos. 4,362,663 and 4,322,348) (prepared, for example, by the demethylation of maytansinol by Streptomyces); and 4,5-deoxy (U.S. Pat. No. 4,371,533) (prepared, for example, by the titanium trichloride/LAH reduction of maytansinol).

Many positions on maytansinoid compounds are useful as the linkage position. For example, an ester linkage may be formed by reaction with a hydroxyl group using conventional coupling techniques. In some embodiments, the reaction may occur at the C-3 position having a hydroxyl group, the C-14 position modified with hydroxymethyl, the C-15 position modified with a hydroxyl group, and the C-20 position having a hydroxyl group. In some embodiments, the linkage is formed at the C-3 position of maytansinol or a maytansinol analogue.

Maytansinoid drug moieties include those having the structure:

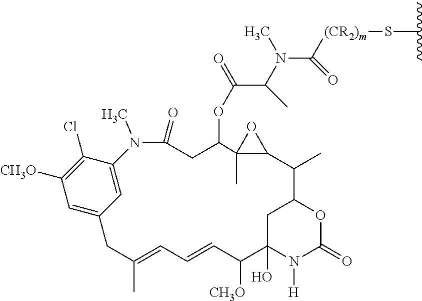

wherein the wavy line indicates the covalent attachment of the sulfur atom of the maytansinoid drug moiety to a linker of an anti-CD79b immunoconjugate. Each R may independently be H or a C₁-C₆ alkyl. The alkylene chain attaching the amide group to the sulfur atom may be methanyl, ethanyl, or propyl, i.e., m is 1, 2, or 3 (U.S. Pat. No. 633,410; U.S. Pat. No. 5,208,020; Chari et al (1992) Cancer Res. 52:127-131; Liu et al (1996) Proc. Natl. Acad. Sci USA 93:8618-8623).

All stereoisomers of the maytansinoid drug moiety are contemplated for the anti-CD79b immunoconjugate used in a method provided herein, i.e. any combination of R and S configurations at the chiral carbons (U.S. Pat. Nos. 7,276,497; 6,913,748; 6,441,163; U.S. Pat. No. 633,410 (RE39151); U.S. Pat. No. 5,208,020; Widdison et al (2006) J. Med. Chem. 49:4392-4408, which are incorporated by reference in their entirety). In some embodiments, the maytansinoid drug moiety has the following stereochemistry:

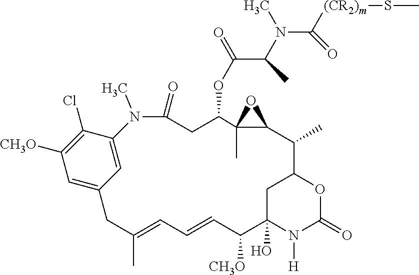

Exemplary embodiments of maytansinoid drug moieties include, but are not limited to, DM1; DM3; and DM4, having the structures:

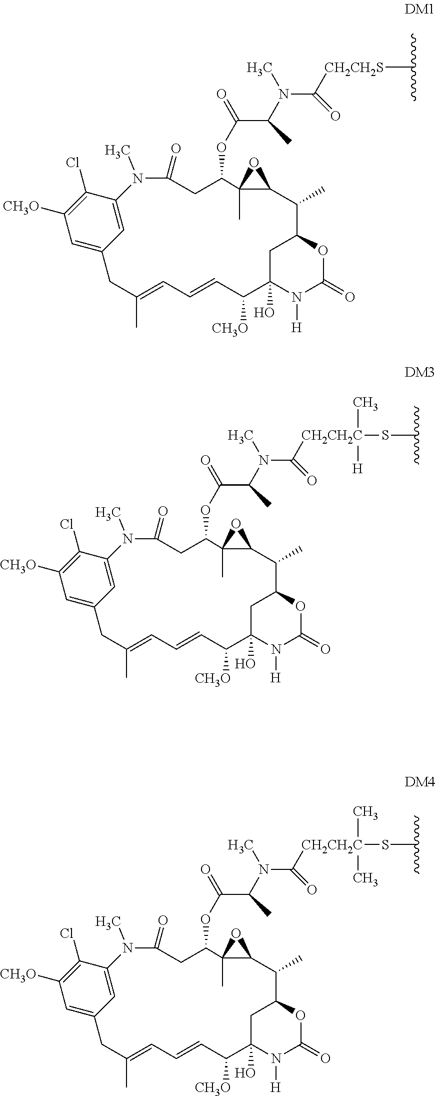

wherein the wavy line indicates the covalent attachment of the sulfur atom of the drug to a linker (L) of an anti-CD79b immunoconjugate.

Other exemplary maytansinoid anti-CD79b immunoconjugates have the following structures and abbreviations (wherein Ab is an anti-CD79b antibody and p is 1 to about 20. In some embodiments, p is 1 to 10, p is 1 to 7, p is 1 to 5, or p is 1 to 4):

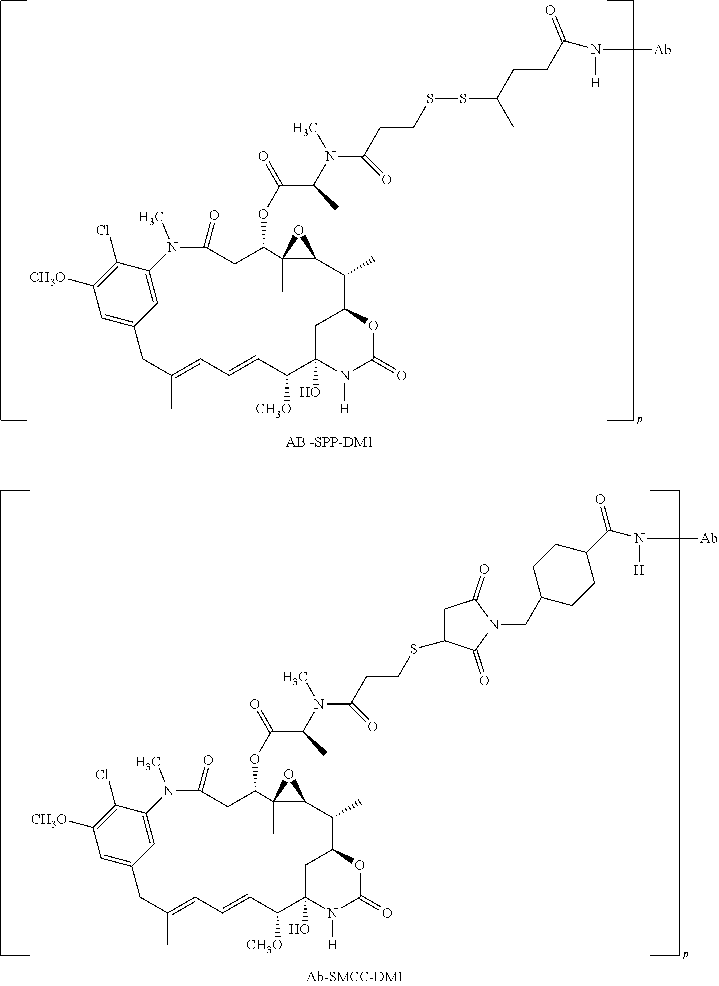

Exemplary antibody-drug conjugates wherein DM1 is linked through a BMPEO linker to a thiol group of the antibody have the structure and abbreviation:

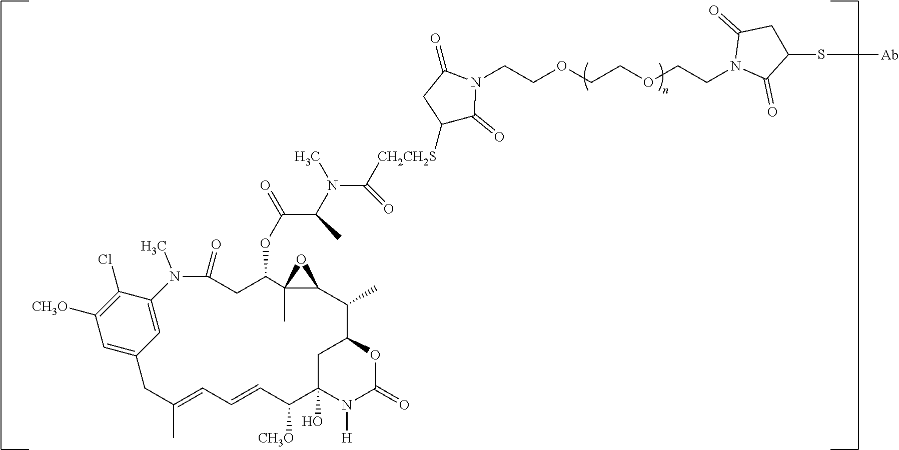

wherein Ab is an anti-CD79b antibody; n is 0, 1, or 2; and p is 1 to about 20. In some embodiments, p is 1 to 10, p is 1 to 7, p is 1 to 5, or p is 1 to 4.

Immunoconjugates containing maytansinoids, methods of making the same, and their therapeutic use are disclosed, for example, in U.S. Pat. Nos. 5,208,020 and 5,416,064; US 2005/0276812 A1; and European Patent EP 0 425 235 B1, the disclosures of which are hereby expressly incorporated by reference. See also Liu et al. Proc. Natl. Acad. Sci. USA 93:8618-8623 (1996); and Chari et al. Cancer Research 52:127-131 (1992).

In some embodiments, anti-CD79b antibody-maytansinoid conjugates may be prepared by chemically linking an anti-CD79b antibody to a maytansinoid molecule without significantly diminishing the biological activity of either the antibody or the maytansinoid molecule. See, e.g., U.S. Pat. No. 5,208,020 (the disclosure of which is hereby expressly incorporated by reference). In some embodiments, an anti-CD79b immunoconjugate with an average of 3-4 maytansinoid molecules conjugated per antibody molecule has shown efficacy in enhancing cytotoxicity of target cells without negatively affecting the function or solubility of the antibody. In some instances, even one molecule of toxin/antibody is expected to enhance cytotoxicity over the use of naked anti-CD79b antibody.

Exemplary linking groups for making antibody-maytansinoid conjugates include, for example, those described herein and those disclosed in U.S. Pat. No. 5,208,020; EP Patent 0 425 235 B1; Chari et al. Cancer Research 52:127-131 (1992); US 2005/0276812 A1; and US 2005/016993 A1, the disclosures of which are hereby expressly incorporated by reference.

### (2) Auristatins and Dolastatins

Drug moieties include dolastatins, auristatins, and analogs and derivatives thereof (U.S. Pat. Nos. 5,635,483; 5,780,588; 5,767,237; 6,124,431). Auristatins are derivatives of the marine mollusk compound dolastatin-10. While not intending to be bound by any particular theory, dolastatins and auristatins have been shown to interfere with microtubule dynamics, GTP hydrolysis, and nuclear and cellular division (Woyke et al (2001) Antimicrob. Agents and Chemother. 45 (12): 3580-3584) and have anticancer (U.S. Pat. No. 5,663,149) and antifungal activity (Pettit et al (1998) Antimicrob. Agents Chemother. 42:2961-2965). The dolastatin/auristatin drug moiety may be attached to the antibody through the N (amino) terminus or the C (carboxyl) terminus of the peptidic drug moiety (WO 02/088172; Doronina et al (2003) Nature Biotechnology 21 (7): 778-784; Francisco et al (2003) Blood 102 (4): 1458-1465).

Exemplary auristatin embodiments include the N-terminus linked monomethylauristatin drug moieties D_{E} and D_{F}, disclosed in U.S. Pat. Nos. 7,498,298 and 7,659,241, the disclosures of which are expressly incorporated by reference in their entirety:

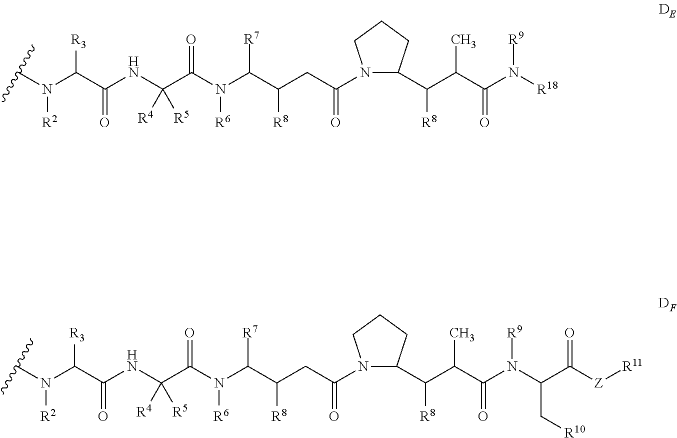

wherein the wavy line of D_{E} and D_{F} indicates the covalent attachment site to an antibody or antibody-linker component, and independently at each location:
R² is selected from H and C₁-C₈ alkyl;
R³ is selected from H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle and C₁-C₈ alkyl-(C₃-C₈ heterocycle);
R⁴ is selected from H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle and C₁-C₈ alkyl-(C₃-C₈ heterocycle);
R⁵ is selected from H and methyl;
or R⁴ and R⁵ jointly form a carbocyclic ring and have the formula-(CR^{a}R^{b})ₙ— wherein R^{a} and R^{b} are independently selected from H, C₁-C₈ alkyl and C₃-C₈ carbocycle and n is selected from 2, 3, 4, 5 and 6;
R⁶ is selected from H and C₁-C₈ alkyl;
R⁷ is selected from H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle and C₁-C₈ alkyl-(C₃-C₈ heterocycle);
each R⁸ is independently selected from H, OH, C₁-C₈ alkyl, C₃-C₈ carbocycle and O—(C₁-C₈ alkyl);
R⁹ is selected from H and C₁-C₈ alkyl;
R¹⁰ is selected from aryl or C₃-C₈ heterocycle;
Z is O, S, NH, or NR₁₂, wherein R¹² is C₁-C₈ alkyl;
R¹¹ is selected from H, C₁-C₂₀ alkyl, aryl, C₃-C₈ heterocycle, —(R¹³O)ₘ—R¹⁴, or —(R¹³O)ₘ—CH(R¹⁵)₂;
m is an integer ranging from 1-1000;
R¹³ is C₂-C₈ alkyl;
R¹⁴ is H or C₁-C₈ alkyl;
each occurrence of R¹⁵ is independently H, COOH, —(CH₂)ₙ—N(R¹⁶)₂, —(CH₂)ₙ—SO₃H, or —(CH₂)ₙ—SO₃—C₁-C₈ alkyl;
each occurrence of R¹⁶ is independently H, C₁-C₈ alkyl, or —(CH₂) n-COOH;
R¹⁸ is selected from —C(R⁸)₂—C(R⁸)₂-aryl, —C(R⁸)₂—C(R⁸)₂—(C₃-C₈ heterocycle), and —C(R⁸)₂—C(R⁸)₂—(C₃-C₈ carbocycle); and
n is an integer ranging from 0 to 6.

In one embodiment, R³, R⁴ and R⁷ are independently isopropyl or sec-butyl and R⁵ is —H or methyl. In an exemplary embodiment, R³ and R⁴ are each isopropyl, R⁵ is —H, and R⁷ is sec-butyl.

In yet another embodiment, R² and R⁶ are each methyl, and R⁹ is —H.

In still another embodiment, each occurrence of R⁸ is —OCH₃.

In an exemplary embodiment, R³ and R⁴ are each isopropyl, R² and R⁶ are each methyl, R⁵ is —H, R⁷ is sec-butyl, each occurrence of R⁸ is —OCH₃, and R⁹ is —H.

In one embodiment, Z is —O— or —NH—.

In one embodiment, R¹⁰ is aryl.

In an exemplary embodiment, R¹⁰ is -phenyl.

In an exemplary embodiment, when Z is —O—, R¹¹ is —H, methyl or t-butyl.

In one embodiment, when Z is —NH, R¹¹ is —CH(R¹⁵)₂, wherein R¹⁵ is —(CH₂)ₙ—N(R¹⁶)₂, and R¹⁶ is —C₁-C₈ alkyl or —(CH₂)ₙ—COOH.

In another embodiment, when Z is —NH, R¹¹ is —CH(R¹⁵)₂, wherein R¹⁵ is —(CH₂)ₙ—SO₃H.

An exemplary auristatin embodiment of formula D_{E} is MMAE, wherein the wavy line indicates the covalent attachment to a linker (L) of an anti-CD79b immunoconjugate:

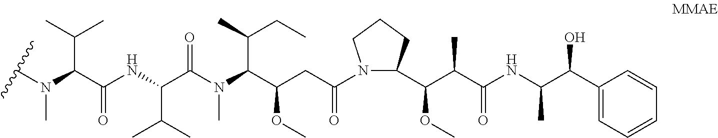 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

An exemplary auristatin embodiment of formula D_{F} is MMAF, wherein the wavy line indicates the covalent attachment to a linker (L) of an anti-CD79b immunoconjugate:

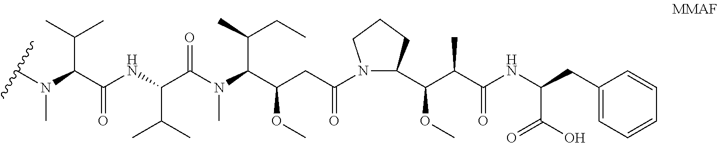 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)O)OC)C(C)C)C(C)C]

Other exemplary embodiments include monomethylvaline compounds having phenylalanine carboxy modifications at the C-terminus of the pentapeptide auristatin drug moiety (WO 2007/008848) and monomethylvaline compounds having phenylalanine sidechain modifications at the C-terminus of the pentapeptide auristatin drug moiety (WO 2007/008603).

Nonlimiting exemplary embodiments of an anti-CD79b immunoconjugate of Formula I comprising MMAE or MMAF and various linker components have the following structures and abbreviations (wherein “Ab” is an anti-CD79b antibody; p is 1 to about 8, “Val-Cit” is a valine-citrulline dipeptide; and “S” is a sulfur atom:

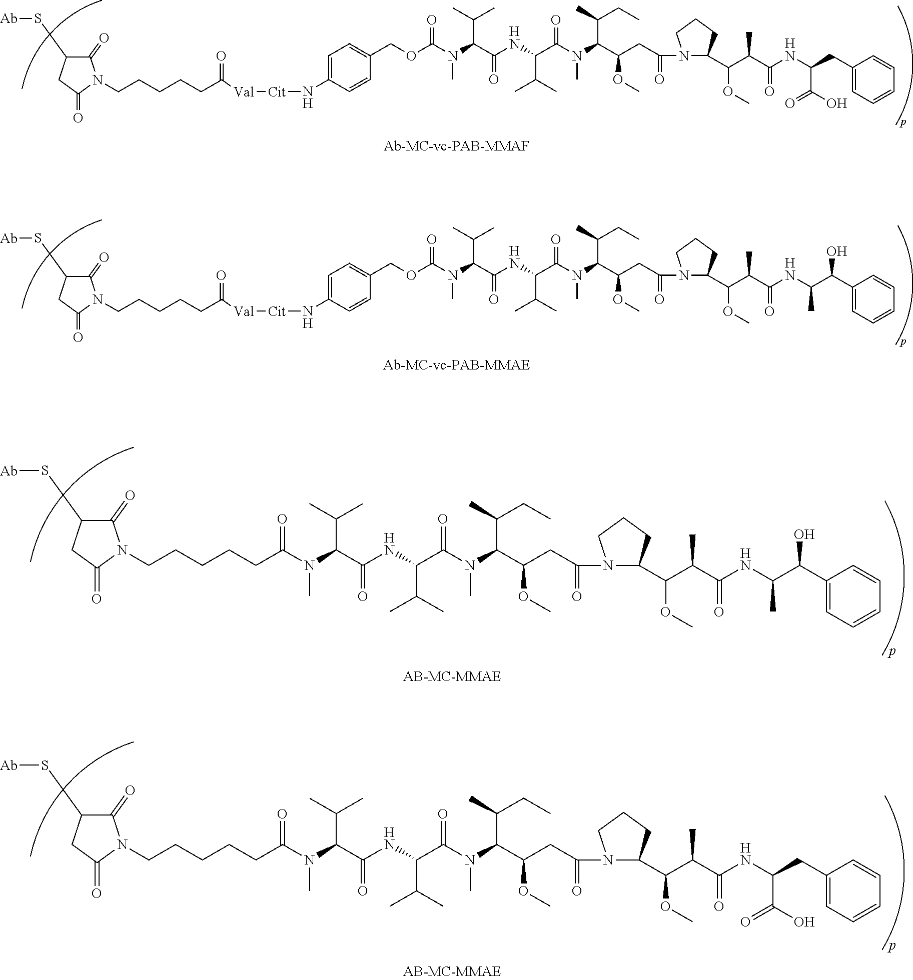

In certain embodiments, the anti-CD79b immunoconjugate comprises the structure of Ab-MC-vc-PAB-MMAE, wherein p is, e.g., about 1 to about 8; about 2 to about 7; about 3 to about 5; about 3 to about 4; or about 3.5. In some embodiments, the anti-CD79b immunoconjugate is huMA79bv28-MC-vc-PAB-MMAE, e.g., an anti-CD79b immunoconjugate comprising the structure of MC-vc-PAB-MMAE, wherein p is, e.g., about 1 to about 8: about 2 to about 7: about 3 to about 5: about 3 to about 4; or about 3.5, wherein the anti-CD79b antibody comprises a heavy chain comprising the amino acid sequence of SEQ ID NO: 36, and wherein the light chain comprises the amino acid sequence of SEQ ID NO: 35. In some embodiments, the anti-CD79b immunoconjugate is polatuzumab vedotin (CAS Number 1313206-42-6). Polatuzumab vedotin has the IUPHAR/BPS Number 8404, the KEGG Number D10761, the INN number 9714, and can also be referred to as “DCDS4501A,” or “RG7596.”

Nonlimiting exemplary embodiments of anti-CD79b immunoconjugates of Formula I comprising MMAF and various linker components further include Ab-MC-PAB-MMAF and Ab-PAB-MMAF. Immunoconjugates comprising MMAF attached to an antibody by a linker that is not proteolytically cleavable have been shown to possess activity comparable to immunoconjugates comprising MMAF attached to an antibody by a proteolytically cleavable linker (Doronina et al. (2006) Bioconjugate Chem. 17:114-124). In some such embodiments, drug release is believed to be effected by antibody degradation in the cell.

Typically, peptide-based drug moieties can be prepared by forming a peptide bond between two or more amino acids and/or peptide fragments. Such peptide bonds can be prepared, for example, according to a liquid phase synthesis method (see. e.g., E. Schröder and K. Lübke, “The Peptides”, volume 1, pp 76-136, 1965, Academic Press). Auristatin/dolastatin drug moieties may, in some embodiments, be prepared according to the methods of: U.S. Pat. No. 7,498,298: U.S. Pat. No. 5,635,483: U.S. Pat. No. 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725: Pettit et al (1996) J. Chem. Soc. Perkin Trans. 15:859-863; and Doronina (2003) Nat. Biotechnol. 21 (7): 778-784.

In some embodiments, auristatin/dolastatin drug moieties of formulas D_{E} such as MMAE, and D_{F}, such as MMAF, and drug-linker intermediates and derivatives thereof, such as MC-MMAF, MC-MMAE, MC-vc-PAB-MMAF, and MC-vc-PAB-MMAE, may be prepared using methods described in U.S. Pat. No. 7,498,298; Doronina et al. (2006) Bioconjugate Chem. 17:114-124; and Doronina et al. (2003) Nat. Biotech. 21:778-784 and then conjugated to an antibody of interest.

### (3) Calicheamicin

In some embodiments, the anti-CD79b immunoconjugate comprises an anti-CD79b antibody conjugated to one or more calicheamicin molecules. The calicheamicin family of antibiotics, and analogues thereof, are capable of producing double-stranded DNA breaks at sub-picomolar concentrations (Hinman et al., (1993) Cancer Research 53:3336-3342; Lode et al., (1998) Cancer Research 58:2925-2928). Calicheamicin has intracellular sites of action but, in certain instances, does not readily cross the plasma membrane. Therefore, cellular uptake of these agents through antibody-mediated internalization may, in some embodiments, greatly enhance their cytotoxic effects. Nonlimiting exemplary methods of preparing anti-CD79b antibody immunoconjugates with a calicheamicin drug moiety are described, for example, in U.S. Pat. Nos. 5,712,374; 5,714,586; 5,739,116; and 5,767,285.

### (4) Other Drug Moieties

In some embodiments, an anti-CD79b immunoconjugate comprises geldanamycin (Mandler et al (2000) J. Nat. Cancer Inst. 92 (19): 1573-1581; Mandler et al (2000) Bioorganic & Med. Chem. Letters 10:1025-1028; Mandler et al (2002) Bioconjugate Chem. 13:786-791); and/or enzymatically active toxins and fragments thereof, including, but not limited to, diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. See, e.g., WO 93/21232.

Drug moieties also include compounds with nucleolytic activity (e.g., a ribonuclease or a DNA endonuclease).

In certain embodiments, an anti-CD79b immunoconjugate comprises a highly radioactive atom. A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. In some embodiments, when an anti-CD79b immunoconjugate is used for detection, it may comprise a radioactive atom for scintigraphic studies, for example Tc⁹⁹ or I¹²³, or a spin label for nuclear magnetic resonance (NMR) imaging (also known as magnetic resonance imaging, MRI), such as zirconium-89, iodine-123, iodine-131, indium-111, fluorine-19, carbon-13, nitrogen-15, oxygen-17, gadolinium, manganese or iron. Zirconium-89 may be complexed to various metal chelating agents and conjugated to antibodies, e.g., for PET imaging (WO 2011/056983).

The radio- or other labels may be incorporated in the anti-CD79b immunoconjugate in known ways. For example, a peptide may be biosynthesized or chemically synthesized using suitable amino acid precursors comprising, for example, one or more fluorine-19 atoms in place of one or more hydrogens. In some embodiments, labels such as Tc⁹⁹, I¹²³, Re¹⁸⁶, Re¹⁸⁸ and In¹¹¹ can be attached via a cysteine residue in the anti-CD79b antibody. In some embodiments, yttrium-90 can be attached via a lysine residue of the anti-CD79b antibody. In some embodiments, the IODOGEN method (Fraker et al (1978) Biochem. Biophys. Res. Commun. 80:49-57 can be used to incorporate iodine-123. “Monoclonal Antibodies in Immunoscintigraphy” (Chatal, CRC Press 1989) describes certain other methods.

In certain embodiments, an anti-CD79b immunoconjugate may comprise an anti-CD79b antibody conjugated to a prodrug-activating enzyme. In some such embodiments, a prodrug-activating enzyme converts a prodrug (e.g., a peptidyl chemotherapeutic agent, see WO 81/01145) to an active drug, such as an anti-cancer drug. Such immunoconjugates are useful, in some embodiments, in antibody-dependent enzyme-mediated prodrug therapy (“ADEPT”). Enzymes that may be conjugated to an anti-CD79b antibody include, but are not limited to, alkaline phosphatases, which are useful for converting phosphate-containing prodrugs into free drugs: arylsulfatases, which are useful for converting sulfate-containing prodrugs into free drugs: cytosine deaminase, which is useful for converting non-toxic 5-fluorocytosine into the anti-cancer drug, 5-fluorouracil: proteases, such as serratia protease, thermolysin, subtilisin, carboxypeptidases and cathepsins (such as cathepsins B and L), which are useful for converting peptide-containing prodrugs into free drugs: D-alanylcarboxypeptidases, which are useful for converting prodrugs that contain D-amino acid substituents: carbohydrate-cleaving enzymes such as β-galactosidase and neuraminidase, which are useful for converting glycosylated prodrugs into free drugs; β-lactamase, which is useful for converting drugs derivatized with β-lactams into free drugs; and penicillin amidases, such as penicillin V amidase and penicillin G amidase, which are useful for converting drugs derivatized at their amine nitrogens with phenoxyacetyl or phenylacetyl groups, respectively, into free drugs. In some embodiments, enzymes may be covalently bound to antibodies by recombinant DNA techniques well known in the art. See. e.g., Neuberger et al., Nature 312:604-608 (1984).

### D. Drug Loading

Drug loading is represented by p, the average number of drug moieties per anti-CD79b antibody in a molecule of Formula I. Drug loading may range from 1 to 20 drug moieties (D) per antibody. Anti-CD79b immunoconjugates of Formula I include collections of anti-CD79b antibodies conjugated with a range of drug moieties, from 1 to 20. The average number of drug moieties per anti-CD79b antibody in preparations of anti-CD79b immunoconjugates from conjugation reactions may be characterized by conventional means such as mass spectroscopy, ELISA assay, and HPLC. The quantitative distribution of anti-CD79b immunoconjugates in terms of p may also be determined. In some instances, separation, purification, and characterization of homogeneous anti-CD79b immunoconjugates where p is a certain value from anti-CD79b immunoconjugates with other drug loadings may be achieved by means such as reverse phase HPLC or electrophoresis.

For some anti-CD79b immunoconjugates, p may be limited by the number of attachment sites on the anti-CD79b antibody. For example, where the attachment is a cysteine thiol, as in certain exemplary embodiments above, an anti-CD79b antibody may have only one or several cysteine thiol groups, or may have only one or several sufficiently reactive thiol groups through which a linker may be attached. In certain embodiments, higher drug loading, e.g., p>5, may cause aggregation, insolubility, toxicity, or loss of cellular permeability of certain anti-CD79b immunoconjugates. In certain embodiments, the average drug loading for an anti-CD79b immunoconjugates ranges from 1 to about 8; from about 2 to about 6: from about 3 to about 5; or from about 3 to about 4. Indeed, it has been shown that for certain antibody-drug conjugates, the optimal ratio of drug moieties per antibody may be less than 8, and may be about 2 to about 5 (U.S. Pat. No. 7,498,298). In certain embodiments, the optimal ratio of drug moieties per antibody is about 3 to about 4. In certain embodiments, the optimal ratio of drug moieties per antibody is about 3.5.

In certain embodiments, fewer than the theoretical maximum of drug moieties are conjugated to the anti-CD79b antibody during a conjugation reaction. An antibody may contain, for example, lysine residues that do not react with the drug-linker intermediate or linker reagent, as discussed below. Generally, antibodies do not contain many free and reactive cysteine thiol groups which may be linked to a drug moiety: indeed most cysteine thiol residues in antibodies exist as disulfide bridges. In certain embodiments, an anti-CD79b antibody may be reduced with a reducing agent such as dithiothreitol (DTT) or tricarbonylethylphosphine (TCEP), under partial or total reducing conditions, to generate reactive cysteine thiol groups. In certain embodiments, an anti-CD79b antibody is subjected to denaturing conditions to reveal reactive nucleophilic groups such as lysine or cysteine.

The loading (drug/antibody ratio) of an anti-CD79b immunoconjugate may be controlled in different ways, and for example, by: (i) limiting the molar excess of drug-linker intermediate or linker reagent relative to antibody, (ii) limiting the conjugation reaction time or temperature, and (iii) partial or limiting reductive conditions for cysteine thiol modification.

It is to be understood that where more than one nucleophilic group reacts with a drug-linker intermediate or linker reagent, then the resulting product is a mixture of anti-CD79b immunoconjugate compounds with a distribution of one or more drug moieties attached to an anti-CD79b antibody. The average number of drugs per antibody may be calculated from the mixture by a dual ELISA antibody assay, which is specific for antibody and specific for the drug. Individual anti-CD79b immunoconjugate molecules may be identified in the mixture by mass spectroscopy and separated by HPLC, e.g., hydrophobic interaction chromatography (see. e.g., McDonagh et al (2006) Prot. Engr. Design & Selection 19 (7): 299-307; Hamblett et al (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004: Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous anti-CD79b immunoconjugate with a single loading value may be isolated from the conjugation mixture by electrophoresis or chromatography.

### E. Methods of Preparing Anti-CD79b Immunoconjugates

An anti-CD79b immunoconjugate of Formula I may be prepared by several routes employing organic chemistry reactions, conditions, and reagents known to those skilled in the art, including, but not limited to, e.g., (1) reaction of a nucleophilic group of an anti-CD79b antibody with a bivalent linker reagent to form Ab-L via a covalent bond, followed by reaction with a drug moiety D; and (2) reaction of a nucleophilic group of a drug moiety with a bivalent linker reagent, to form D-L, via a covalent bond, followed by reaction with a nucleophilic group of an anti-CD79b antibody. Exemplary methods for preparing an anti-CD79b immunoconjugate of Formula I via the latter route are described in U.S. Pat. No. 7,498,298, which is expressly incorporated herein by reference.

Nucleophilic groups on antibodies include, but are not limited to: (i) N-terminal amine groups, (ii) side chain amine groups, e.g., lysine, (iii) side chain thiol groups, e.g., cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; and (iii) aldehydes, ketones, carboxyl, and maleimide groups. Certain antibodies have reducible interchain disulfides, i.e. cysteine bridges. Anti-CD79b antibodies may be made reactive for conjugation with linker reagents by treatment with a reducing agent such as DTT (dithiothreitol) or tricarbonylethylphosphine (TCEP), such that the anti-CD79b antibody is fully or partially reduced. Each cysteine bridge will thus form, theoretically, two reactive thiol nucleophiles. Additional nucleophilic groups can be introduced into anti-CD79b antibodies through modification of lysine residues, e.g., by reacting lysine residues with 2-iminothiolane (Traut's reagent), resulting in conversion of an amine into a thiol. Reactive thiol groups may also be introduced into an anti-CD79b antibody by introducing one, two, three, four, or more cysteine residues (e.g., by preparing variant antibodies comprising one or more non-native cysteine amino acid residues).

Anti-CD79b immunoconjugates described herein may also be produced by reaction between an electrophilic group on an anti-CD79b antibody, such as an aldehyde or ketone carbonyl group, with a nucleophilic group on a linker reagent or drug. Useful nucleophilic groups on a linker reagent include, but are not limited to, hydrazide, oxime, amino, hydrazine, thiosemicarbazone, hydrazine carboxylate, and arylhydrazide. In one embodiment, an anti-CD79b antibody is modified to introduce electrophilic moieties that are capable of reacting with nucleophilic substituents on the linker reagent or drug. In another embodiment, the sugars of glycosylated anti-CD79b antibodies may be oxidized, e.g., with periodate oxidizing reagents, to form aldehyde or ketone groups which may react with the amine group of linker reagents or drug moieties. The resulting imine Schiff base groups may form a stable linkage, or may be reduced, e.g., by borohydride reagents to form stable amine linkages. In one embodiment, reaction of the carbohydrate portion of a glycosylated anti-CD79b antibody with either galactose oxidase or sodium meta-periodate may yield carbonyl (aldehyde and ketone) groups in the anti-CD79b antibody that can react with appropriate groups on the drug (Hermanson, Bioconjugate Techniques). In another embodiment, anti-CD79b antibodies containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such an aldehyde can be reacted with a drug moiety or linker nucleophile.

Exemplary nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

Nonlimiting exemplary cross-linker reagents that may be used to prepare anti-CD79b immunoconjugates are described herein in the section titled “Exemplary Linkers.” Methods of using such cross-linker reagents to link two moieties, including a proteinaceous moiety and a chemical moiety, are known in the art. In some embodiments, a fusion protein comprising an anti-CD79b antibody and a cytotoxic agent may be made, e.g., by recombinant techniques or peptide synthesis. A recombinant DNA molecule may comprise regions encoding the antibody and cytotoxic portions of the conjugate either adjacent to one another or separated by a region encoding a linker peptide which does not destroy the desired properties of the conjugate. In yet another embodiment, an anti-CD79b antibody may be conjugated to a “receptor” (such as streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a drug or radionucleotide). Additional details regarding anti-CD79b immunoconjugates are provided in U.S. Pat. No. 8,545,850 and WO/2016/049214, the contents of which are expressly incorporated by reference herein in their entirety.

In some aspects, provided herein are immunoconjugates comprising the formula:

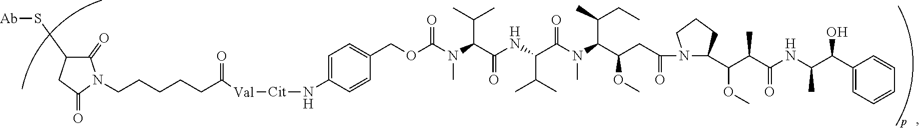

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with an anti-CD20 antibody (e.g., rituximab or obinutuzumab), one or more chemotherapeutic agents (e.g., cyclophosphamide and/or doxorubicin), and a corticosteroid (e.g., prednisone, prednisolone, or methylprednisolone) for treating an individual, e.g., a human patient, in need thereof having diffuse large B-cell lymphoma (DLBCL; e.g., previously untreated DLBCL). In some embodiments, p is between 3 and 4. In some embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the antibody comprises (i) a heavy chain comprising the amino acid sequence of SEQ ID NO: 36 and (ii) a light chain comprising the amino acid sequence of SEQ ID NO: 35. In some embodiments, p is between 2 and 5. In some embodiments, p is 3.4 or 3.5. In some embodiments, the immunoconjugate is for use in a method described herein.

In some aspects, provided herein is a use of an immunoconjugate comprising the formula:

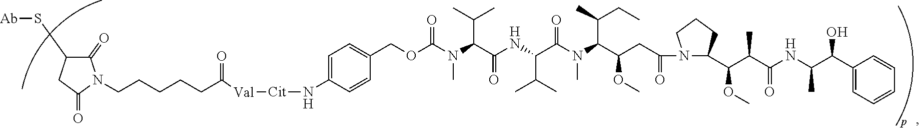

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for the manufacture of a medicament for treating an individual, e.g., a human patient, in need thereof having diffuse large B-cell lymphoma (DLBCL; e.g., previously untreated DLBCL), wherein the medicament is for (e.g., is formulated for) administration in combination with an anti-CD20 antibody (e.g., rituximab or obinutuzumab), one or more chemotherapeutic agents (e.g., cyclophosphamide and/or doxorubicin), and a corticosteroid (e.g., prednisone, prednisolone, or methylprednisolone). In some embodiments, p is between 2 and 5. In some embodiments, p is between 3 and 4. In some embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, p is 3.4 or 3.5. In some embodiments, the antibody comprises (i) a heavy chain comprising the amino acid sequence of SEQ ID NO: 36 and (ii) a light chain comprising the amino acid sequence of SEQ ID NO: 35. In some embodiments, the immunoconjugate is polatuzumab vedotin. In some embodiments, the medicament (i.e., the medicament comprising the immunoconjugate) is for use in a method described herein.

In some aspects, provided herein are immunoconjugates comprising the formula:

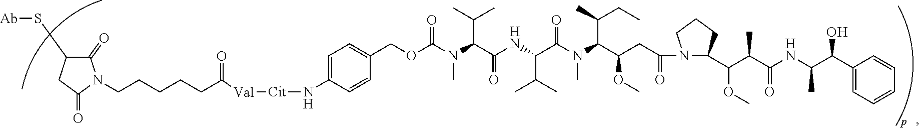

wherein Ab is an anti-CD79b antibody comprising (i) a heavy chain variable domain (VH) comprising the amino acid sequence of SEQ ID NO: 19 and (ii) a light chain variable domain (VL) comprising the amino acid sequence of SEQ ID NO: 20, and wherein p is between 2 and 5, for use in combination with an anti-CD20 antibody (e.g., rituximab or obinutuzumab), one or more chemotherapeutic agents (e.g., cyclophosphamide and/or doxorubicin), and a corticosteroid (e.g., prednisone, prednisolone, or methylprednisolone) for treating an individual, e.g., a human patient, in need thereof having diffuse large B-cell lymphoma (DLBCL; e.g., previously untreated DLBCL). In some embodiments, p is between 3 and 4. In some embodiments, p is 3.4 or 3.5. In some embodiments, the antibody comprises (i) a heavy chain comprising the amino acid sequence of SEQ ID NO: 36 and (ii) a light chain comprising the amino acid sequence of SEQ ID NO: 35. In some embodiments, the immunoconjugate is polatuzumab vedotin. In some embodiments, the immunoconjugate is for use in a method described herein.

In some aspects, provided herein is a use of an immunoconjugate comprising the formula:

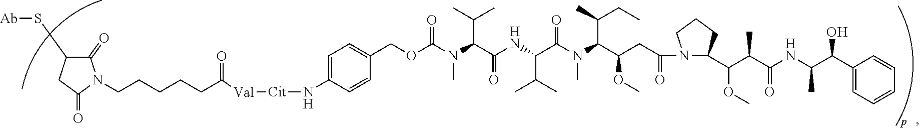

wherein Ab is an anti-CD79b antibody comprising (i) a heavy chain variable domain (VH) comprising the amino acid sequence of SEQ ID NO: 19 and (ii) a light chain variable domain (VL) comprising the amino acid sequence of SEQ ID NO: 20, and wherein p is between 2 and 5, for the manufacture of a medicament for treating an individual, e.g., a human patient, in need thereof having diffuse large B-cell lymphoma (DLBCL; e.g., previously untreated DLBCL), wherein the medicament is for (e.g., is formulated for) administration in combination with an anti-CD20 antibody (e.g., rituximab or obinutuzumab), one or more chemotherapeutic agents (e.g., cyclophosphamide and/or doxorubicin), and a corticosteroid (e.g., prednisone, prednisolone, or methylprednisolone). In some embodiments, p is between 3 and 4. In some embodiments, p is 3.4 or 3.5. In some embodiments, the antibody comprises (i) a heavy chain comprising the amino acid sequence of SEQ ID NO: 36 and (ii) a light chain comprising the amino acid sequence of SEQ ID NO: 35. In some embodiments, the immunoconjugate is polatuzumab vedotin. In some embodiments, the medicament (i.e., the medicament comprising the immunoconjugate) is for use in a method described herein.

In some aspects, provided herein is polatuzumab vedotin for use in combination with an anti-CD20 antibody (e.g., rituximab or obinutuzumab), one or more chemotherapeutic agents (e.g., cyclophosphamide and/or doxorubicin), and a corticosteroid (e.g., prednisone, prednisolone, or methylprednisolone) for treating an individual, e.g., a human patient, in need thereof having diffuse large B-cell lymphoma (DLBCL; e.g., previously untreated DLBCL). In some embodiments, the polatuzumab vedotin is for use in a method described herein.

In some aspects, provided herein is a use of polatuzumab vedotin for the manufacture of a medicament for treating an individual, e.g., a human patient, in need thereof having diffuse large B-cell lymphoma (DLBCL; e.g., previously untreated DLBCL), wherein the medicament is for (e.g., is formulated for) administration in combination with an anti-CD20 antibody (e.g., rituximab or obinutuzumab), one or more chemotherapeutic agents (e.g., cyclophosphamide and/or doxorubicin), and a corticosteroid (e.g., prednisone, prednisolone, or methylprednisolone). In some embodiments, the medicament (i.e., the medicament comprising the polatuzumab vedotin) is for use in a method described herein.

### V. Chemotherapeutic Agents

In some embodiments, the methods for treating DLBCL provided herein comprise administering to an individual, e.g., a human patient, an anti-CD79b immunoconjugate (e.g., huMA79bv28-MC-vc-PAB-MMAE or polatuzumab vedotin), an anti-CD20 antibody (such as obinutuzumab or rituximab), one or more chemotherapeutic agents, and a corticosteroid (e.g., prednisone, prednisolone, or methylprednisolone). In some embodiments, the one or more chemotherapeutic agents comprise cyclophosphamide and/or doxorubicin. In some embodiments, the one or more chemotherapeutic agents comprise cyclophosphamide and doxorubicin. In some embodiments, the methods for treating DLBCL provided herein comprise administering to an individual, e.g., a human patient, an anti-CD79b immunoconjugate (e.g., huMA79bv28-MC-vc-PAB-MMAE or polatuzumab vedotin), an anti-CD20 antibody (such as obinutuzumab or rituximab), cyclophosphamide and doxorubicin, and a corticosteroid (e.g., prednisone, prednisolone, or methylprednisolone). In some embodiments, one or more additional chemotherapeutic agents (e.g., in addition to cyclophosphamide and doxorubicin) may be administered to an individual in any of the methods of treating DLBCL provided herein.

Cyclophosphamide is also known as cytophosphane or the IUPAC name N,N-bis(2-chloroethyl)-2-oxo-1.3.225-oxazaphosphinan-2-amine. Cyclophosphamide is available commercially, e.g., as lyophilized Cytoxan, Endoxan, Cytoxan, Neosar, Procytox, or Cycloblastin. In some embodiments, cyclophosphamide may be administered orally or intravenously, In some embodiments, cyclophosphamide is administered intravenously. Typical dosages of cyclophosphamide that may be used include, for example, between about 375 mg/m² to about 1500 mg/m², between about 563 mg/m² to about 1500 mg/m², between about 600 mg/m² to about 1500 mg/m², between about 375 mg/m² to about 750 mg/m², between about 375 mg/m² to about 563 mg/m², or between about 563 mg/m² to about 750 mg/m², administered intravenously. In some embodiments, the dose of cyclophosphamide is about 375 mg/m², about 563 mg/m², or about 750 mg/m². In some embodiments, doses of cyclophosphamide are administered in 21-day cycles, e.g., on day 1 of each 21-day cycle. In some embodiments, the cyclophosphamide administered to an individual according to any of the methods provided herein is a pharmaceutically acceptable salt or hydrate thereof. In some embodiments, the cyclophosphamide is cyclophosphamide monohydrate. In some embodiments, the cyclophosphamide is cyclophosphamide anhydrous.

Doxorubicin is also known as doxil, doxorubicin, hydroxydaunorubicin, or the IUPAC name (7S,9S)-7-[(2R,4S,5S,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione. Doxorubicin is available commercially, e.g., as Adriamycin, Doxil, or Myocet. In some embodiments, doxorubicin is administered intravenously. Typical dosages of doxorubicin that may be used include, for example, between about 25 mg/m² to about 75 mg/m², between about 37.5 mg/m² to about 75 mg/m², between about 60 mg/m² to about 75 mg/m², between about 25 mg/m² to about 50 mg/m², or between about 37.5 mg/m² to about 50 mg/m², administered intravenously. In some embodiments, the dose of doxorubicin is about 25 mg/m², about 37.5 mg/m², or about 50 mg/m². In some embodiments, doses of doxorubicin are administered in 21-day cycles, e.g., on day 1 of each 21-day cycle. In some embodiments, the doxorubicin administered to an individual according to any of the methods provided herein is a pharmaceutically acceptable salt thereof. In some embodiments, the doxorubicin is doxorubicin hydrochloride.

### VI. Corticosteroids

In some embodiments, the methods for treating DLBCL provided herein comprise administering to an individual, e.g., a human patient, an anti-CD79b immunoconjugate (e.g., huMA79bv28-MC-vc-PAB-MMAE or polatuzumab vedotin), an anti-CD20 antibody (such as obinutuzumab or rituximab), one or more chemotherapeutic agents (e.g., cyclophosphamide and/or doxorubicin), and a corticosteroid. In some embodiments, the corticosteroid is prednisolone, methylprednisolone, or prednisone. In some embodiments, the methods for treating DLBCL provided herein comprise administering to an individual, e.g., a human patient, an anti-CD79b immunoconjugate (e.g., huMA 79bv28-MC-vc-PAB-MMAE or polatuzumab vedotin), an anti-CD20 antibody (such as obinutuzumab or rituximab), one or more chemotherapeutic agents (e.g., cyclophosphamide and/or doxorubicin), and prednisone, prednisolone, or methylprednisolone. In some embodiments, the methods for treating DLBCL provided herein comprise administering to an individual, e.g., a human patient, an anti-CD79b immunoconjugate (e.g., huMA79bv28-MC-vc-PAB-MMAE or polatuzumab vedotin), an anti-CD20 antibody (such as obinutuzumab or rituximab), one or more chemotherapeutic agents (e.g., cyclophosphamide and/or doxorubicin), and prednisone. In some embodiments, the methods for treating DLBCL provided herein comprise administering to an individual, e.g., a human patient, an anti-CD79b immunoconjugate (e.g., huMA79bv28-MC-vc-PAB-MMAE or polatuzumab vedotin), an anti-CD20 antibody (such as obinutuzumab or rituximab), one or more chemotherapeutic agents (e.g., cyclophosphamide and/or doxorubicin), and prednisolone. In some embodiments, the methods for treating DLBCL provided herein comprise administering to an individual, e.g., a human patient, an anti-CD79b immunoconjugate (e.g., huMA79bv28-MC-vc-PAB-MMAE or polatuzumab vedotin), an anti-CD20 antibody (such as obinutuzumab or rituximab), one or more chemotherapeutic agents (e.g., cyclophosphamide and/or doxorubicin), and methylprednisolone. In some embodiments, more than one corticosteroid may be administered to an individual during the course of treatment according any of the methods of treating DLBCL provided herein.

Prednisone is available commercially, e.g., as Deltasone, Prednicot, predniSONE Intensol, Rayos, Sterapred, or Sterapred DS. Typical dosages of prednisone that may be used include, for example, between about 5 mg to about 60 mg per day, between about 10 mg to about 60 mg per day, between about 5 mg to about 100 mg per day, or between about 30 mg to about 100 mg per day, administered orally. In some embodiments, the dose of prednisone is about 100 mg per day. In some embodiments, doses of prednisone are administered in 21-day cycles, e.g., on days 1-5 of each 21-day cycle. In some embodiments, the prednisone administered to an individual according to any of the methods provided herein is a pharmaceutically acceptable salt or ester thereof. In some embodiments, the prednisone is prednisone acetate, prednisone palmitate, or prednisone succinate.

Prednisolone is available commercially, e.g., as Bubbli-Pred, Cotolone, Flo-Pred, Millipred, Millipred DP, Orapred, Orapred ODT, Pediapred, Prelone, or Veripred 20. Typical dosages of prednisolone that may be used include, for example, between about 5 mg to about 60 mg per day, between about 5 mg to about 100 mg per day, or between about 30 mg to about 100 mg per day, administered orally. In some embodiments, the dose of prednisolone is about 100 mg per day. In some embodiments, doses of prednisolone are administered in 21-day cycles, e.g., on days 1-5 of each 21-day cycle. In some embodiments, the prednisolone administered to an individual according to any of the methods provided herein is a pharmaceutically acceptable salt or ester thereof. In some embodiments, the prednisolone is prednisolone sodium phosphate, prednisolone acetate, prednazate (prednisolone succinate and perphenazine compound), prednazoline (prednisolone phosphate and fenoxazoline compound), prednicarbate (prednisolone 17-(ethyl carbonate) 21-propionate), prednimustine (prednisolone chlorambucil ester), prednisolamate (prednisolone diethylaminoacetate), prednisolone hexanoate, prednisolone metasulphobenzoate (prednisolone 21-(3-sulfobenzoate)), prednisolone palmitate, prednisolone phosphate, prednisolone piperidinoacetate, prednisolone pivalate, prednisolone stearoylglycolate, prednisolone tetrahydrophthalate, prednisolone steaglate (prednisolone stearoyl-glycolate), prednisolone succinate (prednisolone hemisuccinate), prednisolone sulfate, prednisolone tebutate (prednisolone tert-butylacetate), prednisolone valerate, or prednisolone valeroacetate.

Methylprednisolone is available commercially, e.g., as A-Methapred, Depo-Medrol, or SoluMedrol. Typical dosages of methylprednisolone that may be used include, for example, between about 2 mg to about 250 mg per day, between about 2 mg to about 60 mg per day, or between about 10 mg to about 80 mg per day. Methylprednisolone may be administered orally or intravenously. In some embodiments, methylprednisolone is administered intravenously. In some embodiments, the dose of methylprednisolone is about 80 mg per day, administered intravenously. In some embodiments, doses of methylprednisolone are administered in 21-day cycles, e.g., on days 1-5 of each 21-day cycle. In some embodiments, the methylprednisolone administered to an individual according to any of the methods provided herein is a pharmaceutically acceptable salt or ester thereof. In some embodiments, the methylprednisolone is methylprednisolone acetate, methylprednisolone succinate, methylprednisolone acetate propionate, methylprednisolone sodium succinate, methylprednisolone hemisuccinate or methylprednisolone hydrogen succinate, methylprednisolone aceponate, methylprednisolone cyclopentylpropionate, methylprednisolone phosphate, methylprednisolone succinate (methylprednisolone hemisuccinate), or methylprednisolone suleptanate.

In some embodiments, the corticosteroid is not hydrocortisone.

### VII. Anti-CD20 Agents

Depending on binding properties and biological activities of anti-CD20 antibodies to the CD20 antigen, two types of anti-CD20 antibodies (type I and type II anti-CD20 antibodies) can be distinguished according to Cragg, M. S., et al., Blood 103 (2004) 2738-2743; and Cragg, M. S., et al., Blood 101 (2003) 1045-1052, see Table A.

TABLE A
Properties of type I and type II anti-CD20 antibodies

| Type I anti-CD20 antibodies | Type II anti-CD20 antibodies |
|---|---|
| type I CD20 epitope | type II CD20 epitope |
| Localize CD20 to lipid rafts | Do not localize CD20 to lipid rafts |
| Increased CDC (if IgG1 isotype) | Decreased CDC (if IgG1 isotype) |
| ADCC activity (if IgG1 isotype) | ADCC activity (if IgG1 isotype) |
| Full binding capacity | Reduced binding capacity |
| Homotypic aggregation | Stronger homotypic aggregation |
| Apoptosis induction upon | Strong cell death induction without |
| cross-linking | cross-linking |

Examples of type I anti-CD20 antibodies include e.g., rituximab, HI47 IgG3 (ECACC, hybridoma), 2C6 IgG1 (as disclosed in WO 2005/103081), 2F2 IgG1 (as disclosed and WO 2004/035607 and WO 2005/103081) and 2H7 IgG1 (as disclosed in WO 2004/056312).

In some embodiments, the anti-CD20 antibody used in a method of treatment provided herein comprises, according to numbering in Kabat et al., the CDR-H1, CDR-H2, CDR-H3, CDR-L1, CDR-L2, and CDR-L3 of rituximab. In some embodiments, the anti-CD20 antibody used in a method of treatment provided herein comprises the VH and the VL of rituximab. In some embodiments, the anti-CD20 antibody used in a method of treatment provided herein comprises the heavy chain and the light chain of rituximab. As used herein, the term “rituximab” refers to an anti-CD20 antibody having the CAS Registry Number 174722-31-7. In some embodiments, the anti-CD20 antibody used in a method of treatment provided herein is rituximab. In some embodiments, the rituximab (reference antibody; example of a type I anti-CD20 antibody) is a genetically engineered chimeric human gamma 1 murine constant domain containing monoclonal antibody directed against the human CD20 antigen. However, this antibody is not glycoengineered and not afucosylated and thus has an amount of fucose of at least 85%. This chimeric antibody comprises human gamma 1 constant domains and is identified by the name “C2B8” in U.S. Pat. No. 5,736,137 (Andersen, et. al.) issued on Apr. 17, 1998, assigned to IDEC Pharmaceuticals Corporation. Rituximab is approved for the treatment of patients with relapsed or refracting low-grade or follicular, CD20 positive, B-cell non-Hodgkin's lymphoma. In vitro mechanism of action studies have shown that rituximab exhibits human complement-dependent cytotoxicity (CDC) (Reff, M. E., et. al, Blood 83 (2) (1994) 435-445). Additionally, it exhibits activity in assays that measure antibody-dependent cellular cytotoxicity (ADCC). The term “rituximab” also refers to all corresponding anti-CD20 antibodies that fulfill the requirements necessary for obtaining a marketing authorization as an identical or biosimilar product in a country or territory selected from the group of countries consisting of the USA, Europe and Japan. Examples of such corresponding anti-CD20 antibodies encompassed by the term “rituximab” as used herein may include, without limitation, Riabni (rituximab-arrx), Ruxience (rituximab-pvvr), Truxima (rituximab-abbs), CT-P10 (Truxima, Ritemvia, Blitzima; Celltrion), GP2013 (Rixathon, Riximyo; Sandoz), and Ruxience (Pfizer).

Rituximab can be provided as a component of a product or composition which comprises rituximab, or any corresponding anti-CD20 antibodies that fulfill the requirements necessary for obtaining a marketing authorization as an identical or biosimilar product to rituximab in a country or territory selected from the group of countries consisting of the USA, Europe and Japan, e.g., as described above. For example, RITUXAN HYCELA® (rituximab/hyaluronidase human) is a fixed combination of rituximab and recombinant hyaluronidase human. Hyaluronidase human is an enzyme that increases permeability of subcutaneous tissue by temporarily depolymerizing hyaluronan, which is a polysaccharide found in the extracellular matrix of subcutaneous tissue. Hyaluronidase human has been shown to increase the absorption rate of antibodies (e.g., rituximab) into systemic circulation. RITUXAN HYCELA® is approved for the treatment of patients with follicular lymphoma (FL), DLBCL, and chronic lymphocytic leukemia (CLL). See, e.g., the website: www.accessdata.fda.gov/drugsatfda_docs/label/2017/761064s0001bl.pdf for additional information about rituxan hycela.

In some embodiments, the anti-CD20 antibody used in a method of treatment provided herein is an afucosylated anti-CD20 antibody.

Examples of type II anti-CD20 antibodies include e.g., humanized B-Ly1 antibody IgG1 (a chimeric humanized IgG1 antibody as disclosed in WO 2005/044859), 11B8 IgG1 (as disclosed in WO 2004/035607), and AT80 IgG1. Typically type II anti-CD20 antibodies of the IgG1 isotype show characteristic CDC properties. Type II anti-CD20 antibodies have a decreased CDC (if IgG1 isotype) compared to type I antibodies of the IgG1 isotype. In some embodiments, the type II anti-CD20 antibody, e.g., a GA101 antibody, has increased antibody dependent cellular cytotoxicity (ADCC). In some embodiments, the anti-CD20 antibody is a type II anti-CD20 antibody, more preferably an afucosylated humanized B-Ly1 antibody as described in WO 2005/044859 and WO 2007/031875.

In some embodiments, the anti-CD20 antibody used in a method of treatment provided herein is the GA101 antibody. In some embodiments, the GA101 antibody as used herein refers to any one of the following antibodies that bind human CD20: (1) an antibody comprising an HVR-H1 comprising the amino acid sequence of SEQ ID NO:5, an HVR-H2 comprising the amino acid sequence of SEQ ID NO: 6, an HVR-H3 comprising the amino acid sequence of SEQ ID NO: 7, an HVR-L1 comprising the amino acid sequence of SEQ ID NO:8, an HVR-L2 comprising the amino acid sequence of SEQ ID NO: 9, and an HVR-L3 comprising the amino acid sequence of SEQ ID NO:10; (2) an antibody comprising a VH domain comprising the amino acid sequence of SEQ ID NO:11 and a VL domain comprising the amino acid sequence of SEQ ID NO: 12, (3) an antibody comprising a heavy chain amino acid sequence of SEQ ID NO:13 and a light chain amino acid sequence of SEQ ID NO: 14; (4) an antibody known as obinutuzumab, or (5) an antibody that comprises a heavy chain amino acid sequence with at least 95%, 96%, 97%, 98% or 99% sequence identity with amino acid sequence of SEQ ID NO: 13 and that comprises a light chain amino acid sequence with at least 95%, 96%, 97%, 98% or 99% sequence identity with an amino acid sequence of SEQ ID NO: 14. In one embodiment, the GA101 antibody is an IgG1 isotype antibody.

In some embodiments, the anti-CD20 antibody used in a method of treatment provided herein is a humanized B-Ly1 antibody. In some embodiments, the humanized B-Ly1 antibody refers to humanized B-Ly1 antibody as disclosed in WO 2005/044859 and WO 2007/031875, which were obtained from the murine monoclonal anti-CD20 antibody B-Ly1 (variable region of the murine heavy chain (VH): SEQ ID NO: 3: variable region of the murine light chain (VL): SEQ ID NO: 4-see Poppema, S. and Visser, L., Biotest Bulletin 3 (1987) 131-139) by chimerization with a human constant domain from IgG1 and following humanization (see WO 2005/044859 and WO 2007/031875). The humanized B-Ly1 antibodies are disclosed in detail in WO 2005/044859 and WO 2007/031875.

In some embodiments, the humanized B-Ly1 antibody has variable region of the heavy chain (VH) selected from group of SEQ ID NO: 15-16 and 40-54 (corresponding to B—HH2 to B—HH9 and B-HL8 to B-HL17 of WO 2005/044859 and WO 2007/031875). In some embodiments, the variable domain is selected from the group consisting of SEQ ID NO: 15, 16, 42, 44, 46, 48 and 50 (corresponding to B—HH2, BHH-3, B—HH6, B—HH8, B-HL8, B-HL11 and B-HL13 of WO 2005/044859 and WO 2007/031875). In some embodiments, the humanized B-Ly1 antibody has a variable region of the light chain (VL) of SEQ ID NO: 55 (corresponding to B—KV1 of WO 2005/044859 and WO 2007/031875). In some embodiments, the humanized B-Ly1 antibody has a variable region of the heavy chain (VH) of SEQ ID NO: 42 (corresponding to B—HH6 of WO 2005/044859 and WO 2007/031875) and a variable region of the light chain (VL) of SEQ ID NO: 55 (corresponding to B—KV1 of WO 2005/044859 and WO 2007/031875). In some embodiments, the humanized B-Ly1 antibody is an IgG1 antibody. Such afucosylated humanized B-Ly1 antibodies are glycoengineered (GE) in the Fc region according to the procedures described in WO 2005/044859, WO 2004/065540, WO 2007/031875, Umana, P. et al., Nature Biotechnol. 17 (1999) 176-180 and WO 99/154342. In some embodiments, the afucosylated glyco-engineered humanized B-Ly1 is B-HH6-B—KV1 GE. In some embodiments, the anti-CD20 antibody is obinutuzumab (recommended INN, WHO Drug Information, Vol. 26, No. 4, 2012, p. 453). As used herein, obinutuzumab is synonymous for GA101 or RO5072759. It is commercially available for therapeutic use under the trade name GAZYVA®, and is provided as a 1000 mg/40 mL (25 mg/mL) single-dose vial. This replaces all previous versions (e.g., Vol. 25, No. 1, 2011, p. 75-76), and is formerly known as afutuzumab (recommended INN, WHO Drug Information, Vol. 23, No. 2, 2009, p. 176: Vol. 22, No. 2, 2008, p. 124). In some embodiments, the humanized B-Ly1 antibody is an antibody comprising a heavy chain comprising the amino acid sequence of SEQ ID NO:17 and a light chain comprising the amino acid sequence of SEQ ID NO:18, or an antigen-binding fragment thereof such antibody. In some embodiments, the humanized B-Ly1 antibody comprises a heavy chain variable region comprising the three heavy chain CDRs of SEQ ID NO:17 and a light chain variable region comprising the three light chain CDRs of SEQ ID NO:18.

In some embodiments, the humanized B-Ly1 antibody is an afucosylated glyco-engineered humanized B-Ly1. Such glycoengineered humanized B-Ly1 antibodies have an altered pattern of glycosylation in the Fc region, preferably having a reduced level of fucose residues. In some embodiments, the amount of fucose is about 60% or less of the total amount of oligosaccharides at Asn297 (in one embodiment the amount of fucose is between about 40% and about 60%, in another embodiment the amount of fucose is about 50% or less, and in still another embodiment the amount of fucose is about 30% or less). In some embodiments, the oligosaccharides of the Fc region are bisected. These glycoengineered humanized B-Ly1 antibodies have an increased ADCC.

The “ratio of the binding capacities to CD20 on Raji cells (ATCC-No. CCL-86) of anti-CD20 antibodies compared to rituximab” is determined by direct immunofluorescence measurement (the mean fluorescence intensities (MFI) is measured) using said anti-CD20 antibody conjugated with Cy5 and rituximab conjugated with Cy5 in a FACSArray (Becton Dickinson) with Raji cells (ATCC-No. CCL-86), calculated as follows:

$$\\ \begin{matrix}Ratio of the binding capacities to CD 20 \\ on Raji cells ( ATCC ‐ No . CCL ‐ 86 )\end{matrix} = \frac{MFI ( Cy 5 ‐ anti ‐ CD 20 antibody )}{MFI ( Cy 5 ‐ rituximab )} \times \frac{Cy 5 ‐ labeling ratio ( Cy 5 ‐ rituximab )}{Cy 5 ‐ labeling ratio ( Cy 5 ‐ anti ‐ CD 20 antibody )}$$

MFI is the mean fluorescent intensity. The “Cy5-labeling ratio” as used herein means the number of Cy5-label molecules per molecule antibody.

Typically said type II anti-CD20 antibodies have a ratio of the binding capacities to CD20 on Raji cells (ATCC-No. CCL-86) of said second anti-CD20 antibody compared to rituximab of 0.3 to 0.6, and in one embodiment, 0.35 to 0.55, and in yet another embodiment, 0.4 to 0.5.

By “antibody having increased antibody dependent cellular cytotoxicity (ADCC)”, it is meant an antibody, as that term is defined herein, having increased ADCC as determined by any suitable method known to those of ordinary skill in the art.

An exemplary accepted in vitro ADCC assay is described below:
1) the assay uses target cells that are known to express the target antigen recognized by the antigen-binding region of the antibody;
2) the assay uses human peripheral blood mononuclear cells (PBMCs), isolated from blood of a randomly chosen healthy donor, as effector cells;
3) the assay is carried out according to following protocol:
   i) the PBMCs are isolated using standard density centrifugation procedures and are suspended at 5×10⁶ cells/ml in RPMI cell culture medium;
   ii) the target cells are grown by standard tissue culture methods, harvested from the exponential growth phase with a viability higher than 90%, washed in RPMI cell culture medium, labeled with 100 micro-Curies of ⁵¹Cr, washed twice with cell culture medium, and resuspended in cell culture medium at a density of 10⁵ cells/ml;
   iii) 100 microliters of the final target cell suspension above are transferred to each well of a 96-well microtiter plate;
   iv) the antibody is serially-diluted from 4000 ng/ml to 0.04 ng/ml in cell culture medium and 50 microliters of the resulting antibody solutions are added to the target cells in the 96-well microtiter plate, testing in triplicate various antibody concentrations covering the whole concentration range above;
   v) for the maximum release (MR) controls, 3 additional wells in the plate containing the labeled target cells, receive 50 microliters of a 2% (VN) aqueous solution of non-ionic detergent (Nonidet, Sigma, St. Louis), instead of the antibody solution (point iv above);
   vi) for the spontaneous release (SR) controls, 3 additional wells in the plate containing the labeled target cells, receive 50 microliters of RPMI cell culture medium instead of the antibody solution (point iv above);
   vii) the 96-well microtiter plate is then centrifuged at 50×g for 1 minute and incubated for 1 hour at 4° C.;
   viii) 50 microliters of the PBMC suspension (point i above) are added to each well to yield an effector:target cell ratio of 25:1 and the plates are placed in an incubator under 5% CO₂ atmosphere at 37° C. for 4 hours;
   ix) the cell-free supernatant from each well is harvested and the experimentally released radioactivity (ER) is quantified using a gamma counter;
   x) the percentage of specific lysis is calculated for each antibody concentration according to the formula (ER−MR)/(MR−SR)×100, where ER is the average radioactivity quantified (see point ix above) for that antibody concentration, MR is the average radioactivity quantified (see point ix above) for the MR controls (see point V above), and SR is the average radioactivity quantified (see point ix above) for the SR controls (see point vi above);
4) “increased ADCC” is defined as either an increase in the maximum percentage of specific lysis observed within the antibody concentration range tested above, and/or a reduction in the concentration of antibody required to achieve one half of the maximum percentage of specific lysis observed within the antibody concentration range tested above. In one embodiment, the increase in ADCC is relative to the ADCC, measured with the above assay, mediated by the same antibody, produced by the same type of host cells, using the same standard production, purification, formulation and storage methods, which are known to those skilled in the art, except that the comparator antibody (lacking increased ADCC) has not been produced by host cells engineered to overexpress GnTIII and/or engineered to have reduced expression from the fucosyltransferase 8 (FUT8) gene (e.g., including, engineered for FUT8 knock out).

In some embodiments, the “increased ADCC” can be obtained by, for example, mutating and/or glycoengineering of said antibodies. In some embodiments, the anti-CD20 antibody is glycoengineered to have a biantennary oligosaccharide attached to the Fc region of the antibody that is bisected by GlcNAc. In some embodiments, the anti-CD20 antibody is glycoengineered to lack fucose on the carbohydrate attached to the Fc region by expressing the antibody in a host cell that is deficient in protein fucosylation (e.g., Lec13 CHO cells or cells having an alpha-1,6-fucosyltransferase gene (FUT8) deleted or the FUT gene expression knocked down). In some embodiments, the anti-CD20 antibody sequence has been engineered in its Fc region to enhance ADCC. In some embodiments, such an engineered anti-CD20 antibody variant comprises an Fc region with one or more amino acid substitutions at positions 298, 333, and/or 334 of the Fc region (EU numbering of residues)).

In some embodiments, the term “complement-dependent cytotoxicity (CDC)” refers to lysis of human cancer target cells by the antibody according to the invention in the presence of complement. CDC can be measured by the treatment of a preparation of CD20 expressing cells with an anti-CD20 antibody according to the invention in the presence of complement. CDC is found if the antibody induces at a concentration of 100 nM the lysis (cell death) of 20% or more of the tumor cells after 4 hours. In some embodiments, the assay is performed with ⁵¹Cr or Eu labeled tumor cells and measurement of released ⁵¹Cr or Eu. Controls include the incubation of the tumor target cells with complement but without the antibody.

In some embodiments, the anti-CD20 antibody is a monoclonal antibody, e.g., a human antibody. In some embodiments, the anti-CD20 antibody is an antibody fragment, e.g., a Fv, Fab, Fab′, scFv, diabody, or F (ab)₂ fragment. In some embodiments, the anti-CD20 antibody is a substantially full length antibody, e.g., an IgG1 antibody, IgG2a antibody or other antibody class or isotype as defined herein.

### VIII. Antibodies

In some embodiments, an anti-CD20 antibody or an anti-CD79b antibody used in a method of treatment provided herein has a dissociation constant (Kd) for binding CD20 (e.g., human CD20) or CD79b (e.g., human CD79b), respectively, of ≤1 μM, ≤100 nM, ≤50 nM, ≤10 nM, ≤5 nM, ≤1 nM, ≤0.1 nM, ≤0.01 nM, or ≤0.001 nM, and optionally is ≥10⁻¹³ M (e.g., 10⁻⁸ M or less, e.g., from 10⁻⁸ M to 10⁻¹³ M, e.g., from 10⁻⁹ M to 10⁻¹³ M). In some embodiments, the anti-CD20 antibody or anti-CD79b antibody is an antibody fragment. In some embodiments, the anti-CD20 antibody or anti-CD79b antibody is a chimeric or a humanized antibody. In some embodiments, the anti-CD20 antibody or anti-CD79b antibody is a human antibody. In some embodiments, the anti-CD20 antibody or anti-CD79b antibody is a multispecific antibody, e.g., a bispecific antibody.

In certain embodiments, amino acid sequence variants of an anti-CD79b antibody or an anti-CD20 antibody used in a method of treatment provided herein are contemplated. For example, it may be desirable to improve the binding affinity and/or other biological properties of the anti-CD79b antibody or anti-CD20 antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### A. Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table B under the heading of “preferred substitutions.” More substantial changes are provided in Table B under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE B

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see. e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H₃ and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### B. Glycosylation Variants

In certain embodiments, an antibody (e.g., an anti-CD79b antibody or an anti-CD20 antibody) used in a method of treatment provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See. e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See. e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see. e.g., Yamane-Ohnuki et al. Biotech. Bioeng. 87:614 (2004); Kanda, Y. et al., Biotechnol. Bioeng., 94 (4): 680-688 (2006); and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.): WO 1998/58964 (Raju, S.); and WO 1999/22764 (Raju, S.).

### C. Fc Variants

In certain embodiments, one or more amino acid modifications may be introduced into the Fc region of an antibody (e.g., an anti-CD79b antibody or an anti-CD20 antibody) used in a method of treatment provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc (RIII only, whereas monocytes express Fc (RI, Fc (RII and Fc (RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see. e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See. e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see. e.g., Petkova, S. B. et al., Int'l. Immunol. 18 (12): 1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See. e.g., U.S. Pat. No. 6,737,056; WO 2004/056312, and Shields et al., J. Biol. Chem. 9 (2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988): U.S. Pat. No. 5,648,260: U.S. Pat. No. 5,624,821; and WO 94/29351 concerning other examples of Fc region variants.

### D. Cysteine Engineered Antibody Variants

In certain embodiments, it may be desirable to create cysteine engineered antibodies, e.g., “thioMAbs,” in which one or more residues of an anti-CD79b antibody or an anti-CD20 antibody used in a method of treatment provided herein are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### E. Antibody Derivatives

In certain embodiments, an antibody (e.g., an anti-CD79b antibody or an anti-CD20 antibody) used in a method of treatment provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly (n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102:11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### IX. Pharmaceutical Formulations

Pharmaceutical formulations of any of the agents described herein (e.g., anti-CD79b immunoconjugates, anti-CD20 antibodies, chemotherapeutic agents, and corticosteroids) for use in any of the methods as described herein are prepared by mixing such agent(s) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride: benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody or immunoconjugate formulations are described in U.S. Pat. No. 6,267,958. Aqueous antibody or immunoconjugate formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody or immunoconjugate, or one or more agents described herein, which matrices are in the form of shaped articles, e.g., films, or microcapsules.

The formulations to be used for in vivo administration are generally sterile. Sterility may be readily accomplished, e.g., by filtration through sterile filtration membranes.

Additional details regarding pharmaceutical formulations comprising an anti-CD79b immunoconjugate are provided in WO 2009/099728 the contents of which are expressly incorporated by reference herein in their entirety.

### X. Kits and Articles of Manufacture

In another embodiment, an article of manufacture or a kit is provided comprising an anti-CD79b immunoconjugate (such as described herein) and at least one additional agent. In some embodiments the at least one additional agent is an anti-CD20 antibody (e.g., rituximab or obinutuzumab), one or more chemotherapeutic agents (e.g., cyclophosphamide and/or doxorubicin), and a corticosteroid (e.g., prednisone, prednisolone, or methylprednisolone). In some embodiments, the article of manufacture or kit further comprises a package insert comprising instructions for using the anti-CD79b immunoconjugate in conjunction at least one additional agent, such as an anti-CD20 antibody (e.g., rituximab or obinutuzumab), one or more chemotherapeutic agents (e.g., cyclophosphamide and/or doxorubicin), and a corticosteroid (e.g., prednisone, prednisolone, or methylprednisolone) to treat or delay progression of a B-cell proliferative disorder (e.g., DLBCL) in an individual. Any of the anti-CD79b immunoconjugates, anti-CD20 antibodies, chemotherapeutic agents, and/or corticosteroids, and optionally one or more additional anti-cancer agents, known in the art or described herein may be included in the article of manufacture or kits. In some embodiments, the kit comprises an immunoconjugate comprising the formula

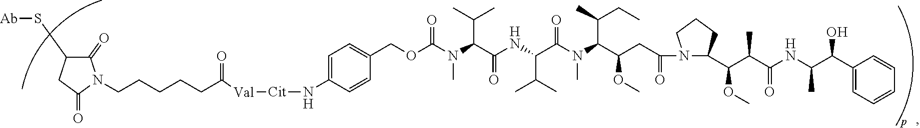

wherein Ab is an anti-CD79b antibody comprising (i) an HVR-H1 that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8. In some embodiments, the kit comprises an immunoconjugate comprising the formula

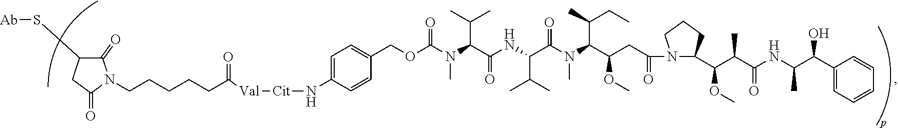

wherein Ab is an anti-CD79b antibody that comprises (i) a heavy chain comprising a VH that comprises the amino acid sequence of SEQ ID NO: 19 and (ii) a light chain comprising a VL that comprises the amino acid sequence of SEQ ID NO: 20, and wherein p is between 2 and 5. In some embodiments, p is between 3 and 4, e.g., 3.4 or 3.5. In some embodiments, the immunoconjugate comprises an anti-CD79b antibody comprising a heavy chain comprising the amino acid sequence of SEQ ID NO: 36, and a light chain comprising the amino acid sequence of SEQ ID NO: 35. In certain embodiments, the anti-CD79b immunoconjugate comprises the structure of Ab-MC-vc-PAB-MMAE. In some embodiments, the anti-CD79b immunoconjugate is polatuzumab vedotin (CAS Number 1313206-42-6). In some embodiments, the at least one additional agent is an anti-CD20 antibody (e.g., rituximab or obinutuzumab), one or more chemotherapeutic agents (e.g., cyclophosphamide and/or doxorubicin), and a corticosteroid (e.g., prednisone, prednisolone, or methylprednisolone). In some embodiments, the kit is for use in the treatment of DLBCL, e.g., previously untreated DLBCL, in an individual, such as a human patient (e.g., a human patient having one or more characteristics described herein) according to a method provided herein.

In some embodiments, the anti-CD79b immunoconjugate, the anti-CD20 antibody (e.g., rituximab or obinutuzumab), one or more chemotherapeutic agents (e.g., cyclophosphamide and/or doxorubicin), and a corticosteroid (e.g., prednisone, prednisolone, or methylprednisolone) are in the same container or in separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation, and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.
Exemplary Embodiment 2: The method of embodiment 1, wherein the PFS or the reference PFS is measured:
   (a) starting from the start of the corresponding treatment to the time of a first occurrence of disease progression, relapse, or death; or
   (b) starting from up to 7 days prior to the start of the corresponding treatment to the time of a first occurrence of disease progression, relapse, or death;
   (c) starting from the time from randomization to the time of a first occurrence of disease progression, relapse, or death.
Exemplary Embodiment 3: The method of embodiment 1 or embodiment 2, wherein the PFS or the reference PFS is the median PFS of the plurality of human patients receiving the corresponding treatment.
Exemplary Embodiment 4: The method of any one of embodiments 1-3, wherein the improvement in PFS is (a) statistically significant; (b) statistically significant with a hazard ratio of no more than 0.75 (95% confidence interval: 0.57, 0.97); (c) statistically significant with a hazard ratio of no more than 0.78 (95% confidence interval: 0.60, 1.00); or (d) statistically significant with a hazard ratio of no more than 0.79 (95% confidence interval: 0.61, 1.02).
Exemplary Embodiment 5: A method for treating diffuse large B-cell lymphoma (DLBCL) in a human patient in need thereof, comprising administering to the human patient an effective amount of:
   (a) an immunoconjugate comprising the formula:

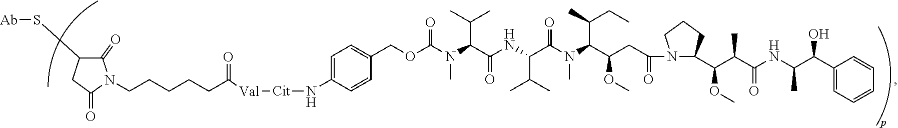

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8,
(b) rituximab,
(c) cyclophosphamide,
(d) doxorubicin, and
(e) prednisone, prednisolone, or methylprednisolone;
   wherein administering such treatment to a plurality of human patients results in: at least a 20% reduction in the risk of disease progression, relapse, or death in the plurality of human patients, or at least a 25% reduction in the risk of disease progression, relapse, or death in the plurality of human patients,
   as compared to a control treatment comprising:
   (a) rituximab,
   (b) cyclophosphamide,
   (c) doxorubicin,
   (d) vincristine, and
   (e) prednisone, prednisolone, or methylprednisolone,
   in the absence of immunoconjugate.
Exemplary Embodiment 6: The method of embodiment 5, wherein said disease progression, relapse, or death are measured:
   (a) starting from the start of the corresponding treatment to the time of a first occurrence of disease progression, relapse, or death; or
   (b) starting from up to 7 days prior to the start of the corresponding treatment to the time of a first occurrence of disease progression, relapse, or death; or
   (c) starting from the time from randomization to the time of a first occurrence of disease progression, relapse, or death.
Exemplary Embodiment 7: The method of embodiment 5 or embodiment 6, wherein the reduction of risk has a 95% confidence interval.
Exemplary Embodiment 8: The method of any one of embodiments 5-7, wherein administering such treatment results in a statistically significant improvement in progression-free survival of the plurality of human patients as compared to the control treatment with: at least 20% reduction in the risk of disease progression, relapse, or death, or at least 25% reduction in the risk of disease progression, relapse, or death.
Exemplary Embodiment 9: The method of any one of embodiments 5-8, wherein the reduction in the risk of disease progression, relapse, or death is statistically significant.
Exemplary Embodiment 10: The method of any one of embodiments 5-9, wherein the reduction in the risk of disease progression, relapse, or death is calculated at 24 months or more, or 36 months or more, measured starting from:
   (a) the start of the corresponding treatment; or
   (b) up to 7 days prior to the start of the corresponding treatment; or
   (c) from the time from randomization to the time of a first occurrence of disease progression, relapse, or death.
Exemplary Embodiment 11: A method for treating diffuse large B-cell lymphoma (DLBCL) in a human patient in need thereof, comprising administering to the human patient an effective amount of:
   (a) an immunoconjugate comprising the formula:

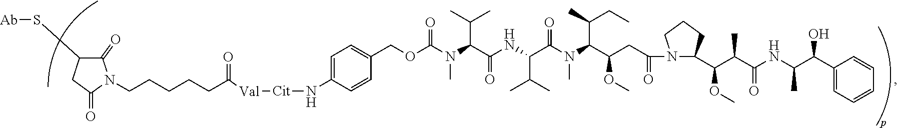

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8,
(b) rituximab,
(d) doxorubicin, and
(e) prednisone, prednisolone, or methylprednisolone;
   wherein administering such treatment to a plurality of human patients results in: a hazard ratio of no more than 0.75 in progression-free survival (PFS) of the plurality of human patients, or a hazard ratio of no more than 0.78 in PFS of the plurality of human patients, or a hazard ratio of no more than 0.79 in PFS of the plurality of human patients,
   as compared to a control treatment comprising:
   (a) rituximab,
   (b) cyclophosphamide,
   (c) doxorubicin,
   (d) vincristine, and
   (e) prednisone, prednisolone, or methylprednisolone, in the absence of immunoconjugate.
Exemplary Embodiment 12: The method of embodiment 11, wherein the PFS is measured:
   (a) starting from the start of the corresponding treatment to the time of a first occurrence of disease progression, relapse, or death; or
   (b) starting from up to 7 days prior to the start of the corresponding treatment to the time of a first occurrence of disease progression, relapse, or death;
   (c) starting from the time from randomization to the time of a first occurrence of disease progression, relapse, or death.
Exemplary Embodiment 13: The method of embodiment 11 or embodiment 12, wherein the hazard ratio has a 95% confidence interval.
Exemplary Embodiment 14: The method of any one of embodiments 11-13, wherein administering such treatment results in a statistically significant improvement in the PFS as compared to the control treatment with: a hazard ratio of no more than 0.75 (95% confidence interval: 0.57, 0.97), or a hazard ratio of no more than 0.78 (95% confidence interval: 0.60, 1.00), or a hazard ratio of no more than 0.79 (95% confidence interval: 0.61, 1.02).
Exemplary Embodiment 15: The method of any one of embodiments 11-14, wherein the hazard ratio is calculated at 24 months or more, or 36 months or more, measured starting from:
   (a) the start of the corresponding treatment; or
   (b) up to 7 days prior to the start of the corresponding treatment; or
Exemplary Embodiment 18: A method for treating diffuse large B-cell lymphoma (DLBCL) in a human patient in need thereof, comprising administering to the human patient an effective amount of:
   (a) an immunoconjugate comprising the formula:

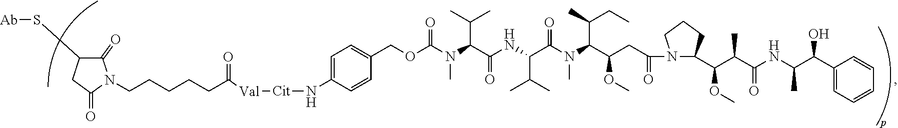

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8,
(b) rituximab,
(c) cyclophosphamide,
(d) doxorubicin, and
(e) prednisone, prednisolone, or methylprednisolone;
   wherein administering such treatment to a plurality of human patients results in an improvement in a 24-month progression-free survival rate (PFS24) of the plurality of human patients as compared to a reference PFS24,
   wherein the reference PFS24 is the 24-month progression-free survival rate of a plurality of human patients who have received a control treatment comprising:
   (a) rituximab,
   (b) cyclophosphamide,
   (c) doxorubicin,
   (d) vincristine, and
   in the absence of immunoconjugate.
Exemplary Embodiment 19: A method for treating diffuse large B-cell lymphoma (DLBCL) in a human patient in need thereof, comprising administering to the human patient an effective amount of:
   (a) an immunoconjugate comprising the formula:

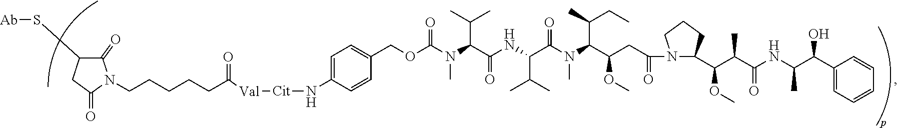

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8,
(b) rituximab,
(c) cyclophosphamide,
(d) doxorubicin, and
(e) prednisone, prednisolone, or methylprednisolone;
   wherein administering such treatment to a plurality of human patients results in an improvement in a 24-month progression-free survival rate (PFS24) of the plurality of human patients of at least about 6%, as compared to a reference PFS24,
   wherein the reference PFS24 is the 24-month progression-free survival rate of a plurality of human patients who have received a control treatment comprising:
   (a) rituximab,
   (b) cyclophosphamide,
   (c) doxorubicin,
   (d) vincristine, and
   (e) prednisone, prednisolone, or methylprednisolone;
   in the absence of immunoconjugate.
Exemplary Embodiment 20: The method of embodiment 18 or embodiment 19, wherein the PFS24 or the reference PFS24 is calculated at 24 months, measured starting from:
   (a) the start of the corresponding treatment; or
   (b) up to 7 days prior to the start of the corresponding treatment; or
   (c) starting from the time from randomization to the time of a first occurrence of disease progression, relapse, or death.
Exemplary Embodiment 21: The method of any one of embodiments 16-20, wherein the PFS24 or the reference PFS24 is a progression-free survival (PFS) rate calculated using a Kaplan-Meier method.
Exemplary Embodiment 22: The method of any one of embodiments 18-21, wherein the improvement in PFS24 is statistically significant.
Exemplary Embodiment 23: A method for treating diffuse large B-cell lymphoma (DLBCL) in a human patient in need thereof, comprising administering to the human patient an effective amount of:
   (a) an immunoconjugate comprising the formula:

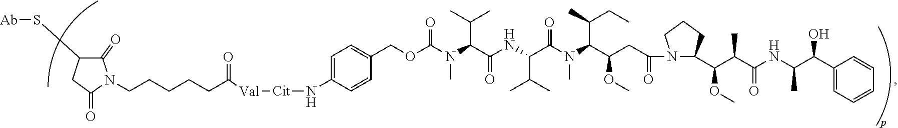

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8,
(b) rituximab,
(c) cyclophosphamide,
(d) doxorubicin, and
(e) prednisone, prednisolone, or methylprednisolone;
   wherein administering such treatment to a plurality of human patients results in a 36-month progression-free survival rate (PFS36) of at least 65%, or 70%, or 75%.
Exemplary Embodiment 24: The method of embodiment 23, wherein the PFS36 is calculated at 36 months, measured starting from:
   (a) the start of treatment; or
   (b) up to 7 days prior to the start of treatment; or
   (c) starting from the time from randomization to the time of a first occurrence of disease progression, relapse, or death.
Exemplary Embodiment 25: A method for treating diffuse large B-cell lymphoma (DLBCL) in a human patient in need thereof, comprising administering to the human patient an effective amount of:
   (a) an immunoconjugate comprising the formula:

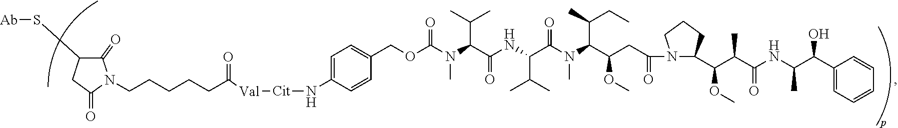

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and
wherein p is between 1 and 8,
(b) rituximab,
(c) cyclophosphamide,
(d) doxorubicin, and
(e) prednisone, prednisolone, or methylprednisolone;
   wherein administering such treatment to a plurality of human patients results in an improvement in a 36-month progression-free survival rate (PFS36) of the plurality of human patients as compared to a reference PFS36,
   wherein the reference PFS36 is the 36-month progression-free survival rate of a plurality of human patients who have received a control treatment comprising:
   (a) rituximab,
   (b) cyclophosphamide,
   (c) doxorubicin,
   (d) vincristine, and
   (e) prednisone, prednisolone, or methylprednisolone,
   in the absence of immunoconjugate.
Exemplary Embodiment 26: A method for treating diffuse large B-cell lymphoma (DLBCL) in a human patient in need thereof, comprising administering to the human patient an effective amount of:
   (a) an immunoconjugate comprising the formula:

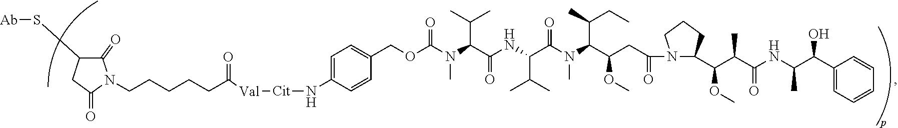

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8,
(b) rituximab,
(c) cyclophosphamide,
(d) doxorubicin, and
(e) prednisone, prednisolone, or methylprednisolone;
   wherein administering such treatment to a plurality of human patients results in an improvement in a 36-month progression-free survival rate (PFS36) of the plurality of human patients of at least about 6%, 7%, 8%, 9%, or 10%, as compared to a reference PFS36,
   wherein the reference PFS36 is the 36-month progression-free survival rate of a plurality of human patients who have received a control treatment comprising:
   (a) rituximab,
   (b) cyclophosphamide,
   (c) doxorubicin,
   (d) vincristine, and
   (e) prednisone, prednisolone, or methylprednisolone,
   in the absence of immunoconjugate.
Exemplary Embodiment 27: The method of embodiment 25 or embodiment 26, wherein the PFS36 or the reference PFS36 is calculated at 36 months, measured starting from:
   (a) the start of the corresponding treatment; or
   (b) up to 7 days prior to the start of the corresponding treatment; or
   (c) starting from the time from randomization to the time of a first occurrence of disease progression, relapse, or death.
Exemplary Embodiment 28: The method of any one of embodiments 23-27, wherein the PFS36 or the reference PFS36 is a progression-free survival (PFS) rate calculated using a Kaplan-Meier method.
Exemplary Embodiment 29: The method of any one of embodiments 25-28, wherein the improvement in PFS36 is statistically significant.
Exemplary Embodiment 30: A method for treating diffuse large B-cell lymphoma (DLBCL) in a human patient in need thereof, comprising administering to the human patient an effective amount of:
   (a) an immunoconjugate comprising the formula:

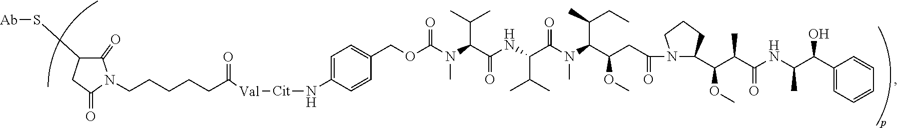

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and
wherein p is between 1 and 8,
(b) rituximab,
(c) cyclophosphamide,
(d) doxorubicin, and
(e) prednisone, prednisolone, or methylprednisolone;
   wherein administering such treatment to a plurality of human patients results in an improvement in overall survival (OS) of the plurality of human patients as compared to a reference OS,
   wherein the reference OS is the OS of a plurality of human patients who have received a control treatment comprising:
   (a) rituximab,
   (b) cyclophosphamide,
   (c) doxorubicin,
   (d) vincristine, and
   (e) prednisone, prednisolone, or methylprednisolone,
   in the absence of immunoconjugate.
Exemplary Embodiment 31: The method of embodiment 30, wherein the OS or the reference OS is measured:
   (a) starting from the start of the corresponding treatment to the time of death from any cause; or
   (b) starting from up to 7 days prior to the start of the corresponding treatment to the time of death from any cause; or
   (c) starting from the time from randomization to the time of death from any cause.
Exemplary Embodiment 32: The method of embodiment 30 or embodiment 31, wherein the improvement in OS is statistically significant.
Exemplary Embodiment 33: A method for treating diffuse large B-cell lymphoma (DLBCL) in a human patient in need thereof, comprising administering to the human patient an effective amount of:
   (a) an immunoconjugate comprising the formula:

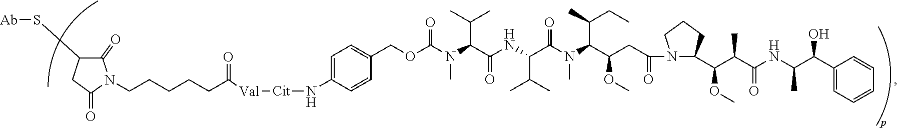

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8,
(b) rituximab,
(c) cyclophosphamide,
(d) doxorubicin, and
(e) prednisone, prednisolone, or methylprednisolone;
   wherein administering such treatment to a plurality of human patients results in a hazard ratio of no more than 1.0, 0.99, 0.98, 0.97, 0.96, 0.95, 0.9, 0.85, or 0.8 in overall survival (OS) of the plurality of human patients, as compared to a control treatment comprising:
   (a) rituximab,
   (b) cyclophosphamide,
   (c) doxorubicin,
   (d) vincristine, and
   (e) prednisone, prednisolone, or methylprednisolone,
   in the absence of immunoconjugate.
Exemplary Embodiment 34: The method of embodiment 33, wherein the OS is measured:
   (a) starting from the start of the corresponding treatment to the time of death from any cause; or
   (b) starting from up to 7 days prior to the start of the corresponding treatment to the time of death from any cause; or
   (c) starting from the time from randomization to the time of death from any cause.
Exemplary Embodiment 35: The method of embodiment 33 or embodiment 34, wherein the hazard ratio has a 95% confidence interval.
Exemplary Embodiment 36: The method of embodiment 35, wherein administering such treatment results in a statistically significant improvement in the OS as compared to the control treatment with a hazard ratio of no more than 1.0, 0.99, 0.98, 0.97, 0.96, 0.95, 0.9, 0.85, or 0.8.
   (d) vincristine, and
   (e) prednisone, prednisolone, or methylprednisolone,
   in the absence of immunoconjugate.
Exemplary Embodiment 39: The method of embodiment 38, wherein the EFS_{eff} or the reference EFS_{eff} is measured:
   (a) starting from the start of the corresponding treatment to the time of a first occurrence of an EFS_{eff} event; or
   (b) starting from up to 7 days prior to the start of the corresponding treatment to the time of a first occurrence of an EFS_{eff} event; or
   (c) starting from the time from randomization to the time of a first occurrence of an EFS_{eff} event.
Exemplary Embodiment 40: The method of embodiment 38 or embodiment 39, wherein the improvement in EFS_{eff} is statistically significant.
Exemplary Embodiment 41: The method of any one of embodiments 38-40, wherein the improvement in EFS_{eff} is calculated at 24 months or more, or 36 months or more, measured starting from:
   (a) the start of the corresponding treatment; or
   (b) up to 7 days prior to the start of the corresponding treatment; or
   (c) the time from randomization to the time of a first occurrence of an EFS_{eff} event.
Exemplary Embodiment 42: A method for treating diffuse large B-cell lymphoma (DLBCL) in a human patient in need thereof, comprising administering to the human patient an effective amount of:
   (a) an immunoconjugate comprising the formula:

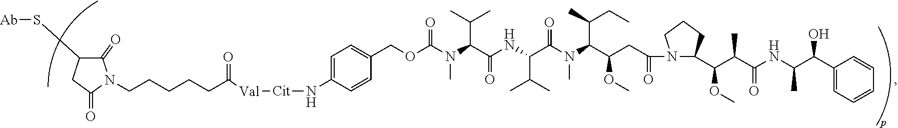

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24;
(v) an HVR-L2 comprising the amino acid sequence of SEQ ID NO: 25; and (vi) an HVR-L3 comprising the amino acid sequence of SEQ ID NO: 26, and wherein p is between 1 and 8,
(b) rituximab,
(c) cyclophosphamide,
(d) doxorubicin, and
(e) prednisone, prednisolone, or methylprednisolone;
   wherein administering such treatment to a plurality of human patients results in: a hazard ratio of no more than 0.77 in event-free survival-efficacy (EFS_{eff}) in the plurality of human patients, or a hazard ratio of no more than 0.81 in EFS_{eff} in the plurality of human patients,
   as compared to a control treatment comprising:
   (a) rituximab,
   (b) cyclophosphamide,
   (c) doxorubicin,
   (d) vincristine, and
   (e) prednisone, prednisolone, or methylprednisolone,
   in the absence of immunoconjugate.
Exemplary Embodiment 43: The method of embodiment 42, wherein the EFS_{eff} is measured:
   (a) starting from the start of the corresponding treatment to the time of a first occurrence of an EFS_{eff} event; or
   (b) starting from up to 7 days prior to the start of the corresponding treatment to the time of a first occurrence of an EFS_{eff} event; or
   (c) starting from the time from randomization to the time of a first occurrence of an EFS_{eff} event.
Exemplary Embodiment 44: The method of embodiment 42 or embodiment 43, wherein the hazard ratio has a 95% confidence interval.
Exemplary Embodiment 45: The method of embodiment 44, wherein administering such treatment results in a statistically significant improvement in the EFS_{eff} as compared to the control treatment with: a hazard ratio of no more than 0.77 (95% confidence interval: 0.59, 1.00); or a hazard ratio of no more than 0.81 (95% confidence interval: 0.63, 1.04).
Exemplary Embodiment 46: The method of any one of embodiments 42-45, wherein the hazard ratio is calculated at 24 months or more, or 36 months or more, measured starting from:
   (a) the start of the corresponding treatment; or
   (b) up to 7 days prior to the start of the corresponding treatment; or
   (c) the time from randomization to the time of a first occurrence of an EFS_{eff} event.
Exemplary Embodiment 47: The method of any one of embodiments 39-41 and 43-46, wherein the EFS_{eff} event is:
   (a) disease progression;
   (b) relapse;
   (c) death;
   (d) a primary efficacy reason that leads to initiation of a non-protocol specified anti-lymphoma treatment (NALT), and that is not disease progression or relapse;
   (e) a biopsy positive for residual disease.
Exemplary Embodiment 48: A method for treating diffuse large B-cell lymphoma (DLBCL) in a human patient in need thereof, comprising administering to the human patient an effective amount of:
   (a) an immunoconjugate comprising the formula:

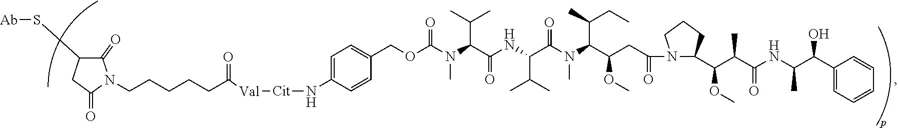

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and
wherein p is between 1 and 8,
(b) rituximab,
(c) cyclophosphamide,
(d) doxorubicin, and of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and
wherein p is between 1 and 8,
(b) rituximab,
(c) cyclophosphamide,
(d) doxorubicin, and
(e) prednisone, prednisolone, or methylprednisolone:
   wherein administering such treatment to a plurality of human patients results in: an objective response rate (ORR) at end of treatment (EOT) in the plurality of human patients of at least about 84%, or an ORR at EOT in the plurality of human patients of at least about 85%,
   wherein the ORR is assessed by positron emission tomography-computed tomography (PET-CT).
Exemplary Embodiment 53: The method of embodiment 52, wherein ORR is assessed by an investigator or by blinded independent central review (BICR).
Exemplary Embodiment 54: The method of embodiment 52 or embodiment 53, wherein administering such treatment to a plurality of human patients results in an improvement in ORR of at least about 2% in the plurality of human patients, as compared to a plurality of human patients who have received a control treatment comprising:
   (a) rituximab,
   (b) cyclophosphamide,
   (c) doxorubicin,
   (d) vincristine, and
   (e) prednisone, prednisolone, or methylprednisolone,
   in the absence of immunoconjugate.
Exemplary Embodiment 55: The method of embodiment 54, wherein the improvement in ORR is statistically significant.
Exemplary Embodiment 56: A method for treating diffuse large B-cell lymphoma (DLBCL) in a human patient in need thereof, comprising administering to the human patient an effective amount of:
   (a) an immunoconjugate comprising the formula:

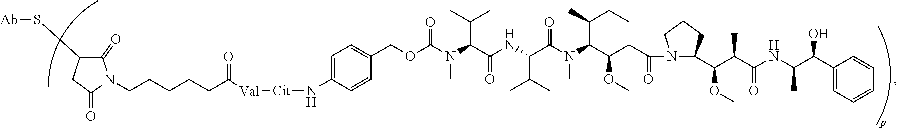

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8,
(b) rituximab,
(c) cyclophosphamide,
(d) doxorubicin, and
(e) prednisone, prednisolone, or methylprednisolone;
   wherein administering such treatment to a plurality of human patients results in a hazard ratio of no more than 1.0, or 0.9, or 0.8 in a 36-month progression-free survival rate (PFS36) of the plurality of human patients, as compared to a reference PFS36,
   wherein the reference PFS36 is the 36-month progression-free survival rate of a plurality of human patients who have received a control treatment comprising:
(a) rituximab,
(b) cyclophosphamide,
(c) doxorubicin,
(d) vincristine, and
(e) prednisone, prednisolone, or methylprednisolone,
in the absence of immunoconjugate.
Exemplary Embodiment 57: The method of any one of embodiments 1-56, wherein p is between 2 and 5.
Exemplary Embodiment 58: The method of any one of embodiments 1-57, wherein p is between 3 and 4.
Exemplary Embodiment 59: The method of any one of embodiments 1-58, wherein the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20.
Exemplary Embodiment 60: The method of any one of embodiments 1-59, wherein the anti-CD79b antibody comprises:
   a heavy chain comprising the amino acid sequence of SEQ ID NO: 36 and a light chain comprising the amino acid sequence of SEQ ID NO: 35;
   a heavy chain comprising the amino acid sequence of SEQ ID NO: 37 and a light chain comprising the amino acid sequence of SEQ ID NO: 35;
   a heavy chain comprising the amino acid sequence of SEQ ID NO: 39 and a light chain comprising the amino acid sequence of SEQ ID NO: 35;
   a heavy chain comprising the amino acid sequence of SEQ ID NO: 36 and a light chain comprising the amino acid sequence of SEQ ID NO: 38;
   a heavy chain comprising the amino acid sequence of SEQ ID NO: 37 and a light chain comprising the amino acid sequence of SEQ ID NO: 38; or
   a heavy chain comprising the amino acid sequence of SEQ ID NO: 39 and a light chain comprising the amino acid sequence of SEQ ID NO: 38.
Exemplary Embodiment 61: The method of any one of embodiments 1-60, wherein the anti-CD79b antibody comprises (i) a heavy chain comprising the amino acid sequence of SEQ ID NO: 36 and (ii) a light chain comprising the amino acid sequence of SEQ ID NO: 35.
Exemplary Embodiment 62: The method of any one of embodiments 1-61, wherein the immunoconjugate is polatuzumab vedotin.
Exemplary Embodiment 63: A method for treating DLBCL in a human patient in need thereof, comprising administering to the human patient at least six 21-day cycles of:
   (a) polatuzumab vedotin at a dose of about 1.8 mg/kg on day 1 of each 21-day cycle,
   (b) rituximab at a dose of about 375 mg/m² on day 1 of each 21-day cycle,
   (c) cyclophosphamide at a dose of about 750 mg/m² on day 1 of each 21-day cycle,
   (d) doxorubicin at a dose of about 50 mg/m² on day 1 of each 21-day cycle, and
   (e) prednisone at a dose of about 100 mg per day on each of days 1-5 of each 21-day cycle, prednisolone at a dose of about 100 mg per day on each of days 1-5 of each 21-day cycle, or methylprednisolone at a dose of about 80 mg per day on each of days 1-5 of each 21-day cycle;
      wherein administering such treatment to a plurality of human patients results in an improvement in progression-free survival (PFS) of the plurality of human patients as compared to a reference PFS,
      wherein the reference PFS is the PFS of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 64: A method for treating diffuse large B-cell lymphoma (DLBCL) in a human patient in need thereof, comprising administering to the human patient at least six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle:
      wherein administering such treatment to a plurality of human patients results in: at least a 20% reduction in the risk of disease progression, relapse, or death in the plurality of human patients, or at least a 25% reduction in the risk of disease progression, relapse, or death in the plurality of human patients,
      as compared to a control treatment comprising:
   (a) rituximab,
   (b) cyclophosphamide,
   (c) doxorubicin,
   (d) vincristine, and
   (e) prednisone, prednisolone, or methylprednisolone,
   in the absence of polatuzumab vedotin.
Exemplary Embodiment 65: A method for treating diffuse large B-cell lymphoma (DLBCL) in a human patient in need thereof, comprising administering to the human patient at least six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle;
      wherein administering such treatment to a plurality of human patients results in: a hazard ratio of no more than 0.75 in progression-free survival (PFS) of the plurality of human patients, or a hazard ratio of no more than 0.78 in progression-free survival (PFS) of the plurality of human patients, or a hazard ratio of no more than 0.79 in progression-free survival (PFS) of the plurality of human patients,
      as compared to a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 66: A method for treating diffuse large B-cell lymphoma (DLBCL) in a human patient in need thereof, comprising administering to the human patient at least six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle:
      wherein administering such treatment to a plurality of human patients results in a 24-month progression-free survival rate (PFS24) of at least 75%.
Exemplary Embodiment 67: A method for treating diffuse large B-cell lymphoma (DLBCL) in a human patient in need thereof, comprising administering to the human patient at least six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle:
      wherein administering such treatment to a plurality of human patients results in an improvement in a 24-month progression-free survival rate (PFS24) of the plurality of human patients as compared to a reference PFS24,
      wherein the reference PFS24 is the 24-month progression-free survival rate of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 68: A method for treating diffuse large B-cell lymphoma (DLBCL) in a human patient in need thereof, comprising administering to the human patient at least six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle;
      wherein administering such treatment to a plurality of human patients results in an improvement in a 24-month progression-free survival rate (PFS24) of the plurality of human patients of at least about 6%, as compared to a reference PFS24,
      wherein the reference PFS24 is the 24-month progression-free survival rate of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 69: A method for treating diffuse large B-cell lymphoma (DLBCL) in a human patient in need thereof, comprising administering to the human patient at least six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle:
      wherein administering such treatment to a plurality of human patients results in a 36-month progression-free survival rate (PFS36) of at least 65%, or 70%, or 75%.
Exemplary Embodiment 70: A method for treating diffuse large B-cell lymphoma (DLBCL) in a human patient in need thereof, comprising administering to the human patient at least six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle:
      wherein administering such treatment to a plurality of human patients results in an improvement in a 36-month progression-free survival rate (PFS36) of the plurality of human patients as compared to a reference PFS36,
      wherein the reference PFS36 is the 36-month progression-free survival rate of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 71: A method for treating diffuse large B-cell lymphoma (DLBCL) in a human patient in need thereof, comprising administering to the human patient at least six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle:
      wherein administering such treatment to a plurality of human patients results in an improvement in a 36-month progression-free survival rate (PFS36) of the plurality of human patients of at least about 6%, 7%, 8%, 9%, or 10%, as compared to a reference PFS36,
      wherein the reference PFS36 is the 36-month progression-free survival rate of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 72: A method for treating diffuse large B-cell lymphoma (DLBCL) in a human patient in need thereof, comprising administering to the human patient at least six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle;
      wherein administering such treatment to a plurality of human patients results in an improvement in overall survival (OS) of the plurality of human patients as compared to a reference OS,
      wherein the reference OS is the OS of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 73: A method for treating diffuse large B-cell lymphoma (DLBCL) in a human patient in need thereof, comprising administering to the human patient at least six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle, cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle;
      wherein administering such treatment to a plurality of human patients results in a hazard ratio of no more than 1.0, 0.99, 0.98, 0.97, 0.96, 0.95, 0.9, 0.85, or 0.8 in overall survival (OS) of the plurality of human patients, as compared to a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 74: A method for treating diffuse large B-cell lymphoma (DLBCL) in a human patient in need thereof, comprising administering to the human patient at least six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle:
      wherein administering such treatment to a plurality of human patients results in an improvement in event-free survival-efficacy (EFS_{eff}) of the plurality of human patients as compared to a reference EFS_{eff},
      wherein the reference EFS_{eff} is the EFS_{eff} of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 75: A method for treating diffuse large B-cell lymphoma (DLBCL) in a human patient in need thereof, comprising administering to the human patient at least six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle;
      wherein administering such treatment to a plurality of human patients results in: a hazard ratio of no more than 0.77 in event-free survival-efficacy (EFS_{eff}) in the plurality of human patients, or a hazard ratio of no more than 0.81 in EFS_{eff} in the plurality of human patients,
      as compared to a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 76: A method for treating diffuse large B-cell lymphoma (DLBCL) in a human patient in need thereof, comprising administering to the human patient at least six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle:
      wherein administering such treatment to a plurality of human patients results in a rate of complete response (CR) at end of treatment (EOT) in the plurality of human patients of at least about 77%, wherein the rate of CR is assessed by positron emission tomography-computed tomography (PET-CT).
Exemplary Embodiment 77: A method for treating diffuse large B-cell lymphoma (DLBCL) in a human patient in need thereof, comprising administering to the human patient at least six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle;
      wherein administering such treatment to a plurality of human patients results in: an objective response rate (ORR) at end of treatment (EOT) in the plurality of human patients of at least about 84%, or an ORR at EOT in the plurality of human patients of at least about 85%,
      wherein the ORR is assessed by positron emission tomography-computed tomography (PET-CT).
Exemplary Embodiment 78: A method for treating diffuse large B-cell lymphoma (DLBCL) in a human patient in need thereof, comprising administering to the human patient at least six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle:
      wherein administering such treatment to a plurality of human patients results in a hazard ratio of no more than 0.75 in a 36-month progression-free survival rate (PFS36) of the plurality of human patients, as compared to a reference PFS36,
      wherein the reference PFS36 is the 36-month progression-free survival rate of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 79: The method of any one of embodiments 62-78, wherein the polatuzumab vedotin, the rituximab, the cyclophosphamide, the doxorubicin, and the prednisone are administered to the human patient.
Exemplary Embodiment 80: The method of any one of embodiments 62-78, wherein the polatuzumab vedotin, the rituximab, the cyclophosphamide, the doxorubicin, and the prednisolone are administered to the human patient.
Exemplary Embodiment 81: The method of any one of embodiments 62-78, wherein the polatuzumab vedotin, the rituximab, the cyclophosphamide, the doxorubicin, and the methylprednisolone are administered to the human patient.
Exemplary Embodiment 82: The method of any one of embodiments 63-81, wherein the polatuzumab vedotin, the rituximab, the cyclophosphamide, the doxorubicin, and the prednisone, prednisolone, or methylprednisolone are administered to the human patient sequentially on day 1 of each 21-day cycle.
Exemplary Embodiment 83: The method of embodiment 82, wherein the prednisone, prednisolone, or methylprednisolone is administered prior to the rituximab; the rituximab is administered prior to the polatuzumab vedotin; and the polatuzumab vedotin is administered prior to the cyclophosphamide and doxorubicin.
Exemplary Embodiment 84: The method of any one of embodiments 63-83, further comprising:
   (a) administering rituximab monotherapy to the human patient during a seventh and eighth 21-day cycle after the sixth 21-day cycle; or
   (b) administering rituximab, cyclophosphamide, doxorubicin, and prednisone, prednisolone, or methylprednisolone to the human patient during a seventh and eighth 21-day cycle after the sixth 21-day cycle.
Exemplary Embodiment 85: The method of embodiment 84, comprising administering rituximab monotherapy to the human patient intravenously at a dose of about 375 mg/m² on day 1 of each of the seventh and eighth 21-day cycles.
Exemplary Embodiment 86: The method of embodiment 84, comprising administering rituximab, cyclophosphamide, doxorubicin, and prednisone, prednisolone, or methylprednisolone to the human patient, wherein:
   (a) the rituximab is administered intravenously at a dose of about 375 mg/m² on day 1 of each of the seventh and eighth 21-day cycles;
   (b) the cyclophosphamide is administered intravenously at a dose of about 750 mg/m² on day 1 of each of the seventh and eighth 21-day cycles;
   (c) the doxorubicin is administered intravenously at a dose of about 50 mg/m² on day 1 of each of the seventh and eighth 21-day cycles; and
   (d) the prednisone is administered orally at a dose of about 100 mg per day on each of days 1-5 of each of the seventh and eighth 21-day cycles; the prednisolone is administered orally at a dose of about 100 mg per day on each of days 1-5 of each of the seventh and eighth 21-day cycles; or the methylprednisolone is administered intravenously at a dose of about 80 mg per day on each of days 1-5 of each of the seventh and eighth 21-day cycles.
Exemplary Embodiment 87: The method of any one of embodiments 1-15, 18-22, 25-47, 50-51, 54-65, 67-68, 70-75, 78-86, wherein the rituximab, the cyclophosphamide, the doxorubicin, the vincristine, and the prednisone, prednisolone, or methylprednisolone are administered sequentially on day 1 of each 21-day cycle.
Exemplary Embodiment 88: The method of embodiment 87, wherein the prednisone, prednisolone, or methylprednisolone is administered prior to the rituximab; and the rituximab is administered prior to the cyclophosphamide, doxorubicin and vincristine.
Exemplary Embodiment 89: The method of any one of embodiments 1-15, 18-22, 25-47, 50-51, 54-65, 67-68, 70-75, 78-88, wherein the control treatment further comprises:
   (a) rituximab monotherapy during a seventh and eighth 21-day cycle after the sixth 21-day cycle; or
   (b) rituximab, cyclophosphamide, doxorubicin, vincristine, and prednisone, prednisolone, or methylprednisolone during a seventh and eighth 21-day cycle after the sixth 21-day cycle.
Exemplary Embodiment 90: The method of embodiment 89, wherein the control treatment further comprises rituximab monotherapy administered intravenously at a dose of about 375 mg/m² on day 1 of each of the seventh and eighth 21-day cycles.
Exemplary Embodiment 91: The method of embodiment 89, wherein the control treatment further comprises rituximab, cyclophosphamide, doxorubicin, vincristine, and prednisone, prednisolone, or methylprednisolone during a seventh and eighth 21-day cycle after the sixth 21-day cycle, wherein:
   (a) the rituximab is administered intravenously at a dose of about 375 mg/m² on day 1 of each of the seventh and eighth 21-day cycles;
   (b) the cyclophosphamide is administered intravenously at a dose of about 750 mg/m² on day 1 of each of the seventh and eighth 21-day cycles;
   (c) the doxorubicin is administered intravenously at a dose of about 50 mg/m² on day 1 of each of the seventh and eighth 21-day cycles;
   (d) the vincristine is administered intravenously at a dose of about 1.4 mg/m² and up to 2 mg each dose on day 1 of each of the seventh and eighth 21-day cycles; and
   (e) the prednisone is administered orally at a dose of about 100 mg per day on each of days 1-5 of each of the seventh and eighth 21-day cycles; the prednisolone is administered orally at a dose of about 100 mg per day on each of days 1-5 of each of the seventh and eighth 21-day cycles; or the methylprednisolone is administered intravenously at a dose of about 80 mg per day on each of days 1-5 of each of the seventh and eighth 21-day cycles.
Exemplary Embodiment 92: A method for treating diffuse large B-cell lymphoma (DLBCL) in a human patient in need thereof, comprising administering to the human patient six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle; and
      wherein the method further comprises administering to the human patient:
      (i) rituximab monotherapy intravenously at a dose of about 375 mg/m² on day 1 of each of a seventh and eighth 21-day cycles; or
      (ii) rituximab intravenously at a dose of about 375 mg/m² on day 1 of each of a seventh and eighth 21-day cycles; cyclophosphamide intravenously at a dose of about 750 mg/m² on day 1 of each of a seventh and eighth 21-day cycles; doxorubicin intravenously at a dose of about 50 mg/m² on day 1 of each of a seventh and eighth 21-day cycles; and prednisone orally at a dose of about 100 mg per day on each of days 1-5 of each of a seventh and eighth 21-day cycles, prednisolone orally at a dose of about 100 mg per day on each of days 1-5 of each of a seventh and eighth 21-day cycles, or methylprednisolone intravenously at a dose of about 80 mg per day on each of days 1-5 of each of a seventh and eighth 21-day cycles;
      wherein administering such treatment to a plurality of human patients results in an improvement in progression-free survival (PFS) of the plurality of human patients as compared to a reference PFS,
      wherein the reference PFS is the PFS of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 93: A method for treating diffuse large B-cell lymphoma (DLBCL) in a human patient in need thereof, comprising administering to the human patient six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle; and
      wherein the method further comprises administering to the human patient:
      (i) rituximab monotherapy intravenously at a dose of about 375 mg/m² on day 1 of each of a seventh and eighth 21-day cycles; or
      (ii) rituximab intravenously at a dose of about 375 mg/m² on day 1 of each of a seventh and eighth 21-day cycles; cyclophosphamide intravenously at a dose of about 750 mg/m² on day 1 of each of a seventh and eighth 21-day cycles; doxorubicin intravenously at a dose of about 50 mg/m² on day 1 of each of a seventh and eighth 21-day cycles; and prednisone orally at a dose of about 100 mg per day on each of days 1-5 of each of a seventh and eighth 21-day cycles, prednisolone orally at a dose of about 100 mg per day on each of days 1-5 of each of a seventh and eighth 21-day cycles, or methylprednisolone intravenously at a dose of about 80 mg per day on each of days 1-5 of each of a seventh and eighth 21-day cycles;
      wherein administering such treatment to a plurality of human patients results in: at least a 20% reduction in the risk of disease progression, relapse, or death in the plurality of human patients, or at least a 25% reduction in the risk of disease progression, relapse, or death in the plurality of human patients,
      as compared to a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 94: A method for treating diffuse large B-cell lymphoma (DLBCL) in a human patient in need thereof, comprising administering to the human patient six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle, cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle; and wherein the method further comprises administering to the human patient:
      (i) rituximab monotherapy intravenously at a dose of about 375 mg/m² on day 1 of each of a seventh and eighth 21-day cycles; or
      (ii) rituximab intravenously at a dose of about 375 mg/m² on day 1 of each of a seventh and eighth 21-day cycles; cyclophosphamide intravenously at a dose of about 750 mg/m² on day 1 of each of a seventh and eighth 21-day cycles; doxorubicin intravenously at a dose of about 50 mg/m² on day 1 of each of a seventh and eighth 21-day cycles; and prednisone orally at a dose of about 100 mg per day on each of days 1-5 of each of a seventh and eighth 21-day cycles, prednisolone orally at a dose of about 100 mg per day on each of days 1-5 of each of a seventh and eighth 21-day cycles, or methylprednisolone intravenously at a dose of about 80 mg per day on each of days 1-5 of each of a seventh and eighth 21-day cycles;
      wherein administering such treatment to a plurality of human patients results in: a hazard ratio of no more than 0.75 in progression-free survival (PFS) of the plurality of human patients, or a hazard ratio of no more than 0.78 in PFS of the plurality of human patients, or a hazard ratio of no more than 0.79 in PFS of the plurality of human patients,
      as compared to a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 95: A method for treating diffuse large B-cell lymphoma (DLBCL) in a human patient in need thereof, comprising administering to the human patient six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle; and
      wherein the method further comprises administering to the human patient:
      (i) rituximab monotherapy intravenously at a dose of about 375 mg/m² on day 1 of each of a seventh and eighth 21-day cycles; or
      (ii) rituximab intravenously at a dose of about 375 mg/m² on day 1 of each of a seventh and eighth 21-day cycles; cyclophosphamide intravenously at a dose of about 750 mg/m² on day 1 of each of a seventh and eighth 21-day cycles; doxorubicin intravenously at a dose of about 50 mg/m² on day 1 of each of a seventh and eighth 21-day cycles; and prednisone orally at a dose of about 100 mg per day on each of days 1-5 of each of a seventh and eighth 21-day cycles, prednisolone orally at a dose of about 100 mg per day on each of days 1-5 of each of a seventh and eighth 21-day cycles, or methylprednisolone intravenously at a dose of about 80 mg per day on each of days 1-5 of each of a seventh and eighth 21-day cycles;
      wherein administering such treatment to a plurality of human patients results in a 24-month progression-free survival rate (PFS24) of at least 75%.
Exemplary Embodiment 96: A method for treating diffuse large B-cell lymphoma (DLBCL) in a human patient in need thereof, comprising administering to the human patient six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle; and
      wherein the method further comprises administering to the human patient:
      (i) rituximab monotherapy intravenously at a dose of about 375 mg/m² on day 1 of each of a seventh and eighth 21-day cycles; or
      (ii) rituximab intravenously at a dose of about 375 mg/m² on day 1 of each of a seventh and eighth 21-day cycles; cyclophosphamide intravenously at a dose of about 750 mg/m² on day 1 of each of a seventh and eighth 21-day cycles; doxorubicin intravenously at a dose of about 50 mg/m² on day 1 of each of a seventh and eighth 21-day cycles; and prednisone orally at a dose of about 100 mg per day on each of days 1-5 of each of a seventh and eighth 21-day cycles, prednisolone orally at a dose of about 100 mg per day on each of days 1-5 of each of a seventh and eighth 21-day cycles, or methylprednisolone intravenously at a dose of about 80 mg per day on each of days 1-5 of each of a seventh and eighth 21-day cycles;
      wherein administering such treatment to a plurality of human patients results in an improvement in a 24-month progression-free survival rate (PFS24) of the plurality of human patients as compared to a reference PFS24,
      wherein the reference PFS24 is the 24-month progression-free survival rate of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 97: A method for treating diffuse large B-cell lymphoma (DLBCL) in a human patient in need thereof, comprising administering to the human patient six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle, cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle; and
      wherein the method further comprises administering to the human patient:
      (i) rituximab monotherapy intravenously at a dose of about 375 mg/m² on day 1 of each of a seventh and eighth 21-day cycles; or
      (ii) rituximab intravenously at a dose of about 375 mg/m² on day 1 of each of a seventh and eighth 21-day cycles; cyclophosphamide intravenously at a dose of about 750 mg/m² on day 1 of each of a seventh and eighth 21-day cycles; doxorubicin intravenously at a dose of about 50 mg/m² on day 1 of each of a seventh and eighth 21-day cycles; and prednisone orally at a dose of about 100 mg per day on each of days 1-5 of each of a seventh and eighth 21-day cycles, prednisolone orally at a dose of about 100 mg per day on each of days 1-5 of each of a seventh and eighth 21-day cycles, or methylprednisolone intravenously at a dose of about 80 mg per day on each of days 1-5 of each of a seventh and eighth 21-day cycles;
      wherein administering such treatment to a plurality of human patients results in an improvement in a 24-month progression-free survival rate (PFS24) of the plurality of human patients of at least about 6%, as compared to a reference PFS24,
      wherein the reference PFS24 is the 24-month progression-free survival rate of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 98: A method for treating diffuse large B-cell lymphoma (DLBCL) in a human patient in need thereof, comprising administering to the human patient six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle; and
      wherein the method further comprises administering to the human patient:
      (i) rituximab monotherapy intravenously at a dose of about 375 mg/m² on day 1 of each of a seventh and eighth 21-day cycles; or
      (ii) rituximab intravenously at a dose of about 375 mg/m² on day 1 of each of a seventh and eighth 21-day cycles; cyclophosphamide intravenously at a dose of about 750 mg/m² on day 1 of each of a seventh and eighth 21-day cycles; doxorubicin intravenously at a dose of about 50 mg/m² on day 1 of each of a seventh and eighth 21-day cycles; and prednisone orally at a dose of about 100 mg per day on each of days 1-5 of each of a seventh and eighth 21-day cycles, prednisolone orally at a dose of about 100 mg per day on each of days 1-5 of each of a seventh and eighth 21-day cycles, or methylprednisolone intravenously at a dose of about 80 mg per day on each of days 1-5 of each of a seventh and eighth 21-day cycles;
      wherein administering such treatment to a plurality of human patients results in a 36-month progression-free survival rate (PFS36) of at least 70%, or 75%, or 80%.
Exemplary Embodiment 99: A method for treating diffuse large B-cell lymphoma (DLBCL) in a human patient in need thereof, comprising administering to the human patient six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle; and
      wherein the method further comprises administering to the human patient:
      (i) rituximab monotherapy intravenously at a dose of about 375 mg/m² on day 1 of each of a seventh and eighth 21-day cycles; or
      (ii) rituximab intravenously at a dose of about 375 mg/m² on day 1 of each of a seventh and eighth 21-day cycles; cyclophosphamide intravenously at a dose of about 750 mg/m² on day 1 of each of a seventh and eighth 21-day cycles; doxorubicin intravenously at a dose of about 50 mg/m² on day 1 of each of a seventh and eighth 21-day cycles; and prednisone orally at a dose of about 100 mg per day on each of days 1-5 of each of a seventh and eighth 21-day cycles, prednisolone orally at a dose of about 100 mg per day on each of days 1-5 of each of a seventh and eighth 21-day cycles, or methylprednisolone intravenously at a dose of about 80 mg per day on each of days 1-5 of each of a seventh and eighth 21-day cycles;
      wherein administering such treatment to a plurality of human patients results in an improvement in a 36-month progression-free survival rate (PFS36) of the plurality of human patients as compared to a reference PFS36,
      wherein the reference PFS36 is the 36-month progression-free survival rate of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 100: A method for treating diffuse large B-cell lymphoma (DLBCL) in a human patient in need thereof, comprising administering to the human patient six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle; and
      wherein the method further comprises administering to the human patient:
      (i) rituximab monotherapy intravenously at a dose of about 375 mg/m² on day 1 of each of a seventh and eighth 21-day cycles; or
      (ii) rituximab intravenously at a dose of about 375 mg/m² on day 1 of each of a seventh and eighth 21-day cycles; cyclophosphamide intravenously at a dose of about 750 mg/m² on day 1 of each of a seventh and eighth 21-day cycles; doxorubicin intravenously at a dose of about 50 mg/m² on day 1 of each of a seventh and eighth 21-day cycles; and prednisone orally at a dose of about 100 mg per day on each of days 1-5 of each of a seventh and eighth 21-day cycles, prednisolone orally at a dose of about 100 mg per day on each of days 1-5 of each of a seventh and eighth 21-day cycles, or methylprednisolone intravenously at a dose of about 80 mg per day on each of days 1-5 of each of a seventh and eighth 21-day cycles;
      wherein administering such treatment to a plurality of human patients results in an improvement in a 36-month progression-free survival rate (PFS36) of the plurality of human patients of at least about 6%, 7%, 8%, 9%, or 10%, as compared to a reference PFS36,
      wherein the reference PFS36 is the 36-month progression-free survival rate of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 101: A method for treating diffuse large B-cell lymphoma (DLBCL) in a human patient in need thereof, comprising administering to the human patient six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle; and
      wherein the method further comprises administering to the human patient:
      (i) rituximab monotherapy intravenously at a dose of about 375 mg/m² on day 1 of each of a seventh and eighth 21-day cycles; or
      (ii) rituximab intravenously at a dose of about 375 mg/m² on day 1 of each of a seventh and eighth 21-day cycles; cyclophosphamide intravenously at a dose of about 750 mg/m² on day 1 of each of a seventh and eighth 21-day cycles; doxorubicin intravenously at a dose of about 50 mg/m² on day 1 of each of a seventh and eighth 21-day cycles; and prednisone orally at a dose of about 100 mg per day on each of days 1-5 of each of a seventh and eighth 21-day cycles, prednisolone orally at a dose of about 100 mg per day on each of days 1-5 of each of a seventh and eighth 21-day cycles, or methylprednisolone intravenously at a dose of about 80 mg per day on each of days 1-5 of each of a seventh and eighth 21-day cycles;
      wherein administering such treatment to a plurality of human patients results in an improvement in overall survival (OS) of the plurality of human patients as compared to a reference OS,
      wherein the reference OS is the OS of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 102: A method for treating diffuse large B-cell lymphoma (DLBCL) in a human patient in need thereof, comprising administering to the human patient six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle; and
      wherein the method further comprises administering to the human patient:
      (i) rituximab monotherapy intravenously at a dose of about 375 mg/m² on day 1 of each of a seventh and eighth 21-day cycles; or
      (ii) rituximab intravenously at a dose of about 375 mg/m² on day 1 of each of a seventh and eighth 21-day cycles; cyclophosphamide intravenously at a dose of about 750 mg/m² on day 1 of each of a seventh and eighth 21-day cycles; doxorubicin intravenously at a dose of about 50 mg/m² on day 1 of each of a seventh and eighth 21-day cycles; and prednisone orally at a dose of about 100 mg per day on each of days 1-5 of each of a seventh and eighth 21-day cycles, prednisolone orally at a dose of about 100 mg per day on each of days 1-5 of each of a seventh and eighth 21-day cycles, or methylprednisolone intravenously at a dose of about 80 mg per day on each of days 1-5 of each of a seventh and eighth 21-day cycles;
      wherein administering such treatment to a plurality of human patients results in a hazard ratio of no more than 1.0 in overall survival (OS) of the plurality of human patients, as compared to a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 103: A method for treating diffuse large B-cell lymphoma (DLBCL) in a human patient in need thereof, comprising administering to the human patient six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle; and
      wherein the method further comprises administering to the human patient:
      (i) rituximab monotherapy intravenously at a dose of about 375 mg/m² on day 1 of each of a seventh and eighth 21-day cycles; or
      (ii) rituximab intravenously at a dose of about 375 mg/m² on day 1 of each of a seventh and eighth 21-day cycles; cyclophosphamide intravenously at a dose of about 750 mg/m² on day 1 of each of a seventh and eighth 21-day cycles; doxorubicin intravenously at a dose of about 50 mg/m² on day 1 of each of a seventh and eighth 21-day cycles; and prednisone orally at a dose of about 100 mg per day on each of days 1-5 of each of a seventh and eighth 21-day cycles, prednisolone orally at a dose of about 100 mg per day on each of days 1-5 of each of a seventh and eighth 21-day cycles, or methylprednisolone intravenously at a dose of about 80 mg per day on each of days 1-5 of each of a seventh and eighth 21-day cycles;
      wherein administering such treatment to a plurality of human patients results in an improvement in event-free survival-efficacy (EFS_{eff}) of the plurality of human patients as compared to a reference EFS_{eff},
      wherein the reference EFS_{eff} is the EFS_{eff} of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 104: A method for treating diffuse large B-cell lymphoma (DLBCL) in a human patient in need thereof, comprising administering to the human patient six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle; and
      wherein the method further comprises administering to the human patient:
      (i) rituximab monotherapy intravenously at a dose of about 375 mg/m² on day 1 of each of a seventh and eighth 21-day cycles; or
      (ii) rituximab intravenously at a dose of about 375 mg/m² on day 1 of each of a seventh and eighth 21-day cycles; cyclophosphamide intravenously at a dose of about 750 mg/m² on day 1 of each of a seventh and eighth 21-day cycles; doxorubicin intravenously at a dose of about 50 mg/m² on day 1 of each of a seventh and eighth 21-day cycles; and prednisone orally at a dose of about 100 mg per day on each of days 1-5 of each of a seventh and eighth 21-day cycles, prednisolone orally at a dose of about 100 mg per day on each of days 1-5 of each of a seventh and eighth 21-day cycles, or methylprednisolone intravenously at a dose of about 80 mg per day on each of days 1-5 of each of a seventh and eighth 21-day cycles;
      wherein administering such treatment to a plurality of human patients results in: a hazard ratio of no more than 0.77 in event-free survival-efficacy (EFS_{eff}) in the plurality of human patients, or a hazard ratio of no more than 0.81 in EFS_{eff} in the plurality of human patients,
      as compared to a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 105: A method for treating diffuse large B-cell lymphoma (DLBCL) in a human patient in need thereof, comprising administering to the human patient six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle; and
      wherein the method further comprises administering to the human patient:
      (i) rituximab monotherapy intravenously at a dose of about 375 mg/m² on day 1 of each of a seventh and eighth 21-day cycles; or
      (ii) rituximab intravenously at a dose of about 375 mg/m² on day 1 of each of a seventh and eighth 21-day cycles; cyclophosphamide intravenously at a dose of about 750 mg/m² on day 1 of each of a seventh and eighth 21-day cycles; doxorubicin intravenously at a dose of about 50 mg/m² on day 1 of each of a seventh and eighth 21-day cycles; and prednisone orally at a dose of about 100 mg per day on each of days 1-5 of each of a seventh and eighth 21-day cycles, prednisolone orally at a dose of about 100 mg per day on each of days 1-5 of each of a seventh and eighth 21-day cycles, or methylprednisolone intravenously at a dose of about 80 mg per day on each of days 1-5 of each of a seventh and eighth 21-day cycles;
      wherein administering such treatment to a plurality of human patients results in a rate of complete response (CR) at end of treatment (EOT) in the plurality of human patients of at least about 77%, wherein the rate of CR is assessed by positron emission tomography-computed tomography (PET-CT).
Exemplary Embodiment 106: A method for treating diffuse large B-cell lymphoma (DLBCL) in a human patient in need thereof, comprising administering to the human patient six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle; and
      wherein the method further comprises administering to the human patient:
      (i) rituximab monotherapy intravenously at a dose of about 375 mg/m² on day 1 of each of a seventh and eighth 21-day cycles; or
      (ii) rituximab intravenously at a dose of about 375 mg/m² on day 1 of each of a seventh and eighth 21-day cycles; cyclophosphamide intravenously at a dose of about 750 mg/m² on day 1 of each of a seventh and eighth 21-day cycles; doxorubicin intravenously at a dose of about 50 mg/m² on day 1 of each of a seventh and eighth 21-day cycles; and prednisone orally at a dose of about 100 mg per day on each of days 1-5 of each of a seventh and eighth 21-day cycles, prednisolone orally at a dose of about 100 mg per day on each of days 1-5 of each of a seventh and eighth 21-day cycles, or methylprednisolone intravenously at a dose of about 80 mg per day on each of days 1-5 of each of a seventh and eighth 21-day cycles;
      wherein administering such treatment to a plurality of human patients results in: an objective response rate (ORR) at end of treatment (EOT) in the plurality of human patients of at least about 84%, or an ORR at EOT in the plurality of human patients of at least about 85%,
      wherein the ORR is assessed by positron emission tomography-computed tomography (PET-CT).
Exemplary Embodiment 107: A method for treating diffuse large B-cell lymphoma (DLBCL) in a human patient in need thereof, comprising administering to the human patient six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle; and
      wherein the method further comprises administering to the human patient:
      (i) rituximab monotherapy intravenously at a dose of about 375 mg/m² on day 1 of each of a seventh and eighth 21-day cycles; or
      (ii) rituximab intravenously at a dose of about 375 mg/m² on day 1 of each of a seventh and eighth 21-day cycles; cyclophosphamide intravenously at a dose of about 750 mg/m² on day 1 of each of a seventh and eighth 21-day cycles; doxorubicin intravenously at a dose of about 50 mg/m² on day 1 of each of a seventh and eighth 21-day cycles; and prednisone orally at a dose of about 100 mg per day on each of days 1-5 of each of a seventh and eighth 21-day cycles, prednisolone orally at a dose of about 100 mg per day on each of days 1-5 of each of a seventh and eighth 21-day cycles, or methylprednisolone intravenously at a dose of about 80 mg per day on each of days 1-5 of each of a seventh and eighth 21-day cycles;
      wherein administering such treatment to a plurality of human patients results in a hazard ratio of no more than 0.7 in a 36-month progression-free survival rate (PFS36) of the plurality of human patients, as compared to a reference PFS36,
      wherein the reference PFS36 is the 36-month progression-free survival rate of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 108: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone;
      wherein administering such treatment to a plurality of human patients results in an improvement in progression-free survival (PFS) of the plurality of human patients as compared to a reference PFS,
      wherein the reference PFS is the PFS of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 109: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone;
      wherein administering such treatment to a plurality of human patients results in: at least a 20% reduction in the risk of disease progression, relapse, or death in the plurality of human patients, or at least a 25% reduction in the risk of disease progression, relapse, or death in the plurality of human patients,
      as compared to a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 110: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone;
      wherein administering such treatment to a plurality of human patients results in: a hazard ratio of no more than 0.75 in progression-free survival (PFS) of the plurality of human patients, or a hazard ratio of no more than 0.78 in PFS of the plurality of human patients, or a hazard ratio of no more than 0.79 in PFS of the plurality of human patients,
      as compared to a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 111: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone;
      wherein administering such treatment to a plurality of human patients results in a 24-month progression-free survival rate (PFS24) of at least 75%.
Exemplary Embodiment 112: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone;
      wherein administering such treatment to a plurality of human patients results in an improvement in a 24-month progression-free survival rate (PFS24) of the plurality of human patients as compared to a reference PFS24,
      wherein the reference PFS24 is the 24-month progression-free survival rate of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 113: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone;
      wherein administering such treatment to a plurality of human patients results in an improvement in a 24-month progression-free survival rate (PFS24) of the plurality of human patients of at least about 6%, as compared to a reference PFS24,
      wherein the reference PFS24 is the 24-month progression-free survival rate of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 114: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone;
      wherein administering such treatment to a plurality of human patients results in an improvement in event-free survival-efficacy (EFS_{eff}) of the plurality of human patients as compared to a reference EFS_{eff},
      wherein the reference EFS_{eff} is the EFS_{eff}r of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 115: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone;
      wherein administering such treatment to a plurality of human patients results in: a hazard ratio of no more than 0.77 in event-free survival-efficacy (EFS_{eff}) in the plurality of human patients, or a hazard ratio of no more than 0.81 in EFS_{eff} in the plurality of human patients,
      as compared to a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 116: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone;
      wherein administering such treatment to a plurality of human patients results in a rate of complete response (CR) at end of treatment (EOT) in the plurality of human patients of at least about 77%, wherein the rate of CR is assessed by positron emission tomography-computed tomography (PET-CT).
Exemplary Embodiment 117: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone;
      wherein administering such treatment to a plurality of human patients results in: an objective response rate (ORR) at end of treatment (EOT) in the plurality of human patients of at least about 84%, or an ORR at EOT in the plurality of human patients of at least about 85%,
      wherein the ORR is assessed by positron emission tomography-computed tomography (PET-CT).
Exemplary Embodiment 118: A method for treating diffuse large B-cell lymphoma (DLBCL) in a human patient in need thereof, comprising administering to the human patient at least six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle;
      wherein administering such treatment to a plurality of human patients results in an improvement in progression-free survival (PFS) of the plurality of human patients as compared to a reference PFS,
      wherein the reference PFS is the PFS of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 119: A method for treating diffuse large B-cell lymphoma (DLBCL) in a human patient in need thereof, comprising administering to the human patient six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle; and
      wherein the method further comprises administering to the human patient:
      (i) rituximab monotherapy intravenously at a dose of about 375 mg/m² on day 1 of each of a seventh and eighth 21-day cycles; or
      (ii) rituximab intravenously at a dose of about 375 mg/m² on day 1 of each of a seventh and eighth 21-day cycles; cyclophosphamide intravenously at a dose of about 750 mg/m² on day 1 of each of a seventh and eighth 21-day cycles; doxorubicin intravenously at a dose of about 50 mg/m² on day 1 of each of a seventh and eighth 21-day cycles; and prednisone orally at a dose of about 100 mg per day on each of days 1-5 of each of a seventh and eighth 21-day cycles;
      wherein administering such treatment to a plurality of human patients results in an improvement in progression-free survival (PFS) of the plurality of human patients as compared to a reference PFS,
      wherein the reference PFS is the PFS of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 120: The method of any one of embodiments 1-119, further comprising administering to the human patient an antihistamine drug, an analgesic, and/or an anti-pyretic drug.
Exemplary Embodiment 121: The method of any one of embodiments 1-120, further comprising administering to the human patient a prophylactic therapy for neutropenia.
Exemplary Embodiment 122: The method of embodiment 121, comprising administering to the human patient granulocyte colony-stimulating factor (G-CSF).
Exemplary Embodiment 123: The method of embodiment 122, wherein the G-CSF is filgrastim, or lenograstim, or peg-filgrastim.
Exemplary Embodiment 124: The method of any one of embodiments 1-123, wherein the human patient has a high tumor burden.
Exemplary Embodiment 125: The method of embodiment 124, wherein the human patient has a lymphocyte count of at least about 25×10⁹/L.
Exemplary Embodiment 126: The method of embodiment 124, wherein the human patient has bulky lymphadenopathy.
Exemplary Embodiment 127: The method of any one of embodiments 1-126, wherein the human patient is at risk for developing tumor lysis syndrome.
Exemplary Embodiment 128: The method of embodiment 127, further comprising administering to the human patient a prophylactic therapy for tumor lysis syndrome.
Exemplary Embodiment 129: The method of embodiment 128, wherein the prophylactic therapy for tumor lysis syndrome comprises administering allopurinol or rasburicase to the human patient.
Exemplary Embodiment 130: The method of embodiment 128 or embodiment 129, wherein the prophylactic therapy for tumor lysis syndrome comprises a hydration regimen.
Exemplary Embodiment 131: The method of embodiment 130, wherein the hydration regimen comprises administering to the human patient about 3 liters per day of fluids starting at between 1 and 2 days prior to the start of treatment for DLBCL.
Exemplary Embodiment 132: The method of any one of embodiments 1-131, wherein the human patient has previously untreated DLBCL.
Exemplary Embodiment 133: The method of any one of embodiments 1-132, wherein the DLBCL is CD20 positive.
Exemplary Embodiment 134: The method of any one of embodiments 1-133, wherein the DLBCL is a DLBCL, not otherwise specified (NOS).
Exemplary Embodiment 135: The method of embodiment 134, wherein the DLBCL is a germinal center B-cell type DLBCL.
Exemplary Embodiment 136: The method of embodiment 134, wherein the DLBCL is an activated B-cell type DLBCL.
Exemplary Embodiment 137: The method of any one of embodiments 1-133, wherein the DLBCL is:
   (a) a T-cell/histiocyte-rich large B-cell lymphoma;
   (b) an Epstein-Barr virus-positive DLBCL, NOS;
   (c) an ALK-positive large B-cell lymphoma;
   (d) an HHV8-positive DLBCL, NOS;
   (e) a high-grade B-cell lymphoma comprising a MYC, a BCL2, and/or a BCL6 rearrangement (a double-hit lymphoma or a triple-hit lymphoma); or
   (h) a high-grade B-cell lymphoma, NOS.
Exemplary Embodiment 138: The method of any one of embodiments 1-137, wherein the human patient has an International Prognostic Index (IPI) score of between 2 and 5.
Exemplary Embodiment 139: The method of embodiment 138, wherein the human patient has an IPI score of 2.
Exemplary Embodiment 140: The method of embodiment 138, wherein the human patient has an IPI score of between 3 and 5.
Exemplary Embodiment 141: The method of any one of embodiments 1-140, wherein the human patient is an adult.
Exemplary Embodiment 142: The method of any one of embodiments 1-141, wherein the human patient has an Eastern Cooperative Oncology Group (ECOG) Performance Status of 0, 1, or 2.
Exemplary Embodiment 143: The method of any one of embodiments 1-142, wherein the human patient has at least one bi-dimensionally measurable lesion.
Exemplary Embodiment 144: The method of embodiment 143, wherein the at least one bi-dimensionally measurable lesion has a size greater than 1.5 cm in its longest dimension, as measured by computed tomography (CT) or magnetic resonance imaging (MRI).
Exemplary Embodiment 145: The method of any one of embodiments 1-144, wherein the human patient does not have peripheral neuropathy of grade greater than 1 prior to the start of treatment for DLBCL.
Exemplary Embodiment 146: The method of any one of embodiments 1-145, wherein the human patient does not have a demyelinating form of Charcot-Marie Tooth disease prior to the start of treatment for DLBCL.
Exemplary Embodiment 147: The method of any one of embodiments 1-146, wherein the human patient does not have history of indolent lymphoma prior to the start of treatment for DLBCL.
Exemplary Embodiment 148: The method of any one of embodiments 1-147, wherein the human patient does not have:
   (a) follicular lymphoma grade 3B,
   (b) B-cell lymphoma, unclassifiable, with features intermediate between DLBCL and classical Hodgkin lymphoma,
   (c) grey-zone lymphoma,
   (d) primary mediastinal (thymic) large B-cell lymphoma,
   (e) Burkitt lymphoma,
   (f) central nervous system (CNS) lymphoma, primary or secondary involvement,
   (g) primary effusion DLBCL, or
   (h) primary cutaneous DLBCL,
   prior to the start of treatment for DLBCL.
Exemplary Embodiment 149: The method of any one of embodiments 1-148, wherein the human patient has not been previously treated for DLBCL.
Exemplary Embodiment 150: The method of any one of the preceding embodiments, wherein the disease progression or relapse is assessed using the 2014 Lugano Classification for Malignant Lymphoma, and the death is from any cause.
Exemplary Embodiment 151: A kit comprising polatuzumab vedotin for use in combination with rituximab, cyclophosphamide, doxorubicin, and prednisone, prednisolone or methylprednisolone for treating a human patient in need thereof having diffuse large B-cell lymphoma (DLBCL) according to the method of any one of embodiments 62-150.
Exemplary Embodiment 152: The kit of embodiment 151, wherein the DLBCL is previously untreated DLBCL.
Exemplary Embodiment 153: Polatuzumab vedotin for use in combination with rituximab, cyclophosphamide, doxorubicin, and prednisone, prednisolone or methylprednisolone for treating a human patient in need thereof having diffuse large B-cell lymphoma (DLBCL) according to the method of any one of embodiments 62-150.
Exemplary Embodiment 154: The polatuzumab vedotin of embodiment 153, wherein the DLBCL is previously untreated DLBCL.
Exemplary Embodiment 155: A kit comprising an immunoconjugate comprising the formula:

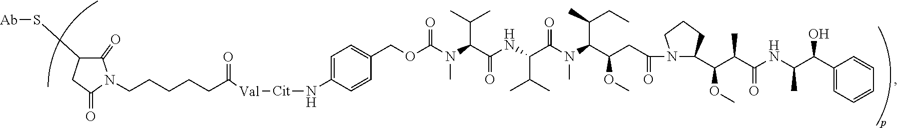

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, for use in combination with rituximab, cyclophosphamide, doxorubicin, and prednisone, prednisolone or methylprednisolone for treating a human patient in need thereof having diffuse large B-cell lymphoma (DLBCL) according to the method of any one of embodiments 1-150.
Exemplary Embodiment 156: The kit of embodiment 155, wherein the DLBCL is previously untreated DLBCL.
Exemplary Embodiment 157: An immunoconjugate comprising the formula:

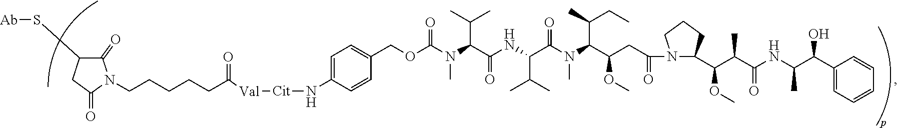

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, for use in combination with rituximab, cyclophosphamide, doxorubicin, and prednisone, prednisolone or methylprednisolone for treating a human patient in need thereof having diffuse large B-cell lymphoma (DLBCL) according to the method of any one of embodiments 1-150.
Exemplary Embodiment 158: The immunoconjugate of embodiment 157, wherein the DLBCL is previously untreated DLBCL.
Exemplary Embodiment 159: The kit of embodiment 155 or embodiment 156, or the immunoconjugate of embodiment 157 or embodiment 158, wherein p is between 2 and 5.
Exemplary Embodiment 160: The kit of embodiment 155 or embodiment 156, or the immunoconjugate of embodiment 157 or embodiment 158, wherein p is between 3 and 4.
Exemplary Embodiment 161: The kit of embodiment 155 or embodiment 156, or the immunoconjugate of embodiment 157 or embodiment 158, wherein the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20.
Exemplary Embodiment 162: The kit of embodiment 155 or embodiment 156, or the immunoconjugate of embodiment 157 or embodiment 158, wherein the anti-CD79b antibody comprises:
   a heavy chain comprising the amino acid sequence of SEQ ID NO: 36 and a light chain comprising the amino acid sequence of SEQ ID NO: 35;
   a heavy chain comprising the amino acid sequence of SEQ ID NO: 37 and a light chain comprising the amino acid sequence of SEQ ID NO: 35;
   a heavy chain comprising the amino acid sequence of SEQ ID NO: 39 and a light chain comprising the amino acid sequence of SEQ ID NO: 35;
   a heavy chain comprising the amino acid sequence of SEQ ID NO: 36 and a light chain comprising the amino acid sequence of SEQ ID NO: 38;
   a heavy chain comprising the amino acid sequence of SEQ ID NO: 37 and a light chain comprising the amino acid sequence of SEQ ID NO: 38; or
   a heavy chain comprising the amino acid sequence of SEQ ID NO: 39 and a light chain comprising the amino acid sequence of SEQ ID NO: 38.
Exemplary Embodiment 163: The kit of embodiment 155 or embodiment 156, or the immunoconjugate of embodiment 157 or embodiment 158, wherein the anti-CD79b antibody comprises (i) a heavy chain comprising the amino acid sequence of SEQ ID NO: 36 and (ii) a light chain comprising the amino acid sequence of SEQ ID NO: 35
Exemplary Embodiment 164: The kit of embodiment 155 or embodiment 156, or the immunoconjugate of embodiment 157 or embodiment 158, wherein the immunoconjugate is polatuzumab vedotin.
Exemplary Embodiment 165: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone, prednisolone, or methylprednisolone:
      wherein administering such treatment to the human patient prolongs the duration of progression-free survival (PFS) of the human patient,
      wherein the human patient has previously untreated DLBCL and at least one of the following characteristics:
      a) <−65 years old, or >65 years old, or at least 60 years old:
      b) the identified histopathologically, high grade b-cell lymphoma, NOS or HGBL with MYC and BCL2 and/or BCL6-rearrangements:
      c) an activated B-cell like (ABC) subtype DLBCL, or a double expressing lymphoma (DEL: overexpression of BCL2 and MYC), or a DLBCL that does not have double-hit or triple-hit lymphoma defined by MYC and BCL2 and/or BCL6-rearrangements:
      d) low Ann-Arbor Stage (I-II), or higher Ann-Arbor Stages (III, IV);
      e) normal baseline LDH levels, or elevated baseline LDH levels:
      f) bone marrow involvement at baseline:
      g) 0-1 extranodal sites or 2+ extranodal sites; and
      h) absence of bulky disease at baseline.
Exemplary Embodiment 166: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone, prednisolone, or methylprednisolone;
      wherein the human patient has an age of greater than 60 years,
      wherein administering such treatment to a plurality of human patients having an age of greater than 60 years results in an improvement in progression-free survival (PFS) of the plurality of human patients as compared to a reference PFS, and
      wherein the reference PFS is the PFS of a plurality of human patients having an age of greater than 60 years who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 167: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone, prednisolone, or methylprednisolone;
      wherein the human patient has an age of greater than 65 years,
      wherein administering such treatment to a plurality of human patients having an age of greater than 65 years results in an improvement in progression-free survival (PFS) of the plurality of human patients as compared to a reference PFS, and
      wherein the reference PFS is the PFS of a plurality of human patients having an age of greater than 65 years who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 168: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone, prednisolone, or methylprednisolone;
      wherein the human patient has an International Prognostic Index (IPI) score between 3 and 5,
      wherein administering such treatment to a plurality of human patients having an IPI score between 3 and 5 results in an improvement in progression-free survival (PFS) of the plurality of human patients as compared to a reference PFS, and
      wherein the reference PFS is the PFS of a plurality of human patients having an IPI score between 3 and 5 who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 169: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone, prednisolone, or methylprednisolone;
      wherein the human patient has an age of greater than 60 years and an International Prognostic Index (IPI) score between 3 and 5,
      wherein administering such treatment to a plurality of human patients having an age of greater than 60 years and an IPI score between 3 and 5 results in an improvement in progression-free survival (PFS) of the plurality of human patients as compared to a reference PFS, and
      wherein the reference PFS is the PFS of a plurality of human patients having an age of greater than 60 years and an IPI score between 3 and 5 who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 170: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone, prednisolone, or methylprednisolone;
      wherein the human patient has an age of greater than 65 years and an International Prognostic Index (IPI) score between 3 and 5,
      wherein administering such treatment to a plurality of human patients having an age of greater than 65 years and an IPI score between 3 and 5 results in an improvement in progression-free survival (PFS) of the plurality of human patients as compared to a reference PFS, and
      wherein the reference PFS is the PFS of a plurality of human patients having an age of greater than 65 years and an IPI score between 3 and 5 who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 171: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone, prednisolone, or methylprednisolone;
      wherein the human patient has an activated B-cell (ABC) type DLBCL,
      wherein administering such treatment to a plurality of human patients having an ABC type DLBCL results in an improvement in progression-free survival (PFS) of the plurality of human patients as compared to a reference PFS, and
      wherein the reference PFS is the PFS of a plurality of human patients having an ABC type DLBCL who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 172: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone, prednisolone, or methylprednisolone;
      wherein the human patient has a double expressing lymphoma (DEL) type DLBCL,
      wherein administering such treatment to a plurality of human patients having a DEL type DLBCL results in an improvement in progression-free survival (PFS) of the plurality of human patients as compared to a reference PFS, and
      wherein the reference PFS is the PFS of a plurality of human patients having a DEL type DLBCL who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 173: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone, prednisolone, or methylprednisolone;
      wherein:
      (i) the human patient has an age greater than 60 years, and wherein administering such treatment to a plurality of human patients having an age greater than 60 years results in a stratified hazard ratio of no more than 0.72 in progression-free survival (PFS) of the plurality of human patients as compared to a control treatment, or
      (ii) the human patient has an age greater than 65 years, and wherein administering such treatment to a plurality of human patients having an age greater than 65 years results in a stratified hazard ratio of no more than 0.79 in PFS of the plurality of human patients as compared to a control treatment;
      wherein the control treatment comprises:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 174: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone, prednisolone, or methylprednisolone;
      wherein the human patient has an International Prognostic Index (IPI) score of between 3 and 5, and
      wherein administering such treatment to a plurality of human patients having an IPI score of between 3 and 5 results in a stratified hazard ratio of no more than 0.68 in progression-free survival (PFS) of the plurality of human patients, as compared to a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 175: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone, prednisolone, or methylprednisolone;
      wherein:
      (i) the human patient has an activated B-cell (ABC) type DLBCL, and wherein administering such treatment to a plurality of human patients having an ABC type DLBCL results in a stratified hazard ratio of no more than 0.31 in progression-free survival (PFS) of the plurality of human patients as compared to a control treatment, or
      (ii) the human patient has a double expressing lymphoma (DEL) type DLBCL, and wherein administering such treatment to a plurality of human patients having a DEL type DLBCL results in a stratified hazard ratio of no more than 0.62 in PFS of the plurality of human patients as compared to a control treatment;
      wherein the control treatment comprises:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 176: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone, prednisolone, or methylprednisolone;
      wherein:
      (i) the human patient has an age greater than 60 years, and wherein administering such treatment to a plurality of human patients having an age greater than 60 years results in: an unstratified hazard ratio of no more than 0.72 in progression-free survival (PFS) of the plurality of human patients as compared to a control treatment, or an unstratified hazard ratio of no more than 0.76 in progression-free survival (PFS) of the plurality of human patients as compared to a control treatment, or
      (ii) the human patient has an age greater than 65 years, and wherein administering such treatment to a plurality of human patients having an age greater than 65 years results in: an unstratified hazard ratio of no more than 0.77 in PFS of the plurality of human patients as compared to a control treatment, or an unstratified hazard ratio of no more than 0.78 in PFS of the plurality of human patients as compared to a control treatment;
      wherein the control treatment comprises:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 177: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone, prednisolone, or methylprednisolone;
      wherein the human patient has an International Prognostic Index (IPI) score between 3 and 5, and
      wherein administering such treatment to a plurality of human patients having an IPI score between 3 and 5 results in: an unstratified hazard ratio of no more than 0.71 in progression-free survival (PFS) of the plurality of human patients, or an unstratified hazard ratio of no more than 0.75 in progression-free survival (PFS) of the plurality of human patients,
      as compared to a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 178: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone, prednisolone, or methylprednisolone:
      wherein:
      (i) the human patient has an activated B-cell (ABC) type DLBCL, and wherein administering such treatment to a plurality of human patients having an ABC type DLBCL results in: an unstratified hazard ratio of no more than 0.36 in progression-free survival (PFS) of the plurality of human patients as compared to a control treatment, or an unstratified hazard ratio of no more than 0.39 in progression-free survival (PFS) of the plurality of human patients as compared to a control treatment, or
      (ii) the human patient has a double expressing lymphoma (DEL) type DLBCL, and wherein administering such treatment to a plurality of human patients having a DEL type DLBCL results in: an unstratified hazard ratio of no more than 0.65 in PFS of the plurality of human patients as compared to a control treatment, or an unstratified hazard ratio of no more than 0.67 in PFS of the plurality of human patients as compared to a control treatment; wherein the control treatment comprises:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 179: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone, prednisolone, or methylprednisolone;
      wherein the human patient has an age of greater than 60 years.
Exemplary Embodiment 180: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone, prednisolone, or methylprednisolone;
      wherein the human patient has an age of greater than 65 years.
Exemplary Embodiment 181: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone, prednisolone, or methylprednisolone;
      wherein the human patient has an International Prognostic Index (IPI) score between 3 and 5.
Exemplary Embodiment 182: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone, prednisolone, or methylprednisolone;
      wherein the human patient has an age of greater than 60 years and an International Prognostic Index (IPI) score between 3 and 5.
Exemplary Embodiment 183: A method for treating diffuse large B-cell lymphoma (DLBCL) in a human patient in need thereof, comprising administering to the human patient an effective amount of:
   (a) polatuzumab vedotin,
   (b) rituximab,
   (c) cyclophosphamide,
   (d) doxorubicin, and
   (e) prednisone, prednisolone, or methylprednisolone;
      wherein the human patient has an age of greater than 65 years and an International Prognostic Index (IPI) score between 3 and 5.
Exemplary Embodiment 184: A method for treating diffuse large B-cell lymphoma (DLBCL) in a human patient in need thereof, comprising administering to the human patient an effective amount of:
   in the absence of immunoconjugate.
Exemplary Embodiment 189: A method for treating diffuse large B-cell lymphoma (DLBCL) in a human patient in need thereof, comprising administering to the human patient at least six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle:
      wherein administering such treatment to a plurality of human patients results in an improvement in a 42-month progression-free survival rate (PFS42) of the plurality of human patients of at least about 6%, as compared to a reference PFS42,
      wherein the reference PFS42 is the 42-month progression-free survival rate of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 190: A method for treating diffuse large B-cell lymphoma (DLBCL) in a human patient in need thereof, comprising administering to the human patient at least six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle;
      wherein administering such treatment to a plurality of human patients results in a 42-month progression-free survival rate (PFS42) of at least 65%, or 70%, or 75%.
Exemplary Embodiment 191: A method for treating diffuse large B-cell lymphoma (DLBCL) in a human patient in need thereof, comprising administering to the human patient at least six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle;
      wherein administering such treatment to a plurality of human patients results in an improvement in a 42-month progression-free survival rate (PFS42) of the plurality of human patients as compared to a reference PFS42,
      wherein the reference PFS42 is the 42-month progression-free survival rate of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 192: A method for treating diffuse large B-cell lymphoma (DLBCL) in a human patient in need thereof, comprising administering to the human patient at least six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle:
      wherein administering such treatment to a plurality of human patients results in an improvement in a 42-month progression-free survival rate (PFS42) of the plurality of human patients of at least about 6%, 7%, 8%, 9%, or 10%, as compared to a reference PFS42,
      wherein the reference PFS42 is the 42-month progression-free survival rate of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 193: A method for treating diffuse large B-cell lymphoma (DLBCL) in a human patient in need thereof, comprising administering to the human patient six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle; and
      wherein the method further comprises administering to the human patient:
      (i) rituximab monotherapy intravenously at a dose of about 375 mg/m² on day 1 of each of a seventh and eighth 21-day cycles; or
      (ii) rituximab intravenously at a dose of about 375 mg/m² on day 1 of each of a seventh and eighth 21-day cycles; cyclophosphamide intravenously at a dose of about 750 mg/m² on day 1 of each of a seventh and eighth 21-day cycles; doxorubicin intravenously at a dose of about 50 mg/m² on day 1 of each of a seventh and eighth 21-day cycles; and prednisone orally at a dose of about 100 mg per day on each of days 1-5 of each of a seventh and eighth 21-day cycles, prednisolone orally at a dose of about 100 mg per day on each of days 1-5 of each of a seventh and eighth 21-day cycles, or methylprednisolone intravenously at a dose of about 80 mg per day on each of days 1-5 of each of a seventh and eighth 21-day cycles;
      wherein administering such treatment to a plurality of human patients results in a 42-month progression-free survival rate (PFS42) of at least 65%, 66%, 67%, 68%, 69%, 70%, 75%, or 80%.
Exemplary Embodiment 194: A method for treating diffuse large B-cell lymphoma (DLBCL) in a human patient in need thereof, comprising administering to the human patient six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle,
   (c) cyclophosphamide intravenously at a dose of about 750 mg/m² on day 1 of each 21-day cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle; and
      wherein the method further comprises administering to the human patient:
      (i) rituximab monotherapy intravenously at a dose of about 375 mg/m² on day 1 of each of a seventh and eighth 21-day cycles; or
      (ii) rituximab intravenously at a dose of about 375 mg/m² on day 1 of each of a seventh and eighth 21-day cycles; cyclophosphamide intravenously at a dose of about 750 mg/m² on day 1 of each of a seventh and eighth 21-day cycles; doxorubicin intravenously at a dose of about 50 mg/m² on day 1 of each of a seventh and eighth 21-day cycles; and prednisone orally at a dose of about 100 mg per day on each of days 1-5 of each of a seventh and eighth 21-day cycles, prednisolone orally at a dose of about 100 mg per day on each of days 1-5 of each of a seventh and eighth 21-day cycles, or methylprednisolone intravenously at a dose of about 80 mg per day on each of days 1-5 of each of a seventh and eighth 21-day cycles;
      wherein administering such treatment to a plurality of human patients results in an improvement in a 42-month progression-free survival rate (PFS42) of the plurality of human patients as compared to a reference PFS42,
      wherein the reference PFS42 is the 42-month progression-free survival rate of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.
Exemplary Embodiment 195: A method for treating diffuse large B-cell lymphoma (DLBCL) in a human patient in need thereof, comprising administering to the human patient six 21-day cycles of:
   (a) polatuzumab vedotin intravenously at a dose of about 1.8 mg/kg on day 1 of each 21-day cycle,
   (b) rituximab intravenously at a dose of about 375 mg/m² on day 1 of each 21-day cycle, cycle,
   (d) doxorubicin intravenously at a dose of about 50 mg/m² on day 1 of each 21-day cycle, and
   (e) prednisone orally at a dose of about 100 mg per day on each of days 1-5 of each 21-day cycle, prednisolone orally at a dose of about 100 mg per day on each of days 1-5 of each 21-day cycle, or methylprednisolone intravenously at a dose of about 80 mg per day on each of days 1-5 of each 21-day cycle; and
      wherein the method further comprises administering to the human patient:
      (i) rituximab monotherapy intravenously at a dose of about 375 mg/m² on day 1 of each of a seventh and eighth 21-day cycles; or
      (ii) rituximab intravenously at a dose of about 375 mg/m² on day 1 of each of a seventh and eighth 21-day cycles; cyclophosphamide intravenously at a dose of about 750 mg/m² on day 1 of each of a seventh and eighth 21-day cycles; doxorubicin intravenously at a dose of about 50 mg/m² on day 1 of each of a seventh and eighth 21-day cycles; and prednisone orally at a dose of about 100 mg per day on each of days 1-5 of each of a seventh and eighth 21-day cycles, prednisolone orally at a dose of about 100 mg per day on each of days 1-5 of each of a seventh and eighth 21-day cycles, or methylprednisolone intravenously at a dose of about 80 mg per day on each of days 1-5 of each of a seventh and eighth 21-day cycles;
      wherein administering such treatment to a plurality of human patients results in an improvement in a 42-month progression-free survival rate (PFS42) of the plurality of human patients of at least about 6%, 7%, 8%, 9%, or 10%, as compared to a reference PFS42,
      wherein the reference PFS42 is the 42-month progression-free survival rate of a plurality of human patients who have received a control treatment comprising:
      (a) rituximab,
      (b) cyclophosphamide,
      (c) doxorubicin,
      (d) vincristine, and
      (e) prednisone, prednisolone, or methylprednisolone,
      in the absence of polatuzumab vedotin.

TABLE C
Amino Acid Sequences

| NAME | SEQUENCE | SEQ ID NO |
|---|---|---|
| Human CD79b | RFIARKRGFT VKMHCYMNSA SGNVSWLWKQ EMDENPQQLK | 1 |
| precursor; Acc. No. | LEKGRMEESQ NESLATLTIQ GIRFEDNGIY FCQQKCNNTS | |
| NP_000617.1; signal | EVYQGCGTEL RVMGFSTLAQ LKQRNTLKDG IIMIQTLLII | |
| sequence = amino | LFIIVPIFLL LDKDDSKAGM EEDHTYEGLD IDQTATYEDI | |
| acids 1 to 28 | VTLRTGEVKW SVGEHPGQE | |

| | | |
|---|---|---|
| Human mature CD79b, | AR SEDRYRNPKG SACSRIWQSP RFIARKRGFT VKMHCYMNSA | 2 |
| without signal | SGNVSWLWKQ EMDENPQQLK LEKGRMEESQ NESLATLTIQ | |
| sequence; amino | GIRFEDNGIY FCQQKCNNTS EVYQGCGTEL RVMGFSTLAQ | |
| acids 29 to 229 | LKQRNTLKDG IIMIQTLLII LFIIVPIFLL LDKDDSKAGM | |
| | EEDHTYEGLD IDQTATYEDI VTLRTGEVKW SVGEHPGQE | |
| VH of mMAb anti- | Gly Pro Glu Leu Val Lys Pro Gly Ala Ser Val | 3 |
| CD20 antibody B-Ly1 | Lys Ile Ser Cys Lys Ala Ser Gly Tyr Ala Phe | |
| | Ser Tyr Ser Trp Met Asn Trp Val Lys Leu Arg | |
| | Pro Gly Gln Gly Leu Glu Trp Ile Gly Arg Ile | |
| | Phe Pro Gly Asp Gly Asp Thr Asp Tyr Asn Gly | |
| | Lys Phe Lys Gly Lys Ala Thr Leu Thr Ala Asp | |
| | Lys Ser Ser Asn Thr Ala Tyr Met Gln Leu Thr | |
| | Ser Leu Thr Ser Val Asp Ser Ala Val Tyr Leu | |
| | Cys Ala Arg Asn Val Phe Asp Gly Tyr Trp Leu | |
| | Val Tyr Trp Gly Gln Gly Thr Leu Val Thr Val | |
| | Ser Ala | |
| VL of mMAb anti- | Asn Pro Val Thr Leu Gly Thr Ser Ala Ser Ile | 4 |
| CD20 antibody B-Ly1 | Ser Cys Arg Ser Ser Lys Ser Leu Leu His Ser | |
| | Asn Gly Ile Thr Tyr Leu Tyr Trp Tyr Leu Gln | |
| | Lys Pro Gly Gln Ser Pro Gln Leu Leu Ile Tyr | |
| | Gln Met Ser Asn Leu Val Ser Gly Val Pro Asp | |
| | Arg Phe Ser Ser Ser Gly Ser Gly Thr Asp Phe | |
| | Thr Leu Arg Ile Ser Arg Val Glu Ala Glu Asp | |
| | Val Gly Val Tyr Tyr Cys Ala Gln Asn Leu Glu | |
| | Leu Pro Tyr Thr Phe Gly Gly Gly Thr Lys Leu | |
| | Glu Ile Lys Arg | |
| GA101 HVR-H1 | Gly Tyr Ala Phe Ser Tyr | 5 |
| GA101 HVR-H2 | Phe Pro Gly Asp Gly Asp Thr Asp | 6 |
| GA101 HVR-H3 | Asn Val Phe Asp Gly Tyr Trp Leu Val Tyr | 7 |
| GA101 HVR-L1 | Arg Ser Ser Lys Ser Leu Leu His Ser Asn Gly | 8 |
| | Ile Thr Tyr Leu Tyr | |
| GA101 HVR-L2 | Gln Met Ser Asn Leu Val Ser | 9 |
| GA101 HVR-L3 | Ala Gln Asn Leu Glu Leu Pro Tyr Thr | 10 |
| GA101 VH | Gln Val Gln Leu Val Gln Ser Gly Ala Glu Val | 11 |
| | Lys Lys Pro Gly Ser Ser Val Lys Val Ser Cys | |
| | Lys Ala Ser Gly Tyr Ala Phe Ser Tyr Ser Trp | |
| | Ile Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| GA101 VL | Asp Ile Val Met Thr Gln Thr Pro Leu Ser Leu | 12 |
| | Pro Val Thr Pro Gly Glu Pro Ala Ser Ile Ser | |
| | Cys Arg Ser Ser Lys Ser Leu Leu His Ser Asn | |
| | Gly Ile Thr Tyr Leu Tyr Trp Tyr Leu Gln Lys | |
| | Pro Gly Gln Ser Pro Gln Leu Leu Ile Tyr Gln | |
| | Met Ser Asn Leu Val Ser Gly Val Pro Asp Arg | |
| | Phe Ser Gly Ser Gly Ser Gly Thr Asp Phe Thr | |
| | Leu Lys Ile Ser Arg Val Glu Ala Glu Asp Val | |
| | Gly Val Tyr Tyr Cys Ala Gln Asn Leu Glu Leu | |
| | Pro Tyr Thr Phe Gly Gly Gly Thr Lys Val Glu | |
| | Ile Lys | |
| GA101 Heavy Chain | Gln Val Gln Leu Val Gln Ser Gly Ala Glu Val | 13 |
| | Lys Lys Pro Gly Ser Ser Val Lys Val Ser Cys | |
| | Lys Ala Ser Gly Tyr Ala Phe Ser Tyr Ser Trp | |
| | Ile Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser Ala Ser | |
| | Thr Lys Gly Pro Ser Val Phe Pro Leu Ala Pro | |
| | Ser Ser Lys Ser Thr Ser Gly Gly Thr Ala Ala | |
| | Leu Gly Cys Leu Val Lys Asp Tyr Phe Pro Glu | |
| | Pro Val Thr Val Ser Trp Asn Ser Gly Ala Leu | |
| | Thr Ser Gly Val His Thr Phe Pro Ala Val Leu | |
| | Gln Ser Ser Gly Leu Tyr Ser Leu Ser Ser Val | |
| | Val Thr Val Pro Ser Ser Ser Leu Gly Thr Gln | |
| | Thr Tyr Ile Cys Asn Val Asn His Lys Pro Ser | |
| | Asn Thr Lys Val Asp Lys Lys Val Glu Pro Lys | |
| | Ser Cys Asp Lys Thr His Thr Cys Pro Pro Cys | |
| | Pro Ala Pro Glu Leu Leu Gly Gly Pro Ser Val | |
| | Phe Leu Phe Pro Pro Lys Pro Lys Asp Thr Leu | |
| | Met Ile Ser Arg Thr Pro Glu Val Thr Cys Val | |
| | Val Val Asp Val Ser His Glu Asp Pro Glu Val | |
| | Lys Phe Asn Trp Tyr Val Asp Gly Val Glu Val | |
| | His Asn Ala Lys Thr Lys Pro Arg Glu Glu Gln | |
| | Tyr Asn Ser Thr Tyr Arg Val Val Ser Val Leu | |
| | Thr Val Leu His Gln Asp Trp Leu Asn Gly Lys | |
| | Glu Tyr Lys Cys Lys Val Ser Asn Lys Ala Leu | |
| | Pro Ala Pro Ile Glu Lys Thr Ile Ser Lys Ala | |
| | Lys Gly Gln Pro Arg Glu Pro Gln Val Tyr Thr | |
| | Leu Pro Pro Ser Arg Asp Glu Leu Thr Lys Asn | |
| | Gln Val Ser Leu Thr Cys Leu Val Lys Gly Phe | |
| | Tyr Pro Ser Asp Ile Ala Val Glu Trp Glu Ser | |
| | Asn Gly Gln Pro Glu Asn Asn Tyr Lys Thr Thr | |
| | Pro Pro Val Leu Asp Ser Asp Gly Ser Phe Phe | |
| | Leu Tyr Ser Lys Leu Thr Val Asp Lys Ser Arg | |
| | Trp Gln Gln Gly Asn Val Phe Ser Cys Ser Val | |
| | Met His Glu Ala Leu His Asn His Tyr Thr Gln | |
| | Lys Ser Leu Ser Leu Ser Pro Gly | |
| GA101 Light Chain | Asp Ile Val Met Thr Gln Thr Pro Leu Ser Leu | 14 |
| | Pro Val Thr Pro Gly Glu Pro Ala Ser Ile Ser | |
| | Cys Arg Ser Ser Lys Ser Leu Leu His Ser Asn | |
| | Gly Ile Thr Tyr Leu Tyr Trp Tyr Leu Gln Lys | |
| | Pro Gly Gln Ser Pro Gln Leu Leu Ile Tyr Gln | |
| | Met Ser Asn Leu Val Ser Gly Val Pro Asp Arg | |
| | Phe Ser Gly Ser Gly Ser Gly Thr Asp Phe Thr | |
| | Leu Lys Ile Ser Arg Val Glu Ala Glu Asp Val | |
| | Gly Val Tyr Tyr Cys Ala Gln Asn Leu Glu Leu | |
| | Pro Tyr Thr Phe Gly Gly Gly Thr Lys Val Glu | |
| | Ile Lys Arg Thr Val Ala Ala Pro Ser Val Phe | |
| | Ile Phe Pro Pro Ser Asp Glu Gln Leu Lys Ser | |
| | Gly Thr Ala Ser Val Val Cys Leu Leu Asn Asn | |
| | Phe Tyr Pro Arg Glu Ala Lys Val Gln Trp Lys | |
| | Val Asp Asn Ala Leu Gln Ser Gly Asn Ser Gln | |
| | Glu Ser Val Thr Glu Gln Asp Ser Lys Asp Ser | |
| | Thr Tyr Ser Leu Ser Ser Thr Leu Thr Leu Ser | |
| | Lys Ala Asp Tyr Glu Lys His Lys Val Tyr Ala | |
| | Cys Glu Val Thr His Gln Gly Leu Ser Ser Pro | |
| | Val Thr Lys Ser Phe Asn Arg Gly Glu Cys | |
| VH of humanized B- | Gln Val Gln Leu Val Gln Ser Gly Ala Glu Val | 15 |
| Ly1 antibody (B- | Lys Lys Pro Gly Ser Ser Val Lys Val Ser Cys | |
| HH2) | Lys Ala Ser Gly Tyr Ala Phe Ser Tyr Ser Trp | |
| | Met Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 16 |
| Ly1 antibody (B- | Lys Lys Pro Gly Ser Ser Val Lys Val Ser Cys | |
| HH3) | Lys Ala Ser Gly Tyr Ala Phe Ser Tyr Ser Trp | |
| | Met Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Leu Cys Ala Arg Asn | |
| | Val Phe Asp Gly Tyr Trp Leu Val Tyr Trp Gly | |
| | Gln Gly Thr Leu Val Thr Val Ser Ser | |
| humanized B-Ly1 | QVQLVQSGAE VKKPGSSVKV SCKASGYAFS YSWINWVRQA | 17 |
| Heavy Chain | PGQGLEWMGR IFPGDGDTDY NGKFKGRVTI TADKSTSTAY | |
| | MELSSLRSED TAVYYCARNV FDGYWLVYWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPELLGGP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSRDEL | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPG | |
| humanized B-Ly1 | DIVMTQTPLS LPVTPGEPAS ISCRSSKSLL HSNGITYLYW | 18 |
| Light Chain | YLQKPGQSPQ LLIYQMSNLV SGVPDRFSGS GSGTDFTLKI | |
| | SRVEAEDVGV YYCAQNLELP YTFGGGTKVE IKRTVAAPSV | |
| | FIFPPSDEQL KSGTASVVCL LNNFYPREAK VQWKVDNALQ | |
| | SGNSQESVTE QDSKDSTYSL SSTLTLSKAD YEKHKVYACE | |
| | VTHQGLSSPV TKSFNRGEC | |
| huMA79bv28 heavy | EVQLVESGGG LVQPGGSLRL SCAASGYTFS SYWIEWVRQA | 19 |
| chain variable | PGKGLEWIGE ILPGGGDTNY NEIFKGRATF SADTSKNTAY | |
| region | LQMNSLRAED TAVYYCTRRV PIRLDYWGQG TLVTVSS | |
| huMA79bv28 light | DIQLTQSPSS LSASVGDRVT ITCKASQSVD YEGDSFLNWY | 20 |
| chain variable | QQKPGKAPKL LIYAASNLES GVPSRFSGSG SGTDFTLTIS | |
| region | SLQPEDFATY YCQQSNEDPL TFGQGTKVEI KR | |
| huMA79bv28 HVR H1 | GYTFSSYWIE | 21 |
| huMA79bv28 HVR H2 | GEILPGGGDTNYNEIFKG | 22 |
| huMA79bv28 HVR H3 | TRRVPIRLDY | 23 |
| huMA79bv28 HVR L1 | KASQSVDYEGDSFLN | 24 |
| huMA79bv28 HVR L2 | AASNLES | 25 |
| huMA79bv28 HVR L3 | QQSNEDPLT | 26 |
| huMA79bv28 heavy | EVQLVESGGGLVQPGGSLRLSCAAS | 27 |
| chain (HC) | | |
| framework region | | |
| (FR) 1 | | |
| huMA79bv28 HC FR2 | WVRQAPGKGLEWI | 28 |
| huMA79bv28 HC FR3 | RATFSADTSKNTAYLQMNSLRAEDTAVYYC | 29 |
| huMA79bv28 HC FR4 | WGQGTLVTVSS | 30 |
| huMA79bv28 light | DIQLTQSPSSLSASVGDRVTITC | 31 |
| chain (LC) FR1 | | |
| huMA79bv28 LC FR2 | WYQQKPGKAPKLLIY | 32 |
| huMA79bv28 LC FR3 | GVPSRFSGSGSGTDFTLTISSLQPEDFATYYC | 33 |
| huMA79bv28 LC FR4 | FGQGTKVEIKR | 34 |
| huMA79bv28 light | DIQLTQSPSS LSASVGDRVT ITCKASQSVD YEGDSFLNWY | 35 |
| chain (Igk) | QQKPGKAPKL LIYAASNLES GVPSRFSGSG SGTDFTLTIS | |
| | SLQPEDFATY YCQQSNEDPL TFGQGTKVEI KRTVAAPSVF | |
| | IFPPSDEQLK SGTASVVCLL NNFYPREAKV QWKVDNALQS | |
| | GNSQESVTEQ DSKDSTYSLS STLTLSKADY EKHKVYACEV | |
| | THQGLSSPVT KSFNRGEC | |
| huMA79bv28 heavy | EVQLVESGGG LVQPGGSLRL SCAASGYTFS SYWIEWVRQA | 36 |
| chain (IgG1) | PGKGLEWIGE ILPGGGDTNY NEIFKGRATF SADTSKNTAY | |
| | LQMNSLRAED TAVYYCTRRV PIRLDYWGQG TLVTVSSAST | |
| | KGPSVFPLAP SSKSTSGGTA ALGCLVKDYF PEPVTVSWNS | |
| | GALTSGVHTF PAVLQSSGLY SLSSVVTVPS SSLGTQTYIC | |
| | NVNHKPSNTK VDKKVEPKSC DKTHTCPPCP APELLGGPSV | |
| | FLFPPKPKDT LMISRTPEVT CVVVDVSHED PEVKFNWYVD | |
| | GVEVHNAKTK PREEQYNSTY RVVSVLTVLH QDWLNGKEYK | |
| | CKVSNKALPA PIEKTISKAK GQPREPQVYT LPPSREEMTK | |
| | NQVSLTCLVK GFYPSDIAVE WESNGQPENN YKTTPPVLDS | |
| | DGSFFLYSKL TVDKSRWQQG NVFSCSVMHE ALHNHYTQKS | |
| | LSLSPG | |
| huMA79bv28 A118C | EVQLVESGGG LVQPGGSLRL SCAASGYTFS SYWIEWVRQA | 37 |
| cysteine engineered | PGKGLEWIGE ILPGGGDTNY NEIFKGRATF SADTSKNTAY | |
| heavy chain (IgG1) | LQMNSLRAED TAVYYCTRRV PIRLDYWGQG TLVTVSSCST | |
| | KGPSVFPLAP SSKSTSGGTA ALGCLVKDYF PEPVTVSWNS | |
| | GALTSGVHTF PAVLQSSGLY SLSSVVTVPS SSLGTQTYIC | |
| | NVNHKPSNTK VDKKVEPKSC DKTHTCPPCP APELLGGPSV | |
| | FLFPPKPKDT LMISRTPEVT CVVVDVSHED PEVKFNWYVD | |
| | GVEVHNAKTK PREEQYNSTY RVVSVLTVLH QDWLNGKEYK | |
| | CKVSNKALPA PIEKTISKAK GQPREPQVYT LPPSREEMTK | |
| | NQVSLTCLVK GFYPSDIAVE WESNGQPENN YKTTPPVLDS | |
| | DGSFFLYSKL TVDKSRWQQG NVFSCSVMHE ALHNHYTQKS | |
| | LSLSPG | |
| huMA79bv28 V205C | DIQLTQSPSS LSASVGDRVT ITCKASQSVD YEGDSFLNWY | 38 |
| cysteine engineered | QQKPGKAPKL LIYAASNLES GVPSRFSGSG SGTDFTLTIS | |
| light chain (Igκ) | SLQPEDFATY YCQQSNEDPL TFGQGTKVEI KRTVAAPSVF | |
| | IFPPSDEQLK SGTASVVCLL NNFYPREAKV QWKVDNALQS | |
| | GNSQESVTEQ DSKDSTYSLS STLTLSKADY EKHKVYACEV | |
| | THQGLSSPCT KSFNRGEC | |
| huMA79bv28 S400C | EVQLVESGGG LVQPGGSLRL SCAASGYTFS SYWIEWVRQA | 39 |
| cysteine engineered | PGKGLEWIGE ILPGGGDTNY NEIFKGRATF SADTSKNTAY | |
| heavy chain (IgG1) | LQMNSLRAED TAVYYCTRRV PIRLDYWGQG TLVTVSSAST | |
| | KGPSVFPLAP SSKSTSGGTA ALGCLVKDYF PEPVTVSWNS | |
| | GALTSGVHTF PAVLQSSGLY SLSSVVTVPS SSLGTQTYIC | |
| | NVNHKPSNTK VDKKVEPKSC DKTHTCPPCP APELLGGPSV | |
| | FLFPPKPKDT LMISRTPEVT CVVVDVSHED PEVKFNWYVD | |
| | GVEVHNAKTK PREEQYNSTY RVVSVLTVLH QDWLNGKEYK | |
| | CKVSNKALPA PIEKTISKAK GQPREPQVYT LPPSREEMTK | |
| | NQVSLTCLVK GFYPSDIAVE WESNGQPENN YKTTPPVLDC | |
| | DGSFFLYSKL TVDKSRWQQG NVFSCSVMHE ALHNHYTQKS | |
| | LSLSPGK | |
| VH of humanized B- | Gln Val Gln Leu Val Gln Ser Gly Ala Glu Val | 40 |
| Ly1 antibody (B- | Lys Lys Pro Gly Ala Ser Val Lys Val Ser Cys | |
| HH4) | Lys Val Ser Gly Tyr Ala Phe Ser Tyr Ser Trp | |
| | Met Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 41 |
| Ly1 antibody (B- | Lys Lys Pro Gly Ser Ser Val Lys Val Ser Cys | |
| HH5) | Lys Ala Ser Gly Tyr Ala Phe Ser Tyr Ser Trp | |
| | Met Ser Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 42 |
| Ly1 antibody (B- | Lys Lys Pro Gly Ser Ser Val Lys Val Ser Cys | |
| HH6) | Lys Ala Ser Gly Tyr Ala Phe Ser Tyr Ser Trp | |
| | Ile Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 43 |
| Ly1 antibody (B- | Lys Lys Pro Gly Ser Ser Val Lys Val Ser Cys | |
| HH7) | Lys Ala Ser Gly Tyr Ala Phe Ser Tyr Ser Trp | |
| | Ile Ser Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 44 |
| Ly1 antibody (B- | Lys Lys Pro Gly Ala Ser Val Lys Val Ser Cys | |
| HH8) | Lys Ala Ser Gly Tyr Thr Phe Thr Tyr Ser Trp | |
| | Met Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 45 |
| Ly1 antibody (B- | Lys Lys Pro Gly Ala Ser Val Lys Val Ser Cys | |
| HH9) | Lys Ala Ser Gly Tyr Thr Phe Ser Tyr Ser Trp | |
| | Met Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | 46 |
| Ly1 antibody (B- | Val Lys Pro Gly Gly Ser Leu Arg Leu Ser Cys | |
| HL8) | Ala Ala Ser Gly Phe Thr Phe Ser Tyr Ser Trp | |
| | Met Asn Trp Val Arg Gln Ala Pro Gly Lys Gly | |
| | Leu Glu Trp Val Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | 47 |
| Ly1 antibody (B- | Val Lys Pro Gly Gly Ser Leu Arg Leu Ser Cys | |
| HL10) | Ala Ala Ser Gly Phe Ala Phe Ser Tyr Ser Trp | |
| | Met Asn Trp Val Arg Gln Ala Pro Gly Lys Gly | |
| | Leu Glu Trp Val Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Glu Ser Gly Gly Gly Leu | 48 |
| Ly1 antibody (B- | Val Lys Pro Gly Gly Ser Leu Arg Leu Ser Cys | |
| HL11) | Ala Ala Ser Gly Phe Thr Phe Ser Tyr Ser Trp | |
| | Met Asn Trp Val Arg Gln Ala Pro Gly Lys Gly | |
| | Leu Glu Trp Val Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Ala Gly Leu | 49 |
| Ly1 antibody (B- | Val Lys Pro Gly Gly Ser Leu Arg Leu Ser Cys | |
| HL12) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Val | 50 |
| Ly1 antibody (B- | Val Lys Pro Gly Gly Ser Leu Arg Leu Ser Cys | |
| HL13) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | 51 |
| Ly1 antibody (B- | Lys Lys Pro Gly Gly Ser Leu Arg Leu Ser Cys | |
| HL14) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | 52 |
| Ly1 antibody (B- | Val Lys Pro Gly Ser Ser Leu Arg Leu Ser Cys | |
| HL15) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | 53 |
| Ly1 antibody (B- | Val Lys Pro Gly Gly Ser Leu Arg Val Ser Cys | |
| HL16) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | 54 |
| Ly1 antibody (B- | Val Lys Pro Gly Gly Ser Leu Arg Leu Ser Cys | |
| HL17) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VL of humanized B- | Asp Ile Val Met Thr Gln Thr Pro Leu Ser Leu | 55 |
| Ly1 antibody (B- | Pro Val Thr Pro Gly Glu Pro Ala Ser Ile Ser | |
| KVI) | Cys Arg Ser Ser Lys Ser Leu Leu His Ser Asn | |
| | Gly Ile Thr Tyr Leu Tyr Trp Tyr Leu Gln Lys | |
| | Pro Gly Gln Ser Pro Gln Leu Leu Ile Tyr Gln | |
| | Met Ser Asn Leu Val Ser Gly Val Pro Asp Arg | |
| | Phe Ser Gly Ser Gly Ser Gly Thr Asp Phe Thr | |
| | Leu Lys Ile Ser Arg Val Glu Ala Glu Asp Val | |
| | Gly Val Tyr Tyr Cys Ala Gln Asn Leu Glu Leu | |
| | Pro Tyr Thr Phe Gly Gly Gly Thr Lys Val Glu | |
| | Ile Lys Arg Thr Val | |

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The following are examples of methods and compositions of the disclosure. It is understood that various other embodiments may be practiced, given the general description provided above.

### Example 1: A Phase III Study of Polatuzumab Vedotin in Combination with Rituximab and Cyclophosphamide, Doxorubicin, and Prednisone (Pola-R-CHP) Compared to Rituximab, Cyclophosphamide, Doxorubicin, Vincristine, and Prednisone (R-CHOP) in Previously Untreated Diffuse Large B-Cell Lymphoma (DLBCL)

This Example describes a Phase III, multicenter, randomized, double-blind, placebo-controlled study of the efficacy and safety of polatuzumab vedotin (Pola) in combination with rituximab and cyclophosphamide, doxorubicin, and prednisone (R-CHP) compared to rituximab, cyclophosphamide, doxorubicin, vincristine, and prednisone (R-CHOP) in patients with previously untreated diffuse large B-cell lymphoma (DLBCL).

### I. Study Objectives and Endpoints

This study evaluates the efficacy, safety, pharmacokinetics, and patient reported outcomes (PROs) of polatuzumab vedotin at 1.8 mg/kg plus chemoimmunotherapy (R-CHP) compared with standard of care (SOC) chemoimmunotherapy (R-CHOP) in previously untreated patients with CD20-positive diffuse large B-cell lymphoma (DLBCL). The primary study endpoint is progression-free survival (PFS) as assessed by the investigator. Specific objectives and corresponding endpoints for the study are outlined in Table 1.

TABLE 1
Study Objectives and Corresponding Endpoints.

| | |
|---|---|
| Objective | Corresponding Endpoints |

Primary Efficacy Objective

| | |
|---|---|
| Evaluate the efficacy of | PFS, defined as the time from randomization to the first occurrence of |
| polatuzumab vedotin | disease progression or relapse as assessed by the investigator, using |
| plus R-CHP compared | the Lugano Response Criteria for Malignant Lymphoma (Cheson et |
| with R-CHOP with | al., J Clin Oncol (2014) 32: 1-9; see Table 2), or death from any |
| respect to progression- | cause, whichever occurrs earlier. |
| free survival (PFS). | |

Secondary Efficacy Objective

| | |
|---|---|
| Evaluate the efficacy of | Key secondary endpoints included in hierarchical testing procedureª: |
| polatuzumab vedotin | Event-free survival-efficacy (EFS_{eff}) as determined by the |
| plus R-CHP compared | investigator. EFS_{eff} reflects EFS events that are primarily due to |
| with R-CHOP with | efficacy and is defined as time from randomization to the earliest |
| respect to secondary | occurrence of disease progression/relapse; death due to any cause; |
| efficacy endpoints. | an efficacy reason determined by the investigator, other than |
| | disease progression/relapse, that leads to initiation of any non- |
| | protocol-specified anti-lymphoma treatment (NALT); or if biopsy |
| | is obtained after treatment completion, and is positive for residual |
| | disease, regardless of whether NALT is initiated or not. |
| | Complete response (CR) rate at end of treatment^{b} by |
| | fluorodeoxyglucose positron emission tomography (FDG-PET) as |
| | determined by blinded independent central review (BICR). |
| | Overall survival (OS), defined as the period from the date of |
| | randomization until the date of death from any cause. |
| | Secondary endpoints not adjusted for testing multiplicityª: |
| | CR rate at end of treatment^{b} by FDG-PET as determined by the |
| | investigator. |
| | 2-year progression-free survival rate (PFS24) as determined by the |
| | investigator. |
| | Disease-free survival (DFS), defined as the time from the date of the |
| | first occurrence of a documented CR, to the date of relapse or |
| | death from any cause for the subgroup of patients with a best |
| | overall response (BOR) of CR. |
| | Duration of response (DOR), defined as the time from the date of |
| | the first occurrence of a documented response to the date of |
| | progression, relapse, or death from any cause for the subgroup of |
| | patients with a BOR of CR or PR. |
| | EFS-all causes (EFSₐₗₗ), defined as the time from randomization to |
| | disease progression or relapse, as assessed by the investigator; |
| | death from any cause; or initiation of any new anti-lymphoma |
| | therapy (NALT). |
| | Patient-reported outcome (PRO) endpoints: time to deterioration in |
| | European Organisation for Research and Treatment of Cancer |
| | Quality of Life-Core 30 questionnaire (EORTC QLQ-C30) |
| | physical functioning and fatigue, and Functional Assessment of |
| | Cancer Therapy-Lymphoma Lymphoma Subscale (FACT-Lym |
| | LymS); proportion of patients achieving meaningful improvement |
| | in EORTC QLQ-C30 physical functioning and fatigue, and FACT- |
| | Lym LymS. |
| | EORTC QLQ-C30 rate of treatment-related symptoms and Functional |
| | Assessment of Cancer Treatment/Gynecologic Oncology Group- |
| | Neurotoxicity (FACT/GOG-NTX) peripheral neuropathy rate. |

Exploratory Efficacy Objective

| | |
|---|---|
| Evaluate the efficacy of | PRO endpoints: all scales of the EORTC QLQ-C30, the FACT-Lym |
| polatuzumab vedotin | LymS, and FACT/GOG-NTX peripheral neuropathy. |
| plus R-CHP compared | Selected primary and secondary endpoints (e.g. PFS, CR rate) by |
| with R-CHOP with | alternative response criteria including, but not limited to RECIL |
| respect to exploratory | 2017^{c}, and by radiographic measurements of tumor volume and tumor |
| endpoints. | metabolic volume by BICR. |

Safety Objective

| | |
|---|---|
| Evaluate the safety of | Incidence, nature, and severity of adverse events, with severity |
| polatuzumab vedotin | determined through use of National Cancer Institute Common |
| plus R-CHP compared | Terminology Criteria for Adverse Events, Version 4.0 (NCI CTCAE |
| with R-CHOP. | v4.0). |
| | Incidence of peripheral neuropathy rates and severity determined |
| | through use of NCI CTCAE v4.0. |
| | Incidence and nature of study drug discontinuation, dose reduction, |
| | and dose delay due to adverse events. |
| | Dose intensities of study drugs. |

Secondary Pharmacokinetic Objective

| | |
|---|---|
| Characterize the | Plasma and/or serum concentration of polatuzumab vedotin-related |
| pharmacokinetics (PK) | analytes at specified time points. |
| of polatuzumab vedotin. | |

Exploratory Pharmacokinetic Objectives

| | |
|---|---|
| Evaluate potential | Relationship between selected covariates and plasma and/or serum |
| relationships between | concentration or PK parameters of polatuzumab vedotin. |
| selected covariates and | Relationship between plasma and/or serum concentration or PK |
| exposure to polatuzumab | parameters of polatuzumab vedotin-related analytes and efficacy |
| vedotin. | endpoints. |
| Evaluate potential | Relationship between plasma and serum concentration or PK |
| relationships between | parameters of polatuzumab vedotin-related analytes and safety |
| drug exposure and the | endpoints. |
| efficacy and safety of | Evaluate pharmacokinetics for polatuzumab vedotin-related analytes |
| polatuzumab vedotin. | in patients with normal versus mild hepatic function impairment |
| Assess pharmacokinetics | based on National Cancer Institute (NCI) criteria for hepatic function |
| in patients with hepatic | impairment. |
| impairment. | Evaluate pharmacokinetics for polatuzumab vedotin-related analytes |
| Assess pharmacokinetics | in patients with normal versus mild or moderate renal function |
| in patients with renal | impairment based on the calculated creatinine clearance. |
| impairment. | |

Immunogenicity Objective

| | |
|---|---|
| Evaluate the immune | Incidence of anti-drug antibodies (ADAs) to polatuzumab vedotin |
| response to polatuzumab | during the study relative to the prevalence of ADAs to polatuzumab |
| vedotin. | vedotin at baseline. |

Exploratory Immunogenicity Objective

| | |
|---|---|
| Evaluate potential | Relationship between ADA status and efficacy, safety, or PK |
| effects of ADAs to | endpoints. |
| polatuzumab vedotin. | |

Exploratory Biomarker Objective

| | |
|---|---|
| Identify biomarkers that | PFS, EFS_{eff}, PFS24, CR rate, OS, DFS, DOR, and PRO endpoints by |
| are predictive of | exploratory biomarkers and molecular DLBCL prognostic subtypes |
| response to polatuzumab | such as cell of origin. |
| vedotin (i.e., predictive | Circulating tumor DNA (ctDNA) detectability in conjunction with |
| biomarkers), are | FDG-PET response. |
| associated with | ctDNA as a method of molecular disease detection. |
| progression to a more | ctDNA identification of emerging resistance. |
| severe disease state (i.e., | Association of biomarkers (including molecular and proteomic |
| prognostic biomarkers), | subtypes and genomic profiles at baseline) with efficacy and/or |
| are associated with | adverse events associated with R-CHOP and R-CHP + polatuzumab |
| acquired resistance to | vedotin treatment. |
| polatuzumab vedotin, are | |
| associated with | |
| susceptibility to | |
| developing adverse | |
| events, can provide | |
| evidence of polatuzumab | |
| vedotin activity, or can | |
| increase the knowledge | |
| and understanding of | |
| disease biology. | |

Exploratory Health Status Utility Objective

| | |
|---|---|
| Assess health status of | Health status based on EuroQol 5-Dimension, 5-Level questionnaire |
| patients treated with | (EQ-5D-5L). |
| polatuzumab vedotin | |
| with R-CHP compared | |
| with R-CHOP. | |

ªAll analyses based on the investigator's assessment unless otherwise specified, using the Lugano Response Criteria for Malignant Lymphoma. See, Table 2 and Cheson et al., J Clin Oncol (2014) 32: 1-9.
^{b}End of treatment defined as all planned chemoimmunotherapy treatment only; should any radiotherapy be administered, end of treatment tumor assessment occurred prior to initiating radiotherapy.
^{c}Younes et al., International Working Group consensus response evaluation criteria in lymphoma (RECIL 2017). Ann Oncol (2017) 28(7): 1436-1447.

### A. Lugano Response Criteria

An overview of the Lugano Response Criteria for Malignant Lymphoma (Cheson et al., J Clin Oncol (2014) 32:1-9) is provided in Table 2.

### Target and Non-Target Lesions

Up to six of the largest target nodes, nodal masses, or other lymphomatous lesions that are measurable in two diameters are identified from different body regions representative of the patient's overall disease burden and included mediastinal and retroperitoneal disease, if involved. At baseline, a measurable node is required to be greater than 15 mm in longest diameter (LDi). Measurable extranodal disease is permitted to be included in the six representative, measured lesions. At baseline, measurable extranodal lesions are required to be greater than 10 mm LDi. All other lesions (including nodal, extranodal, and assessable disease) are followed as non-measured disease, as non-target lesions (e.g. cutaneous, gastrointestinal [GI], bone, spleen, liver, kidneys, pleural or pericardial effusions, ascites, bone, or bone marrow).

### Split Lesions and Confluent Lesions

Lesions may split or may become confluent over time. In the case of split lesions, the individual product of the perpendicular diameters (PPDs) of the nodes are summed together to represent the PPD of the split lesion; this PPD is added to the sum of the PPDs of the remaining lesions to measure response. If subsequent growth of any or all of these discrete nodes occurs, the nadir of each individual node is used to determine progression. In the case of confluent lesions, the PPD of the confluent mass is compared with the sum of the PPDs of the individual nodes, with more than 50% increase in PPD of the confluent mass compared with the sum of individual nodes necessary to indicate progressive disease. The LDi and smallest diameter (SDi) are not needed to determine progression.

TABLE 2
Lugano Response Criteria for Malignant Lymphoma (Cheson et al., J Clin Oncol (2014) 32: 1-9).
Revised Criteria for Response Assessment

| Response and Site | PET-CT-Based Response | CT-Based Response |
|---|---|---|
| Complete | Complete metabolic response | Complete radiologic response (all of |
| | | the following). |
| Lymph nodes and | Score 1, 2, or 3ª with or without a | Target nodes/nodal masses must |
| extralymphatic | residual mass on 5PS^{b}. | regress to ≤1.5 cm in LDi. |
| sites | It is recognized that in Waldeyer's ring | No extralymphatic sites of disease. |
| | or extranodal sites with high | |
| | physiologic uptake or with activation | |
| | within spleen or marrow (e.g., with | |
| | chemotherapy or myeloid colony- | |
| | stimulating factors), uptake may be | |
| | greater than normal mediastinum | |
| | and/or liver. In this circumstance, | |
| | complete metabolic response may be | |
| | inferred if uptake at sites of initial | |
| | involvement is no greater than | |
| | surrounding normal tissue even if the | |
| | tissue has high physiologic uptake. | |
| Non-measured | Not applicable | Absent |
| lesion | | |
| Organ enlargement | Not applicable | Regress to normal |
| New lesions | None | None |
| Bone marrow | No evidence of FDG-avid disease in | Normal by morphology; if |
| | marrow. | indeterminate, IHC negative. |
| Partial | Partial metabolic response | Partial remission (all of the following). |
| Lymph nodes and | Score 4 or 5^{b} with reduced uptake | ≥50% decrease in SPD of up to 6 |
| extralymphatic | compared with baseline and residual | target measurable nodes and |
| sites | mass(es) of any size. | extranodal sites. |
| | At interim, these findings suggest | When a lesion is too small to measure |
| | responding disease. | on CT, assign 5 mm × 5 mm as the |
| | | default value. |
| | At end of treatment, these findings | When no longer visible, 0 x 0 mm. |
| | indicate residual disease. | For a node >5 mm x 5 mm, but |
| | | smaller than normal, use actual |
| | | measurement for calculation. |
| Non-measured | Not applicable | Absent/normal, regressed, but no |
| lesion | | increase. |
| Organ enlargement | Not applicable | Spleen must have regressed by >50% |
| | | in length beyond normal. |
| New lesions | None | None |
| Bone marrow | Residual uptake higher than uptake in | Not applicable |
| | normal marrow but reduced compared | |
| | with baseline (diffuse uptake | |
| | compatible with reactive changes from | |
| | chemotherapy allowed). If there are | |
| | persistent focal changes in the marrow | |
| | in the context of a nodal response, | |
| | consideration should be given to | |
| | further evaluation with MRI or biopsy | |
| | or an interval scan. | |
| No response or | No metabolic response | Stable disease |
| stable disease | | |
| Target nodes/nodal | Score 4 or 5^{b} with no significant | <50% decrease from baseline in SPD |
| masses, extranodal | change in FDG uptake from baseline at | of up to 6 dominant, measurable nodes |
| lesions | interim or end of treatment. | and extranodal sites; no criteria for |
| | | progressive disease are met. |
| Non-measured | Not applicable | No increase consistent with |
| lesion | | progression. |
| Organ enlargement | Not applicable | No increase consistent with |
| | | progression. |
| New lesions | None | None |
| Bone marrow | No change from baseline | Not applicable |
| Progressive disease | Progressive metabolic disease | Progressive disease requires at least 1 |
| | | of the following. |
| Individual target | Score 4 or 5^{b} with an increase in | PPD progression: |
| nodes/nodal masses | intensity of uptake from baseline, | |
| | and/or | |
| Extranodal lesions | New FDG-avid foci consistent with | An individual node/lesion must be |
| | lymphoma at interim or end-of- | abnormal with: |
| | treatment assessment. | LDi >1.5 cm and |
| | | Increase by ≥50% from PPD nadir and |
| | | An increase in LDi or SDi from nadir |
| | | 0.5 cm for lesions ≤2 cm |
| | | 1.0 cm for lesions >2 cm |
| | | In the setting of splenomegaly (>13 |
| | | cm), the splenic length must increase |
| | | by >50% of the extent of its prior |
| | | increase beyond baseline (e.g., a 15- |
| | | cm spleen must increase to >16 cm). |
| | | If no prior splenomegaly, must |
| | | increase by at least 2 cm from |
| | | baseline. |
| | | New or recurrent splenomegaly. |
| | | New or clear progression of |
| | | preexisting non-measured lesions. |
| New lesions | New FDG-avid foci consistent with | Regrowth of previously resolved |
| | lymphoma rather than another etiology | lesions. |
| | (e.g., infection, inflammation); if | A new node >1.5 cm in any axis. |
| | uncertain regarding etiology of new | A new extranodal site >1.0 cm in any |
| | lesions, biopsy or interval scan may be | axis; if <1.0 cm in any axis, its |
| | considered. | presence must be unequivocal and |
| | | must be attributable to lymphoma. |
| | | Assessable disease of any size |
| | | unequivocally attributable to |
| | | lymphoma. |
| Bone marrow | New or recurrent FDG-avid foci. | New or recurrent involvement. |

5PS = 5-point scale;
CT = computed tomography;
FDG = fluorodeoxyglucose;
IHC = immunohistochemistry;
LDi = longest transverse diameter of a lesion;
MRI = magnetic resonance imaging;
PET = positron emission tomography;
PPD = cross product of the LDi and perpendicular diameter;
SDi = shortest axis perpendicular to the LDi;
SPD = sum of the product of the perpendicular diameters for multiple lesions.
ªA score of 3 in many patients indicates a good prognosis with standard treatment, especially if at the time of an interim scan. However, in trials involving PET where de-escalation is investigated, it may be preferable to consider a score of 3 as inadequate response (to avoid under-treatment). Measured dominant lesions: Up to six of the largest dominant nodes, nodal masses, and extranodal lesions selected to be clearly measurable in two diameters. Nodes should preferably be from disparate regions of the body and should include, where applicable, mediastinal and retroperitoneal areas. Non-nodal lesions include those in solid organs (e.g., liver, spleen, kidneys, lungs), gastrointestinal involvement, cutaneous lesions, or those noted on palpation. Non-measured lesions: Any disease not selected as measured; dominant disease and truly assessable disease should be considered not measured. These sites include any nodes, nodal masses, and extranodal sites not selected as dominant or measurable or that do not meet the requirements for measurability but are still considered abnormal, as well as truly assessable disease, which is any site of suspected disease that would be difficult to follow quantitatively with measurement, including pleural effusions, ascites, bone lesions, leptomeningeal disease, abdominal masses, and other lesions that cannot be confirmed and followed by imaging. In Waldeyer's ring or in extranodal sites (e.g., GI tract, liver, bone marrow), FDG uptake may be greater than in the mediastinum with complete metabolic response, but should be no higher than surrounding normal physiologic uptake (e.g., with marrow activation as a result of chemotherapy or myeloid growth factors).
^{b}PET 5PS: 1 = no uptake above background; 2 = uptake ≤ mediastinum; 3 = uptake > mediastinum but ≤ liver; 4 = uptake moderately > liver; 5 = uptake markedly higher than liver and/or new lesions; X = new areas of uptake unlikely to be related to lymphoma.

### II. Study Design

This study is a Phase III, multicenter, randomized, double-blind, placebo-controlled trial comparing the efficacy and safety of polatuzumab vedotin plus R-CHP versus R-CHOP in patients with previously untreated CD20-positive DLBCL with IPI 2-5.

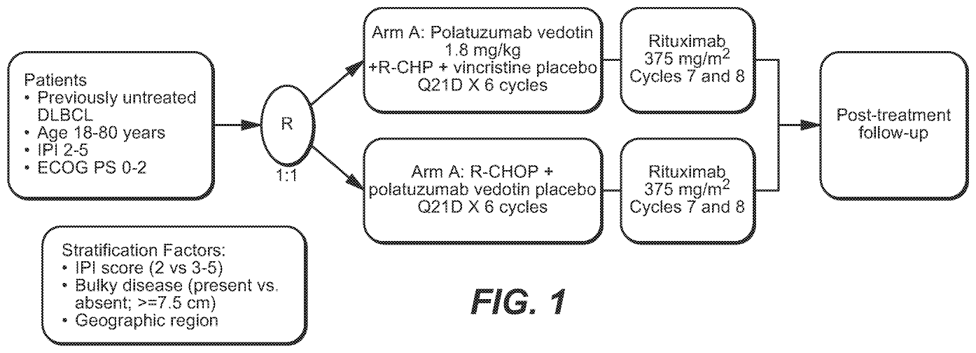

Patients receive six cycles of polatuzumab vedotin plus R-CHP or standard R-CHOP chemotherapy at 21-day intervals. Both arms then receive two additional cycles of single agent rituximab. A study schematic is shown in FIG. 1.

Patients are randomized in a 1:1 ratio to either Arm A or Arm B, as defined below:
Arm A, R-CHP+polatuzumab vedotin; polatuzumab vedotin 1.8 mg/kg intravenously (IV), placebo for vincristine IV, rituximab 375 mg/m² IV, cyclophosphamide 750 mg/m² IV, and doxorubicin 50 mg/m² IV each given on Day 1, and prednisone 100 mg/day orally (PO) given on Days 1-5 of every 21-day cycle for 6 cycles. Rituximab 375 mg/m² IV given as monotherapy in Cycles 7 and 8.
Arm B, R-CHOP: placebo for polatuzumab vedotin, rituximab 375 mg/m² IV, cyclophosphamide 750 mg/m² IV, doxorubicin 50 mg/m² IV, and vincristine 1.4 mg/m² IV (maximum 2 mg/dose) each given on Day 1, and prednisone 100 mg/day PO given on Days 1-5 of every 21-day cycle for 6 cycles. Rituximab 375 mg/m² IV given as monotherapy in Cycles 7 and 8.

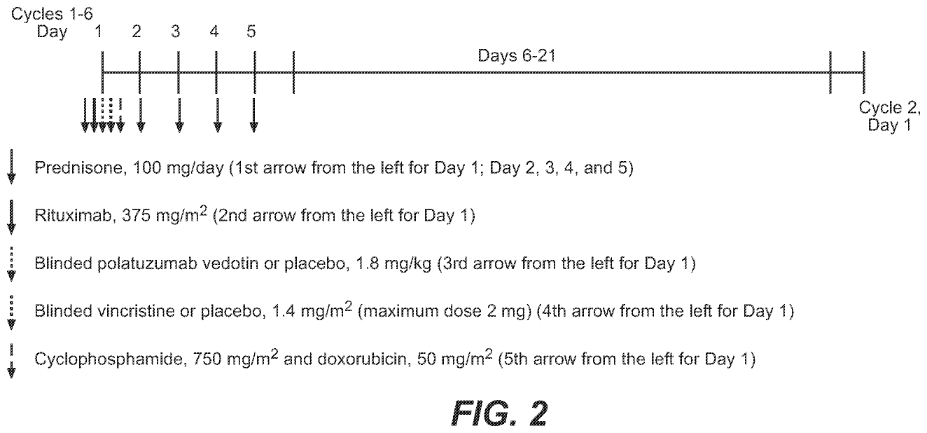

An overview of the treatment regimens in this study is provided in FIG. 2.

### A. Randomization

During randomization, permuted blocks are employed using the following stratification factors:
IPI score (IPI 2 versus IPI 3-5)
Bulky disease, defined as one lesion ≥7.5 cm (present versus absent)
Geographical region

Patients are assessed for disease response by the investigator using regular clinical and laboratory examinations, fluorodeoxyglucose positron emission tomography (FDG-PET, also referred to as PET-CT), and dedicated computed tomography (CT) scans (magnetic resonance imaging [MRI] scans are performed if CT scans with contrast are contraindicated in the patient), according to the Lugano Response Criteria for Malignant Lymphoma. See, Table 2. PET-CT and dedicated CT scans are obtained at screening and 6-8 weeks after completion of study treatment.

Responses are evaluated at the end of study treatment, or sooner in the event that a patient discontinues early.

Safety is evaluated by monitoring all adverse events, serious adverse events, and abnormalities identified through physical examinations, vital signs, and laboratory assessments. Such events are graded using the National Cancer Institute Common Terminology Criteria for Adverse Events, Version 4.0 (NCI CTCAE v4.0). Laboratory safety assessments include routine monitoring of hematology and blood chemistry, and tests of immunologic parameters.

Study treatment begins within 7 days of randomization, unless otherwise approved by a Medical Monitor.

### B. Study Treatment Administration

In Cycles 1-6, rituximab infusion is completed prior to starting any other agent administered by infusion (i.e., blinded polatuzumab vedotin/placebo; blinded vincristine/placebo, doxorubicin, and cyclophosphamide). The order of administration for Cycles 1-6 is: first prednisone, second rituximab, and third blinded polatuzumab vedotin/placebo. Subsequent infusions of blinded vincristine/placebo, cyclophosphamide, and doxorubicin are administered according to institutional preference. Cycle 7 and Cycle 8 consist of rituximab as monotherapy.

### Polatuzumab Vedotin and Placebo

The initial dose of polatuzumab vedotin is administered to patients who are well hydrated over 90 (+/−10) minutes. Premedication (e.g., 500-1000 mg of oral acetaminophen or paracetamol and 50-100 mg diphenhydramine) is permitted to be administered before administration of polatuzumab vedotin/placebo (which may have already been administered as a premedication for rituximab). If infusion-related reactions (IRRs) are observed with the first infusion of polatuzumab vedotin in the absence of premedication, premedication is administered before subsequent doses as described below. The polatuzumab vedotin/placebo infusion is slowed or interrupted for patients who experienced infusion-associated symptoms. If prior infusions are well tolerated, subsequent doses of polatuzumab vedotin are administered over 30 (+/−10) minutes, followed by a 30-minute observation period after the infusion. Dose modifications for polatuzumab vedotin/placebo are described in Table 3.

TABLE 3
Steps of Dose Reduction for Blinded Polatuzumab
Vedotin/Placebo and Blinded Vincristine/Placebo.

| | Blinded Polatuzumab | Blinded Vincristine |
|---|---|---|
| Dose Level | Vedotin or Placeboª | or Placeboª |
| Starting dose | 1.8 mg/kg per cycle | 100% of starting |
| | | dose per cycle |
| First dose reduction | 1.4 mg/kg per cycle | 75% of starting |
| | | dose per cycle |
| Second dose reduction | 1.0 mg/kg per cycle | 50% of starting |
| | | dose per cycle |
| Third dose reduction | Discontinue drug | Discontinue drug |

ªPlacebo contains no active medicinal product but due to the blinded nature of the study, dosing ofplacebo is modified per protocol guidelines.

### Vincristine and Placebo

The dose of vincristine for each patient is 1.4 mg/m² (maximum dose 2 mg). Vincristine is given on Day 1 of Cycles 1-6, e.g., as an IV infusion via minibag over approximately 10-30 minutes through a dedicated line. Dose modifications for vincristine/placebo are described in Table 3 (above).

### Rituximab

Rituximab is administered by IV infusion at the dose of 375 mg/m² on Day 1 of each cycle. No dose modifications of rituximab are allowed. Rituximab is administered after prednisone dosing, and before the cyclophosphamide, doxorubicin, polatuzumab vedotin/placebo, and vincristine/placebo infusions. Once the rituximab infusion is completed, patients are observed for 30 minutes before the start of the other infusions.

The infusion of rituximab is permitted to be split over multiple days, e.g., 2 days, if the patient is at increased risk for an IRR (high tumor burden or high peripheral lymphocyte count). For patients who experience an adverse event during a rituximab infusion, administration of rituximab, cyclophosphamide, doxorubicin, polatuzumab vedotin/placebo, and vincristine/placebo are continued on the following day if required. If a dose of rituximab is split over multiple days, all infusions occur with appropriate premedication (including prednisone) and at the first infusion rate. All initial rituximab infusions are administered to patients after premedication with oral acetaminophen (e.g., 650-1000 mg) and an antihistamine such as diphenhydramine hydrochloride (50-100 mg)≥30 minutes before starting each infusion (unless contraindicated). For patients who do not experience infusion-related symptoms with their previous infusion, premedication at subsequent infusions is permitted to be omitted at the investigator's discretion.

Blinded polatuzumab vedotin/placebo is permitted to be administered on Day 2 per investigator preference due to infusion times for rituximab and blinded polatuzumab vedotin/placebo. In this instance, blinded vincristine/placebo, cyclophosphamide, and doxorubicin are also permitted to be administered on Day 1 following the completion of rituximab, and blinded polatuzumab vedotin/placebo is permitted to be administered on Day 2 after prednisone.

Rituximab infusions are administered according to the instructions outlined in Table 4. If a patient tolerates the first cycle of study treatment without significant infusion reactions, rituximab is permitted to be administered as a rapid infusion (e.g., over 60-90 minutes).

TABLE 4
Administration of First and Subsequent Infusions of Rituximab.

| First Infusion (Day 1) | Subsequent Infusions |
|---|---|
| Begin infusion at an initial rate of 50 | If the patient experiences an infusion-related |
| mg/hr. | or hypersensitivity reaction during the prior |
| If no infusion-related or | infusion, begin infusion at an initial rate of |
| hypersensitivity reaction occurs, | 50 mg/hr and follow instructions for the first infusion. |
| increase the infusion rate in 50-mg/hr | If the patient tolerates the prior infusion well (defined as an |
| increments every 30 minutes, to a | absence of Grade 2 reactions during a final infusion rate |
| maximum of 400 mg/hr. | of ≥100 mg/hour), begin the infusion at a rate of 100 mg/hour. |
| If an infusion reaction develops, stop or | If no infusion reaction occurs, increase the |
| slow the infusion. Administer infusion- | infusion rate in 100-mg/hr increments every |
| reaction medications and supportive | 30 minutes, to a maximum of 400 mg/hr. |
| care in accordance with institutional | If an infusion reaction develops, stop or slow the infusion. |
| guidelines. If the reaction resolves, | Administer infusion-reaction medications and supportive |
| resume the infusion at a 50% reduction | care in accordance with institutional guidelines. |
| in rate (i.e., 50% of rate being used at | If the reaction resolves, resume the infusion at a 50% |
| the time that the reaction occurred). | reduction in rate (i.e., 50% of rate being used at the time that |
| | the reaction occurred). |

Cyclophosphamide. Doxorubicin, and Prednisone

CHP chemotherapy consists of cyclophosphamide and doxorubicin administered via IV and oral prednisone. Doxorubicin and cyclophosphamide are administered after both rituximab and polatuzumab vedotin/placebo infusions unless otherwise indicated.

The dosages are based on the standard CHP doses:
Cyclophosphamide: 750 mg/m² administered IV on Day 1 of Cycles 1-6.
Doxorubicin: 50 mg/m² administered IV on Day 1 of Cycles 1-6.
Prednisone: 100 mg/day PO on Days 1-5 of Cycles 1-6.

Prednisone is permitted to be replaced with prednisolone (100 mg/day) or IV methylprednisolone (80 mg/day). Hydrocortisone is not permitted to be used as a substitute.

Patients who require lymphoma symptom control during screening are allowed to receive steroids in the following manner, which is not considered part of study treatment:
Up to 30 mg/day of prednisone or equivalent for lymphoma symptom control during screening, including prior to imaging assessments or staging (not included as part of pre-phase treatment).
If glucocorticoid treatment is urgently required at higher doses for lymphoma symptom control prior to the start of study treatment, prednisone >30 mg-100 mg/day or equivalent is permitted to be given for a maximum of 7 days as a pre-phase treatment; tumor assessments are completed prior to steroid treatment at >30 mg/day of prednisone or equivalent. Vincristine is not administered as part of the pre-phase treatment.

If CHP is started later than Day 1 of the cycle, then planned Day 1 of the next cycle is calculated from the day when CHP is actually initiated, in order to maintain the regular chemotherapy interval of 21 days.

### C. Premedication

Patients receive premedication as outlined in Table 5.

TABLE 5
Premedication for Rituximab and Blinded Polatuzumab Vedotin/Placebo.

| | Patients Who Require | | |
|---|---|---|---|
| Timepoint | Premedication | Premedication | Administration |
| Cycle 1, Day 1 | All patients | Corticosteroidª | Complete ≥1 hour prior to |
| | | | rituximab infusion and |
| | | | polatuzumab vedotin/placebo. |
| | | Antihistamine | Administer ≥30 minutes |
| | | drug^{b} | prior to rituximab infusion; |
| | | Analgesic/ | may be administered to |
| | | anti-pyretic^{c} | patients prior to |
| | | | administration of any |
| | | | polatuzumab vedotin/placebo |
| | | | as well. |
| Cycles 2 and | Patients with no IRR | Corticosteroidª | Complete ≥1 hour prior to |
| beyond, | during the previous | | rituximab and polatuzumab |
| Day 1 | infusion | | vedotin/placebo infusion. |
| | | Antihistamine | Administer ≥30 minutes |
| | | drug^{b} | prior to infusion. These may |
| | | Analgesic/ | be omitted or adapted at the |
| | | anti-pyretic^{c} | investigator's discretion. |
| | Patients with Grade 1 | Corticosteroidª | Complete ≥1 hour prior to |
| | or 2 IRR during the | | rituximab and polatuzumab |
| | previous infusion. | | vedotin/placebo infusion. |
| | | Antihistamine | Administer ≥30 minutes |
| | | drug^{b} | prior to rituximab and/or |
| | | Analgesic/ | polatuzumab vedotin/placebo |
| | | anti-pyretic^{c} | infusion. |
| | Patients with Grade 3 | Corticosteroidª | Complete ≥1 hour prior to |
| | IRR, wheezing, | | rituximab and/or |
| | urticaria, or other | | polatuzumab vedotin/placebo |
| | symptoms of | | infusion. |
| | anaphylaxis during | Antihistamine | Administer ≥30 minutes |
| | the previous infusion. | drug^{b} | prior to rituximab and/or |
| | Patients with bulky | Analgesic/ | polatuzumab vedotin/placebo |
| | disease. | anti-pyretic^{c} | infusion. |

IRR = infusion-related reaction.
^{a}Part of study treatment: 100 mg of prednisone. May be substituted with 100 mg of prednisolone or 80 mg of methylprednisolone. Hydrocortisone should not be used, as it has not been effective in reducing rates of IRRs. In Cycle 7 and Cycle 8, corticosteroid used as premedication is administered according to institutional standard.
^{b}For example, 50-100 mg of diphenhydramine.
^{c}For example, 650-1000 mg of acetaminophen/paracetamol.

Pre-phase treatment: Administration of up to 7 days of pre-phase treatment (e.g., up to 100 mg PO daily of prednisone/prednisolone or equivalent) prior to day 1 of cycle 1 is given at the discretion of the treating physician.

### D. Concomitant Therapy

Concomitant therapy consists of any medication (e.g., prescription drugs, over-the-counter drugs, vaccines, herbal or homeopathic remedies, nutritional supplements) used by a patient in addition to protocol-mandated treatment from 7 days prior to initiation of study drug to the study completion/discontinuation visit.

### Permitted Therapies

Patients are permitted to use oral contraceptives and hormone-replacement therapy during the study. Premedication with antihistamines, antipyretics, and/or analgesics is administered at the discretion of the investigator. Other than the prednisone given as study treatment and prednisone given as pre-phase treatment at the discretion of the treating investigator physician, corticosteroids are used only for the treatment of conditions other than lymphoma (e.g., asthma). Patients who experience infusion-associated symptoms are treated symptomatically with acetaminophen, ibuprofen, diphenhydramine, and/or H₂-receptor antagonists (e.g., famotidine, cimetidine), or equivalent medications per local standard practice. Serious infusion-associated events manifested by dyspnea, hypotension, wheezing, bronchospasm, tachycardia, reduced oxygen saturation, or respiratory distress are managed with supportive therapies as clinically indicated (e.g., supplemental oxygen and β₂-adrenergic agonists).

CNS Prophylaxis: CNS prophylaxis with intrathecal chemotherapy is given according to institutional practice. CNS prophylaxis using high-dose IV methotrexate (e.g., 1 g/m² per cycle) is not permitted and is considered a new anti-lymphoma therapy.

Prophylaxis for Hemorrhagic Cystitis: Patients are adequately hydrated before and after cyclophosphamide administration and are instructed to void frequently. Mesna is used as prophylaxis according to institutional practice.

Treatment and Prophylaxis of Neutropenia: Granulocyte colony-stimulating factor (G-CSF) is required as primary prophylaxis in each cycle of therapy during Cycles 1-6, typically starting 1 to 3 days after administration of myelotoxic chemotherapy (doxorubicin, cyclophosphamide, and polatuzumab vedotin). Dosing of G-CSF follows institutional standards or at the investigator's discretion. An example of appropriate G-CSF dosing for prophylaxis is the American Society of Clinical Oncology (ASCO) recommended guidelines (Smith et al., J Clin Oncol (2015) 33:3199-212). For patients who develop neutropenia despite prophylaxis, G-CSF is not routinely recommended for the treatment of uncomplicated neutropenia. However, G-CSF is considered in patients with fever and neutropenia who are at high risk for infection-associated complications or who have prognostic factors predictive of poor clinical outcomes (Smith et al., J Clin Oncol (2015) 33:3199-212).

Premedication before Rituximab: All rituximab infusions are administered to patients after premedication as described in Table 5.

Infection Prophylaxis: Anti-infective prophylaxis for viral, fungal, bacterial, or Pneumocystis infections is permitted and instituted per institutional practice or investigator preference based on individual patient risk factors. Prophylactic anti-viral medications for hepatitis B reactivation are administered, e.g., as described in Flowers et al., J Clin Oncol (2013) 31:794-810; National Comprehensive Cancer Network®. NCCN clinical practice guidelines in oncology (NCCN Guidelines®); Prevention and treatment of cancer-related infections, version 2 [resource on the Internet]. 2017 [cited 9 Jun. 2017]. Available from: www[dot]nccn[dot]org/professionals/physician_gls/f_guidelines.asp; and Reddy et al., Gastroenterology (2015) 148:215-19.

Pre-Planned Radiotherapy: Pre-planned radiotherapy (i.e., radiation that is planned before randomization to be given at the end of study treatment) is permitted to be administered to initial sites of bulky or extranodal disease according to institutional practice. If indicated, pre-planned radiotherapy is initiated within 8 weeks after the last study drug treatment and after all end of treatment assessments, including PET-CT scans for disease response assessment, are completed. All unplanned radiotherapy administered to patients are considered a new anti-lymphoma treatment.

### Prohibited Therapies

Treatment with other concomitant anti-tumor agents not defined as study treatment, radiotherapy, or other concurrent investigational agents of any type resulted in withdrawal of patients from study treatment.

Use of the following therapies is prohibited during the study:
Cytotoxic therapies (other than intrathecal CNS prophylaxis and study treatment) intended for the treatment of lymphoma whether EMA- or U.S. FDA-approved or experimental.
Immunotherapy or immunosuppressive therapy other than study treatments used for the intention of treating lymphoma (e.g., for an adverse event).
Any unplanned radiotherapy.
Hormone therapy other than contraceptives, hormone-replacement therapy, or megestrol acetate.
Biologic agents used for the intention of treating lymphoma other than clinically indicated hematopoietic growth factors.
Pre-phase therapy, other than the use of prednisone as described herein.

Immunizations: Patients are not permitted to receive either primary or booster vaccination with live virus vaccines at any time during study treatment.

Cautionary Therapy: The use of any calcium channel entry blockers given concomitantly with an anthracycline drug may potentially increase the risk of cardiac toxicity associated with anthracycline administration. It is recommended that calcium channel blockers be avoided within 30 days of the administration of an anthracycline drug when possible and clinically appropriate.

Medications Given with Precaution due to Effects Related to Cytochrome P450 Enzymes and P-glycoprotein: In vitro data suggest that unconjugated MMAE is mainly metabolized by CYP3A4 and, to a lesser extent, by CYP2D6. Based on a validated physiological-based PK model simulation, strong CYP3A4 inhibitors may increase the exposure (e.g., area under the concentration-time curve) of unconjugated MMAE by ˜50% while acMMAE PK is not affected. Concomitant medications that are strong CYP3A4 inhibitors are considered cautionary as they may potentially lead to adverse reactions, which require close monitoring. MMAE is a P-glycoprotein (P-gp) substrate but not a P-gp inhibitor. Concomitant medications that are P-gp inhibitors are considered cautionary as they may potentially lead to adverse reactions, which require close monitoring.

### III. Study Participants

This study includes patients with previously untreated DLBCL.

### A. Inclusion Criteria

Patients who meet the following criteria are included in this study:
Previously untreated patients with CD20-positive DLBCL, including one of the following diagnoses by the 2016 World Health Organization (WHO) classification of lymphoid neoplasms:
   DLBCL, not otherwise specified (NOS), including germinal center B-cell type, activated B-cell type.
   T-cell/histiocyte-rich large B-cell lymphoma.
   Epstein-Barr virus-positive DLBCL, NOS.
   ALK-positive large B-cell lymphoma.
   HHV8-positive DLBCL, NOS.
   High-grade B-cell lymphoma with MYC and BCL2 and/or BCL6 rearrangements (double-hit or triple-hit lymphoma).
   High-grade B-cell lymphoma, NOS.
International Prognostic Index (IPI) score of 2-5.
Age 18-80 years.
Eastern Cooperative Oncology Group (ECOG) Performance Status of 0, 1, or 2.
Life expectancy ≥12 months.
At least one bi-dimensionally measurable lesion, defined as >1.5 cm in its longest dimension as measured by CT or MRI.
Left ventricular ejection fraction (LVEF)≥50% on cardiac multiple-gated acquisition (MUGA) scan or cardiac echocardiogram (ECHO).
Adequate hematologic function (unless due to underlying disease, as established for example, by extensive bone marrow involvement or due to hypersplenism secondary to the involvement of the spleen by DLBCL per the investigator), defined as follows:
   Hemoglobin ≥9.0 g/dL without packed red blood cell (RBC) transfusion during 14 days before first treatment.
   Absolute neutrophil count (ANC)≥1,000/μL.
   Platelet count ≥75,000/μL.

### B. Exclusion Criteria

Patients who meet any of the following criteria are not included in this study:
Contraindication to any of the individual components of R-CHOP, including prior receipt of anthracyclines, or history of severe allergic or anaphylactic reactions to humanized or murine monoclonal antibodies, or known sensitivity or allergy to murine products.
Prior organ transplantation.
Current Grade >1 peripheral neuropathy by clinical examination or demyelinating form of Charcot-Marie-Tooth disease.
History of indolent lymphoma.
Current diagnosis of the following: follicular lymphoma grade 3B: B-cell lymphoma, unclassifiable, with features intermediate between DLBCL and classical Hodgkin lymphoma (grey-zone lymphoma): primary mediastinal (thymic) large B-cell lymphoma; Burkitt lymphoma; central nervous system (CNS) lymphoma (primary or secondary involvement), primary effusion DLBCL, and primary cutaneous DLBCL.
Prior treatment with cytotoxic drugs within 5 years of screening for any condition (e.g., cancer, rheumatoid arthritis) or prior use of any anti-CD20 antibody.
Prior use of any monoclonal antibody within 3 months of the start of Cycle 1: any investigational therapy within 28 days prior to the start of Cycle 1; vaccination with live vaccines within 28 days prior the start of Cycle 1.
Prior radiotherapy to the mediastinal/pericardial region.
Prior therapy for DLBCL (corticosteroids addressed below).
Corticosteroid use >30 mg/day of prednisone or equivalent, for purposes other than lymphoma symptom control.
   Patients receiving corticosteroid treatment with ≤30 mg/day of prednisone or equivalent for reasons other than lymphoma symptom control (e.g., rheumatoid arthritis) are required to be on a stable dose of at least 4 weeks' duration prior to the start of Cycle 1.
   Patients who require lymphoma symptom control during screening are permitted to receive steroids in the following manner: up to 30 mg/day of prednisone or equivalent used for lymphoma symptom control during screening, including prior to finalization of staging (not included as part of pre-phase treatment). If glucocorticoid treatment is urgently required at higher doses for lymphoma symptom control prior to the start of study treatment, tumor assessments are completed prior to initiation of >30-100 mg/day of prednisone or equivalent. Prednisone >30-100 mg/day or equivalent is permitted for a maximum of 7 days as a pre-phase treatment. As part of the pre-phase treatment, vincristine is not administered.
History of other malignancy that could affect compliance with the protocol or interpretation of results.
   Patients with a history of curatively treated basal or squamous cell carcinoma or melanoma of the skin or in situ carcinoma of the cervix at any time prior to the study are eligible.
   Patients with any malignancy appropriately treated with curative intent and the malignancy is in remission without treatment for ≥2 years prior to enrollment are eligible.
   Patients with low-grade, early-stage prostate cancer (Gleason score 6 or below, Stage 1 or 2) with no requirement for therapy at any time prior to study are eligible.
Evidence of significant, uncontrolled, concomitant diseases that could affect compliance with the protocol or interpretation of results, including significant cardiovascular disease (such as New York Heart Association Class III or IV cardiac disease, myocardial infarction within the last 6 months, unstable arrhythmias, or unstable angina) or pulmonary disease (including obstructive pulmonary disease and history of bronchospasm).
Recent major surgery (within 4 weeks prior to the start of Cycle 1), other than for diagnosis.
History or presence of an abnormal electrocardiogram (ECG) that is clinically significant, including complete left bundle branch block, second- or third-degree heart block, or evidence of prior myocardial infarction.
Known active bacterial, viral, fungal, mycobacterial, parasitic, or other infection (excluding fungal infections of nail beds) at study enrollment or significant infections within 2 weeks before the start of Cycle 1.
Clinically significant liver disease, including active viral or other hepatitis, current alcohol abuse, or cirrhosis.
Illicit drug or alcohol abuse within 12 months prior to screening.
Any of the following abnormal laboratory values (unless any of these abnormalities are due to underlying lymphoma):
   International normalized ratio (INR) or prothrombin time (PT)>1.5× upper limit of normal (ULN) in the absence of therapeutic anticoagulation.
   Partial thromboplastin time (PTT) or activated PTT (aPTT)>1.5×ULN in the absence of a lupus anticoagulant.
   Serum aspartate aminotransferase (AST) and alanine aminotransferase (ALT)≥2.5×ULN
   Total bilirubin ≥1.5×ULN; patients with documented Gilbert disease are enrolled if total bilirubin is ≥3.0×ULN.
   Serum creatinine clearance <40 mL/min (using Cockcroft-Gault formula).
Patients with suspected active or latent tuberculosis (as confirmed by a positive interferon gamma release assay); positive test results for chronic hepatitis B infection (defined as positive hepatitis B surface antigen [HBsAg] serology (patients with occult or prior hepatitis B infection, defined as positive total hepatitis B core antibody and negative HBsAg, are included if hepatitis B virus (HBV) DNA is undetectable at the time of screening, and patients are willing to undergo monthly DNA testing and appropriate anti-viral therapy as indicated); positive test results for hepatitis C (hepatitis C virus [HCV] antibody serology testing; patients positive for HCV antibody are eligible only if polymerase chain reaction (PCR) is negative for HCV RNA); known history of HIV seropositive status: positive results for the human T-lymphotrophic 1 virus (HTLV-1)
Patients with a history of progressive multifocal leukoencephalopathy.

### IV. Study Assessments

Medical history is recorded, including B-symptoms (i.e., weight loss, night sweats, or fever), clinically significant diseases, surgeries, cancer history (including prior cancer therapies and procedures), reproductive status, and all medications (e.g., prescription drugs, over-the-counter drugs, herbal or homeopathic remedies, nutritional supplements) used by the patient within 7 days prior to initiation of study treatment. At the time of each follow-up physical examination, an interval medical history is obtained and recorded. Demographic data include age, sex, and self-reported race/ethnicity.

A complete physical examination, performed at screening and other visits, includes an evaluation of the head, eyes, ears, nose, and throat, and the cardiovascular, dermatological, musculoskeletal, respiratory, gastrointestinal, genitourinary, and neurological systems. As part of the complete physical examination, the presence and degree of enlarged lymph nodes, hepatomegaly, and splenomegaly are recorded. A targeted physical examination limited to systems associated with symptoms is performed at post baseline visits and as clinically indicated. As part of tumor assessment, targeted physical examination also include the evaluation of the presence and degree of enlarged lymph nodes, hepatomegaly, splenomegaly, or other findings of concern for lymphoma. Clinical assessments of peripheral neuropathy are performed at screening, at Day 1 of each cycle, and at the treatment completion visit. These are permitted to be performed within 48 hours prior to the study visit date. New or worsened clinically significant abnormalities are recorded as adverse events.

Vital signs include systolic and diastolic blood pressure, pulse rate, respiratory rate, oxygen saturation as measured by pulse oximetry, and body temperature. Weight, height, and BSA are recorded. During rituximab administration visits, vital signs are obtained prior to the start of the infusion of rituximab as well as at the end of the rituximab infusion. During the administration of polatuzumab vedotin, vital signs are assessed before the start of the infusion, every 15 (+/−5) minutes during the infusion, at the end of the infusion, and every 30 (+/−10) minutes for 90 minutes following completion of dosing at Cycle 1 and 30 (+/−10) minutes following completion of dosing in subsequent cycles. Additional monitoring of vital signs is performed as clinically indicated.

### A. Tumor and Response Evaluations

All evaluable or measurable disease is documented at screening and re-assessed at each subsequent tumor evaluation. Response assessments are assessed by the investigator, on the basis of physical examinations, diagnostic CT scans (or MRI scans), PET-CT scans, and bone marrow examinations, through use of the Lugano Response Criteria for Malignant Lymphoma (Table 2). The same criteria are used to evaluate CR at treatment completion by PET-CT as assessed by BICR.

### Radiographic Assessments

Diagnostic contrast enhanced CT scans are currently the best available and most reproducible methods for measuring target lesions selected for response assessment: conventional CT scans (MRI scans if CT is contraindicated) are performed with contiguous cuts of less than or equal to 8 mm in slice thickness and contrast enhanced if not medically contraindicated. In patients for whom contrast is contraindicated (e.g., patients with contrast allergy or impaired renal clearance), CT or combined PET-CT scans without contrast or MRI scans are permitted so long as they permit consistent and precise measurement of target lesions during the study treatment period.

### Bone Marrow Assessments

Bone marrow examinations are required at screening, and include biopsy for morphology. Bone marrow assessments are performed prior to pre-phase steroids unless the result does not impact stratification (i.e., determination of IPI 2 versus IPI 3-5).

### Laboratory, Biomarker, and Other Biological Samples

Samples for the following tests are obtained and analyzed:
Hematology: hemoglobin, platelet count, white blood cell (WBC) count, and ANC; WBC differential is performed if clinically indicated.
Chemistry panel: glucose (fasting or random), sodium, potassium, chloride, bicarbonate (or CO₂), urea nitrogen (or urea), creatinine, calcium, phosphorous, total bilirubin, direct bilirubin (if total bilirubin level is abnormal), total protein, albumin, ALT, AST, lactate dehydrogenase (LDH), alkaline phosphatase (ALP), amylase, lipase, uric acid, 25-hydroxyvitamin D (either total or fractionated 25-hydroxyvitamin D3 are acceptable) and hemoglobin Alc.
Coagulation: aPTT or PTT, and PT or INR.
Viral serology and detection: Hepatitis B (HBsAg and hepatitis B core antibody); HBV DNA; HCV antibody; HCV RNA by PCR if the patient is HCV-antibody positive.
Quantitative immunoglobulins: IgG, IgA, and IgM
CSF assessment, as clinical indicated, for detection of CNS lymphoma.
Blood samples for PK analysis of polatuzumab vedotin: Serum polatuzumab vedotin total antibody (which includes all drug-to-antibody ratio [DAR] species, including DAR 0 and DAR≥ 1), plasma polatuzumab vedotin conjugate (evaluated as acMMAE), and plasma unconjugated MMAE concentrations are quantified with the use of validated methods.
Serum samples for anti-polatuzumab vedotin antibody: A validated antibody-bridging ELISA is used to screen for and confirm the presence of anti-polatuzumab vedotin antibodies in patient samples as well as to characterize and determine the titer of confirmed ADA-positive samples.
Peripheral blood flow cytometry (T cells, B cells, natural killer cells).
Analysis of biomarker testing, gene expression, genetic mutations, and epigenetic assessment of blood and tissue samples.
A peripheral blood sample for minimal residual disease (MRD) analysis is required for all patients at Cycle 1, Day 1: Cycle 2, Day 1: between Cycle 4, Day 15 and Cycle 5, Day 1; and at the treatment completion visit. A peripheral blood sample for MRD analysis is also required at the following post-treatment follow-up visits: 6 months, 12 months, 18 months, and 24 months.
Archival or newly collected tumor tissue samples obtained at baseline for pathology review determination of molecular profiling including, but not limited to, gene expression profiling, expression of oncogenic proteins, and biomarkers.
Tumor tissue samples and the corresponding pathology report, for retrospective review of the diagnosis of DLBCL and for biomarkers. The specimen is required to contain adequate evaluable tumor cells (e.g., at least 20% for excisional biopsy and at least 50% for core biopsy).

### B. Pharmacokinetic Analyses

The PK population includes all patients who have at least one evaluable PK sample post-dose for at least one analyte. Individual and mean serum concentrations of total polatuzumab vedotin antibody (fully conjugated, partially deconjugated and fully deconjugated antibody), plasma concentrations of polatuzumab vedotin conjugate (evaluated as acMMAE), and unconjugated MMAE versus time data are tabulated and plotted. The pharmacokinetics of the above analytes are summarized by estimating selected PK parameters. The population PK analysis investigates the effects of certain covariates on the pharmacokinetics of polatuzumab vedotin-related analytes, including renal and hepatic impairment. Exposure-response (safety and efficacy) analyses are conducted using plasma/serum concentrations or relevant PK parameters and available drug effect data (e.g., CR rate, PFS, and/or toxicity data). To assess potential PK drug interactions, PK parameters for each analyte of polatuzumab vedotin are compared with historical data.

### C. Electrocardiograms and Echocardiogram or MUGA

Single 12-lead ECG recordings are obtained at screening, at the early treatment termination/study treatment completion visits, and at unscheduled timepoints as indicated. For assessment of cardiac function (LVEF), an echocardiogram (ECHO) or multiple-gated acquisition (MUGA) scan is also obtained at screening and early treatment termination/study treatment completion visits, and as clinically indicated. During infusion of doxorubicin, ECG monitoring is performed per clinical practice.

### D. Patient-Reported Outcomes (PRO)

PRO data are collected to document the treatment benefit and more fully characterize the safety profile of polatuzumab vedotin. PRO data are obtained through use of the following instruments: European Organisation for Research and Treatment of Cancer Quality of Life-Core 30 (EORTC QLQ-C30) questionnaire, Functional Assessment of Cancer Therapy-Lymphoma Lymphoma Subscale (FACT-Lym LymS), Functional Assessment of Cancer Treatment/Gynecologic Oncology Group-Neurotoxicity (FACT/GOG-NTX), and EuroQol 5-Dimension, 5-Level (EQ-5D-5L) questionnaire.

The EORTC QLQ-C30, FACT-Lym LymS, FACT/GOG-NTX, and EQ-5D-5L are administered at Cycle 1, Day 1 (baseline): Cycle 2, Day 1: Cycle 3, Day 1; and Cycle 5, Day 1. The FACT/GOG-NTX is administered at baseline and Day 1 of every cycle. Patients complete all PRO measures (EORTC QLQ-C30, FACT-Lym LymS, FACT/GOG-NTX, and EQ-5D-5L) at treatment discontinuation and at planned post-treatment visits thereafter until the close of the study.

### EORTC QLQ-C30

The EORTC QLQ-C30 is a validated, reliable self-report measure (Aaronson et al., J Natl Cancer Inst (1993) 85:365-76; Fitzsimmons et al., Eur J Cancer (1999) 35:939-41). It consists of 30 questions that assess five aspects of patient functioning (physical, emotional, role, cognitive, and social), three symptom scales (fatigue, nausea and vomiting, and pain), global health/QOL, and six single items (dyspnea, insomnia, appetite loss, constipation, diarrhea, and financial difficulties) with a recall period of the previous week. Scale scores can be obtained for the multi-item scales. The first 28 items are scored on a 4-point scale that ranges from “not at all” to “very much,” and the last two items are scores on a 7-point scale that ranges from “very poor” to “excellent.” Higher scores indicate higher response levels (i.e., higher health-related quality of life [HRQoL], higher symptom severity).

### FACT-Lym LymS

The FACT-Lym is a validated, reliable self-report measure of HRQoL aspects relevant to lymphoma patients (Hlubocky et al., Lymphoma (2013) 2013:1-9). The full measure consists of the FACT-G physical, social/family, emotional, and functional well-being scales (27 items), as well as a lymphoma-specific symptoms scale (15 items). For this study, only the items that comprise the lymphoma-specific symptoms (LymS) scale are administered to patients. Each item is rated on a 5-point response scale that ranges from “not at all” to “very much,” with higher scores indicative of better HRQoL.

### FACT/GOG-NTX

The FACT/GOG-NTX is a validated self-report measure for assessing platinum/paclitaxel-induced peripheral neuropathy (Huang et al., Int J Gynecol Cancer (2007) 17:387-93). This measure is used to assess vincristine- and polatuzumab vedotin-induced neuropathy, as symptoms of chemotherapy-induced neuropathy caused by microtubule inhibitors do overlap with those seen in platinum/paclitaxel-containing regimens. The full measure consists of the FACT-G physical, social/family, emotional, and functional well-being scales (27 items), as well as a peripheral neuropathy symptoms scale (11 items). For this study, only the items that comprise the peripheral neuropathy scale are administered to patients. The scale contains 4 subscales that assess sensory neuropathy (4 items), hearing neuropathy (2 items), motor neuropathy (3 items), and dysfunction associated with neuropathy (2 items), which can be summed to create a total score. Each item is rated on a 5-point response scale that ranges from “not at all” to “very much,” with higher scores indicative of more extreme neuropathy.

### EQ-5D-5L

The EQ-5D-5L is a validated self-report health status questionnaire that is used to calculate a health status utility score for use in health economic analyses (EuroQol Group. EuroQol: a new facility for the measurement of health-related quality of life. Health Policy (1990) 16:199-208; Brooks R., Health Policy (1996) 37:53-72; Herdman et al., Qual Life Res (2011) 20:1727-36; Janssen et al., Qual Life Res (2013) 22:1717-27). There are two components to the EQ-5D-5L: a five-item health state profile that assesses mobility, self-care, usual activities, pain or discomfort, and anxiety or depression; and a visual analogue scale that measures overall health state. Published weighting systems allow for creation of a single composite score of the patient's health status. The EQ-5D-5L takes approximately 3 minutes to complete. It is utilized in this study for informing pharmacoeconomic evaluations.

### E. Whole Genome Sequencing

Tumor tissue and blood samples are collected for DNA extraction to enable whole genome sequencing (WGS) to identify somatic mutations that may be predictive of response to study treatments, are associated with progression to a more severe disease state, are associated with acquired resistance to study treatments, or can increase the knowledge and understanding of disease biology.

### F. Safety

Patients undergo safety monitoring during the study, including assessment of the nature, frequency, and severity of adverse events. Guidelines for managing adverse events, including criteria for dosage modification and treatment interruption or discontinuation, are provided below.

### Polatuzumab Vedotin

Identified and potential risks of polatuzumab vedotin, and guidelines around the management of these risks, are described below.

Myelosuppression: Neutropenia, neutropenia-associated events, thrombocytopenia, and anemia, including serious and severe cases, have been reported in patients receiving polatuzumab vedotin. Adequate hematologic function is confirmed before initiation of study treatment. Patients receiving study treatment are regularly monitored for evidence of marrow toxicity with complete blood counts. Treatment is delayed or modified for hematologic toxicities as described in Table 6. Primary G-CSF prophylaxis is required for neutropenia. Transfusion support for anemia and thrombocytopenia is permitted at the discretion of the investigator.

TABLE 6
Dose Interruptions, Reductions, and Discontinuations of Polatuzumab Vedotin and R-CHP or R-CHOP.

| Event(s) | Dose Delay or Modification |
|---|---|
| Grade 3 or 4 neutropenia | Delay all study treatment for a maximum of 14 days. |
| with or without infection | Growth factors, e.g., G-CSF, for neutropenia are permitted (in addition to |
| or fever, first delay^{a} | primary prophylaxis). |
| | If ANC recovers to ≥1000/uL ≤7 days after the scheduled date for the |
| | next cycle, administer full dose of all study treatment. |
| | If ANC recovers to ≥1000/uL ≥8 days after the scheduled date for the |
| | next cycle, consider reducing the dose of cyclophosphamide and/or |
| | doxorubicin to a lower dose level. |
| | Dose of blinded polatuzumab vedotin/placebo, blinded |
| | vincristine/placebo, rituximab, and prednisone is not modified for this |
| | reason. |
| Recurrent Grade 3 or 4 | Delay all study treatment for a maximum of 14 days. |
| neutropenia with or | Growth factors, e.g., G-CSF, for neutropenia are permitted (in addition to |
| without infection or | primary prophylaxis). |
| fever | If ANC recovers to ≥1000/uL ≤7 days after the scheduled date for the |
| | next cycle, administer current dose of cyclophosphamide and/or |
| | doxorubicin. |
| | If ANC recovers to ≥1000/uL ≥8 days after the scheduled date for the |
| | next cycle, consider reducing the doses of cyclophosphamide and |
| | doxorubicin to the next dose level. |
| | Dose of blinded polatuzumab vedotin/placebo, blinded |
| | vincristine/placebo, rituximab, and prednisone are not modified for this |
| | reason. |
| | If Grade 3 or 4 neutropenia persists despite growth factor support and |
| | following cyclophosphamide and doxorubicin dose reductions, in the |
| | absence of fever, patient may continue study treatment. |
| Grade 3 or 4 | Delay all study treatment for a maximum of 14 days. |
| thrombocytopenia, first | If platelet count recovers to ≥75 × 10⁹/L ≤ Day 7 after the scheduled date |
| episode | of the next cycle, administer full dose of all study drugs. |
| | If platelet count recovers to ≥75 × 10⁹/L ≥ Day 8 after the scheduled date |
| | of the next cycle, consider reducing the dose of cyclophosphamide and/or |
| | doxorubicin to a lower dose level. Full dose/current dose of all other |
| | study drugs may be given. |
| | If the primary cause of thrombocytopenia is thought to be lymphoma |
| | infiltration into the bone marrow, the investigator may elect not to reduce |
| | the dose of cyclophosphamide and/or doxorubicin. |
| Recurrent Grade 3 or 4 | If patient develops recurrent Grade 3-4 thrombocytopenia following |
| thrombocytopenia | cyclophosphamide and/or doxorubicin dose reductions, consider reducing |
| | the dose of cyclophosphamide and doxorubicin to the next dose level. |
| Hemorrhagic cystitis | Patients should be adequately hydrated before and after |
| | cyclophosphamide administration and should be instructed to void |
| | frequently. |
| | If gross hematuria develops, cyclophosphamide should be withheld until |
| | resolution of cystitis. |
| | A dose reduction of 50% for cyclophosphamide may be considered at the |
| | next cycle. |
| | Re-escalation of cyclophosphamide to the initial full dose is |
| | recommended if symptoms do not recur. |
| Grade 2-4 heart | Discontinue all study treatment permanently. |
| failure or Grade 3 or 4 | |
| LVSD | |
| Bilirubin between 1.5 | Dose reduction should be avoided if hyperbilirubinemia is not related to |
| and 3.0 mg/dL | hepatic injury (i.e., hemolysis or Gilbert's disease). In these cases, dose |
| | reduction considerations should be guided by direct bilirubin levels. |
| | Reduce doxorubicin dose by at least 25% of baseline and polatuzumab |
| | vedotin/placebo and vincristine/placebo should be reduced to the next |
| | level. With subsequent courses of treatment, if bilirubin has returned |
| | to ≤1 mg/dL, full doses may be given. Evaluate for causality. |
| Bilirubin > 3.0 mg/dL | Dose delay should be avoided if hyperbilirubinemia is not related to |
| | hepatic injury (i.e., hemolysis or Gilbert's disease). In these cases, dose |
| | delay considerations should be guided by direct bilirubin levels. |
| | Withhold doxorubicin, blinded polatuzumab vedotin/placebo, and blinded |
| | vincristine/placebo until improvement to Grade ≤1. Evaluate for |
| | causality. Dosing of rituximab, cyclophosphamide, and prednisone may |
| | continue. |
| Grade 1 g | No study treatment modification is recommended for Grade 1 sensory or |
| | motor peripheral neuropathy. |
| Grade 2 sensory | If the AE severity remains unchanged at the time of the next scheduled |
| peripheral neuropathy | cycle, administer R-CHP at the recommended doses. The blinded |
| | polatuzumab vedotin/placebo and blinded vincristine/placebo should be |
| | reduced one dose level. |
| | Should the AE remain Grade 2 in future cycles, further dose reductions |
| | should occur. If the AE improves to Grade 1 by the start of the next cycle, |
| | administer the blinded polatuzumab vedotin/placebo and blinded |
| | vincristine/placebo at the most recent dose. |
| Grade 3 sensory | If the AE severity remains unchanged by the start of the next cycle, hold |
| peripheral neuropathy, | the blinded polatuzumab vedotin/placebo and blinded vincristine/placebo, |
| or Grade 2 or 3 motor | and administer R-CHP components at the recommended doses. The |
| peripheral neuropathy | withheld doses of blinded polatuzumab vedotin/placebo and blinded |
| | vincristine/placebo are not made up at a later date. |
| | When the AE improves to Grade ≤2 peripheral sensory neuropathy and/or |
| | Grade ≤1 peripheral motor neuropathy, the blinded polatuzumab |
| | vedotin/placebo and blinded vincristine/placebo can be restarted at a |
| | reduced dose. |
| Grade 4 neuropathy | Discontinue blinded polatuzumab vedotin/placebo and blinded |
| (including peripheral | vincristine/placebo treatment permanently. |
| sensory or motor | Patients should be evaluated regarding the continuation of R-CHP on the |
| neuropathy) | basis of their risk/benefit. |
| Grade 3 or 4 | Blinded polatuzumab vedotin/placebo and blinded vincristine/placebo |
| constipation or ileus | should be held until improvement to Grade ≤2. All other study drugs may |
| | be continued, or delayed at the discretion of the investigator. |
| | Consider reducing blinded polatuzumab vedotin/placebo and blinded |
| | vincristine/placebo to the next dose level after improvement to Grade ≤2. |
| Grade 3 or 4 tumor | Following complete resolution of tumor lysis syndrome, study treatment |
| lysis syndrome | may be re-administered at the full/current dose during the next scheduled |
| | infusion in conjunction with prophylactic therapy. |
| Grade 3 IRR, second | Discontinue rituximab or blinded polatuzumab vedotin/placebo |
| episode | permanently. |
| | If IRR is attributed to rituximab, continue blinded polatuzumab |
| | vedotin/placebo, blinded vincristine/placebo, and CHP. |
| | If IRR is attributed to blinded polatuzumab vedotin/placebo, continue |
| | rituximab, blinded vincristine/placebo, and CHP. |
| Anaphylaxis or Grade 4 | Discontinue rituximab or blinded polatuzumab vedotin/placebo |
| IRR | permanently. |
| | If anaphylaxis is attributed to rituximab, continue blinded polatuzumab |
| | vedotin/placebo, blinded vincristine/placebo, and CHP. |
| | If anaphylaxis is attributed to blinded polatuzumab vedotin/placebo, |
| | continue rituximab, blinded vincristine/placebo, and CHP. |
| Grade 2 to 4 non- | Consider delaying all study treatment for a maximum of 14 days. |
| hematologic toxicity | Subsequent recurrence: Based on the nature of the toxicity, decrease one |
| not otherwise specified | or more study drugs (blinded polatuzumab vedotin/placebo, blinded |
| Event(s) | Dose Delay or Modification |
| (excluding nausea, | vincristine/placebo, cyclophosphamide, or doxorubicin) to a lower dose. |
| vomiting, and diarrhea) | Prednisone dose may be modified based on investigator preference. |
| | Second and subsequent recurrence: Based on the nature of the toxicity |
| | and if the event is not clinically manageable and resolving within 14 days |
| | of the date of the next scheduled cycle, consider discontinuation of |
| | suspect study treatment permanently. |

G-CSF = granulocyte colony-stimulating factor;
HBV = hepatitis B virus;
IRR = infusion-related reaction;
LVSD = left ventricular systolic dysfunction.
ªBased on laboratory results obtained within 72 hours before study treatment administration on Day 1of each cycle of study treatment.

Peripheral Neuropathy (Sensory and/or Motor): Patients receiving polatuzumab vedotin may develop peripheral neuropathy (sensory and/or motor). Patients receiving study treatment are monitored for symptoms of neuropathy, including hypoesthesia, hyperesthesia, paresthesia, dysesthesia, discomfort, a burning sensation, weakness, gait disturbance, or neuropathic pain. New or worsening peripheral neuropathy is managed by delay, change in dose, or discontinuation of treatment (see, Table 6). Supportive care measures are implemented per investigator discretion (e.g., gabapentin).

Infections: Patients receiving polatuzumab vedotin may be at a higher risk of developing infections. Serious infections, including opportunistic infections, such as pneumonia (including pneumocystis jirovecii and other fungal pneumonia), bacteremia, sepsis, herpes infection, and cytomegalovirus infection have been reported in patients treated with polatuzumab vedotin. Several other risk factors in the patient population under study influencing patients' vulnerability to a higher risk of infections, particularly serious and opportunistic infection, include predisposition of the indication disease to infections, elderly population, and comorbidity. In addition, neutropenia is a known risk for polatuzumab vedotin. Granulocytopenia is a major predisposing factor to infections in patients with B-cell lymphoma. The reported incidence of infection in chemotherapy courses for B-cell lymphoma associated with <500 granulocytes/μL was higher than those with ≥500 granulocytes/μL. Neutropenia events are monitored closely and any signs of infection are treated as appropriate. Anti-infective prophylaxis is administered as appropriate. See. Flowers et al., J Clin Oncol (2013) 31:794-810; National Comprehensive Cancer Network®. NCCN clinical practice guidelines in oncology (NCCN Guidelines®); Prevention and treatment of cancer-related infections, version 2 [resource on the Internet]. 2017 [cited 9 Jun. 2017]. Available from: www[dot]nccn[dot]org/professionals/physician_gls/f_guidelines.asp.

Infusion-Related Reactions: IRRs have been reported in patients receiving polatuzumab vedotin. Commonly experienced events included nausea, vomiting, chills, fever, pruritus, hypotension, flushing, and other symptoms. In the majority of the patients, the events were Grade 1-2. Premedications for polatuzumab vedotin infusion administration are outlined in Table 5. Close monitoring throughout the infusion is required, and IRRs are managed as outlined in Table 7.

TABLE 7
Management of Infusion-Related Symptoms:
Rituximab and Polatuzumab Vedotin.

| Infusion-Related | |
|---|---|
| Symptoms | Guidance |
| Grade 1-2 | Slow or hold infusion. |
| | Give supportive treatment.ª |
| | Upon symptom resolution, may resume infusion- |
| | rate escalation at the investigator's discretion. |
| | For Grade 2 wheezing or urticaria, patient must |
| | be pre-medicated for any subsequent doses. |
| | If symptoms recur, the infusion must be |
| | stopped immediately and patient permanently |
| | discontinued from study drug. |
| Grade 3 | Discontinue infusion. |
| | Give supportive treatment.ª |
| | Upon symptom resolution, may resume infusion- |
| | rate escalation, at investigator discretion.^{b} |
| | If the same adverse event recurs with same |
| | severity, treatment must be permanently |
| | discontinued. |
| | For Grade 3 hypotension or fever, patient must |
| | be pre-medicated before re-treatment. If symptoms |
| | recur, then patient must be permanently |
| | discontinued from study drug. |
| | If patient has Grade 3 wheezing, bronchospasm, |
| | or generalized urticaria at first occurrence, |
| | patient must be permanently discontinued |
| | from study drug. |
| Grade 4 | Discontinue infusion immediately, treat symptoms |
| | aggressively, and permanently discontinue |
| | study drug. |

NCI CTCAE v4.0 = National Cancer Institute Common Terminology Criteria for Adverse Events, Version 4.0. Refer to the NCI-CTCAE v4.0 scale for the grading of symptoms. Management of IgE- mediated allergic reactions is as described below.
ªSupportive treatment: Patients are treated with acetaminophen/paracetamol and an antihistamine such as diphenhydramine if they have not been received in the previous 4 hours. IV saline may be indicated. For bronchospasm, urticaria, or dyspnea, patients may require antihistamines, oxygen, corticosteroids (e.g., 100 mg IV prednisolone or equivalent), and/or bronchodilators. Patients with hypotension who require vasopressor support must be permanently discontinued from study drug.
^{b}Infusion rate escalation after re-initiation: Upon complete resolution of symptoms, the infusion may be resumed at 50% of the rate achieved prior to interruption. In the absence of infusion-related symptoms, the rate of infusion may be escalated in increments of 50 mg/hr every 30 minutes.

Gastrointestinal Toxicity (Diarrhea, Nausea, Vomiting, Constipation, and Anorexia): Diarrhea, nausea, vomiting, constipation, and abdominal pain are reported frequently, with diarrhea and nausea being the most common (≥20%) treatment-emergent adverse events in Phase I and II clinical studies with polatuzumab vedotin. Diarrhea has been responsible for study drug modification and discontinuation. Most cases were low grade, with more serious cases being confounded by polypharmacy, comorbidities, or disease under study.

Tumor Lysis Syndrome (TLS): There is a potential risk of TLS if treatment with polatuzumab vedotin results in the rapid destruction of a large number of tumor cells. If any evidence of TLS occurs during the study, tumor lysis prophylaxis measures are instituted. Patients who are considered to have a high tumor burden (e.g., lymphocyte count ≥25×10⁹/L or bulky lymphadenopathy) and who are considered to be at risk for TLS by the investigator receive tumor lysis prophylaxis (e.g., allopurinol ≥300 mg/day PO or a suitable alternative treatment such as rasburicase before study treatment) and are well hydrated before the initiation of study treatment at Cycle 1, Day 1. These patients continue to receive repeated prophylaxis and adequate hydration, as deemed appropriate by the investigator.

Immunogenicity (Anti-Drug Antibodies): As with any recombinant antibody, polatuzumab vedotin may elicit an immune response, and patients may develop antibodies against it. Patients are closely monitored for any potential immune response to polatuzumab vedotin. Appropriate screening, confirmatory, and characterization assays are employed to assess ADAs before, during, and after the treatment with polatuzumab vedotin. Given the historically low immunogenicity rate of rituximab in patients with Non-Hodgkin lymphoma (NHL), ADAs against rituximab are not monitored in this study.

Reproductive Toxicity: Adverse effects on human reproduction and fertility are anticipated with the administration of polatuzumab vedotin, given the mechanism of action of MMAE. Standard exclusion criteria are used to ensure that patients of childbearing potential (male or female) are using adequate contraceptive methods.

Hyperglycemia: Hyperglycemia has been observed in patients treated with polatuzumab vedotin, as well as with other antibody drug conjugates (ADCs) that use the same valine-citrulline-MMAE platform. Hyperglycemia has been reversible upon holding or discontinuing treatment of the ADCs and/or initiation or adjustment of anti-hyperglycemic medications.

Hepatotoxicity: Hepatotoxicity has been observed in patients treated with polatuzumab vedotin in both Phase I and Phase II trials. Although the relationship between hepatotoxicity and polatuzumab vedotin has not been definitively determined, transient, dose-related increases in hepatic enzymes were noted in nonclinical rat studies. No hepatotoxicity was noted following administration of the surrogate ADC in cynomolgus monkeys. Elevations of transaminases have been reported in patients receiving polatuzumab vedotin and have ranged in intensity from Grades 1-4. These have been reversible with and without dose modification/discontinuation, e.g., as described herein.

Carcinogenicity: Polatuzumab vedotin may have carcinogenic potential given the mechanism of action of MMAE, the cytotoxic component of polatuzumab vedotin. Myelodysplastic syndrome and other second malignancies have been reported in Phase I and II clinical studies with polatuzumab vedotin. The majority of these patients had received multiple prior lines of anti-cancer therapy, and this was considered as a significant contributory factor.

### Rituximab

Rituximab dose delay, modification and discontinuation instructions are provided in Tables 6 and 7.

### Cyclophosphamide, Doxorubicin, Vincristine, and Prednisone

For CHOP, CHP, and blinded vincristine dose delay, modification, and discontinuation instructions, see Table 6.

The recommended steps for dose reductions of cyclophosphamide are provided in Table 8.

TABLE 8
Recommended Steps of Dose Reduction for Cyclophosphamide.

| Dose Level | Cyclophosphamide |
|---|---|
| Starting dose | 100% of starting dose per cycle |
| First dose reductionª | 75% of starting dose per cycle |
| Maximum dose reductionª | 50% of starting dose per cycle or |
| | discontinue drug |
| Subsequent dose reduction | Discontinue drug |

ªSteps of dose reduction listed are suggested dose changes. Investigators could opt for alternative levels of dose reduction as clinically indicated.

The recommended steps for dose reductions of doxorubicin are provided in Table 9.

TABLE 9
Recommended Steps of Dose Reduction for Doxorubicin.

| Dose Level | Doxorubicin |
|---|---|
| Starting dose | 100% of starting dose per cycle |
| First dose reductionª | 75% of starting dose per cycle |
| Maximum dose reductionª | 50% of starting dose per cycle or |
| | discontinue drug |
| Subsequent dose reduction | Discontinue drug |

ªSteps of dose reduction listed are suggested dose changes. Investigators could opt for alternative levels of dose reduction as clinically indicated.

### Management of Specific Adverse Events

Guidelines for management of specific adverse events are outlined in Table 6. Additional guidelines are provided below.

Guidelines for dose delays and modifications of R-CHP, blinded polatuzumab vedotin/placebo, and blinded vincristine/placebo are shown in Tables 3, 6, 8, and 9. Dose delays and dose modifications due to adverse events not specified in Table 6 proceed on the basis of the principle of maintaining the dose intensity of R-CHP or R-CHOP. Dose modifications and interruptions for prednisone are made at the discretion of the investigator. Cyclophosphamide or doxorubicin are permitted to be reduced separately; that is, one or both agents are reduced in 25%-50% increments per investigator discretion. Cyclophosphamide and doxorubicin doses are allowed to be re-escalated (even to the full dose).

The dose of blinded polatuzumab vedotin/placebo and blinded vincristine/placebo and chemotherapy (cyclophosphamide or doxorubicin) is permitted to be reduced stepwise to a maximum of two levels for management of drug-related toxicities. If further dose reduction is indicated after two dose reductions, the patient discontinues the specific study drug and is permitted to continue treatment with the remaining study drugs. If administration of R-CHP or R-CHOP is delayed, the administration of polatuzumab vedotin and R-CHP/R-CHOP is delayed for the same time frame; that is, all study drugs are delayed for the same time frame so that they are all given together beginning on Day 1 of the same cycle.

Study treatment is temporarily suspended in patients who experience toxicity considered to be related to study drug (see Table 6). Aside from the withholding of blinded polatuzumab vedotin/blinded vincristine for neuropathy according to Table 6, study drugs withheld for >14 days because of toxicity are discontinued, unless otherwise indicated.

### (i) Tumor Lysis Syndrome (TLS)

Patients with high tumor burden and who are considered to be at risk for TLS receive tumor lysis prophylaxis before the initiation of treatment. Patients are well hydrated, e.g., maintenance of a fluid intake of approximately 3 L/day starting 1 or 2 days before the first dose of study treatment. In addition, all patients with high tumor burden and who are considered to be at risk for TLS are treated with 300 mg/day of allopurinol PO or a suitable alternative treatment (e.g., rasburicase) starting 48-72 hours before Cycle 1, Day 1 of treatment and hydration. Patients continue to receive repeated prophylaxis and adequate hydration before each subsequent infusion, if deemed appropriate by the investigator. For patients with evidence of TLS, all study treatment is suspended and the patient is treated as clinically indicated. Following the complete resolution of TLS complications, treatment is permitted to be resumed at the full dose at the next scheduled infusion in conjunction with prophylactic therapy.

### (ii) Infusion-Related Reactions (IRR) and Anaphylaxis

Management of infusion-related symptoms for rituximab is summarized in Table 7. In the event of a life-threatening IRR (including pulmonary or cardiac events) or IgE-mediated anaphylactic reaction, study treatment is discontinued and no additional drug is administered. Patients who experience any of these reactions receive aggressive symptomatic treatment and are discontinued from study treatment. Patients who experience rituximab- or polatuzumab vedotin-associated infusion-related temperature elevations of >38.5° C. (101.3° F.) or other minor infusion-related symptoms are treated symptomatically with acetaminophen (≥500 mg) and/or H1- and H2-histamine-receptor antagonists (e.g., diphenhydramine hydrochloride, ranitidine). Serious infusion-related events, manifested by dyspnea, hypotension, wheezing, bronchospasm, tachycardia, reduced oxygen saturation, or respiratory distress, are managed with additional supportive therapies (e.g., supplemental oxygen, β₂-agonists, epinephrine, and/or corticosteroids) as clinically indicated according to standard clinical practice. Guidelines for the management of IRRs and anaphylaxis are detailed in Table 7 and dose modifications are detailed in Table 6.

(iii) Neutropenia

Because neutropenia is a known risk of polatuzumab vedotin and the other components of R-CHP, the use of growth factor support (G-CSF) as prophylaxis and as a therapeutic indication is implemented in order to allow continued dosing of polatuzumab vedotin and all other study drugs. Dose and schedule modifications for neutropenia are detailed in Table 6.

### (iv) Infections (Including Hepatitis B Virus Reactivation)

Infection is a known risk for R-CHOP and for polatuzumab vedotin. Risk factors for viral reactivation and opportunistic infections are taken into consideration when implementing prophylactic anti-infective agents where indicated and according to institutional guidelines. Dose and schedule modifications for infections are detailed in the section for non-hematologic toxicity not otherwise specified in Table 6.

Hepatitis B virus reactivation is a potential risk for R-CHOP. Patients who are both HBsAg-negative and anti-hepatitis B core positive are included in this study. Prophylactic antiviral therapy is considered for these patients (for example, American Gastroenterology Association guidelines [Reddy et al., Gastroenterology (2015) 148:215-19]). These patients have HBV DNA levels obtained monthly by means of real-time PCR using an assay with a sensitivity of at least 10 IU/mL for at least 12 months after the last cycle of therapy, as follows:
If the HBV DNA assay becomes positive and is within the WHO-recommended range of 10-100 IU/mL, the patient is re-tested within 2 weeks. If the assay is still positive, all study treatment is held and the patient is treated with an appropriate nucleoside analog (for at least 1 year after the last dose of rituximab or polatuzumab vedotin) and referred to a gastroenterologist or hepatologist for management.
If the HBV DNA assay becomes positive and is above the WHO cutoff of 100 IU/mL, study treatment chemotherapy is held and the patient is treated (for at least 1 year after the last dose of rituximab or polatuzumab vedotin) with an appropriate nucleoside analog and referred to a gastroenterologist or hepatologist for management. Patients are permitted to resume study treatment once HBV DNA levels decrease to undetectable levels.
If a patient's HBV DNA level exceeds 100 IU/mL while the patient is receiving antiviral medication, study treatment is discontinued.

### G. Biomarkers

A summary of biomarkers assessed in this study is provided in Table 10.

TABLE 10
Biomarkers.

| Sample Type | Timing | Biomarkers |
|---|---|---|
| DNA extracted from whole | Baseline | Gene mutations by NGS. |
| blood | | MRD index clone by NGS. |
| | | Genomic evaluation |
| | | information, disease biology, |
| | | safety, companion assay |
| | | development. |
| Circulating tumor DNA isolated | Baseline, interim, end of | ctDNA level and clonal |
| from plasma | treatment, and at follow up | mutation profile. |
| | with correlation to response | ctDNA as a peripheral |
| | by the Lugano Response | measure of disease biology, |
| | Criteria for Malignant | prognosis, subsets and |
| | Lymphoma. | treatment response. |
| Peripheral blood mononuclear | Baseline and subsequent | Lymphocyte subsets |
| cells isolated from whole blood | timepoints during and after | |
| | treatment. | |
| Tumor tissue | Archival or at baseline | RNA-based gene expression |
| | | profiling including, but not |
| | | limited to, cell of origin gene |
| | | signature analysis. |
| | | Mutational profiling by NGS, |
| | | including, but not limited to, |
| | | MYD88 and CD79B. |
| | | IHC and proteomic profiling |
| | | including BCL2, MYC. |
| | | Translocation profiles |
| | | including MYC with |
| | | BCL2/BCL6. |
| | | Baseline index clone for |
| | | MRD. |
| | Time of progression | RNA/DNA molecular profiles, |
| | | proteomic profiles. |
| DNA extracted from tumor | Prior to study or at baseline | Gene mutations by NGS. |
| tissue | | MRD index clone by NGS. |

ctDNA = circulating tumor DNA;
IHC = immunohistochemistry;
MRD = minimal residual disease;
NGS = next-generation sequencing.

### Example 2: Efficacy and Safety Results of a Phase III Study of Polatuzumab Vedotin in Combination with Rituximab and Cyclophosphamide, Doxorubicin, and Prednisone (Pola-R-CHP) Compared to Rituximab, Cyclophosphamide, Doxorubicin, Vincristine, and Prednisone (R-CHOP) in Previously Untreated Diffuse Large B-Cell Lymphoma (DLBCL)

This Example describes efficacy and safety results of the Phase III study described in Example 1.

Patients are randomized 1:1 in this Phase III study to receive either a fixed-dose of Pola-R-CHP plus a vincristine placebo for six cycles, followed by rituximab for two cycles; or R-CHOP plus a polatuzumab vedotin placebo for six cycles, followed by two cycles of rituximab. The primary outcome measure is progression-free survival as assessed by the investigator using the Lugano Response Criteria for Malignant Lymphoma.

The efficacy and safety of Pola-R-CHP compared to R-CHOP are assessed as described below in patients with previously untreated DLBCL.

### A. Efficacy

### (i) Primary Efficacy Endpoint

The primary efficacy endpoint is progression-free survival (PFS), as determined by the investigator, defined as the time from the date of randomization until the first occurrence of disease progression or relapse as assessed by the investigator using the 2014 Lugano Classification for Malignant Lymphoma (Cheson et al., 2014), or death from any cause, whichever occurs earlier. For patients who have not progressed, relapsed, or died as of the clinical cutoff date for analysis, PFS is censored on the date of last disease assessment when the patient is known to be progression-free. If no tumor assessments are performed after the baseline visit or all post-baseline tumor assessment results have overall responses of “not evaluable,” PFS is censored on the date of randomization. The Kaplan-Meier method is used to estimate the PFS distribution for each treatment arm. The Kaplan-Meier curve provides a visual description of the differences across treatment arms. Estimates of the treatment effect are expressed as hazard ratios using a stratified Cox proportional-hazards analysis, including 95% confidence intervals. Stratification factors include: International Prognostic Index (IPI) score of 2 versus 3-5; presence or absence of bulky disease, defined as a lesion ≥7.5 cm; and geographic region (Asia, Western Europe/USA/Canada/Australia, or the rest of the world). Estimates of the treatment effect are also expressed as unstratified hazard ratios, including 95% confidence intervals. In addition, the 1-year, 2-year, or 3-year PFS rates may be used to describe PFS in addition to the hazard ratio.

### (ii) Secondary Efficacy Endpoints

Complete response (CR) rate at end-of-treatment by PET-CT by blinded independent central review (BICR) or by the investigator is defined as the percentage of patients with CR at the end of treatment by PET-CT, as assessed by BICR or by the investigator.

Event-free survival-efficacy (EFS_{eff}) is used to reflect event-free survival (EFS) events that are primarily due to efficacy and is defined as the time from date of randomization to the earliest occurrence of the below cases:
1. Disease progression/relapse.
2. Death due to any cause.
3. The primary efficacy reason determined by the investigator, other than disease progression/relapse, that leads to initiation of any non-protocol specified anti-lymphoma treatment (NALT).
4. If biopsy is obtained after treatment completion, and is positive for residual disease, regardless of whether NALT is initiated or not.

For case 3 above, the efficacy reason includes instances where a PET-CT scan, bone marrow test, CT/MRI, or physical finding is suggestive of residual disease; or instances where a biopsy confirms residual disease. EFS_{eff} event timing is at the time of the test or biopsy leading to NALT, rather than the date of NALT initiation.

For patients without the occurrence of any above cases (no EFS_{eff} event) at the time of analysis, EFS_{eff} is censored on the date of last tumor assessment when the patient is known to be progression-free. For patients with no EFS_{eff} event, who do not have post-baseline tumor assessments or all post-baseline tumor assessment results have overall responses of ‘not evaluable’, EFS_{eff} is censored on the date of randomization.

24-month progression-free survival (PFS24) is defined as the PFS rate calculated through Kaplan-Meier method at 24 months after randomization.

Overall survival (OS) is defined as the period from the date of randomization until the date of death from any cause. For patients who have not died at the clinical cutoff date for analysis, OS is censored on the last date when the patients are known to be alive, as documented by investigator.

EFS-all causes (EFSₐₗₗ) differs from EFS_{eff}, and is defined as the time from randomization to disease progression or relapse, as assessed by the investigator, death from any cause, or initiation of any new anti-lymphoma therapy (NALT). If the specified event (disease progression or relapse, death, or initiation of a NALT) does not occur, EFSₐₗₗ is censored at the date of last tumor assessment. For patients without an event who have not had post-baseline tumor assessments, EFSₐₗₗ is censored at the time of randomization.

EFS_{eff} and OS are analyzed using the same statistical methods as those described for PFS. PFS24 is estimated using the Kaplan-Meier method, and 95% confidence intervals are calculated based on the normal approximation with standard errors via the Greenwood method. The difference of PFS24 between the two treatment groups is tested using z-test with the standard errors for the KM-estimates computed via the Greenwood method.

CR rates at end-of-treatment by PET-CT are compared between the two treatment groups using CMH test stratified by randomization stratification factors. In addition, rates and 95% confidence intervals are reported for each treatment group. Patients with no response assessments are considered non-responders.

### B. Safety

All verbatim adverse event terms occurring on or after first study treatment are mapped to Medical Dictionary for Regulatory Activities (MedDRA) thesaurus terms, and adverse event severity is graded according to NCI CTCAE v4.0.

### C. Subgroup Analyses

Patient subgroups classified according to certain baseline characteristics and biomarker subgroups are analyzed using stratified and unstratified analyses to assess the benefit of Pola-R-CHP as compared to R-CHOP in those patient subgroups. Patient subgroups include: patients <=65 years old, >65 years old, at least 60 years old, or >60 years old: identified histopathologically, high grade b-cell lymphoma, NOS or HGBL with MYC and BCL2 and/or BCL6-rearrangements; specific subtypes of DLBCL such as activated B-cell like (ABC) subtype, a double expressing lymphoma (DEL: overexpression of BCL2 and MYC), and DLBCL that does not have double-hit or triple-hit lymphoma defined by MYC and BCL2 and/or BCL6-rearrangements; low Ann-Arbor Stage (I-II) and higher Ann-Arbor Stages (III, IV); normal baseline LDH levels and elevated baseline LDH levels; bone marrow involvement at baseline; 0-1 and 2+ extranodal sites; and an International Prognostic Index (IPI) score between 3-5.

Cell-of-origin subtypes are assessed by RNA expression using the NanoString Lymph 2Cx assay. MYC, BCL2, and BCL6 rearrangements are examined by fluorescence in situ hybridization (FISH) using Vysis LSI MYC, BCL2, and BCL6 Dual Color Break Apart Probes, respectively. BCL2 and MYC protein expression is assessed by immunohistochemistry (IHC) using anti-BCL2 (124) mouse monoclonal antibody, and clone Y69 Epitomics antibody, respectively. BCL2 IHC scoring incorporates the percentage of positive stained tumor cells and the intensity of tumor cell staining. BCL2 IHC+ is defined as ≥50% of tumor cells having moderate or strong expression compared with mantle zone B cells and paracortical T cells in normal tonsils (used as references for “moderate” BCL2 IHC staining intensity), see, e.g., Morschhauser et al., Blood 2021:137:600-9. Tumors are classified as MYC IHC+ if ≥40% of cells show MYC nuclear staining above background intensity, see, e.g., Morschhauser et al., Blood 2021:137:600-9; and Punnoose et al., Clin Lymphoma Myeloma Leuk 2021:21:267-78.e10.

### D. Results

### Patients

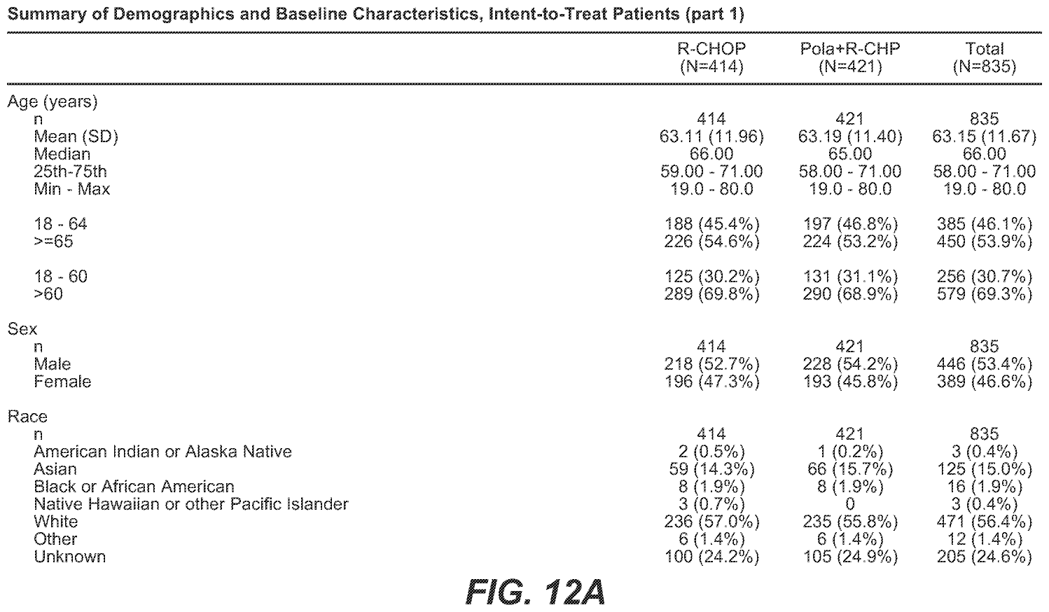

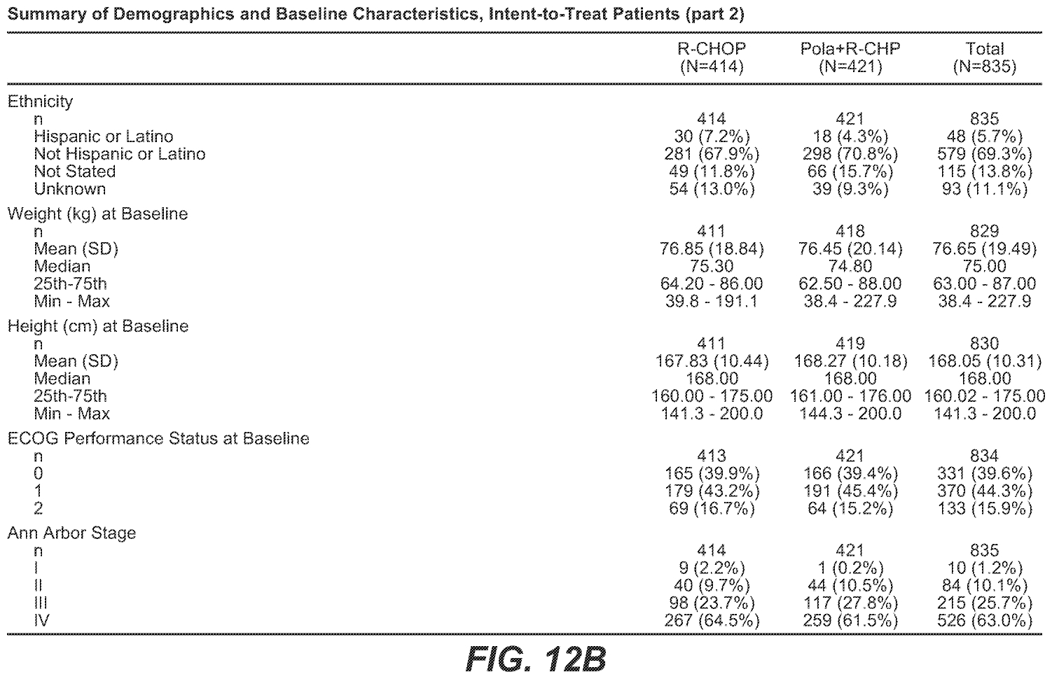

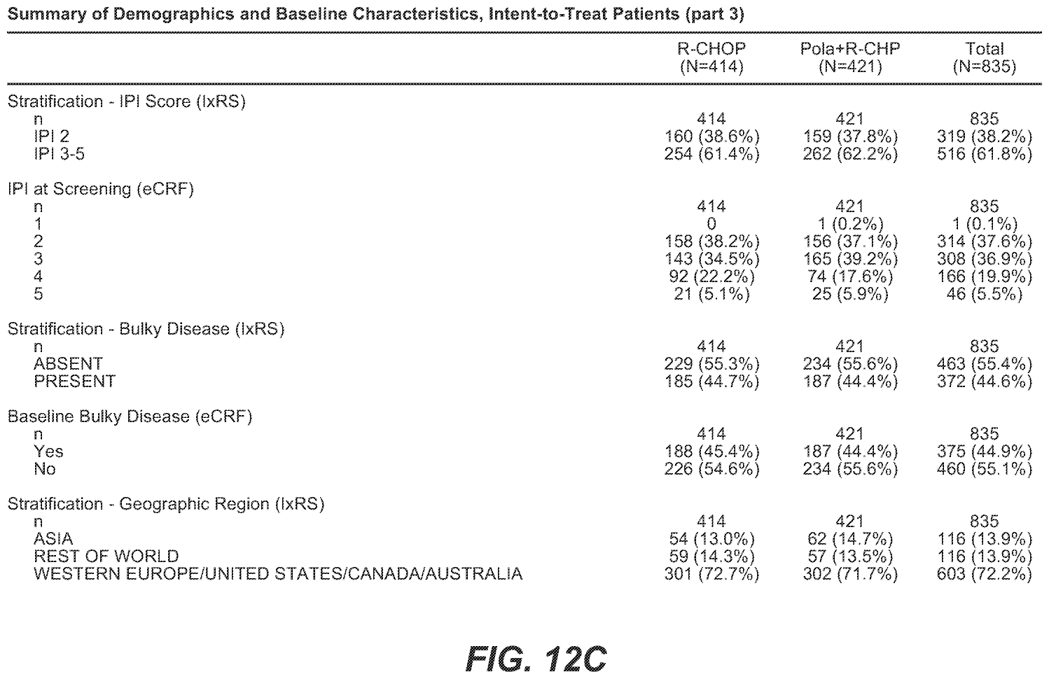

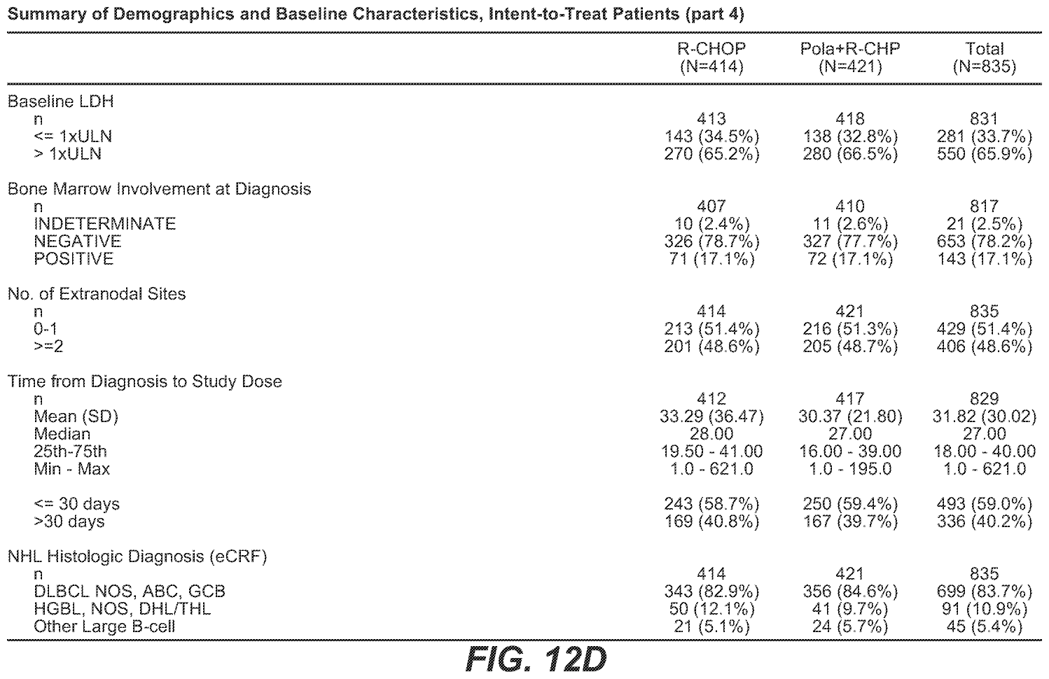

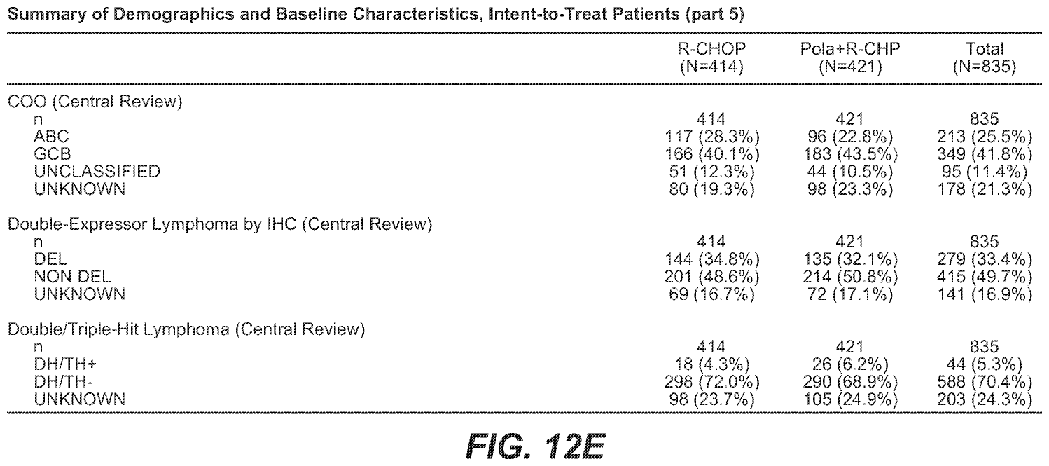

A summary of baseline demographics and patient characteristics for patients in this study is provided in FIGS. 12A-12E. Baseline patient characteristics were generally well balanced between the treatment arms.

The median interval between diagnosis, defined by the date of biopsy, and initiation of treatment was similar in the two treatment groups (27 and 28 days in the Pola-R-CHP and R-CHOP arms, respectively).

The median follow-up time for patients in this study was 28.3 months.

### Primary Efficacy Endpoint

Efficacy analyses were conducted using the intention-to-treat (ITT) population, defined as all randomized patients.

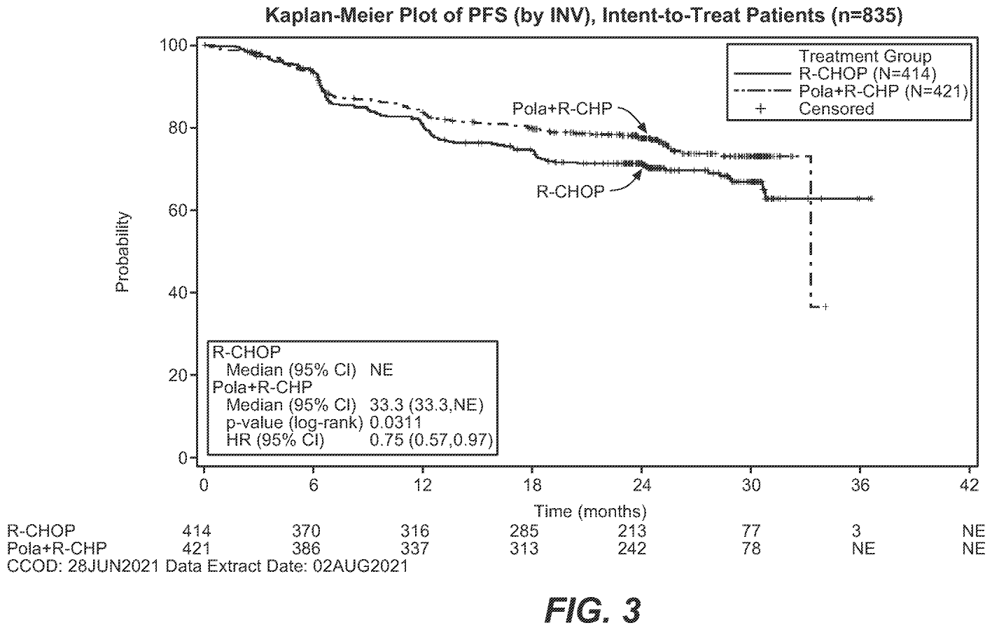

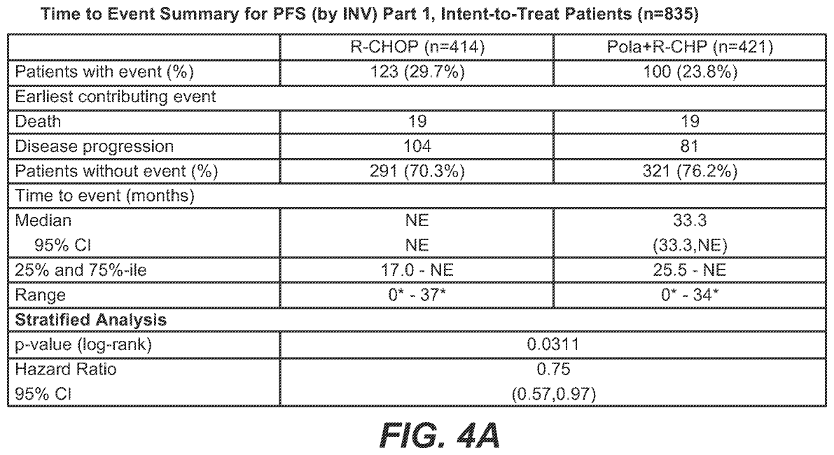

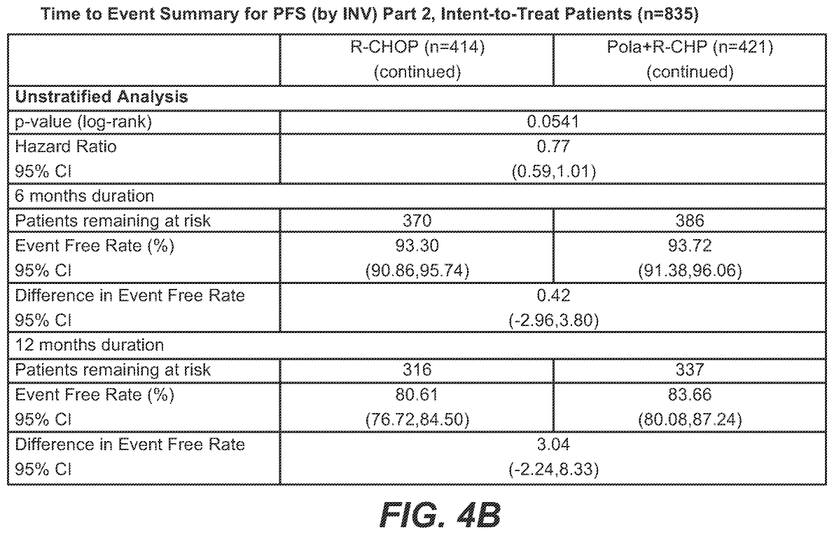

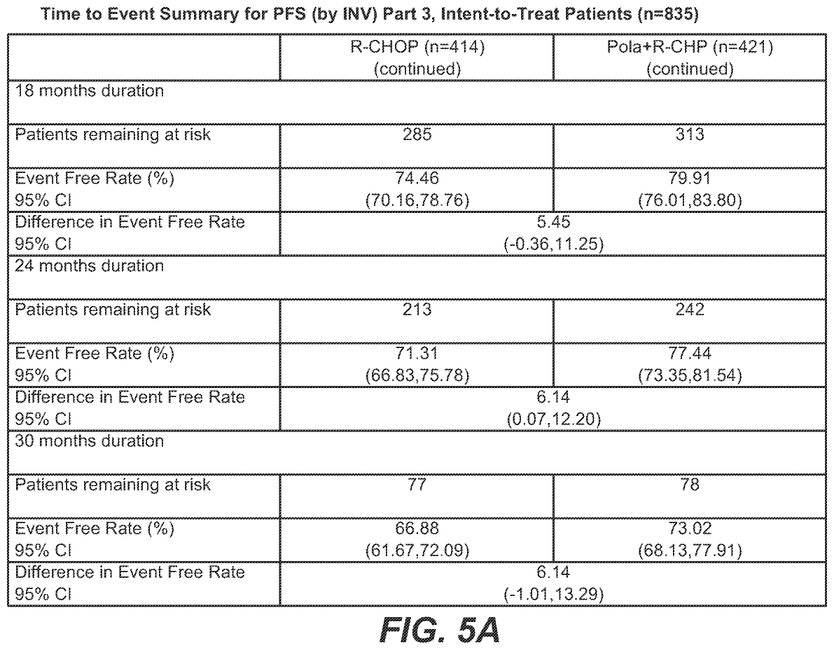

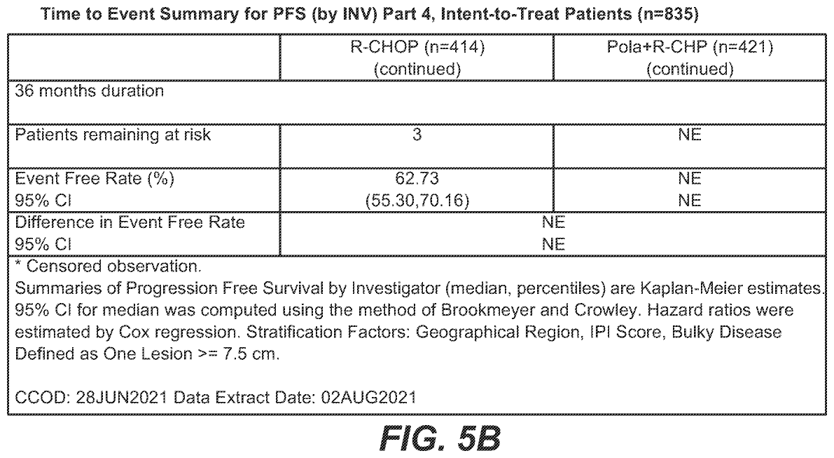

This study met its primary endpoint by demonstrating significantly improved progression-free survival (PFS) in patients with previously untreated diffuse large B-cell lymphoma (DLBCL). The results of this Phase III study showed a statistically significant and clinically meaningful improvement in investigator-assessed PFS when treated with polatuzumab vedotin in combination with rituximab plus cyclophosphamide, doxorubicin, and prednisone (Pola-R-CHP) compared to standard of care rituximab plus cyclophosphamide, doxorubicin, vincristine, and prednisone (R-CHOP). 24-month PFS rate favors Pola-R-CHP versus R-CHOP. See Table 11 and FIG. 3, FIGS. 4A, 4B, 5A and 5B.

TABLE 11

| Investigator- | Intent-To-Treat Population (N = 835) | |
|---|---|---|
| assessed PFS | R-CHOP (n = 414) | Pola-R-CHP (n = 421) |
| Patients with | 123 (29.7%) | 100 (23.8%) |
| event (%) | | |

Earliest contributing event

| | | |
|---|---|---|
| Death | 19 | 19 |
| Disease | 104 | 81 |
| progression | | |

Stratified Analysis

| Stratified Log- | 0.03 | |
|---|---|---|
| rank p-value | | |
| Stratified Hazard | 0.75 (0.57, 0.97) | |
| Ratio (95% CI) | | |
| 6-month PFS rate | 93.3% (90.9, 95.7) | 93.7% (91.4, 96.1) |
| (95% CI) | | |
| 12-month PFS rate | 80.6% (76.7, 84.5) | 83.7% (80.1, 87.2) |
| (95% CI) | | |
| Difference in | 3.1% | |
| 12-month PFS rate | | |
| (95% CI) | | |
| 24-month PFS rate | 71.3% (66.8, 75.8) | 77.4% (73.4, 81.5) |
| (95% CI) | | |
| Difference in | 6.1% | |
| 24-month PFS rate | | |
| (95% CI) | | |

Summaries of Progression Free Survival by Investigator (median, percentiles) are Kaplan-Meier estimates.
95% CI for median was computed using the method of Brookmeyer and Crowley.
Hazard ratios were estimated by Cox regression. Stratification factors: geographical region, IPI Score (IPI 2 vs. IPI 3-5), bulky disease (present vs. absent; defined as one lesion >=7.5 cm).

### Subgroup Analysis

In addition, subgroup analyses consistently or generally favored Pola-R-CHP compared to R-CHOP (including baseline characteristics subgroups and biomarker subgroups).

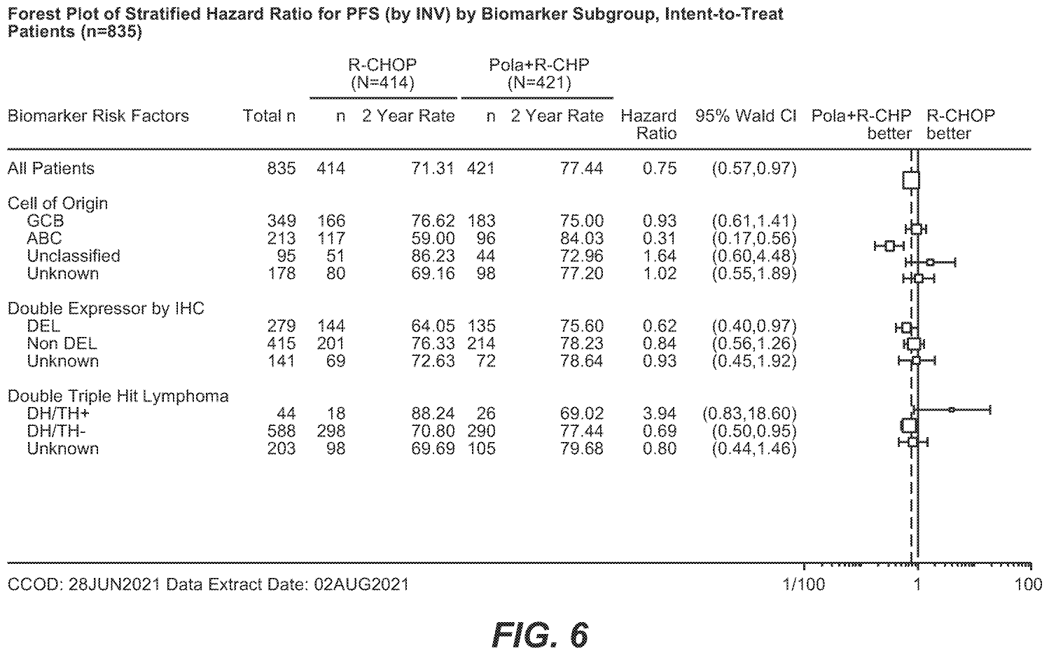

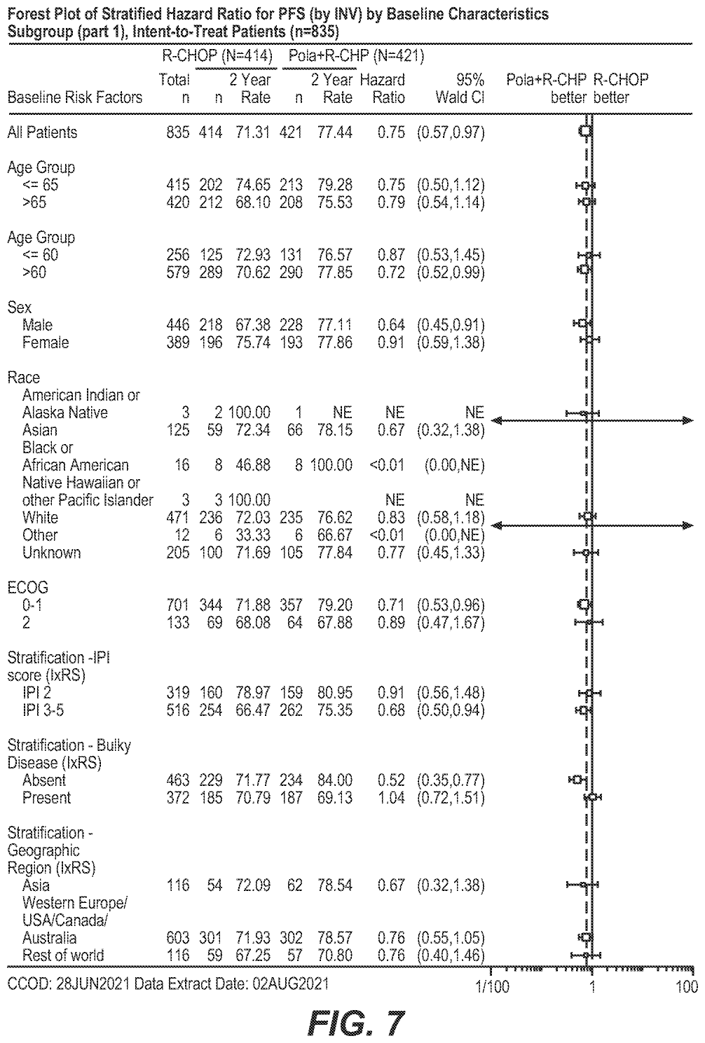

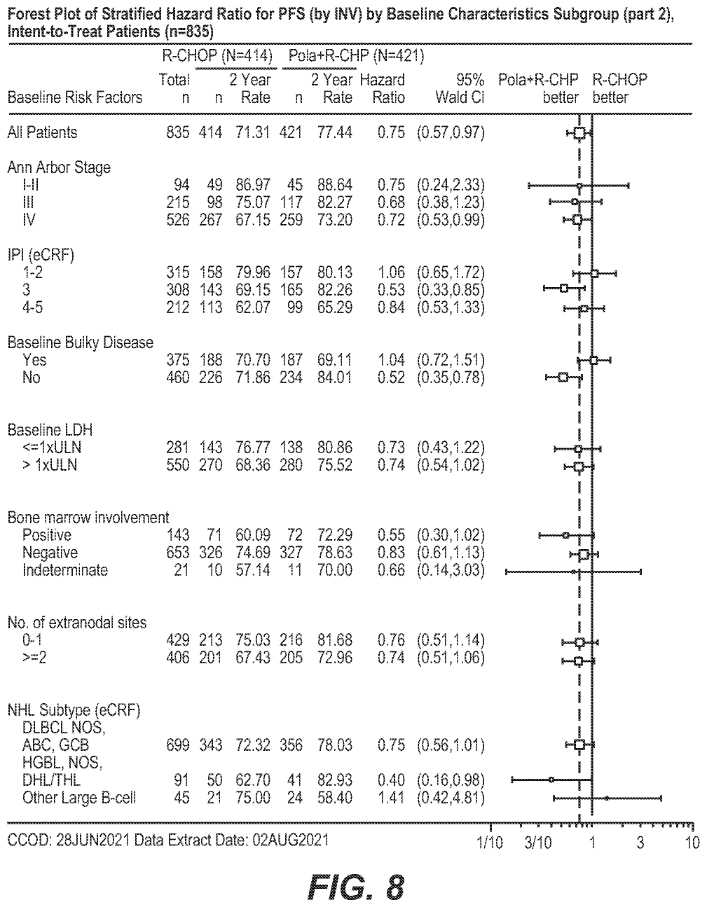

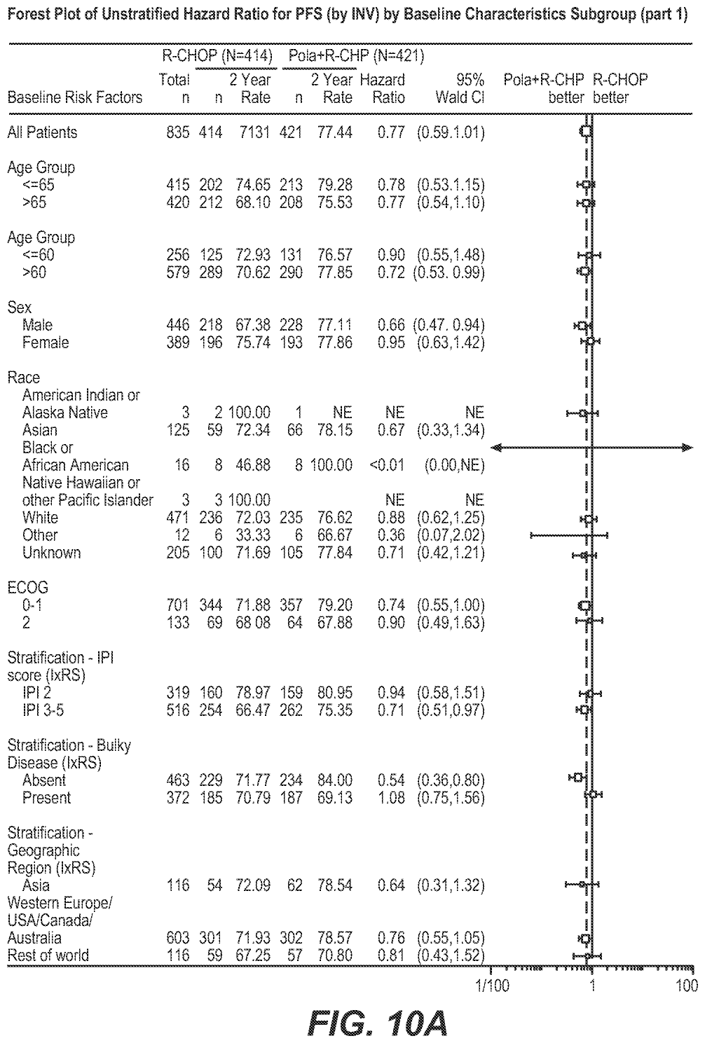

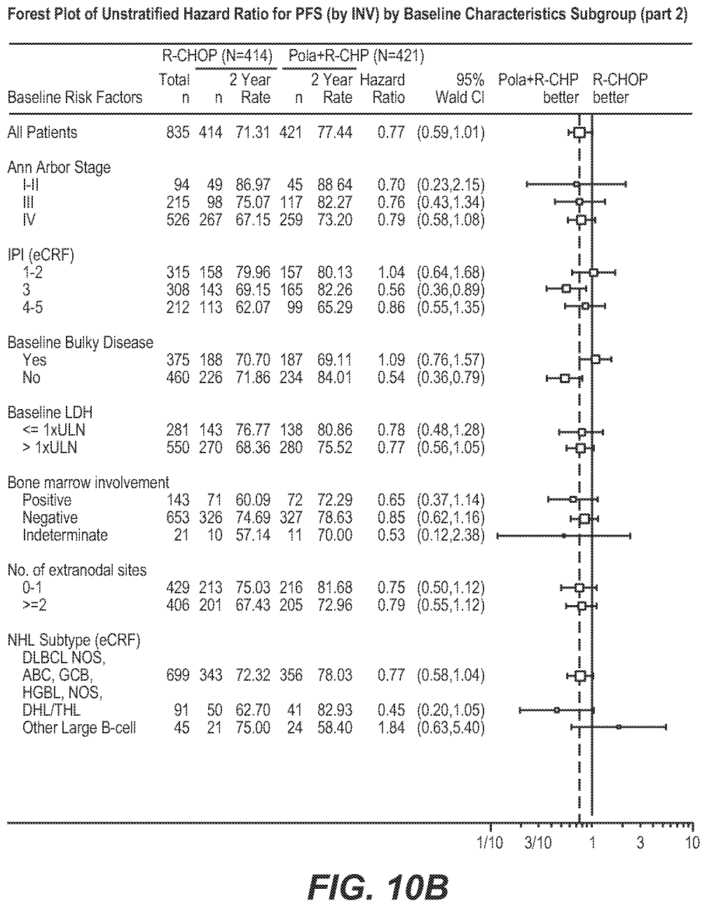

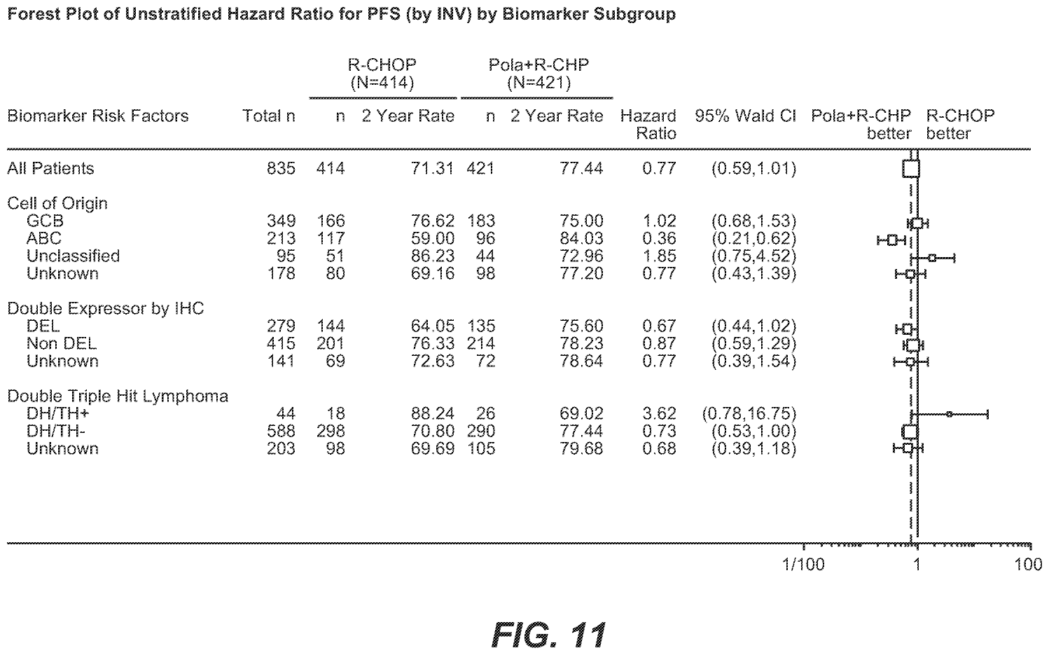

Subgroups analysis also identified several baseline characteristics and biomarker subgroups that appear to strongly favor Pola-R-CHP over R-CHOP using both a stratified and an unstratified analysis as described above, including a) patients <=65 years old, >65 years old, at least 60 years old, or >60 years old; b) the identified histopathologically, high grade b-cell lymphoma, NOS or HGBL with MYC and BCL2 and/or BCL6-rearrangements; c) specific subtypes of DLBCL such as activated B-cell like (ABC) subtype, a double expressing lymphoma (DEL; overexpression of BCL2 and MYC), and DLBCL that does not have double-hit or triple-hit lymphoma defined by MYC and BCL2 and/or BCL6-rearrangements; d) low Ann-Arbor Stage (I-II) and higher Ann-Arbor Stages (III, IV); e) normal baseline LDH levels and elevated baseline LDH levels; f) bone marrow involvement at baseline; g) 0-1 and 2+ extranodal sites; h) an International Prognostic Index (IPI) score between 3-5; and i) absence of bulky disease at baseline. See FIG. 6, FIG. 7, FIG. 8, FIGS. 10A-10B, and FIG. 11. Sensitivity analyses on PFS are consistent with the primary analysis.

Overall, the results of the subgroup analyses suggest that patients with certain characteristics as described above, e.g., an ABC or DEL subtype of DLBCL, an IPI score between 3-5, and/or age greater than 60 or 65 years, are likely to benefit from treatment with Pola-R-CHP compared to the standard of care treatment with R-CHOP.

### Safety

Safety analyses were conducted using the safety analysis population, which included all randomized patients who received at least one dose of any study drug, and specifically for the Pola-R-CHP arm, any exposure to polatuzumab vedotin treatment.

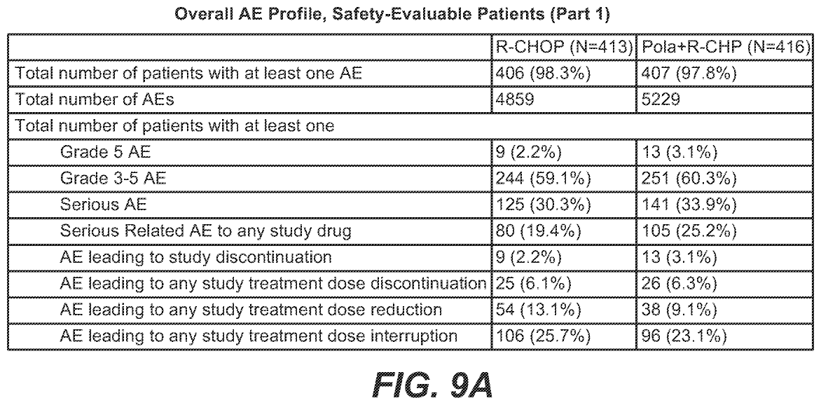

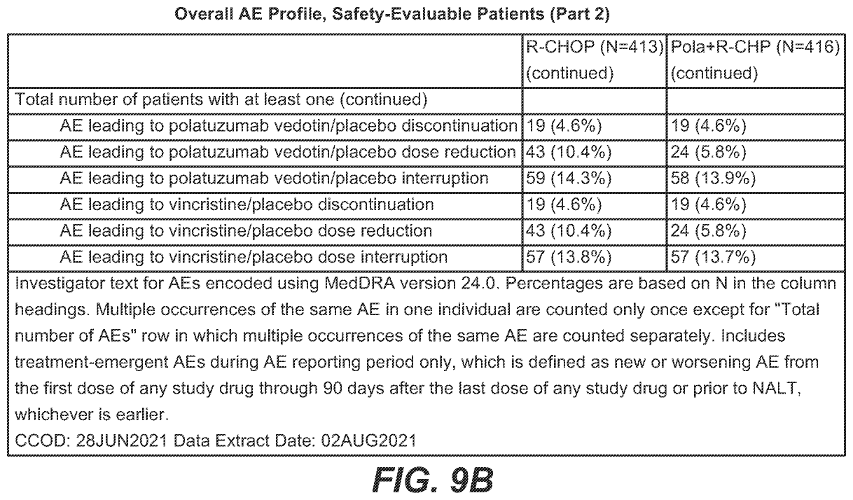

Safety results were consistent with those seen in previous trials. Overall the safety profile of Pola-R-CHP was comparable to R-CHOP and consistent with the known risk of individual study drugs. The combination of Pola-R-CHP was generally well tolerated and the toxicities were manageable. Tolerability of study treatment favored Pola-R-CHP versus R-CHOP, for example, lower incidence of AEs leading to any dose reduction in Pola-R-CHP. No new safety signals were detected. See FIGS. 9A and 9B.

No cases of progressive multifocal leukoencephalopathy (PML) were observed in patients treated with Pola-R-CHP.

### Treatment Exposure

Most patients received all six doses of the active blinded agents, polatuzumab vedotin or vincristine (91.8% and 89.3%, in the Pola-R-CHP and R-CHOP arms, respectively). In addition, 89.4% and 86.9% of patients treated with Pola-R-CHP and R-CHOP, respectively, received all eight cycles of treatment.

### Secondary Efficacy Endpoints

The outcome of EFS_{efficacy} is consistent with PFS (ITT n=835); stratified Hazard Ratio is 0.77 (95% CI; [0.59, 1.00]); stratified p-value (log-rank): 0.05. The 24-month event free survival rate (EFS rate) is 76.3% (72.09, 80.42) for Pola-R-CHP, while 70.7% (66.2, 75.2) for R-CHOP.

The PET-CT CR rate at EOT assessed by BICR (ITT n=835) favors Pola-R-CHP (77.4%) compared to R-CHOP (73.9%).

The Objective Response Rate by PET-CT (by investigators) at EOT (ITT n=835) favors Pola-R-CHP (85.5%) compared to R-CHOP (83.1%).

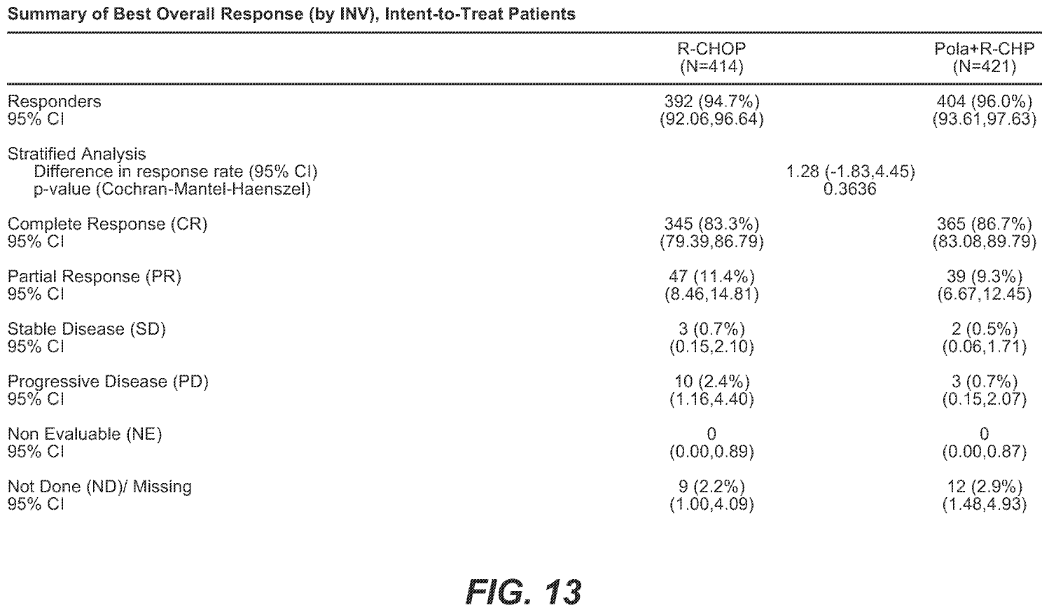

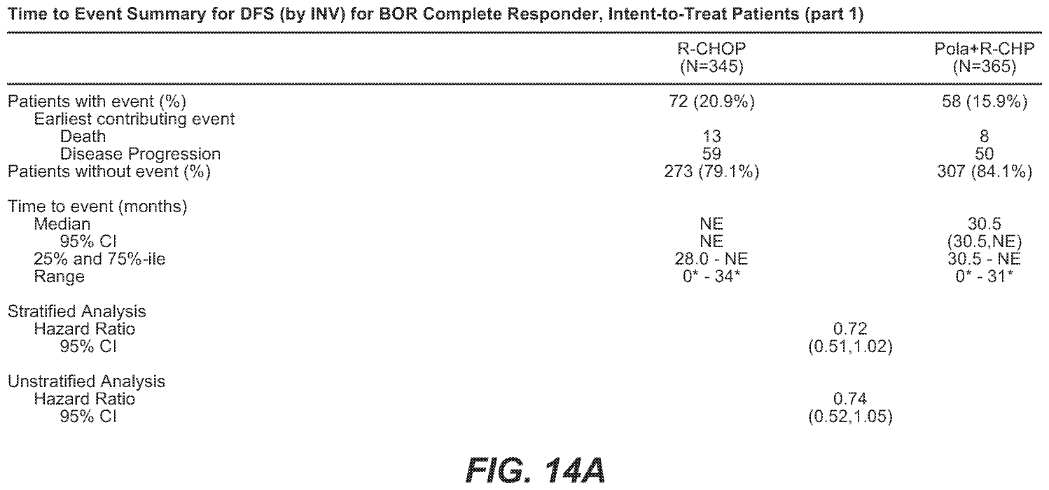

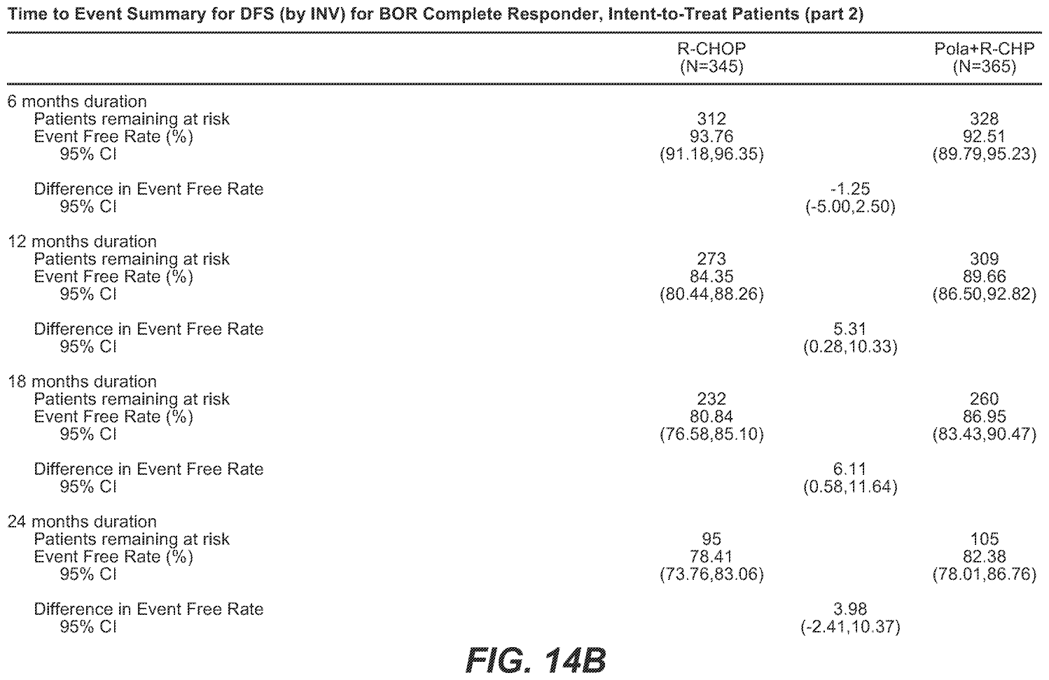

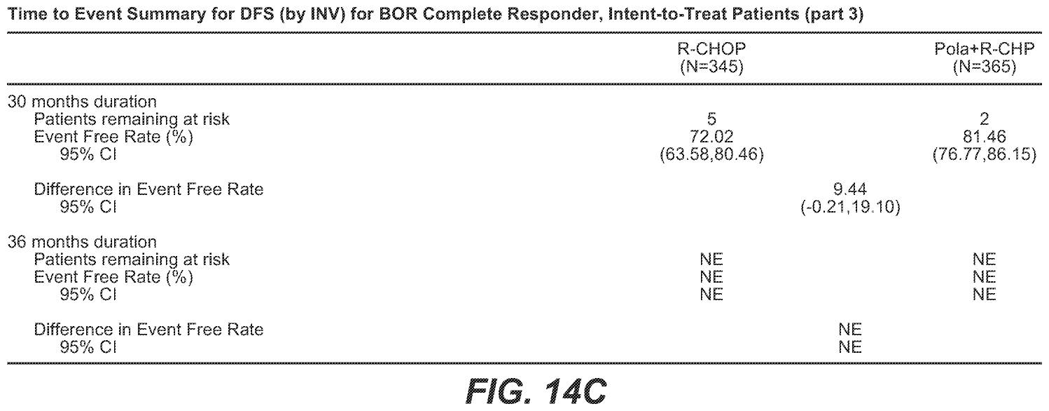

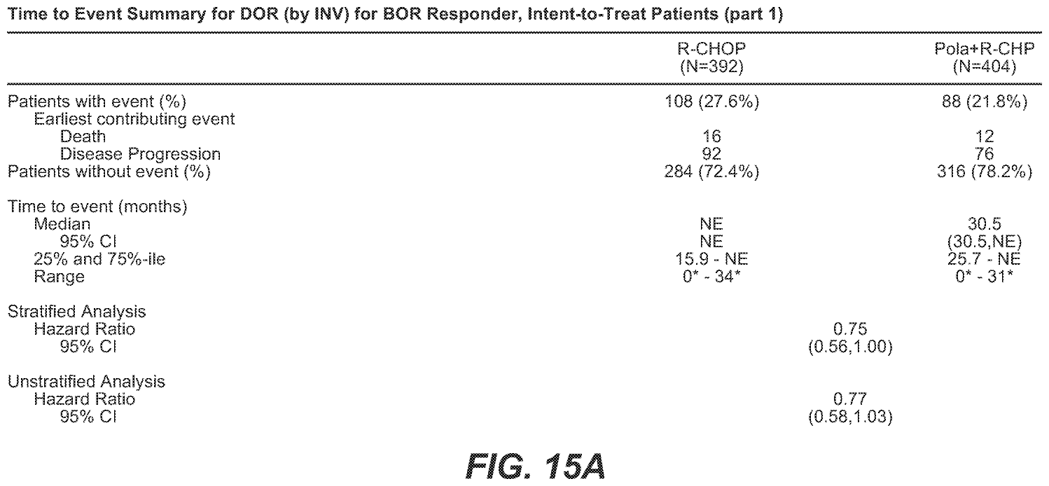

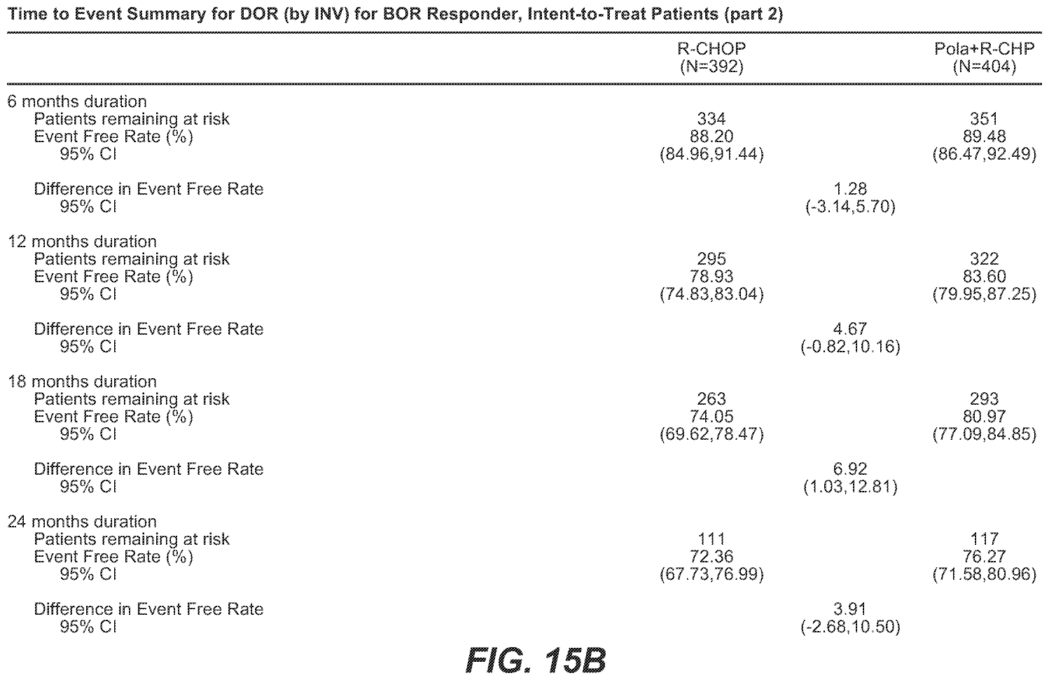

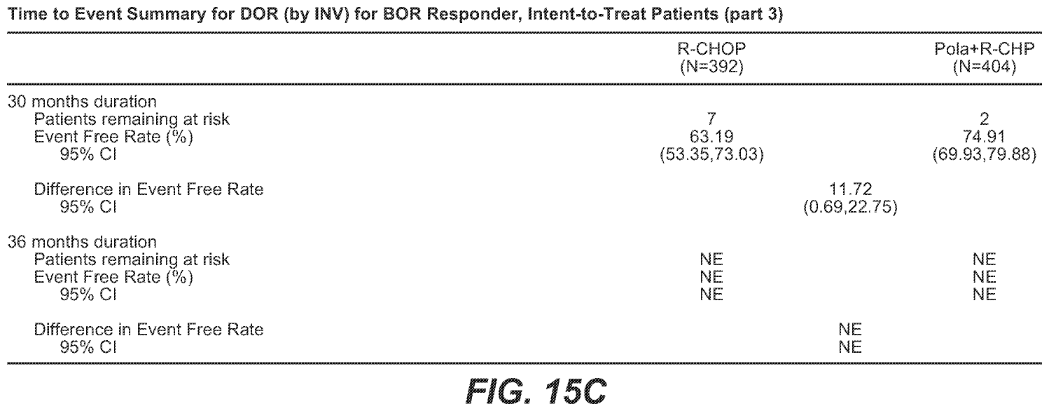

As shown in FIG. 13, the numbers of responders, assessed by best overall response (BOR; by INV), were high and comparable between treatment arms. However, a lower proportion of patients in the Pola-R-CHP arm progressed or died subsequent to achieving a CR, compared to patients in the R-CHOP arm (see, FIGS. 14A-14C, which provide a time-to-event summary for disease-free survival [DFS; by INV] for BOR complete responders). In addition, in patients who achieved CR or PR, treatment with Pola-R-CHP led to more durable responses and reduced the risk of progression or death by about 25% compared to R-CHOP treatment (see, FIGS. 15A-15C, which provide a time-to-event summary for duration of response [DOR; by INV] for BOR responders).

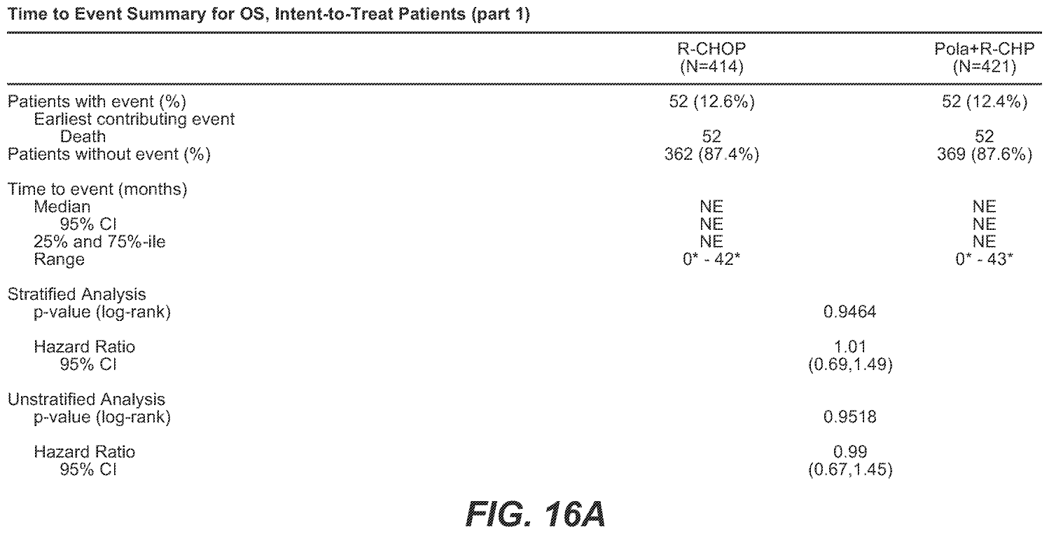

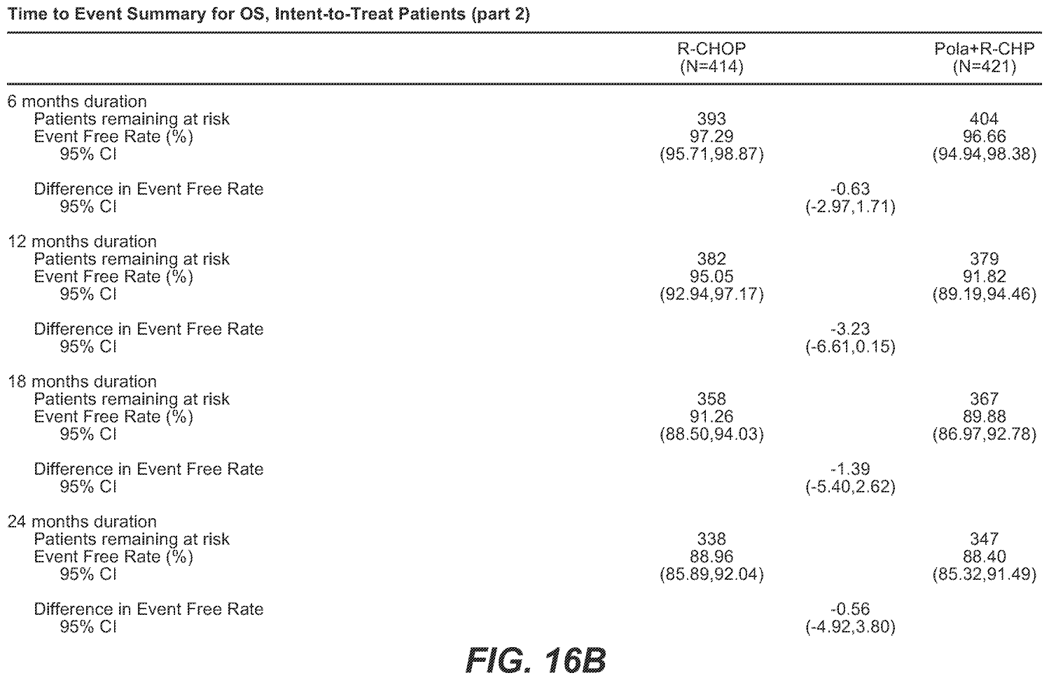

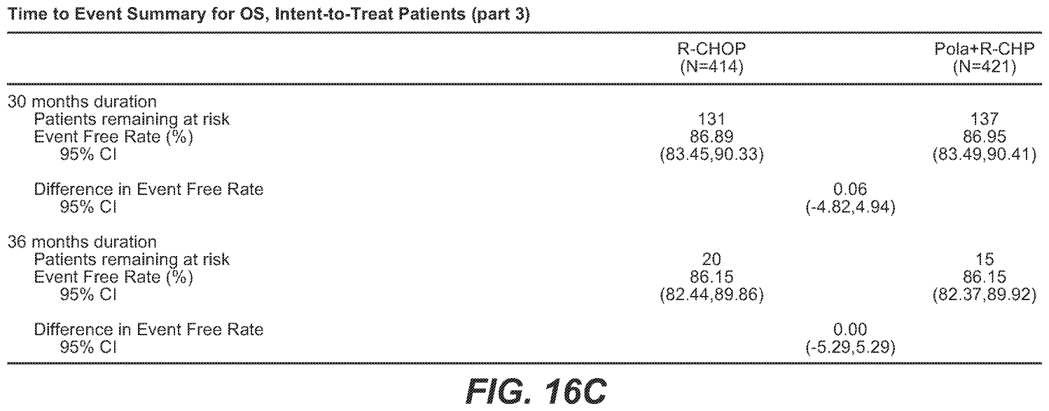

As shown in FIGS. 16A-16C, the frequencies of OS events (deaths) were low in both arms (less than 13%), and no difference in OS duration was observed between the treatment arms in the study.

### New Anti-Lymphoma Therapies

The proportion of patients receiving certain subsequent therapies was lower in patients treated with Pola-R-CHP as compared to patients treated with R-CHOP.

### Conclusions

DLBCL is an aggressive, fast-growing blood cancer, with a median survival of less than one year if left untreated. Even with treatment, as many as 40% of patients relapse or have refractory disease, at which point treatment options are limited and survival is often short.

Thus, this regimen (Pola-R-CHP) is the first regimen in 20 years to prolong survival without disease advancement in first line DLBCL compared to the standard of care. Prolonging survival without disease advancement is transformative for newly diagnosed DLBCL patients, as currently, 40% of patients relapse after disease progression.

### Example 3: Additional Efficacy and Safety Results of a Phase III Study of Polatuzumab Vedotin in Combination with Rituximab and Cyclophosphamide, Doxorubicin, and Prednisone (Pola-R-CHP) Compared to Rituximab, Cyclophosphamide, Doxorubicin, Vincristine, and Prednisone (R-CHOP) in Previously Untreated Diffuse Large B-Cell Lymphoma (DLBCL)

This Example provides additional efficacy and safety results of the Phase III study described in Examples 1 and 2, above, at a median follow-up time of approximately 40 months (with a minimum follow-up time of 36 months and a maximum follow-up time of up to about 54 months).

### Subgroup Analysis

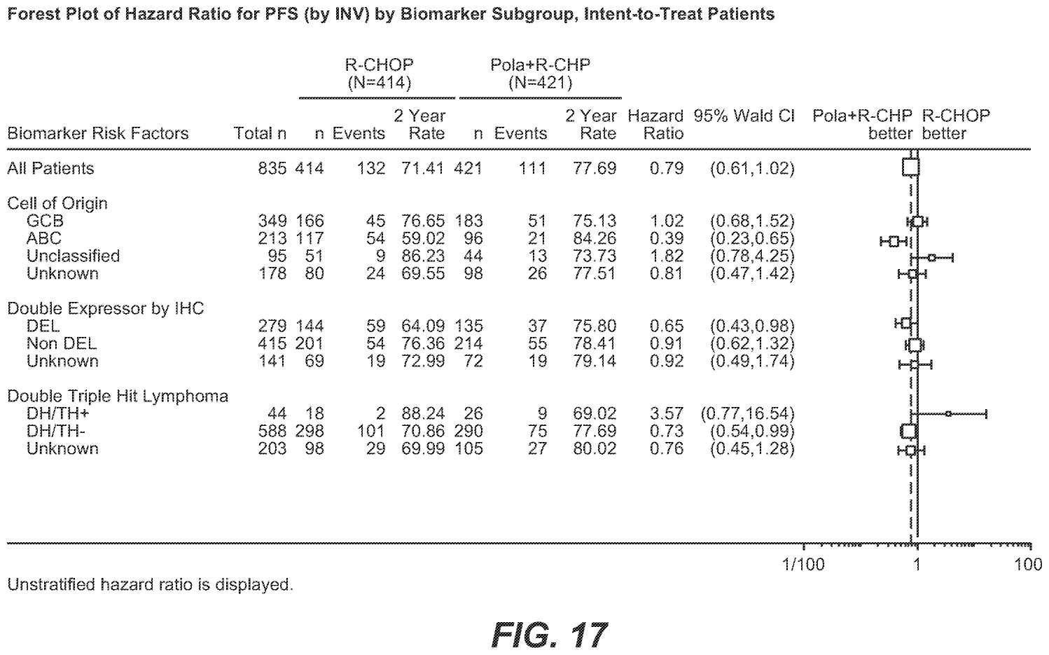

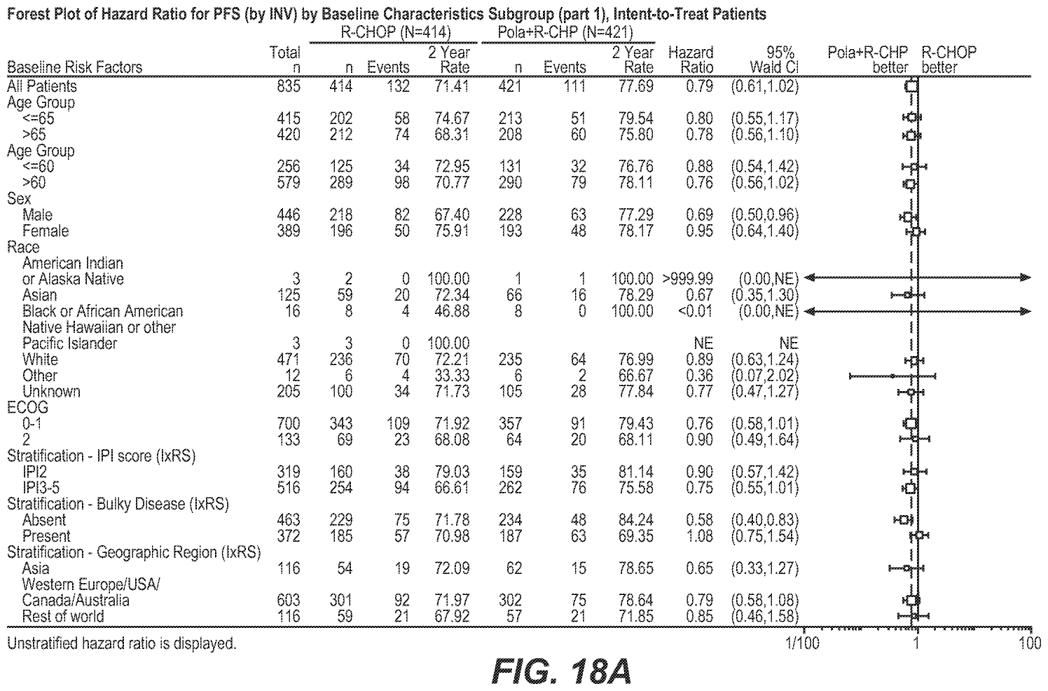

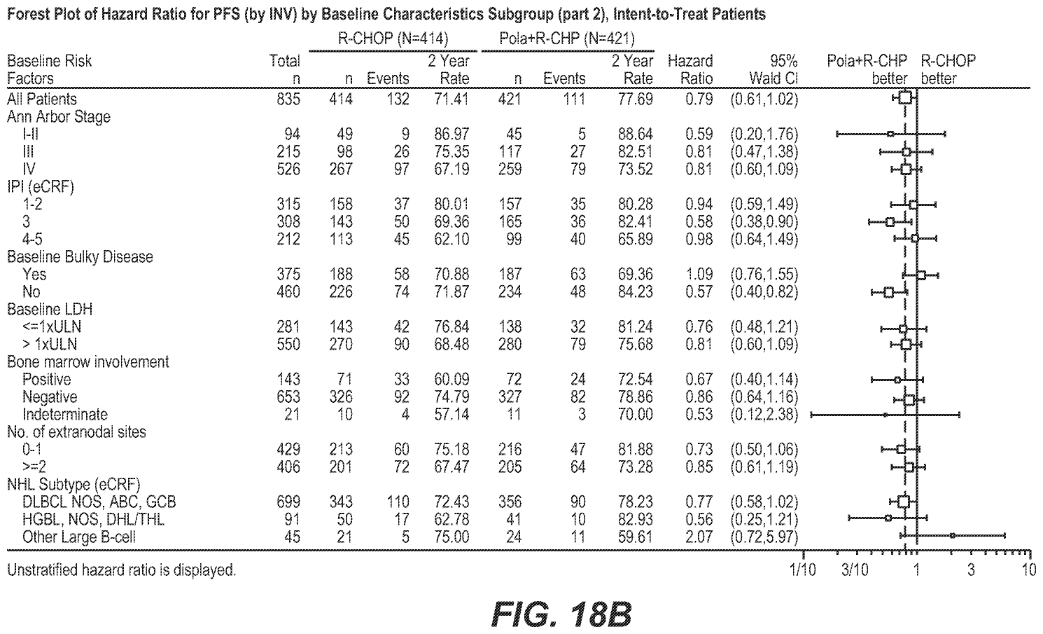

Subgroup analyses at the median follow-up time of approximately 40 months identified several baseline characteristics and biomarker subgroups that appear to strongly favor Pola-R-CHP over R-CHOP, consistent with the subgroup analysis results provided in Example 2, above. These included, for example, an ABC or DEL subtype of DLBCL, an IPI score between 3-5, and/or age greater than 60 or 65 years. See. FIG. 17 and FIGS. 18A-18B.

### Treatment Exposure

Treatment exposure at the median follow-up time of approximately 40 months was consistent with the treatment exposure results as described in Example 2, above.

### Primary Efficacy Endpoint

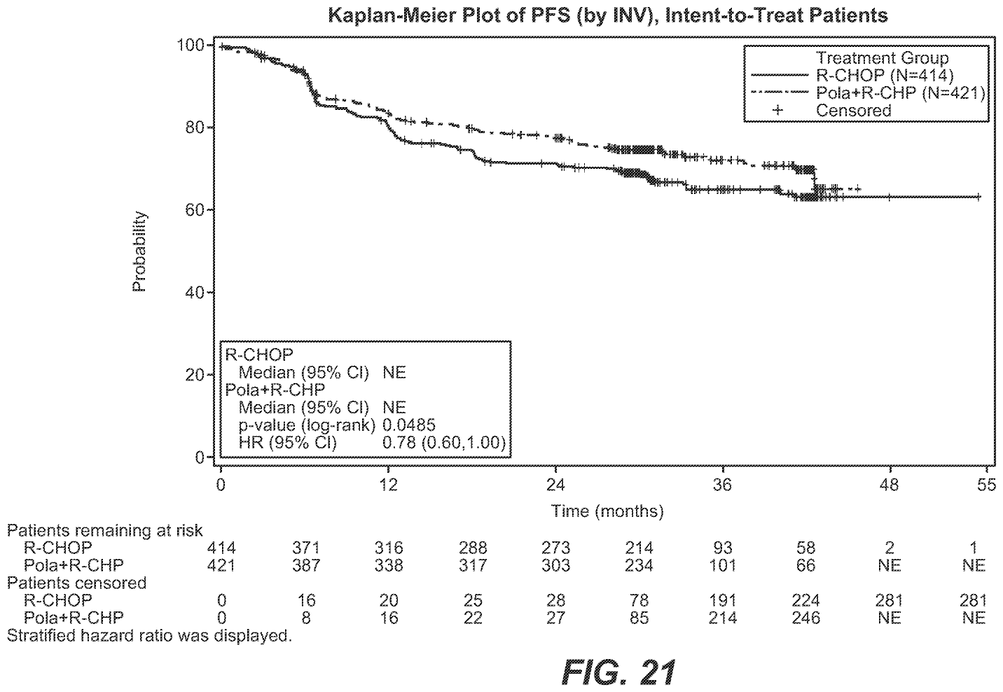

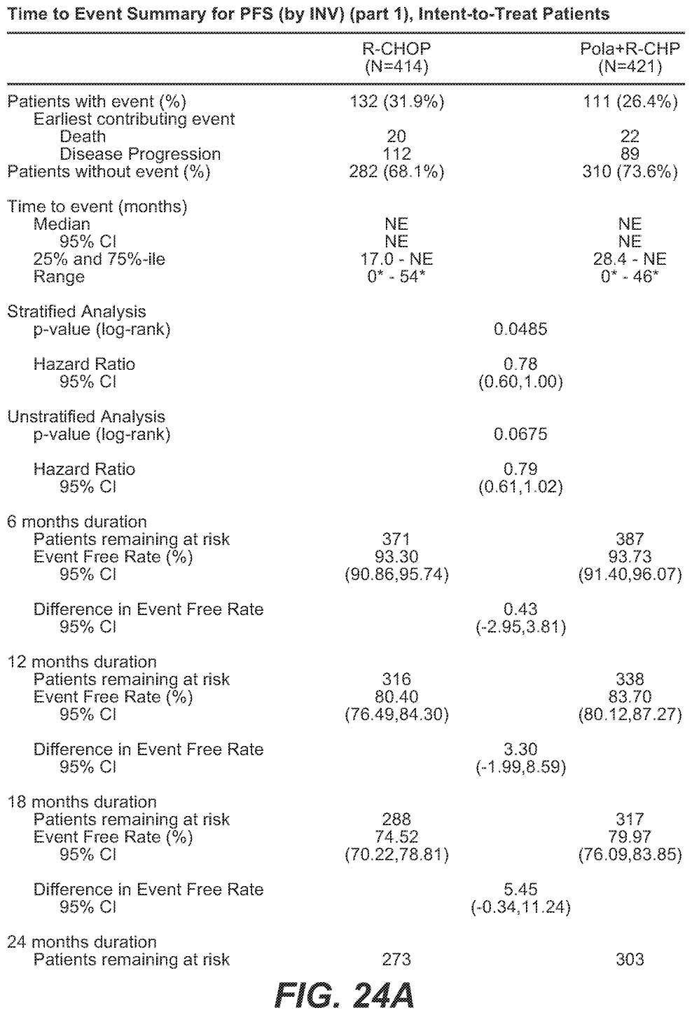

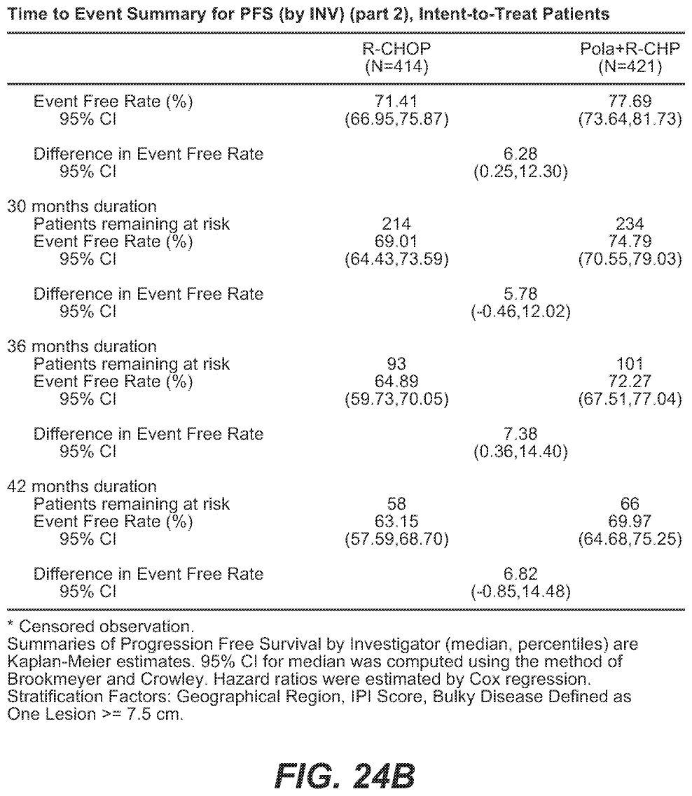

A further analysis of progression-free survival (PFS) at a median follow-up time of approximately 40 months showed that treatment with Pola-R-CHP resulted in an improvement in PFS compared to R-CHOP, with a stratified hazard ratio of 0.78 (95% CI: 0.60, 1.00), and p-value (log-rank) of 0.0485; and with an unstratified hazard ratio of 0.79 (95% CI: 0.61, 1.02), and p-value (log-rank) of 0.0675. Treatment with Pola-R-CHP also resulted in a 42-month PFS rate of 69.97% (95% CI: 64.68, 75.25), as compared to the 42-month PFS rate of 63.15% (95% CI: 57.59, 68.70) with R-CHOP. See, FIG. 21 and FIGS. 24A-24B.

In addition, Pola-R-CHP treatment reduced the risk of progression or death by about 21% or about 22% compared to R-CHOP treatment. See, FIGS. 24A-24B.

### Secondary Efficacy Endpoints

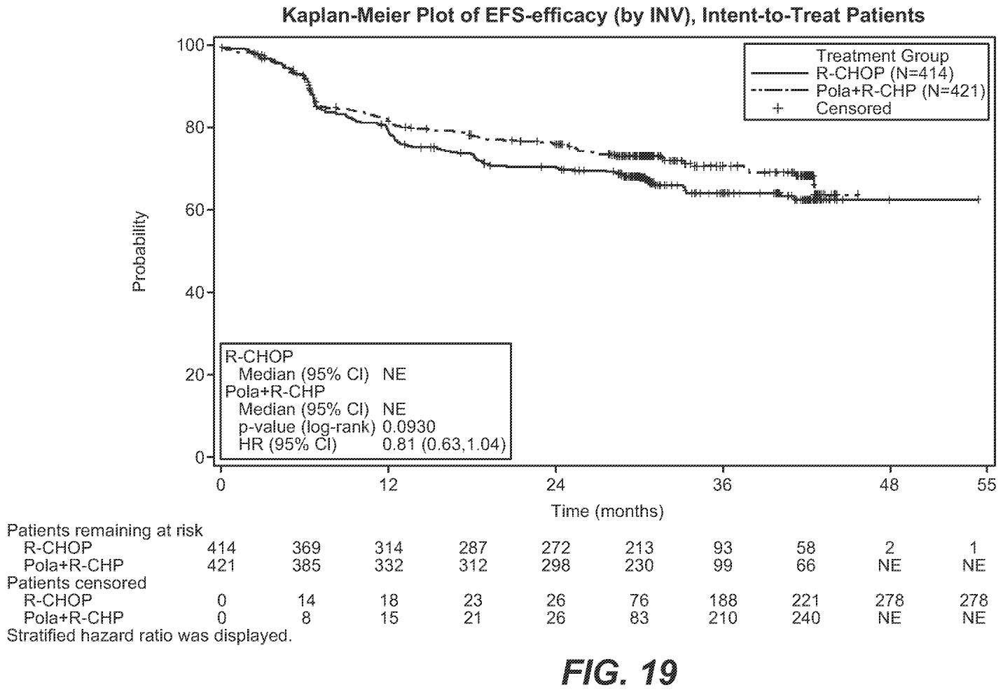

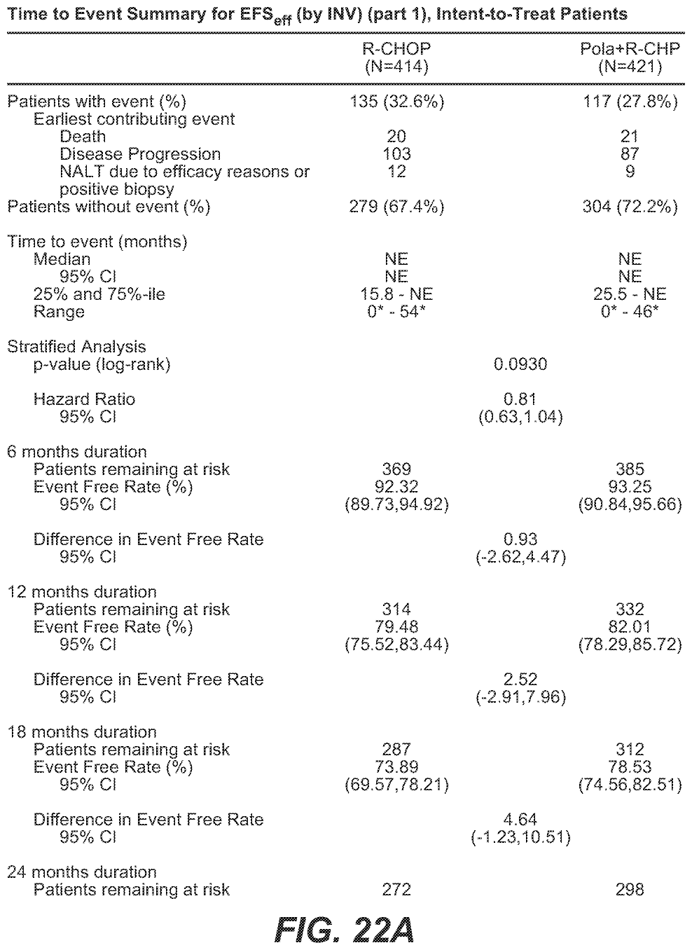

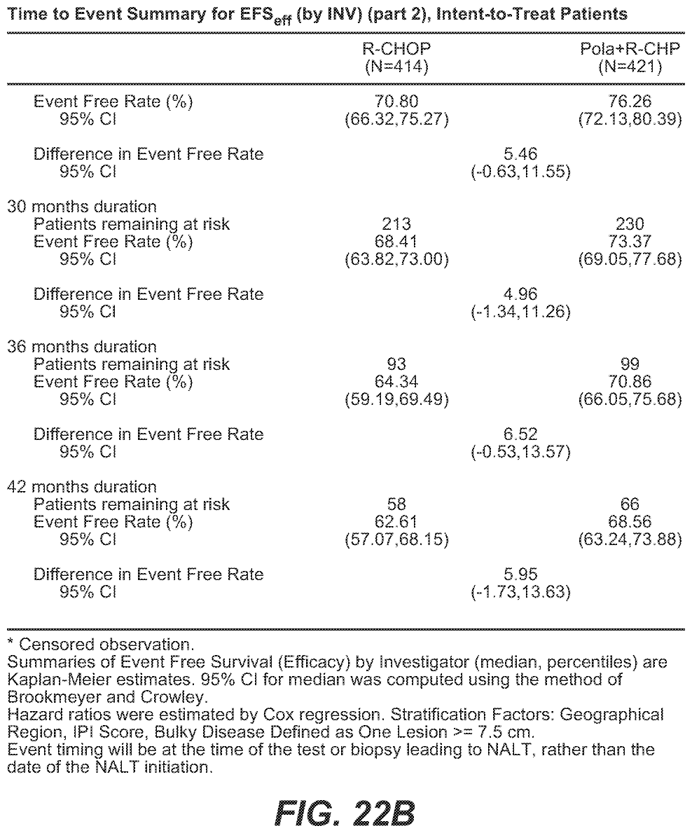

The EFS_{efficacy} (EFS_{eff}) was improved in patients treated with Pola-R-CHP compared to R-CHOP, with a stratified Hazard Ratio of 0.81 (95% CI: 0.63, 1.04), and p-value (log-rank) of 0.0930 (FIG. 19). In addition, the 24-month EFS_{eff} rate was 76.26% with Pola-R-CHP versus 70.80% with R-CHOP, the 36-month EFS_{eff} rate was 70.86% with Pola-R-CHP versus 64.34% with R-CHOP, and the 42-month EFS_{eff} rate was 68.56% with Pola-R-CHP versus 62.61% with R-CHOP (FIGS. 22A-22B).

The PET-CT complete response (CR) rate at end of treatment (EOT), assessed by BICR (ITT population; n=835), favors Pola-R-CHP (77.7%; 95% CI: 73.39, 81.56) compared to R-CHOP (73.9%; 95% CI: 69.40, 78.08).

The objective response rate (ORR) by PET-CT was 84.3% (95% CI: 80.49, 87.66) for Pola-R-CHP and 81.4% (95% CI: 77.31, 85.03) for R-CHOP when assessed by the investigators (ITT population; n=835), and 85.0% (95% CI: 81.26, 88.31) for Pola-R-CHP and 84.3% (95% CI: 80.43, 87.67) for R-CHOP when assessed by the BICR (ITT population; n=835), both of which favor Pola-R-CHP compared to R-CHOP.

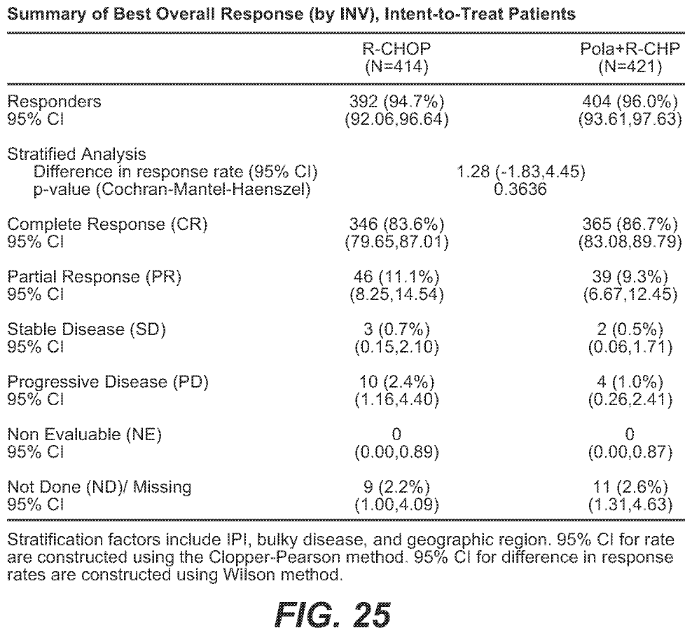

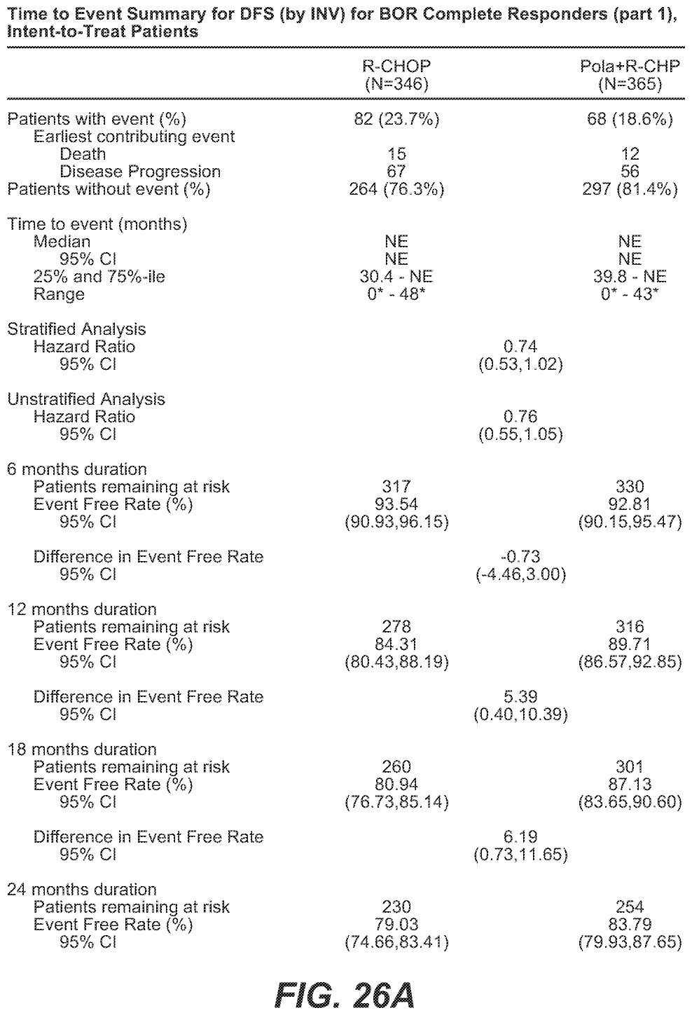

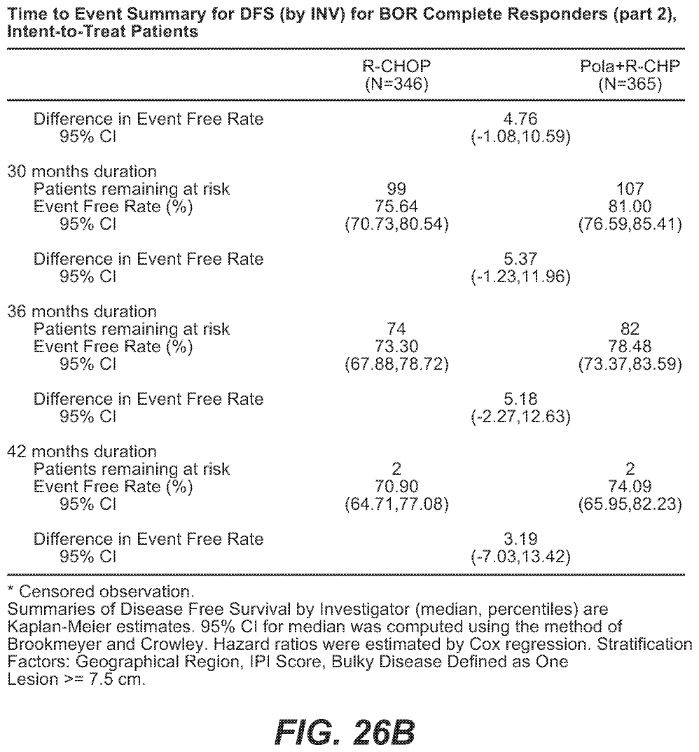

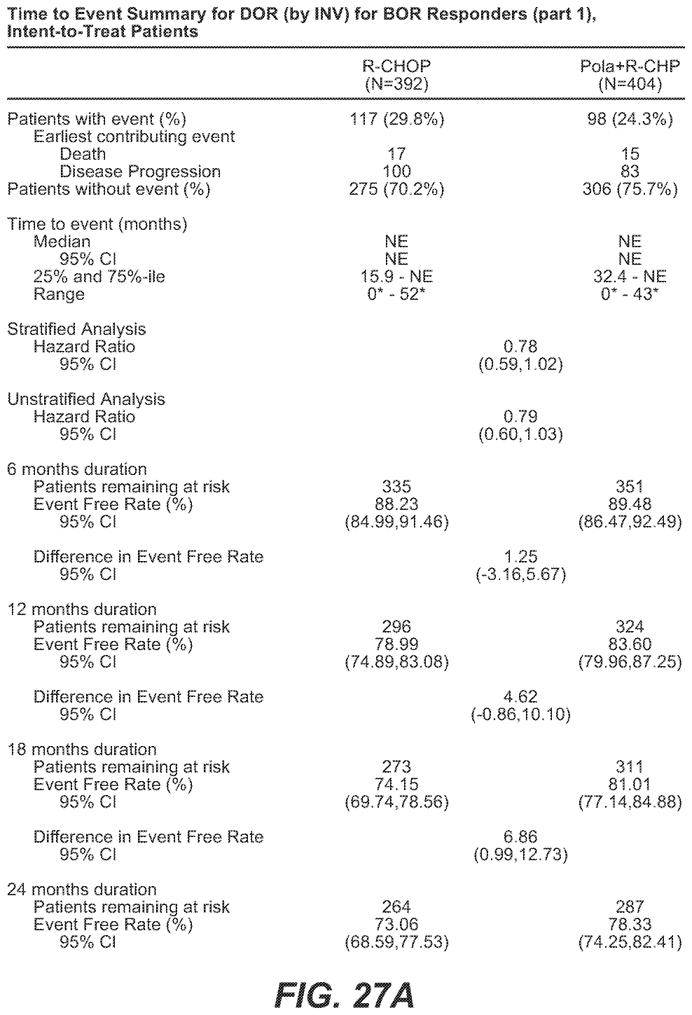

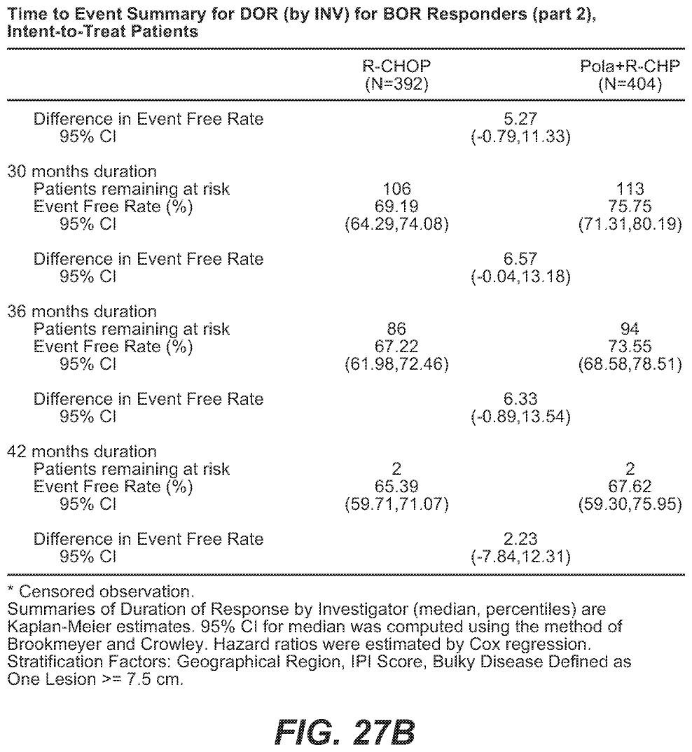

As shown in FIG. 25, the numbers of responders, assessed by best overall response (BOR; by INV), were high and comparable between treatment arms. A lower proportion of patients in the Pola-R-CHP arm progressed or died subsequent to achieving a CR, compared to patients in the R-CHOP arm. See, FIGS. 26A-26B, which provide a time-to-event summary for disease-free survival (DFS) assessed by the investigators for BOR complete responders. In addition, in patients who achieved CR or PR, treatment with Pola-R-CHP led to more durable responses. The risk of progression or death was reduced by about 21% or 22% compared to R-CHOP treatment (see, FIGS. 27A-27B, which provide a time-to-event summary for duration of response [DOR; by INV] for BOR responders).

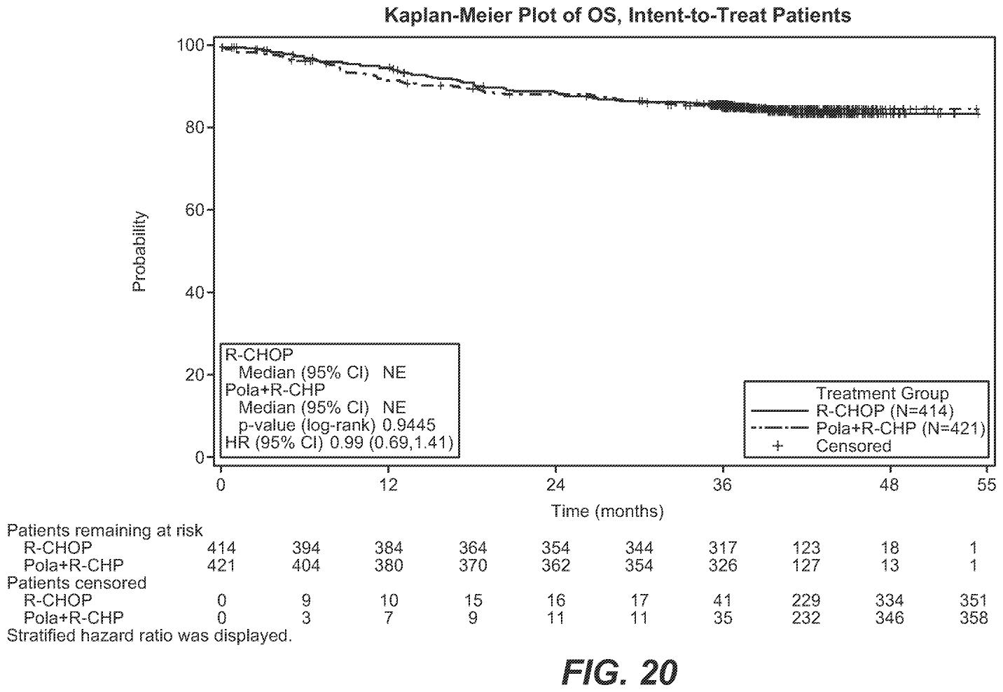

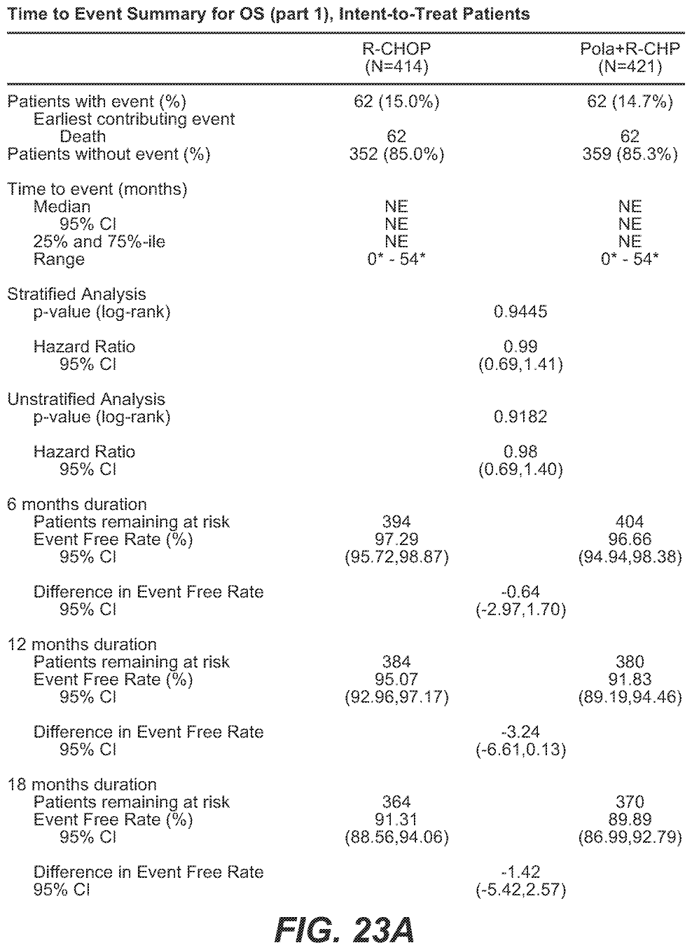

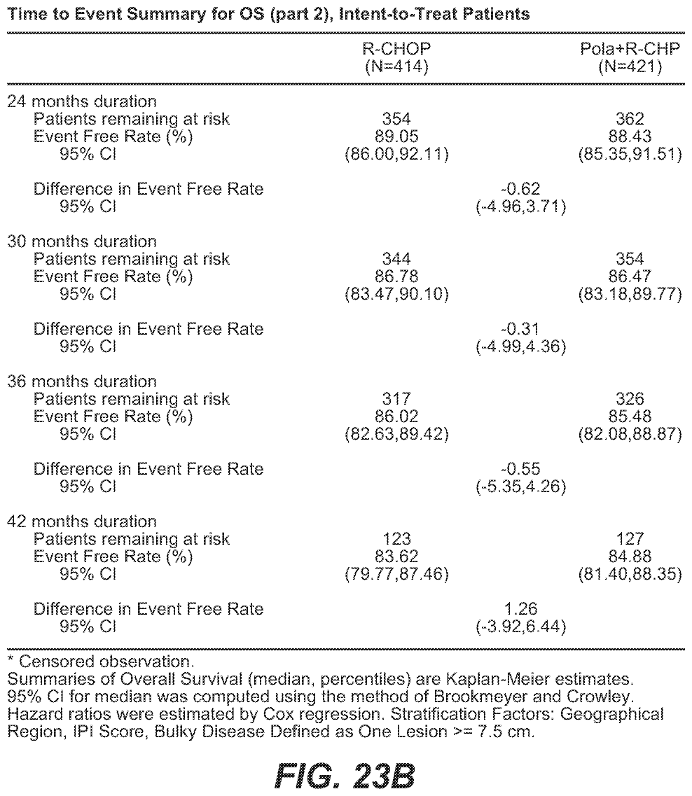

As shown in FIG. 20 and FIGS. 23A-23B, the frequencies of overall survival (OS) events (deaths) were low in both arms (15% or less), and no difference in OS duration was observed between the treatment arms in the study.

### New Anti-Lymphoma Therapies

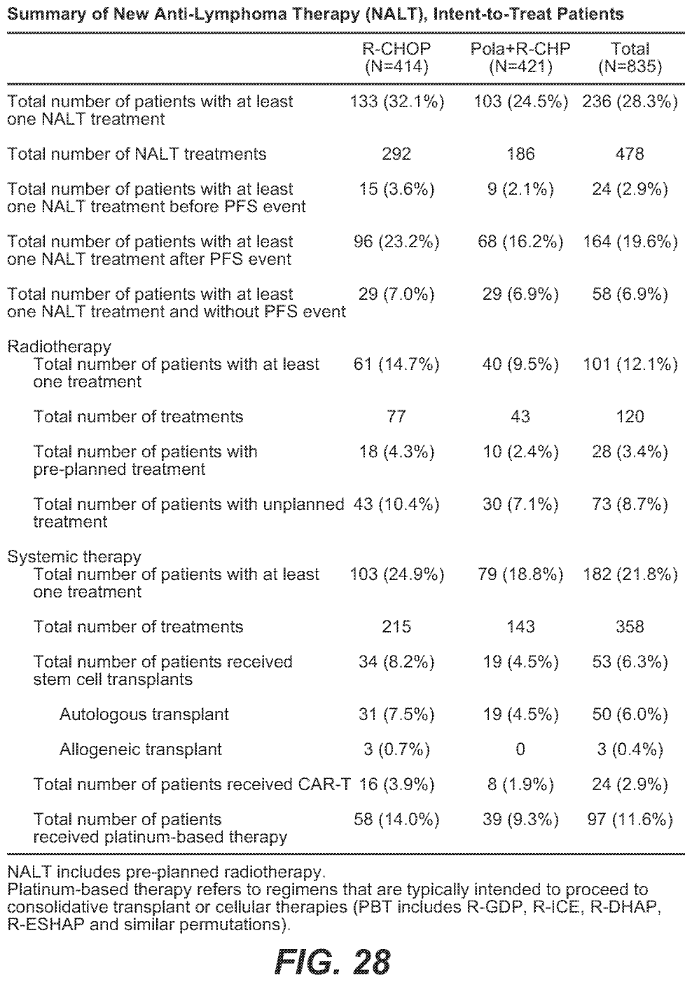

The proportion of patients receiving certain subsequent therapies was lower in patients treated with Pola-R-CHP as compared to patients treated with R-CHOP. See, FIG. 28.

### Safety

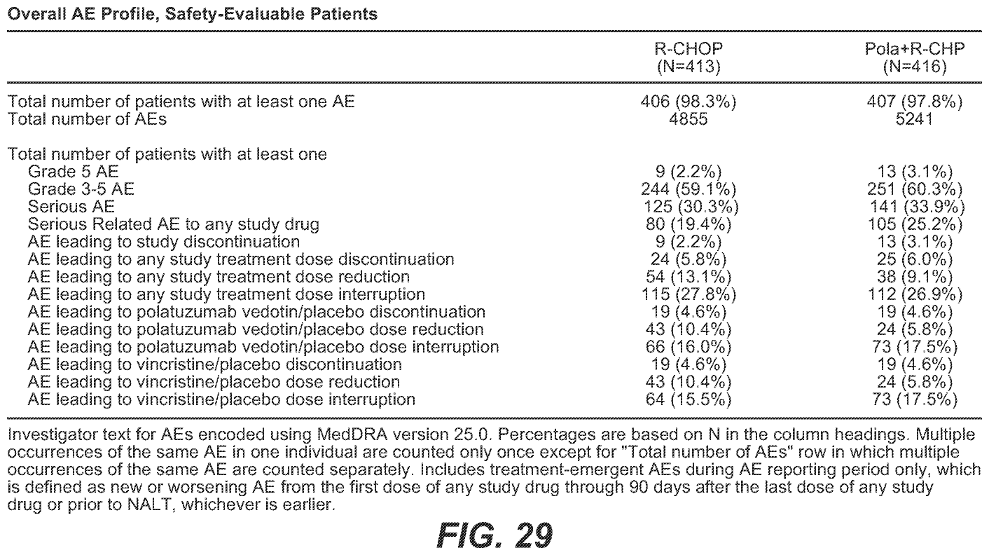

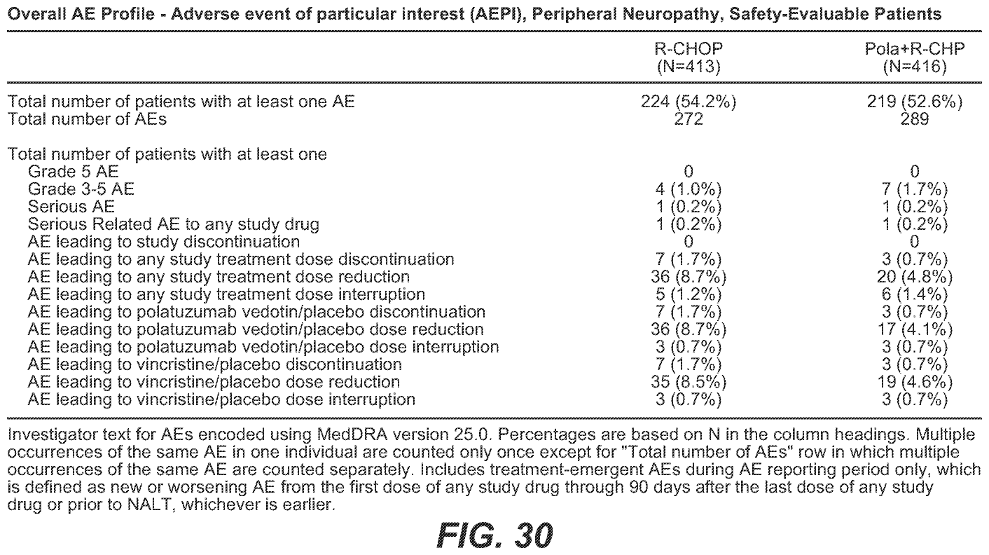

Safety results were consistent with the safety results as described in Example 2, above. See, FIGS. 29 and 30.

### Conclusions

The safety and efficacy results obtained at a median follow-up time of approximately 40 months (with a minimum follow-up time of 36 months and a maximum follow-up time of up to about 54 months) further confirmed that the Pola-R-CHP regimen prolongs survival without disease advancement in first line DLBCL, as compared to the standard of care.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein administering such treatment to a plurality of human patients results in an improvement in progression-free survival (PFS) of the plurality of human patients as compared to a reference PFS,
wherein the reference PFS is the PFS of a plurality of human patients who have received a control treatment comprising:
(a) rituximab,
(b) cyclophosphamide,
(c) doxorubicin,
(d) vincristine, and
(e) prednisone, prednisolone, or methylprednisolone,
in the absence of polatuzumab vedotin.

2. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein the human patient has an age of greater than 60 years,
wherein administering such treatment to a plurality of human patients having an age of greater than 60 years results in an improvement in progression-free survival (PFS) of the plurality of human patients as compared to a reference PFS, and
wherein the reference PFS is the PFS of a plurality of human patients having an age of greater than 60 years who have received a control treatment comprising:
(a) rituximab,
(b) cyclophosphamide,
(c) doxorubicin,
(d) vincristine, and
(e) prednisone, prednisolone, or methylprednisolone,
in the absence of polatuzumab vedotin.

3. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein the human patient has an age of greater than 65 years,
wherein administering such treatment to a plurality of human patients having an age of greater than 65 years results in an improvement in progression-free survival (PFS) of the plurality of human patients as compared to a reference PFS, and
wherein the reference PFS is the PFS of a plurality of human patients having an age of greater than 65 years who have received a control treatment comprising:
(a) rituximab,
(b) cyclophosphamide,
(c) doxorubicin,
(d) vincristine, and
(e) prednisone, prednisolone, or methylprednisolone,
in the absence of polatuzumab vedotin.

4. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein the human patient has an International Prognostic Index (IPI) score between 3 and 5,
wherein administering such treatment to a plurality of human patients having an IPI score between 3 and 5 results in an improvement in progression-free survival (PFS) of the plurality of human patients as compared to a reference PFS, and
wherein the reference PFS is the PFS of a plurality of human patients having an IPI score between 3 and 5 who have received a control treatment comprising:
(a) rituximab,
(b) cyclophosphamide,
(c) doxorubicin,
(d) vincristine, and
(e) prednisone, prednisolone, or methylprednisolone,
in the absence of polatuzumab vedotin.

5. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein the human patient has an age of greater than 60 years and an International Prognostic Index (IPI) score between 3 and 5,
wherein administering such treatment to a plurality of human patients having an age of greater than 60 years and an IPI score between 3 and 5 results in an improvement in progression-free survival (PFS) of the plurality of human patients as compared to a reference PFS, and
wherein the reference PFS is the PFS of a plurality of human patients having an age of greater than 60 years and an IPI score between 3 and 5 who have received a control treatment comprising:
(a) rituximab,
(b) cyclophosphamide,
(c) doxorubicin,
(d) vincristine, and
(e) prednisone, prednisolone, or methylprednisolone,
in the absence of polatuzumab vedotin.

6. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein the human patient has an age of greater than 65 years and an International Prognostic Index (IPI) score between 3 and 5,
wherein administering such treatment to a plurality of human patients having an age of greater than 65 years and an IPI score between 3 and 5 results in an improvement in progression-free survival (PFS) of the plurality of human patients as compared to a reference PFS, and
wherein the reference PFS is the PFS of a plurality of human patients having an age of greater than 65 years and an IPI score between 3 and 5 who have received a control treatment comprising:
(a) rituximab,
(b) cyclophosphamide,
(c) doxorubicin,
(d) vincristine, and
(e) prednisone, prednisolone, or methylprednisolone,
in the absence of polatuzumab vedotin.

7. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein the human patient has an activated B-cell (ABC) type DLBCL,
wherein administering such treatment to a plurality of human patients having an ABC type DLBCL results in an improvement in progression-free survival (PFS) of the plurality of human patients as compared to a reference PFS, and
wherein the reference PFS is the PFS of a plurality of human patients having an ABC type DLBCL who have received a control treatment comprising:
(a) rituximab,
(b) cyclophosphamide,
(c) doxorubicin,
(d) vincristine, and
(e) prednisone, prednisolone, or methylprednisolone,
in the absence of polatuzumab vedotin.

8. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein the human patient has a double expressing lymphoma (DEL) type DLBCL,
wherein administering such treatment to a plurality of human patients having a DEL type DLBCL results in an improvement in progression-free survival (PFS) of the plurality of human patients as compared to a reference PFS, and
wherein the reference PFS is the PFS of a plurality of human patients having a DEL type DLBCL who have received a control treatment comprising:
(a) rituximab,
(b) cyclophosphamide,
(c) doxorubicin,
(d) vincristine, and
(e) prednisone, prednisolone, or methylprednisolone,
in the absence of polatuzumab vedotin.

9. The method of any one of claims 1-8, wherein the PFS or the reference PFS is measured:
(a) starting from the start of the corresponding treatment to the time of a first occurrence of disease progression, relapse, or death; or
(b) starting from up to 7 days prior to the start of the corresponding treatment to the time of a first occurrence of disease progression, relapse, or death; or
(c) starting from the time from randomization to the time of a first occurrence of disease progression, relapse, or death.

10. The method of any one of claims 1-9, wherein the PFS or the reference PFS is the median PFS of the plurality of human patients receiving the corresponding treatment.

11. The method of any one of claims 1-10, wherein the improvement in PFS is statistically significant.

12. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein administering such treatment to a plurality of human patients results in: at least a 20% reduction in the risk of disease progression, relapse, or death in the plurality of human patients, or at least a 25% reduction in the risk of disease progression, relapse, or death in the plurality of human patients,
as compared to a control treatment comprising:
(a) rituximab,
(b) cyclophosphamide,
(c) doxorubicin,
(d) vincristine, and
(e) prednisone, prednisolone, or methylprednisolone,
in the absence of polatuzumab vedotin.

13. The method of claim 12, wherein said disease progression, relapse, or death are measured:
(a) starting from the start of the corresponding treatment to the time of a first occurrence of disease progression, relapse, or death; or
(b) starting from up to 7 days prior to the start of the corresponding treatment to the time of a first occurrence of disease progression, relapse, or death; or
(c) starting from the time from randomization to the time of a first occurrence of disease progression, relapse, or death.

14. The method of any one of claims 12-13, wherein the reduction in the risk of disease progression, relapse, or death is calculated at 12 months, 24 months, or more, measured starting from:
(a) the start of the corresponding treatment; or
(b) up to 7 days prior to the start of the corresponding treatment; or
(c) the time from randomization to the time of a first occurrence of disease progression, relapse, or death.

15. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone:
wherein administering such treatment to a plurality of human patients results in: a stratified hazard ratio of no more than 0.75 in progression-free survival (PFS) of the plurality of human patients, or a stratified hazard ratio of no more than 0.78 in progression-free survival (PFS) of the plurality of human patients, or an unstratified hazard ratio of no more than 0.79 in progression-free survival (PFS) of the plurality of human patients,
as compared to a control treatment comprising:
(a) rituximab,
(b) cyclophosphamide,
(c) doxorubicin,
(d) vincristine, and
(e) prednisone, prednisolone, or methylprednisolone,
in the absence of polatuzumab vedotin.

16. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein:
(i) the human patient has an age greater than 60 years, and wherein administering such treatment to a plurality of human patients having an age greater than 60 years results in a stratified hazard ratio of no more than 0.72 in progression-free survival (PFS) of the plurality of human patients as compared to a control treatment, or
(ii) the human patient has an age greater than 65 years, and wherein administering such treatment to a plurality of human patients having an age greater than 65 years results in a stratified hazard ratio of no more than 0.79 in PFS of the plurality of human patients as compared to a control treatment;
wherein the control treatment comprises:
(a) rituximab,
(b) cyclophosphamide,
(c) doxorubicin,
(d) vincristine, and
(e) prednisone, prednisolone, or methylprednisolone,
in the absence of polatuzumab vedotin.

17. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein the human patient has an International Prognostic Index (IPI) score of between 3 and 5, and
wherein administering such treatment to a plurality of human patients having an IPI score of between 3 and 5 results in a stratified hazard ratio of no more than 0.68 in progression-free survival (PFS) of the plurality of human patients, as compared to a control treatment comprising:
(a) rituximab,
(b) cyclophosphamide,
(c) doxorubicin,
(d) vincristine, and
(e) prednisone, prednisolone, or methylprednisolone,
in the absence of polatuzumab vedotin.

18. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein:
(i) the human patient has an activated B-cell (ABC) type DLBCL, and wherein administering such treatment to a plurality of human patients having an ABC type DLBCL results in a stratified hazard ratio of no more than 0.31 in progression-free survival (PFS) of the plurality of human patients as compared to a control treatment, or
(ii) the human patient has a double expressing lymphoma (DEL) type DLBCL, and wherein administering such treatment to a plurality of human patients having a DEL type DLBCL results in a stratified hazard ratio of no more than 0.62 in PFS of the plurality of human patients as compared to a control treatment;
wherein the control treatment comprises:
(a) rituximab,
(b) cyclophosphamide,
(c) doxorubicin,
(d) vincristine, and
(e) prednisone, prednisolone, or methylprednisolone,
in the absence of polatuzumab vedotin.

19. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein:
(i) the human patient has an age greater than 60 years, and wherein administering such treatment to a plurality of human patients having an age greater than 60 years results in: an unstratified hazard ratio of no more than 0.72 in progression-free survival (PFS) of the plurality of human patients as compared to a control treatment, or an unstratified hazard ratio of no more than 0.76 in progression-free survival (PFS) of the plurality of human patients as compared to a control treatment, or
(ii) the human patient has an age greater than 65 years, and wherein administering such treatment to a plurality of human patients having an age greater than 65 years results in: an unstratified hazard ratio of no more than 0.77 in PFS of the plurality of human patients as compared to a control treatment, or an unstratified hazard ratio of no more than 0.78 in PFS of the plurality of human patients as compared to a control treatment;
wherein the control treatment comprises:
(a) rituximab,
(b) cyclophosphamide,
(c) doxorubicin,
(d) vincristine, and
(e) prednisone, prednisolone, or methylprednisolone,
in the absence of polatuzumab vedotin.

20. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein the human patient has an International Prognostic Index (IPI) score between 3 and 5, and
wherein administering such treatment to a plurality of human patients having an IPI score between 3 and 5 results in: an unstratified hazard ratio of no more than 0.71 in progression-free survival (PFS) of the plurality of human patients, or an unstratified hazard ratio of no more than 0.75 in progression-free survival (PFS) of the plurality of human patients,
as compared to a control treatment comprising:
(a) rituximab,
(b) cyclophosphamide,
(c) doxorubicin,
(d) vincristine, and
(e) prednisone, prednisolone, or methylprednisolone,
in the absence of polatuzumab vedotin.

21. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein:
(i) the human patient has an activated B-cell (ABC) type DLBCL, and wherein administering such treatment to a plurality of human patients having an ABC type DLBCL results in: an unstratified hazard ratio of no more than 0.36 in progression-free survival (PFS) of the plurality of human patients, or an unstratified hazard ratio of no more than 0.39 in progression-free survival (PFS) of the plurality of human patients,
as compared to a control treatment, or
(ii) the human patient has a double expressing lymphoma (DEL) type DLBCL, and wherein administering such treatment to a plurality of human patients having a DEL type DLBCL results in: an unstratified hazard ratio of no more than 0.65 in PFS of the plurality of human patients, or an unstratified hazard ratio of no more than 0.67 in PFS of the plurality of human patients,
as compared to a control treatment:
wherein the control treatment comprises:
(a) rituximab,
(b) cyclophosphamide,
(c) doxorubicin,
(d) vincristine, and
(e) prednisone, prednisolone, or methylprednisolone,
in the absence of polatuzumab vedotin.

22. The method of any one of claims 15-21, wherein the PFS is measured:
(a) starting from the start of the corresponding treatment to the time of a first occurrence of disease progression, relapse, or death; or
(b) starting from up to 7 days prior to the start of the corresponding treatment to the time of a first occurrence of disease progression, relapse, or death; or
(c) starting from the time from randomization to the time of a first occurrence of disease progression, relapse, or death.

23. The method of any one of claims 15-18 and 22, wherein the stratified hazard ratio is stratified by:
(a) geographical region selected from the group consisting of (i) Asia, (ii) Western Europe, United States of America, Canada, or Australia, and (iii) the rest of the world excluding (i)-(ii);
(b) International Prognostic Index (IPI) score of 2 versus between 3 and 5; and/or (c) the presence or absence of bulky disease.

24. The method of any one of claims 15 and 22-23, wherein administering such treatment results in a statistically significant improvement in the PFS as compared to the control treatment with: a stratified hazard ratio of no more than 0.75 (95% confidence interval: 0.57, 0.97), or a stratified hazard ratio of no more than 0.78 (95% confidence interval: 0.60, 1.00), or an unstratified hazard ratio of no more than 0.79 (95% confidence interval: 0.61, 1.02).

25. The method of any one of claims 16 and 22-23, wherein: (a) administering such treatment to a plurality of human patients having an age greater than 60 years results in an improvement in the PFS as compared to the control treatment with a stratified hazard ratio of no more than 0.72 (95% confidence interval: 0.52, 0.99); or (b) administering such treatment to a plurality of human patients having an age greater than 65 years results in an improvement in the PFS as compared to the control treatment with a stratified hazard ratio of no more than 0.79 (95% confidence interval: 0.54, 1.14).

26. The method of any one of claims 17 and 22-23, wherein administering such treatment to a plurality of human patients having an IPI score between 3 and 5 results in an improvement in the PFS as compared to the control treatment with a stratified hazard ratio of no more than 0.68 (95% confidence interval: 0.50, 0.94).

27. The method of any one of claims 18 and 22-23, wherein: (a) administering such treatment to a plurality of human patients having an ABC type DLBCL results in an improvement in the PFS as compared to the control treatment with a stratified hazard ratio of no more than 0.31 (95% confidence interval: 0.17, 0.56); or (b) administering such treatment to a plurality of human patients having a DEL type DLBCL results in an improvement in the PFS as compared to the control treatment with a stratified hazard ratio of no more than 0.62 (95% confidence interval: 0.40, 0.97).

28. The method of claim 19 or claim 22, wherein: (a) administering such treatment to a plurality of human patients having an age greater than 60 years results in an improvement in the PFS as compared to the control treatment with an unstratified hazard ratio of no more than 0.72 (95% confidence interval: 0.53, 0.99); (b) administering such treatment to a plurality of human patients having an age greater than 65 years results in an improvement in the PFS as compared to the control treatment with an unstratified hazard ratio of no more than 0.77 (95% confidence interval: 0.54, 1.10); (c) administering such treatment to a plurality of human patients having an age greater than 60 years results in an improvement in the PFS as compared to the control treatment with an unstratified hazard ratio of no more than 0.76 (95% confidence interval: 0.56, 1.02); or
(d) administering such treatment to a plurality of human patients having an age greater than 65 years results in an improvement in the PFS as compared to the control treatment with an unstratified hazard ratio of no more than 0.78 (95% confidence interval: 0.56, 1.10).

29. The method of claim 20 or claim 22, wherein administering such treatment to a plurality of human patients having an IPI score between 3 and 5 results in: an improvement in the PFS as compared to the control treatment with an unstratified hazard ratio of no more than 0.71 (95% confidence interval: 0.51, 0.97); or an improvement in the PFS as compared to the control treatment with an unstratified hazard ratio of no more than 0.75 (95% confidence interval: 0.55, 1.01).

30. The method of claim 21 or claim 22, wherein: (a) administering such treatment to a plurality of human patients having an ABC type DLBCL results in an improvement in the PFS as compared to the control treatment with an unstratified hazard ratio of no more than 0.36 (95% confidence interval: 0.21, 0.62); (b) administering such treatment to a plurality of human patients having an ABC type DLBCL results in an improvement in the PFS as compared to the control treatment with an unstratified hazard ratio of no more than 0.39 (95% confidence interval: 0.23, 0.65); (c) administering such treatment to a plurality of human patients having a DEL type DLBCL results in an improvement in the PFS as compared to the control treatment with an unstratified hazard ratio of no more than 0.65 (95% confidence interval: 0.43, 0.98); or (d) administering such treatment to a plurality of human patients having a DEL type DLBCL results in an improvement in the PFS as compared to the control treatment with an unstratified hazard ratio of no more than 0.67 (95% confidence interval: 0.44, 1.02).

31. The method of any one of claims 15-30, wherein the stratified or unstratified hazard ratio is calculated at 12 months, 24 months, or more, measured starting from:
(a) the start of the corresponding treatment; or
(b) up to 7 days prior to the start of the corresponding treatment; or
(c) the time from randomization to the time of a first occurrence of disease progression, relapse, or death.

32. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein administering such treatment to a plurality of human patients results in a 24-month progression-free survival rate (PFS24) of at least 75%.

33. The method of claim 32, wherein the PFS24 is calculated at 24 months, measured starting from:
(a) the start of treatment; or
(b) up to 7 days prior to the start of treatment; or
(c) the time from randomization to the time of a first occurrence of disease progression, relapse, or death.

34. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein administering such treatment to a plurality of human patients results in an improvement in a 24-month progression-free survival rate (PFS24) of the plurality of human patients as compared to a reference PFS24,
wherein the reference PFS24 is the 24-month progression-free survival rate of a plurality of human patients who have received a control treatment comprising:
(a) rituximab,
(b) cyclophosphamide,
(c) doxorubicin,
(d) vincristine, and
(e) prednisone, prednisolone, or methylprednisolone,
in the absence of polatuzumab vedotin.

35. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein administering such treatment to a plurality of human patients results in an improvement in a 24-month progression-free survival rate (PFS24) of the plurality of human patients of at least about 6%, as compared to a reference PFS24,
wherein the reference PFS24 is the 24-month progression-free survival rate of a plurality of human patients who have received a control treatment comprising:
(a) rituximab,
(b) cyclophosphamide,
(c) doxorubicin,
(d) vincristine, and
(e) prednisone, prednisolone, or methylprednisolone,
in the absence of polatuzumab vedotin.

36. The method of claim 34 or claim 35, wherein the PFS24 or the reference PFS24 is calculated at 24 months, measured starting from:
(a) the start of the corresponding treatment; or
(b) up to 7 days prior to the start of the corresponding treatment; or
(c) starting from the time from randomization to the time of a first occurrence of disease progression, relapse, or death.

37. The method of any one of claims 32-36, wherein the PFS24 or the reference PFS24 is a progression-free survival (PFS) rate calculated using a Kaplan-Meier method.

38. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein administering such treatment to a plurality of human patients results in a 12-month progression-free survival (PFS) rate of at least 83%.

39. The method of claim 38, wherein the 12-month PFS is calculated at 12 months, measured starting from:
(a) the start of treatment; or
(b) up to 7 days prior to the start of treatment; or
(c) the time from randomization to the time of a first occurrence of disease progression, relapse, or death.

40. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein administering such treatment to a plurality of human patients results in an improvement in a 12-month progression-free survival (PFS) rate of the plurality of human patients as compared to a reference 12-month PFS rate,
wherein the reference 12-month PFS rate is the 12-month PFS rate of a plurality of human patients who have received a control treatment comprising:
(a) rituximab,
(b) cyclophosphamide,
(c) doxorubicin,
(d) vincristine, and
(e) prednisone, prednisolone, or methylprednisolone,
in the absence of polatuzumab vedotin.

41. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein administering such treatment to a plurality of human patients results in an improvement in a 12-month progression-free survival (PFS) rate of the plurality of human patients of at least about 3%, as compared to a reference 12-month PFS rate,
wherein the reference 12-month PFS rate is the 12-month PFS rate of a plurality of human patients who have received a control treatment comprising:
(a) rituximab,
(b) cyclophosphamide,
(c) doxorubicin,
(d) vincristine, and
(e) prednisone, prednisolone, or methylprednisolone,
in the absence of polatuzumab vedotin.

42. The method of claim 40 or claim 41, wherein the 12-month PFS rate or the reference 12-month PFS rate is calculated at 12 months, measured starting from:
(a) the start of the corresponding treatment; or
(b) up to 7 days prior to the start of the corresponding treatment; or
(c) starting from the time from randomization to the time of a first occurrence of disease progression, relapse, or death.

43. The method of any one of claims 38-42, wherein the 12-month PFS rate or the reference 12-month PFS rate is a progression-free survival (PFS) rate calculated using a Kaplan-Meier method.

44. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein administering such treatment to a plurality of human patients results in an improvement in event-free survival-efficacy (EFS_{eff}) of the plurality of human patients as compared to a reference EFS_{eff},
wherein the reference EFS_{eff} is the EFS_{eff} of a plurality of human patients who have received a control treatment comprising:
(a) rituximab,
(b) cyclophosphamide,
(c) doxorubicin,
(d) vincristine, and
(e) prednisone, prednisolone, or methylprednisolone,
in the absence of polatuzumab vedotin.

45. The method of claim 44, wherein the EFS_{eff} or the reference EFS_{eff} is measured:
(a) starting from the start of the corresponding treatment to the time of a first occurrence of an EFS_{eff} event; or
(b) starting from up to 7 days prior to the start of the corresponding treatment to the time of a first occurrence of an EFS_{eff} event; or
(c) starting from the time from randomization to the time of a first occurrence of an EFS_{eff} event.

46. The method of claim 44 or claim 45, wherein the improvement in EFS_{eff} is statistically significant.

47. The method of any one of claims 44-46, wherein the improvement in EFS_{eff} is calculated at 12 months, 24 months, or more, measured starting from:
(a) the start of the corresponding treatment; or
(b) up to 7 days prior to the start of the corresponding treatment; or
(c) the time from randomization to the time of a first occurrence of an EFS_{eff} event.

48. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein administering such treatment to a plurality of human patients results in: a stratified hazard ratio of no more than 0.77 in event-free survival-efficacy (EFS_{eff}) in the plurality of human patients, or a stratified hazard ratio of no more than 0.81 in EFS_{eff} in the plurality of human patients,
as compared to a control treatment comprising:
(a) rituximab,
(b) cyclophosphamide,
(c) doxorubicin,
(d) vincristine, and
(e) prednisone, prednisolone, or methylprednisolone,
in the absence of polatuzumab vedotin.

49. The method of claim 48, wherein the EFS_{eff} is measured:
(a) starting from the start of the corresponding treatment to the time of a first occurrence of an EFS_{eff} event; or
(b) starting from up to 7 days prior to the start of the corresponding treatment to the time of a first occurrence of an EFS_{eff} event; or
(c) starting from the time from randomization to the time of a first occurrence of an EFS_{eff} event.

50. The method of claim 48 or claim 49, wherein administering such treatment results in a statistically significant improvement in the EFS_{eff} as compared to the control treatment with: a stratified hazard ratio of no more than 0.77 (95% confidence interval: 0.59, 1.00); or a stratified hazard ratio of no more than 0.81 (95% confidence interval: 0.63, 1.04).

51. The method of any one of claims 48-50, wherein the hazard ratio is calculated at 12 months, 24 months, or more, measured starting from:
(a) the start of the corresponding treatment; or
(b) up to 7 days prior to the start of the corresponding treatment; or
(c) the time from randomization to the time of a first occurrence of an EFS_{eff} event.

52. The method of any one of claims 45-47 and 49-51, wherein the EFS_{eff} event is:
(a) disease progression;
(b) disease relapse;
(c) death;
(d) a primary efficacy reason that leads to initiation of a non-protocol specified anti-lymphoma treatment (NALT), and that is not disease progression or relapse; or
(e) a biopsy positive for residual disease.

53. The method of any one of claims 48-52, wherein the stratified hazard ratio is stratified by: (a) geographical region selected from the group consisting of (i) Asia, (ii) Western Europe, United States of America, Canada, or Australia, and (iii) the rest of the world excluding (i)-(ii); (b) International Prognostic Index (IPI) score of 2 versus between 3 and 5; and/or (c) the presence or absence of bulky disease.

54. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone:
wherein administering such treatment to a plurality of human patients results in a rate of complete response (CR) at end of treatment (EOT) in the plurality of human patients of at least about 77%, wherein the rate of CR is assessed by positron emission tomography-computed tomography (PET-CT).

55. The method of claim 54, wherein the CR is assessed by an investigator or by blinded independent central review (BICR).

56. The method of claim 54 or claim 55, wherein administering such treatment to a plurality of human patients results in an improvement in the rate of CR of at least about 3% in the plurality of human patients, as compared to a plurality of human patients who have received a control treatment comprising:
(a) rituximab,
(b) cyclophosphamide,
(c) doxorubicin,
(d) vincristine, and
(e) prednisone, prednisolone, or methylprednisolone,
in the absence of polatuzumab vedotin.

57. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein administering such treatment to a plurality of human patients results in: an objective response rate (ORR) at end of treatment (EOT) in the plurality of human patients of at least about 84%, or an ORR at EOT in the plurality of human patients of at least about 85%,
wherein the ORR is assessed by positron emission tomography-computed tomography (PET-CT).

58. The method of claim 57, wherein the ORR is assessed by an investigator or by blinded independent central review (BICR).

59. The method of claim 57 or claim 58, wherein administering such treatment to a plurality of human patients results in an improvement in ORR of at least about 2% in the plurality of human patients, as compared to a plurality of human patients who have received a control treatment comprising:
(a) rituximab,
(b) cyclophosphamide,
(c) doxorubicin,
(d) vincristine, and
(e) prednisone, prednisolone, or methylprednisolone,
in the absence of polatuzumab vedotin.

60. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein the human patient has an age of greater than 60 years.

61. A method for treating diffuse large B-cell lymphoma (DLBCL) in a human patient in need thereof, comprising administering to the human patient an effective amount of:
(a) polatuzumab vedotin,
(b) rituximab,
(c) cyclophosphamide,
(d) doxorubicin, and
(e) prednisone, prednisolone, or methylprednisolone;
wherein the human patient has an age of greater than 65 years.

62. The method of any one of claims 1-61, wherein polatuzumab vedotin is administered at a dose of about 1.8 mg/kg.

63. The method of any one of claims 1-62, wherein rituximab is administered at a dose of about 375 mg/m².

64. The method of any one of claims 1-63, wherein cyclophosphamide is administered at a dose of about 750 mg/m².

65. The method of any one of claims 1-64, wherein doxorubicin is administered at a dose of about 50 mg/m².

66. The method of any one of claims 1-31, 34-37, 40-53, 56, 59, and 62-65, wherein vincristine is administered at a dose of about 1.4 mg/m² and up to 2 mg each dose.

67. The method of any one of claims 1-66, wherein:
(a) prednisone is administered at a dose of about 100 mg;
(b) prednisolone is administered at a dose of about 100 mg; or
(c) methylprednisolone is administered at a dose of about 80 mg.

68. The method of any one of claims 1-67, wherein:
(a) the polatuzumab vedotin is administered to the human patient at a dose of about 1.8 mg/kg;
(b) the rituximab is administered to the human patient at a dose of about 375 mg/m²;
(c) the cyclophosphamide is administered to the human patient at a dose of about 750 mg/m²;
(d) the doxorubicin is administered to the human patient at a dose of about 50 mg/m²; and
(e) the prednisone is administered to the human patient at a dose of about 100 mg; the prednisolone is administered to the human patient at a dose of about 100 mg; or the methylprednisolone is administered to the human patient at a dose of about 80 mg.

69. The method of any one of claims 1-68, wherein:
(a) the polatuzumab vedotin is administered to the human patient intravenously at a dose of about 1.8 mg/kg;
(b) the rituximab is administered to the human patient intravenously at a dose of about 375 mg/m²;
(c) the cyclophosphamide is administered to the human patient intravenously at a dose of about 750 mg/m²;
(d) the doxorubicin is administered to the human patient intravenously at a dose of about 50 mg/m²; and
(e) the prednisone is administered to the human patient orally at a dose of about 100 mg: the prednisolone is administered to the human patient orally at a dose of about 100 mg; or the methylprednisolone is administered to the human patient intravenously at a dose of about 80 mg.

70. The method of any one of claims 1-61, wherein:
(a) the polatuzumab vedotin is administered to the human patient at a dose of about 1.0 mg/kg to about 1.8 mg/kg;
(b) the rituximab is administered to the human patient at a dose of about 375 mg/m²;
(c) the cyclophosphamide is administered to the human patient at a dose of about 375 mg/m² to about 750 mg/m²;
(d) the doxorubicin is administered to the human patient at a dose of about 25 mg/m² to about 50 mg/m²; and
(e) the prednisone is administered to the human patient at a dose of about 100 mg: the prednisolone is administered to the human patient at a dose of about 100 mg; or the methylprednisolone is administered to the human patient at a dose of about 80 mg.

71. The method of any one of claims 1-61 and 70, wherein:
(a) the polatuzumab vedotin is administered to the human patient intravenously at a dose of about 1.0 mg/kg to about 1.8 mg/kg;
(b) the rituximab is administered to the human patient intravenously at a dose of about 375 mg/m²;
(c) the cyclophosphamide is administered to the human patient intravenously at a dose of about 375 mg/m² to about 750 mg/m²;
(d) the doxorubicin is administered to the human patient intravenously at a dose of about 25 mg/m² to about 50 mg/m²; and
(e) the prednisone is administered to the human patient orally at a dose of about 100 mg: the prednisolone is administered to the human patient orally at a dose of about 100 mg; or the methylprednisolone is administered to the human patient intravenously at a dose of about 80 mg.

72. The method of any one of claims 1-71, wherein the polatuzumab vedotin, rituximab, cyclophosphamide, doxorubicin, and prednisone, prednisolone, or methylprednisolone are administered to the human patient in 21-day cycles.

73. The method of claim 72, wherein:
the polatuzumab vedotin, the rituximab, the cyclophosphamide, and the doxorubicin are administered on day 1 of each 21-day cycle; and
the prednisone, prednisolone, or methylprednisolone is administered on days 1-5 of each 21-day cycle.

74. The method of claim 72 or claim 73, wherein:
(a) the polatuzumab vedotin is administered to the human patient intravenously at a dose of about 1.8 mg/kg on day 1 of each 21-day cycle;
(b) the rituximab is administered to the human patient intravenously at a dose of about 375 mg/m² on day 1 of each 21-day cycle;
(c) the cyclophosphamide is administered to the human patient intravenously at a dose of about 750 mg/m² on day 1 of each 21-day cycle;
(d) the doxorubicin is administered to the human patient intravenously at a dose of about 50 mg/m² on day 1 of each 21-day cycle; and
(e) the prednisone is administered to the human patient orally at a dose of about 100 mg per day on each of days 1-5 of each 21-day cycle; the prednisolone is administered orally at a dose of about 100 mg per day on each of days 1-5 of each 21-day cycle; or the methylprednisolone is administered intravenously at a dose of about 80 mg per day on each of days 1-5 of each 21-day cycle.

75. The method of any one of claims 1-74, wherein the polatuzumab vedotin, the rituximab, the cyclophosphamide, the doxorubicin, and the prednisone, prednisolone, or methylprednisolone are administered for one, two, three, four, five, or six 21-day cycles.

76. The method of any one of claims 1-74, wherein the polatuzumab vedotin, the rituximab, the cyclophosphamide, the doxorubicin, and the prednisone, prednisolone, or methylprednisolone are administered for at least six 21-day cycles.

77. The method of any one of claims 1-74, wherein the polatuzumab vedotin, the rituximab, the cyclophosphamide, the doxorubicin, and the prednisone, prednisolone, or methylprednisolone are administered for six 21-day cycles.

78. The method of any one of claims 1-31, 34-37, 40-53, 56, 59, and 62-77, wherein the rituximab, cyclophosphamide, doxorubicin, vincristine, and prednisone, prednisolone, or methylprednisolone of the control treatment are administered in 21-day cycles.

79. The method of claim 78, wherein:
the rituximab, cyclophosphamide, doxorubicin, and vincristine are administered on day 1 of each 21-day cycle; and
the prednisone, prednisolone, or methylprednisolone is administered on days 1-5 of each 21-day cycle.

80. The method of claim 78 or claim 79, wherein:
(a) the rituximab is administered intravenously at a dose of about 375 mg/m² on day 1 of each 21-day cycle;
(b) the cyclophosphamide is administered intravenously at a dose of about 750 mg/m² on day 1 of each 21-day cycle;
(c) the doxorubicin is administered intravenously at a dose of about 50 mg/m² on day 1 of each 21-day cycle;
(d) the vincristine is administered intravenously at a dose of about 1.4 mg/m² and up to 2 mg each dose on day 1 of each 21-day cycle; and
(e) the prednisone is administered orally at a dose of about 100 mg per day on each of days 1-5 of each 21-day cycle; the prednisolone is administered orally at a dose of about 100 mg per day on each of days 1-5 of each 21-day cycle; or the methylprednisolone is administered intravenously at a dose of about 80 mg per day on each of days 1-5 of each 21-day cycle.

81. The method of any one of claims 78-80, wherein the rituximab, the cyclophosphamide, the doxorubicin, the vincristine, and the prednisone, prednisolone, or methylprednisolone are administered for one, two, three, four, five, or six 21-day cycles.

82. The method of any one of claims 78-80, wherein the rituximab, the cyclophosphamide, the doxorubicin, the vincristine, and the prednisone, prednisolone, or methylprednisolone are administered for at least six 21-day cycles.

83. The method of any one of claims 78-80, wherein the rituximab, the cyclophosphamide, the doxorubicin, the vincristine, and the prednisone, prednisolone, or methylprednisolone are administered for six 21-day cycles.

84. The method of any one of claims 1-83, wherein the polatuzumab vedotin, the rituximab, the cyclophosphamide, the doxorubicin, and the prednisone are administered to the human patient.

85. The method of any one of claims 1-83, wherein the polatuzumab vedotin, the rituximab, the cyclophosphamide, the doxorubicin, and the prednisolone are administered to the human patient.

86. The method of any one of claims 1-83, wherein the polatuzumab vedotin, the rituximab, the cyclophosphamide, the doxorubicin, and the methylprednisolone are administered to the human patient.

87. The method of any one of claims 72-86, wherein the polatuzumab vedotin, the rituximab, the cyclophosphamide, the doxorubicin, and the prednisone, prednisolone, or methylprednisolone are administered to the human patient sequentially on day 1 of each 21-day cycle.

88. The method of claim 87, wherein:
(a) the prednisone, prednisolone, or methylprednisolone is administered prior to the rituximab;
the rituximab is administered prior to the polatuzumab vedotin; and the polatuzumab vedotin is administered prior to the cyclophosphamide and doxorubicin; or
(b) the rituximab, polatuzumab vedotin, cyclophosphamide and doxorubicin are administered in any order after administration of the prednisone, prednisolone, or methylprednisolone.

89. The method of any one of claims 75-88, further comprising:
(a) administering rituximab monotherapy to the human patient during a seventh and eighth 21-day cycle after the sixth 21-day cycle; or
(b) administering rituximab, cyclophosphamide, doxorubicin, and prednisone, prednisolone, or methylprednisolone to the human patient during a seventh and eighth 21-day cycle after the sixth 21-day cycle.

90. The method of claim 89, comprising administering rituximab monotherapy to the human patient intravenously at a dose of about 375 mg/m² on day 1 of each of the seventh and eighth 21-day cycles.

91. The method of claim 89, comprising administering rituximab, cyclophosphamide, doxorubicin, and prednisone, prednisolone, or methylprednisolone to the human patient, wherein:
(a) the rituximab is administered intravenously at a dose of about 375 mg/m² on day 1 of each of the seventh and eighth 21-day cycles;
(b) the cyclophosphamide is administered intravenously at a dose of about 750 mg/m² on day 1 of each of the seventh and eighth 21-day cycles;
(c) the doxorubicin is administered intravenously at a dose of about 50 mg/m² on day 1 of each of the seventh and eighth 21-day cycles; and
(d) the prednisone is administered orally at a dose of about 100 mg per day on each of days 1-5 of each of the seventh and eighth 21-day cycles; the prednisolone is administered orally at a dose of about 100 mg per day on each of days 1-5 of each of the seventh and eighth 21-day cycles; or the methylprednisolone is administered intravenously at a dose of about 80 mg per day on each of days 1-5 of each of the seventh and eighth 21-day cycles.

92. The method of any one of claims 78-91, wherein the rituximab, the cyclophosphamide, the doxorubicin, the vincristine, and the prednisone, prednisolone, or methylprednisolone of the control treatment are administered sequentially on day 1 of each 21-day cycle.

93. The method of claim 92, wherein: (a) the prednisone, prednisolone, or methylprednisolone is administered prior to the rituximab; and the rituximab is administered prior to the cyclophosphamide, doxorubicin and vincristine; or (b) the rituximab, cyclophosphamide, doxorubicin and vincristine are administered in any order after administration of the prednisone, prednisolone, or methylprednisolone.

94. The method of any one of claims 81-93, wherein the control treatment further comprises:
(a) rituximab monotherapy during a seventh and eighth 21-day cycle after the sixth 21-day cycle;
or
(b) rituximab, cyclophosphamide, doxorubicin, vincristine, and prednisone, prednisolone, or methylprednisolone during a seventh and eighth 21-day cycle after the sixth 21-day cycle.

95. The method of claim 94, wherein the control treatment further comprises rituximab monotherapy administered intravenously at a dose of about 375 mg/m² on day 1 of each of the seventh and eighth 21-day cycles.

96. The method of claim 94, wherein the control treatment further comprises rituximab, cyclophosphamide, doxorubicin, vincristine, and prednisone, prednisolone, or methylprednisolone during a seventh and eighth 21-day cycle after the sixth 21-day cycle, wherein:
(a) the rituximab is administered intravenously at a dose of about 375 mg/m² on day 1 of each of the seventh and eighth 21-day cycles;
(b) the cyclophosphamide is administered intravenously at a dose of about 750 mg/m² on day 1 of each of the seventh and eighth 21-day cycles;
(c) the doxorubicin is administered intravenously at a dose of about 50 mg/m² on day 1 of each of the seventh and eighth 21-day cycles;
(d) the vincristine is administered intravenously at a dose of about 1.4 mg/m² and up to 2 mg each dose on day 1 of each of the seventh and eighth 21-day cycles; and
(e) the prednisone is administered orally at a dose of about 100 mg per day on each of days 1-5 of each of the seventh and eighth 21-day cycles; the prednisolone is administered orally at a dose of about 100 mg per day on each of days 1-5 of each of the seventh and eighth 21-day cycles; or the methylprednisolone is administered intravenously at a dose of about 80 mg per day on each of days 1-5 of each of the seventh and eighth 21-day cycles.

97. The method of any one of claims 1-96, further comprising administering to the human patient an antihistamine drug, an analgesic, and/or an anti-pyretic drug.

98. The method of any one of claims 1-97, further comprising administering to the human patient a prophylactic therapy for neutropenia.

99. The method of claim 98, comprising administering to the human patient granulocyte colony-stimulating factor (G-CSF).

100. The method of claim 99, wherein the G-CSF is filgrastim, or lenograstim, or peg-filgrastim.

101. The method of any one of claims 1-100, wherein the human patient has a high tumor burden.

102. The method of claim 101, wherein the human patient has a lymphocyte count of at least about 25×10⁹/L.

103. The method of claim 102, wherein the human patient has bulky lymphadenopathy.

104. The method of any one of claims 1-103, wherein the human patient is at risk for developing tumor lysis syndrome.

105. The method of any one of claims 1-104, further comprising administering to the human patient a prophylactic therapy for tumor lysis syndrome.

106. The method of claim 105, wherein the prophylactic therapy for tumor lysis syndrome comprises administering allopurinol or rasburicase to the human patient.

107. The method of claim 105 or claim 106, wherein the prophylactic therapy for tumor lysis syndrome comprises a hydration regimen.

108. The method of claim 107, wherein the hydration regimen comprises administering to the human patient about 3 liters per day of fluids starting at between 1 and 2 days prior to the start of treatment for DLBCL.

109. The method of any one of claims 1-108, wherein the human patient has previously untreated DLBCL.

110. The method of any one of claims 1-109, wherein the DLBCL is CD20 positive.

111. The method of any one of claims 1-6, 9-17, 19-20, 22-26, 28-29, and 31-110, wherein the DLBCL is a DLBCL, not otherwise specified (NOS).

112. The method of any one of claims 1-6, 9-17, 19-20, 22-26, 28-29, and 31-110, wherein the DLBCL is a germinal center B-cell type DLBCL.

113. The method of any one of claims 1-7 and 9-110, wherein the DLBCL is an activated B-cell (ABC) type DLBCL.

114. The method of any one of claims 1-6 and 8-110, wherein the DLBCL is a double expressing (DEL) type DLBCL.

115. The method of any one of claims 1-6, 9-17, 19-20, 22-26, 28-29, and 31-110, wherein the DLBCL is:
(a) a T-cell/histiocyte-rich large B-cell lymphoma;
(b) an Epstein-Barr virus-positive DLBCL, NOS;
(c) an ALK-positive large B-cell lymphoma;
(d) an HHV8-positive DLBCL, NOS;
(e) a high-grade B-cell lymphoma comprising a MYC, a BCL2, and/or a BCL6 rearrangement (double-hit lymphoma or a triple-hit lymphoma); or
(h) a high-grade B-cell lymphoma, NOS.

116. The method of any one of claims 1-3, 7-16, 18-19, 21-25, 27-28, and 30-115, wherein the human patient has an International Prognostic Index (IPI) score of between 2 and 5.

117. The method of any one of claims 1-3, 7-16, 18-19, 21-25, 27-28, and 30-116, wherein the human patient has an IPI score of 2.

118. The method of any one of claims 1-115, wherein the human patient has an IPI score of between 3 and 5.

119. The method of any one of claims 1-118, wherein the human patient is an adult.

120. The method of any one of claims 1-119, wherein the human patient has an Eastern Cooperative Oncology Group (ECOG) Performance Status of 0, 1, or 2.

121. The method of any one of claims 1-120, wherein the human patient has at least one bi-dimensionally measurable lesion.

122. The method of claim 121, wherein the at least one bi-dimensionally measurable lesion has a size greater than 1.5 cm in its longest dimension, as measured by computed tomography (CT) or magnetic resonance imaging (MRI).

123. The method of any one of claims 1-122, wherein the human patient does not have peripheral neuropathy of grade greater than 1 prior to the start of treatment for DLBCL.

124. The method of any one of claims 1-122, wherein the human patient does not have a demyelinating form of Charcot-Marie Tooth disease prior to the start of treatment for DLBCL.

125. The method of any one of claims 1-122, wherein the human patient does not have history of indolent lymphoma prior to the start of treatment for DLBCL.

126. The method of any one of claims 1-125, wherein the human patient does not have:
(a) follicular lymphoma grade 3B,
(b) B-cell lymphoma, unclassifiable, with features intermediate between DLBCL and classical Hodgkin lymphoma,
(c) grey-zone lymphoma,
(d) primary mediastinal (thymic) large B-cell lymphoma,
(e) Burkitt lymphoma,
(f) central nervous system (CNS) lymphoma, primary or secondary involvement,
(g) primary effusion DLBCL, or
(h) primary cutaneous DLBCL,
prior to the start of treatment for DLBCL.

127. The method of any one of claims 1-126, wherein the human patient has not been previously treated for DLBCL.

128. The method of any one of claims 9-14, 22-31, 33, 36-37, 39, 42-43, 52-53, and 62-127, wherein the disease progression or relapse is assessed using the 2014 Lugano Classification for Malignant Lymphoma, and the death is from any cause.

129. A kit comprising polatuzumab vedotin for use in combination with rituximab, cyclophosphamide, doxorubicin, and prednisone, prednisolone or methylprednisolone for treating a human patient in need thereof having diffuse large B-cell lymphoma (DLBCL) according to the method of any one of claims 1-128.

130. The kit of claim 129, wherein the DLBCL is previously untreated DLBCL.

131. Polatuzumab vedotin for use in combination with rituximab, cyclophosphamide, doxorubicin, and prednisone, prednisolone or methylprednisolone for treating a human patient in need thereof having diffuse large B-cell lymphoma (DLBCL) according to the method of any one of claims 1-128.

132. The polatuzumab vedotin of claim 131, wherein the DLBCL is previously untreated DLBCL.
